(19) United States
(12) Patent Application Publication
(10) Pub. No.: US 2023/0270871 A1
(43) Pub. Date: Aug. 31, 2023

(54) MARKERS FOR USE IN METHODS FOR TREATING CANCERS WITH ANTIBODY DRUG CONJUGATES (ADC)

(71) Applicants: AGENSYS, INC., Santa Monica, CA (US); SEAGEN INC., Bothell, WA (US)

(72) Inventors: Timothy Shaun LEWIS, Edmonds, WA (US); Bernard Arthur LIU, Seattle, WA (US)

(73) Assignees: AGENSYS, INC., Santa Monica, CA (US); SEAGEN INC., Bothell, WA (US)

(21) Appl. No.: 18/010,970

(22) PCT Filed: Jun. 18, 2021
(86) PCT No.: PCT/US21/37994

§ 371 Date: Dec. 16, 2022

Related U.S. Application Data
(60) Provisional application No. 63/041,636, filed on Jun. 19, 2020.

(51) Int. Cl. A61K 47/68; C07K 16/30; A61K 9/00; A61P 35/00

(52) U.S. Cl. CPC A61K 47/6803; C07K 16/30; A61K 47/6861; A61K 47/6855; A61K 9/0019; A61P 35/00

(57) ABSTRACT

Provided herein are methods for the treatment of cancers with antibody drug conjugates (ADC) using provided markers.

### CROSS REFERENCE TO RELATED APPLICATIONS

This application claims the benefit of priority to U.S. Provisional Patent Application No. 63/041,636, filed Jun. 19, 2020, the disclosure of which is incorporated by reference herein in its entirety.

### SEQUENCE LISTING

The present specification is being filed with a computer readable form (CRF) copy of the Sequence Listing. The CRF entitled “14369-256-228_SEQ_LISTING.txt,” which was created on Jun. 8, 2021, is 39,661 bytes in size, and is incorporated herein by reference in its entirety.

### 1. FIELD

Provided herein are markers for use in methods for treating cancers with antibody drug conjugates (ADC).

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. who.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for advanced and/or metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a type I transmembrane protein and member of a family of related immunoglobulin-like adhesion molecules implicated in cell-to-cell adhesion. 191P4D12 belongs to the Nectin family of adhesion molecules. 191P4D12 is composed of an extracellular domain (ECD) containing 3 Ig-like subdomains, a transmembrane helix, and an intracellular region (Takai Y et al, Annu Rev Cell Dev Biol 2008; 24:309-42). Nectins are thought to mediate Ca2+-independent cell-cell adhesion via both homophilic and heterophilic trans interactions at adherens junctions where they can recruit cadherins and modulate cytoskeletal rearrangements (Rikitake & Takai, Cell Mol Life Sci. 2008; 65(2):253-63). Sequence identity of 191P4D12 to other Nectin family members is low and ranges between 25% to 30% in the ECD (Reymond N et al, J Biol Chem 2001; 43205-15). Nectin-facilitated adhesion supports several biological processes, such as immune modulation, host-pathogen interaction, and immune evasion (Sakisaka T et al, Current Opinion in Cell Biology 2007; 19:593-602).

Bladder Cancer

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. American Cancer Society (cancer.org) estimates that there are 81,400 new cases annually, including 62,100 in men and 19,300 in women, which accounts for 4.5% of all cancer cases. The age-adjusted incidence in the United States is 20 per 100,000 for men and women. There are an estimated 17,980 deaths from bladder cancer in annually (13,050 in men and 4,930 in women), which accounts for 3% of cancer related deadths. Bladder cancer incidence and mortality strongly increase with age and will be an increasing problem as the population becomes more elderly.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

Breast Cancer

Globally, there will be approximately 2.1 million newly diagnosed female breast cancer cases in 2018, accounting for almost 1 in 4 cancer cases among women. The disease is the most frequently diagnosed cancer in the vast majority of countries and is also the leading cause of cancer-related death in women. Following metastatic diagnosis, prognosis is poor with a with a 5-year survival rate of approximately 15%.

The selection of appropriate therapy for metastatic breast cancer is complex because of the many treatment options and biologic heterogeneity of the disease. The potential treatment options are influenced by estrogen and progesterone receptors and by human epidermal growth factor receptor 2 (HER2) status of the tumor. Treatment options for subjects presenting with metastatic breast cancer may also be influenced by what adjuvant therapy was used, how soon after adjuvant therapy the subject relapses, and by sites of metastasis.

Hormone Receptor Positive, Human Epidermal Growth Factor Receptor 2 Negative Breast Cancer

Hormone receptor positive (HR+)/HER2-negative breast cancer is the most common breast cancer subtype (>70%), occurring predominantly in postmenopausal women. The initial treatment for women with metastatic disease consists primarily of endocrine therapy. This is usually administered alone, in combination with a CDK4/6 inhibitor, or as dual endocrine blockade. For women who are endocrine refractory or women who have symptomatic visceral disease, systemic chemotherapy is recommended.

Several cytotoxic chemotherapy agents have shown activity in metastatic breast cancer, including anthracyclines, taxanes, gemcitabine, capecitabine, vinorelbine, eribulin and ixabepilone. The response rates with these agents vary depending on the type of prior therapy, as well as the breast cancer subtype. In general, anthracycline-based combination therapy and taxanes such as paclitaxel and docetaxel are thought to be the most active (Piccart M, Clin Breast Cancer 2008; 100-13). Given the wide use of anthracyclines in the adjuvant setting and the increased risk of cardiotoxicity, the use of anthracyclines in the metastatic setting is limited. Taxanes are the most commonly used agent for patients with locally advanced or metastatic disease, particularly in the front-line setting (Greene & Hennessy, J Oncol Pharm Pract 2015; 201-12). Sequential single agent therapies are recommended over combinations due to lower toxicities and limited survival benefit. Responses to commonly used single agent chemotherapy patients with HR+/HER2-negative breast cancer are primarily limited to subgroup analysis, these have ranged between 11% to 36% (Robson M et al, N Engl J Med. 2017; 377(18):1792-3; Kaufman P A et al, J Clin Onco. 2015; 33(6):594-601; Cortes J et al, Lancet. 2011; 377:914-23). In general, responses tended to be lower in pretreated patients with reported ranges between 10% to 13% (Perez E A et al, J Clin Oncol. 2007; 25:3407-14; Jones S et al, J Clin Oncol. 1995; 13(10):2567-74).

Triple Negative Breast Cancer

Triple negative breast cancer (TNBC) is defined by the absence of immunostaining for estrogen receptor (ER), progesterone receptor (PR) and HER2. Overall, approximately 15% to 20% of breast cancers are classified as TNBC. TNBC is associated with aggressive tumor biology, visceral metastasis, and a poor prognosis (Plasilova M L et al, Medicine (Baltimore). 2016; 95(35):e4614).

Taxane-based regimens are considered a standard of care in first-line therapy for patients with metastatic breast cancer, including TNBC. More recently the FDA granted accelerated approval for atezolizumab in combination with nab-paclitaxel for the treatment of patients with unresectable locally advanced or metastatic TNBC whose tumors express programmed death-ligand 1 (PD-L1; median progression-free survival [PFS] of 7.5 months versus 5.0 months; objective response rate (ORR) of 56% versus 46%) (Schmid P et al, N Engl J Med. 2019; 380(10):987-988). No standard approach exists for second- or further-line treatment, and options for chemotherapy are the same as those for other subtypes. Single-agent cytotoxic chemotherapeutic agents are generally preferred over combination chemotherapy due to the lack of survival benefit and increased toxicity except in the setting of aggressive disease and visceral involvement (Cardoso F et al, Ann Oncol. 2017; 28(2):208-217; National Comprehensive Cancer Network, 2017, Non-small cell lung cancer, NCCN clinical practice guidelines in oncology (NCCN guidelines), nccn.org/professionals/physician_gls/pdf/nscl.pdf. Accessed 5 Jun. 2019). Standard chemotherapy among pretreated patients is associated with low response rates (10% to 15%) and short progression-free survival (2 to 3 months) (Hurvitz & Mead, Curr Opin Obstet Gynecol. 2016; 28(1):59-69).

Non-Small Cell Lung Cancer

Lung cancer (both small cell and non-small cell) is the leading cause of cancer deaths in the US (American Cancer Society. Key Statistics for Lung Cancer. 8 Jan. 2019a. cancer. org/cancer/non-small-cell-lung-cancer/about/key-statistics html. Accessed 5 Jun. 2019]. Most patients diagnosed with lung cancer are 65 years of age or older and, the average age at the time of diagnosis is approximately 70 years of age.

Non-small cell lung cancer (NSCLC) accounts for approximately 85% of all lung cancers (Tan & Huq, Non-Small Cell Lung Cancer (NSCLC), Apr. 13, 2019, emedicine.medscape.com/article/279960-overview, accessed 5 Jun. 2019; American Cancer Society: What is non-small cell lung cancer, 16 May 2016, cancer.org/cancer/non-small-cell-lung-cancer/about/what-is-non-small-cell-lung-cancer.html, accessed 5 Jun. 2019) and can be subclassified as squamous (approximately 30% of NSCLC cases) and non-squamous (approximately 40% of NSCLC cases) histological types (American Cancer Society. Non-Small Cell Lung Cancer. 2019b. cancer.org/Cancer/LungCancer-Non-SmallCell/DetailedGuide/lung-cancer-non-small-cell-non-small-cell-lung-cancer. Accessed 5 Jun. 2019).

Squamous Non-small Cell Lung Cancer

Squamous NSCLC is a distinct histological subtype of NSCLC that is challenging to treat as a result of specific patient and disease characteristics, which include older age, metastatic (including malignant or metastatic malignant) disease at diagnosis, comorbid disease, and the central location of tumors (Socinski M et al, Cell Lung Cancer 2018; 165-183). These characteristics have a bearing on treatment outcomes in metastatic (including malignant or metastatic malignant) squamous NSCLC, resulting in a median survival rate of approximately 30% shorter than for patients with other NSCLC subtypes.

There are limited treatment options, especially for first-line treatment of metastatic (including malignant or metastatic malignant) squamous NSCLC, with a resuRant impact on survival outcomes (National Comprehensive Cancer Network. 2017, Non-small cell lung cancer, NCCN clinical practice guidelines in oncology (NCCN guidelines), nccn.org/professionals/physician_gls/pdf/nscl.pdf, accessed 5 Jun. 2019; Novello S et al, Ann Oncol 2016; 27 (Supple 5):v1-v27; Masters G A et al, J Clin Oncol 2015; 33(30):3488-3515). Given the recent approvals of targeted therapies and immunotherapies for metastatic (including malignant or metastatic malignant) NSCLC and continuation towards personalization of lung cancer treatment, there is also a need to evaluate the effectiveness of these new treatments for metastatic (including malignant or metastatic malignant) squamous NSCLC.

Non-squamous Non-small Cell Lung Cancer

Non-squamous NSCLC is a heterogeneous disease with multiple treatment options dependent upon staging, presence of metastasis, and patient factors, including presence of comorbidities among other considerations. As such, current treatment options include surgical resection, chemotherapy, radiation, immunotherapy, and targeted therapy. Currently, the first-line therapy for patients with metastatic (including malignant or metastatic malignant) non-squamous NSCLC without targetable genetic aberrations is platinum-doublet chemotherapy. With the exception of bevacizumab, and despite extensive study of multiple targeted and cytotoxic agents, the addition of a third agent to platinum-doublet chemotherapy has not been shown to improve progression-free or OS over platinum-doublet chemotherapy alone in randomized studies (Reck M et al, Ann Oncol 2010; 1804-09; Sandler A et al, N Engl J Med 2006; 355:2542-50).

Head and Neck Cancer

Head and neck cancer is a group of cancers that starts in the mouth, nose, throat, larynx, sinuses, or salivary glands (National Cancer Institute, Head and Neck Cancers, 29 Mar. 2017, https://www.cancer.gov/types/head-and-neck/head-neck-fact-sheet, accessed 5 Jun. 2019). Worldwide, head and neck cancers have affected more than 5.5 million people (mouth 2.4 million, throat 1.7 million, and larynx 1.4 million) and caused over 379000 deaths (GBD. 2016a. Global, regional, and national incidence, prevalence, and years lived with disability for 310 diseases and injuries, 1990-2015: a systematic analysis for the Global Burden of Disease Study 2015, accessed 5 Jun. 2019, thelancet.com/journals/lancet/article/PIIS0140-6736(16)31678-6/fulltext; GBD. 2016b. Global, regional, and national life expectancy, all-cause mortality, and cause-specific mortality for 249 causes of death, 1980-2015: a systematic analysis for the Global Burden of Disease Study 2015, sciencedirect.com/science/article/pii/S0140673616310121, accessed 5 Jun. 2019). Worldwide, approximately 600000 cases of head and neck cancers will arise this year, and only 40% to 60% of patients will survive for 5 years (Rene Leemans C, et al. The molecular biology of head and neck cancer, Nature Reviews Cancer, 16 Dec. 2011, accessed 5 Jun. 2019, nature.com/articles/nrc2982).

The most important risk factors are tobacco use and alcohol consumption, which seem to have a synergistic effect (Decker & Goldstein, N Engl J Med. 1982; 1151-1155). A subgroup of head and neck cancers, particularly those of the oropharynx, are caused by infection with high-risk types of human papillomavirus (HPV) (Rene Leemans C, et al. The molecular biology of head and neck cancer, Nature Reviews Cancer, 16 Dec. 2011, accessed 5 Jun. 2019, nature.com/articles/nrc2982).

Treatment is largely determined by the stage at presentation, but may include a combination of surgery, radiation therapy, chemotherapy, and targeted therapy (National Cancer Institute, 2019, Cancer Stat Facts: Cancer of Any Site, seer.cancer.gov/statfacts/html/all.html, accessed 5 Jun. 2019). Survival, however, has not markedly improved in recent decades because patients often develop locoregional recurrences, distant metastases and second primary tumors. The limited information available on the molecular carcinogenesis of head and neck cancers, and the genetic and biological heterogeneity of the disease has hampered the development of new therapeutic strategies.

Gastric or Esophageal Cancer

An estimated 17650 adult patients in the US will be diagnosed with gastric cancer and approximately 16080 deaths will occur from this disease in 2019 (American Cancer Society, Survival Rates for Esophageal Cancer, 31 Jan. 2019c, cancer.org/cancer/esophagus-cancer/detection-diagnosis-staging/survival-rates.html, accessed 6 Jun. 2019). An estimated 27510 adults in the US will be diagnosed with esophageal cancer and approximately 11140 deaths will occur from this disease in 2019 (American Cancer Society, Key Statistics About Stomach Cancer, 9 Jan. 2019d, cancer.org/cancer/stomach-cancer/about/key-statistics.html, accessed 6 Jun. 2019). Rates of esophageal adenocarcinoma and gastric cardia adenocarcinoma have increased, while rates of esophageal squamous cell carcinoma and gastric noncardia adenocarcinoma have decreased, suggesting distinct etiologies (Crew & Neugut, World J Gastroenterol. 2016; 354-362).

Chemotherapy can provide a significant decrease in symptoms for patients with unresectable, locally advanced, or metastatic disease. Single agents that produce partial response (PR) rates (cisplatin, doxorubicin, and mitomycin) are considered the most active in gastrointestinal (GI) cancers (Preusser P et al, Oncology 1998; 99-102). Combination regimens employing these agents result in higher response rates (30% to 50%) but are associated with a greater degree of toxicity and produce similar OS (ranging from 6 to 10 months), as compared with single-agent therapy (Preusser P et al, Oncology 1998; 99-102). The identification of new agents is therefore essential if prolongation of patient survival is to be reached.

There is a significant need for additional therapeutic methods for cancers. These include the use of antibodies and antibody drug conjugates as treatment modalities.

### 3. Summary

Embodiment 1. A method for treating cancer in a subject in need thereof comprising:
(1) administering to the subject an antibody drug conjugate (ADC) comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
   (b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more major histocompatibility complex (MHC) signature genes, one or more toll-like receptor (TLR) family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 2. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
   (b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 3. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 4. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 5. A method for inducing immunogenic cell death (ICD) in a cancer in a subject in need thereof comprising:
(1) administering to the subject an ADC comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
   (b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 6. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC,
   (b) or administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 7. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC,
   (b) or administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 8. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 9. A method for inducing immune cell migration to a cancer in a subject in need thereof comprising:
(1) administering to the subject an ADC comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
   (b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 10. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
   (b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 11. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 12. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 13. A method for increasing expression of one or more ADC Set I Marker genes in a cancer in a subject in need thereof comprising:
(1) administering to the subject an ADC comprising an antibody or antigen binding fragment thereof conjugated to one or more units of a cytotoxic agent via a linker,
(2) determining an increase of the expression of the one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
   (b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 14. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
   (b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 15. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 16. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
   (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more WIC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

Embodiment 17. The method of any one of embodiments 1 to 16, wherein the antibody or antigen binding fragment thereof is an anti-nectin-4 antibody or antigen binding fragment thereof.

Embodiment 18. The method of any one of embodiments 1 to 17, wherein the cytotoxic agent is a tubulin disrupting agent.

Embodiment 19. The method of embodiment 18, wherein the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane.

Embodiment 20. The method of embodiment 18 or 19, wherein the tubulin disrupting agent is an auristatin.

Embodiment 21. The method of embodiment 19 or 20, wherein the auristatin is monomethyl auristatin E (MMAE), monomethyl auristatin F (MMAF), AFP, or auristain T.

Embodiment 22. The method of any one of embodiments 19 to 21, wherein the auristatin is MMAE.

Embodiment 23. The method of any one of embodiments 1 to 22, wherein the antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 22 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 23, and wherein the antibody or antigen binding fragment thereof is conjugated to 1 to 20 units of MMAE via a linker.

Embodiment 24. The method of any one of embodiments 1 to 23, wherein the one or more ADC Set I Marker genes comprise one or more MHC signature genes.

Embodiment 25. The method of any one of embodiments 1 to 23, wherein the one or more ADC Set I Marker genes consist of one or more MHC signature genes.

Embodiment 26. The method of any one of embodiments 1 to 25, wherein the one or more MHC signature genes comprise one or more MHC class genes.

Embodiment 27. The method of embodiment 26, wherein the one or more MHC class genes comprise one or more MHC class I genes.

Embodiment 28. The method of embodiment 27, wherein the one or more MHC class I genes comprise one or more genes selected from the group consisting of human leukocyte antigens-A (HLA-A), HLA-B, HLA-C, HLA-E, HLA-F, and Transporter 2, ATP binding cassette subfamily B member (TAP2).

Embodiment 29. The method of any one of embodiments 26 to 28, wherein the one or more MHC class genes comprise one or more MHC class II genes.

Embodiment 30. The method of embodiment 29, wherein the one or more MHC class II genes comprise one or more genes selected from the group consisting of HLA-DMA, HLA-DMB, HLA-DRB1, HLA-DRA, and HLA-DPA1.

Embodiment 31. The method of any one of embodiments 26 to 30, wherein the one or more MHC class genes or the one or more MHC class II genes do not comprise HLA-DPB1.

Embodiment 32. The method of any one of embodiments 26 to 30, wherein the MHC signature gene, the MHC class gene or the MHC class II gene is not HLA-DPB1.

Embodiment 33. The method of any one of embodiments 26 to 32, wherein the one or more MHC class genes comprise one or more MHC class III genes.

Embodiment 34. The method of embodiment 33, wherein the one or more MHC class III genes comprise one or more genes selected from the group consisting of LST1, LTB, AIF1, and TNF.

Embodiment 35. The method of any one of embodiments 1 to 34, wherein the one or more MHC signature genes comprise one or more MHC regulator genes.

Embodiment 36. The method of embodiment 35, wherein the one or more MHC regulator genes comprise one or more genes selected from the group consisting of interferon regulatory factor (IRF) genes, nuclear factor kappa-light-chain-enhancer of activated B cells (NF-κB) family genes, signal transducer and activator of transcription (STAT) family genes, CTCF, CIITA, RFX transcription factor family genes, SPI1, and nuclear transcription factor Y (NFY) genes.

Embodiment 37. The method of embodiment 36, wherein the NF-κB family genes comprise one or more genes selected from the group consisting of nuclear factor kappa B subunit 1 (NFKB1), NFKB2, RELA, RELB, and REL.

Embodiment 38. The method of embodiment 36 or 37, wherein the NF-κB family genes comprise NFKB2, RELA, or both NFKB2 and RELA

Embodiment 39. The method of any one of embodiments 36 to 38, wherein the STAT family genes comprise one or more genes selected from the group consisting of STAT1, STAT2, STAT3, STAT4, STAT5, and STAT6.

Embodiment 40. The method of any one of embodiments 36 to 39, wherein the STAT family gene is STAT2.

Embodiment 41. The method of any one of embodiments 36 to 40, wherein the RFX transcription factor family genes comprise one or more genes selected from the group consisting of RFX1, RFX5, RFX7, RFXAP and RFXANK.

Embodiment 42. The method of any one of embodiments 36 to 41, wherein the IRF genes comprise IRF7, IRF8, or both IRF7 and IRF8.

Embodiment 43. The method of any one of embodiments 35 to 42, wherein the one or more MHC regulator genes comprise CTCF.

Embodiment 44. The method of any one of embodiments 35 to 43, wherein the one or more MHC regulator genes comprise CIITA.

Embodiment 45. The method of any one of embodiments 35 to 44, wherein the one or more MHC regulator genes comprise SPI1.

Embodiment 46. The method of any one of embodiments 36 to 45, wherein the NFY genes comprise NFYA, NFYC, or both NFYA and NFYC.

Embodiment 47. The method of any one of embodiments 1 to 46, wherein the one or more ADC Set I Marker genes comprise one or more TLR family genes.

Embodiment 48. The method of any one of embodiments 1 to 47, wherein the one or more TLR family genes comprise one or more genes selected from the group consisting of TLR9, TLR8, and TLR7.

Embodiment 49. The method of any one of embodiments 1 to 48, wherein the one or more TLR family genes do not comprise TLR3.

Embodiment 50. The method of any one of embodiments 1 to 49, wherein the one or more ADC Set I Marker genes comprise one or more interleukin receptor family genes.

Embodiment 51. The method of any one of embodiments 1 to 50, wherein the one or more interleukin receptor family genes comprise one or more genes selected from the group consisting of IL2RA, IL2RB, IL2RG, IL21R, IL27R, IL1RN, IL17RA, IL3RA, IL1R1, IL17RC, IL20RA, and IL22RA1.

Embodiment 52. The method of any one of embodiments 1 to 51, wherein the one or more interleukin receptor family genes comprise IL2RA.

Embodiment 53. The method of any one of embodiments 1 to 52, wherein the one or more interleukin receptor family genes consist of IL2RA.

Embodiment 54. The method of any one of embodiments 1 to 53, wherein the one or more ADC Set I Marker genes comprise one or more immune checkpoint receptor genes.

Embodiment 55. The method of any one of embodiments 1 to 54, wherein one or more immune checkpoint receptor genes comprise one or more B7 family genes, one or more Ig superfamily genes, or both one or more B7 family genes and one or more Ig superfamily genes.

Embodiment 56. The method of embodiment 55, wherein the B7 family genes comprise VTCN1, CD276, or both VTCN1 and CD276.

Embodiment 57. The method of embodiment 55 or 56, wherein the B7 family genes comprise VTCN1.

Embodiment 58. The method of any one of embodiments 55 to 57, wherein the B7 family genes consist of VTCN1.

Embodiment 59. The method of embodiment 55, wherein the Ig superfamily genes comprise nectin family genes.

Embodiment 60. The method of embodiment 55 or 59, wherein the Ig superfamily genes consist of nectin family genes.

Embodiment 61. The method of embodiment 55 or 59, wherein the Ig superfamily genes consist of LAG3 and nectin family genes.

Embodiment 62. The method of any one of embodiments 59 to 61, wherein the nectin family genes comprise one or more genes selected from the group consisting of PVRIG, PVRL2, and TIGIT.

Embodiment 63. The method of any one of embodiments 59 to 62, wherein the nectin family genes comprise TIGIT.

Embodiment 64. The method of any one of embodiments 59 to 63, wherein the nectin family genes consist of TIGIT.

Embodiment 65. The method of any one of embodiments 55 to 64, wherein the Ig superfamily genes comprise LAG3.

Embodiment 66. The method of any one of embodiments 55 to 58, wherein the Ig superfamily genes consist of LAG3.

Embodiment 67. The method of any one of embodiments 1 to 66, wherein the one or more ADC Set I Marker genes comprise one or more receptor tyrosin kinase genes.

Embodiment 68. The method of any one of embodiments 1 to 67, wherein the receptor tyrosin kinase genes comprise one or more genes selected from the group consisting of CSF1R, PDGFRB, TEK/TIE2, and FLT3.

Embodiment 69. The method of any one of embodiments 1 to 68, wherein the receptor tyrosin kinase genes consist of CSF1R.

Embodiment 70. The method of any one of embodiments 1 to 68, wherein the receptor tyrosin kinase genes comprise CSF1R.

Embodiment 71. The method of any one of embodiments 1 to 70, wherein the one or more ADC Set I Marker genes comprise one or more TNF family receptor genes.

Embodiment 72. The method of any one of embodiments 1 to 71, wherein the TNF family receptor genes comprise one or more genes selected from the group consisting of CD40, TNFRSF1A, TNFRSF21, and TNFRSF1B.

Embodiment 73. The method of any one of embodiments 1 to 72, wherein the one or more ADC Set I Marker genes comprise one or more IFN receptor family genes.

Embodiment 74. The method of any one of embodiments 1 to 73, wherein the IFN receptor family genes comprise IFNAR1, IFNAR2, or both IFNAR1 and IFNAR2.

Embodiment 75. The method of any one of embodiments 1 to 74, wherein the IFN receptor family genes consist of IFNAR1.

Embodiment 76. The method of any one of embodiments 1 to 74, wherein the IFN receptor family genes comprise IFNAR1.

Embodiment 77. The method of any one of embodiments 1 to 76, wherein the one or more ADC Set I Marker genes comprise one or more inhibitory immunoreceptor genes.

Embodiment 78. The method of any one of embodiments 1 to 77, wherein the inhibitory immunoreceptor genes comprise TIM3, VSIR, or both TIM3 and VSIR.

Embodiment 79. The method of any one of embodiments 1 to 78, wherein the inhibitory immunoreceptor genes comprise VSIR.

Embodiment 80. The method of any one of embodiments 1 to 78, wherein the inhibitory immunoreceptor genes consist of VSIR.

Embodiment 81. The method of any one of embodiments 1 to 79, wherein the inhibitory immunoreceptor genes comprise TIM3.

Embodiment 82. The method of any one of embodiments 1 to 78, wherein the inhibitory immunoreceptor genes consist of TIM3.

Embodiment 83. The method of any one of embodiments 1 to 82, wherein the one or more ADC Set I Marker genes comprise one or more metabolic enzyme genes.

Embodiment 84. The method of any one of embodiments 1 to 83, wherein the metabolic enzyme genes comprise one or more genes selected from the group consisting of indoleamine 2,3-dioxygenase 1 (IDO1), TDO2, EIF2AK2, ACSS1, and ACSS2.

Embodiment 85. The method of any one of embodiments 1 to 84, wherein the metabolic enzyme genes consist of IDO1.

Embodiment 86. The method of any one of embodiments 1 to 84, wherein the metabolic enzyme genes comprise IDO1.

Embodiment 87. The method of any one of embodiments 1 to 86, wherein the method further comprises determining an increase of expression of one or more ADC Set II Marker genes in the subject compared to the expression of the one or more ADC Set II Marker genes in the subject before the administration of the ADC in step (1).

Embodiment 88. The method of 87, wherein the administration in step (3)(a) is further conditioned on the increase of the expression of the one or more ADC Set II Marker genes as determined in embodiment 87.

Embodiment 89. The method of embodiment 87 or 88, wherein the one or more ADC Set II Marker genes comprise one or more genes selected from the group consisting of ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes.

Embodiment 90. The method of embodiment 89, wherein the ER stress genes comprise one or more genes selected from the group consisting of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK.

Embodiment 91. The method of embodiment 89 or 90, wherein the ER stress genes do not comprise EDEM2 or XBP-1L.

Embodiment 92. The method of any one of embodiments 89 to 91, wherein the ER/mitochondria ATPase genes comprise one or more genes selected from the group consisting of ATP2A3, MT-ATP6, and MT-ATP8.

Embodiment 93. The method of any one of embodiments 89 to 92, wherein the cell death genes comprise one or more genes selected from the group consisting of Bax, BCL2L1, BCL2L11, and BOK.

Embodiment 94. The method of any one of embodiments 89 to 93, wherein the cell death genes do not comprise FAS.

Embodiment 95. The method of any one of embodiments 89 to 94, wherein the T cell stimulator genes comprise MIG (CXCL9), IP10 (CXCL10), or both MIG and IP10.

Embodiment 96. The method of any one of embodiments 89 to 95, wherein the macrophage/innate immunity stimulator genes comprise IL-1α, M-CSF (CSF), or both IL-1α and M-CSF.

Embodiment 97. The method of any one of embodiments 89 to 96, wherein the chemoattractant genes comprise one or more genes selected from the group consisting of Eotaxin (CCL11), MIP1α, MIP1β, and MCP1.

Embodiment 98. The method of any one of embodiments 89 to 97, wherein the Rho GTPase genes comprise one or more genes selected from the group consisting of RhoB, RhoF, and RhoG.

Embodiment 99. The method of any one of embodiments 89 to 98, wherein the Rho GTPase genes do not comprise any one of CDC42, RhoA, and RhoC.

Embodiment 100. The method of any one of embodiments 89 to 99, wherein the Rho GTPase regulator genes comprise one or more genes selected from the group consisting of DAP2IP, ARHGEF18, ARHGEF5, and RASAL1.

Embodiment 101. The method of any one of embodiments 89 to 100, wherein the mitotic arrest genes comprise one or more genes selected from the group consisting of CCND1, CDKN1A, GADD45B, E4F1, CDC14B, and DAPK1.

Embodiment 102. The method of any one of embodiments 89 to 101, wherein the mitotic arrest genes do not comprise DDIAS or CDK1.

Embodiment 103. The method of any one of embodiments 89 to 102, wherein the siglec family genes comprise siglec1.

Embodiment 104. The method of any one of embodiments 89 to 103, wherein the GO positive autophagy regulator genes comprise one or more genes selected from the group consisting of BCL2L11, ROCK1, TSC1, TSC2, BAG3, MFN2, RIPK1, RIPK4, HDAC6, STK11, ULK1, FOXO1, FOXO3, and MULL

Embodiment 105. The method of any one of embodiments 89 to 104, wherein the GO positive autophagy regulator genes do not comprise BNIP3 or BNIP3L.

Embodiment 106. The method of any one of embodiments 89 to 105, wherein the GTPase related kinase genes comprise ROCK1, PAK4, or both ROCK1 and PAK4.

Embodiment 107. The method of any one of embodiments 1 to 106, wherein the increase in any of the gene expression is an increase of 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, or more.

Embodiment 108. The method of any one of embodiments 1 to 106, wherein the increase in any of the gene expression is an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 fold or more.

Embodiment 109. The method of any one of embodiments 3, 4, 7, 8, 11, 12, and 15 to 108, wherein the immune checkpoint inhibitor is a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or an therapeutic agent targeting HLA.

Embodiment 110. The method of any one of embodiments 3, 4, 7, 8, 11, 12, and 15 to 109, wherein the immune checkpoint inhibitor is an anti-PD-1 antibody.

Embodiment 111. The method of embodiment 110, wherein the anti-PD-1 antibody is BGB-A317, nivolumab, pembrolizumab, cemiplimab, CT-011, camrelizumab, sintilimab, tislelizumab, TSR-042, PDR001, or toripalimab.

Embodiment 112. The method of any one of embodiments 3, 4, 7, 8, 11, 12, and 15 to 109, wherein the immune checkpoint inhibitor is an anti-PD-L1 antibody.

Embodiment 113. The method of embodiment 112, wherein the anti-PD-L1 antibody is durvalumab, BMS-936559, atezolizumab, MEDI4736, or avelumab.

Embodiment 114. The method of any one of embodiments 3, 4, 7, 8, 11, 12, and 15 to 109, wherein the immune checkpoint inhibitor is an anti-PD-L2 antibody.

Embodiment 115. The method of embodiment 114, wherein the anti-PD-L2 antibody is rHIgM12B7A.

Embodiment 116. The method of any one of embodiments 3, 4, 7, 8, 11, 12, and 15 to 109, wherein the immune checkpoint inhibitor is a VTCN1 inhibitor.

Embodiment 117. The method of embodiment 116, wherein the VTCN1 inhibitor is FPA150.

Embodiment 118. The method of any one of embodiments 3, 4, 7, 8, 11, 12, and 15 to 109, wherein the immune checkpoint inhibitor is an IDO1 inhibitor.

Embodiment 119. The method of embodiment 118, wherein the IDO1 inhibitor is Epacadostat, BMS986205, Navoximod, PF-06840003, KHK2455, RG70099, IOM-E, or IOM-D.

Embodiment 120. The method of any one of embodiments 3, 4, 7, 8, 11, 12, and 15 to 109, wherein the immune checkpoint inhibitor is a TIGIT inhibitor.

Embodiment 121. The method of embodiment 120, wherein the a TIGIT inhibitor is MTIG7192A, BMS-986207, OMP-313M32, MK-7684, AB154, CGEN-15137, SEA-TIGIT, ASP8374, or AJUD008.

Embodiment 122. The method of any one of embodiments 3, 4, 7, 8, 11, 12, and 15 to 109, wherein the immune checkpoint inhibitor is a VSIR inhibitor.

Embodiment 123. The method of embodiment 122, wherein the VSIR inhibitor is CA-170, JNJ 61610588, or HMBD-002.

Embodiment 124. The method of any one of embodiments 3, 4, 7, 8, 11, 12, and 15 to 109, wherein the immune checkpoint inhibitor is a TIM3 inhibitor.

Embodiment 125. The method of embodiment 124, wherein the TIM3 inhibitor is AJUD009.

Embodiment 126. The method of any one of embodiments 3, 4, 7, 8, 11, 12, and 15 to 109, wherein the immune checkpoint inhibitor is a CD25 (IL2RA) inhibitor.

Embodiment 127. The method of embodiment 126, wherein the CD25 (IL2RA) inhibitor is daclizumab or basiliximab.

Embodiment 128. The method of any one of embodiments 3, 4, 7, 8, 11, 12, and 15 to 109, wherein the immune checkpoint inhibitor is an IFNAR1 inhibitor.

Embodiment 129. The method of embodiment 128, wherein the IFNAR1 inhibitor is anifrolumab or sifalimumab.

Embodiment 130. The method of any one of embodiments 3, 4, 7, 8, 11, 12, and 15 to 109, wherein the immune checkpoint inhibitor is a CSF1R inhibitor.

Embodiment 131. The method of embodiment 130, wherein the CSF1R inhibitor is pexidartinib, emactuzumab, cabiralizumab, ARRY-382, BLZ945, AJUD010, AMG820, IMC-CS4, JNJ-40346527, PLX5622, or FPA008.

Embodiment 132. The method of any one of embodiments 3, 4, 7, 8, 11, 12, and 15 to 109, wherein the immune checkpoint inhibitor is a therapeutic agent targeting HLA.

Embodiment 133. The method of embodiment 132, wherein the therapeutic agent targeting HLA is GSK01, IMC-C103C, IMC-F106C, IMC-G107C, or ABBV-184.

Embodiment 134. The method of any one of embodiments 1 to 133, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

Embodiment 135. The method of any one of embodiments 1 to 134, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 136. The method of any one of embodiments 1 to 135, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 137. The method of any one of embodiments 1 to 136, wherein the antigen binding fragment is an Fab, F(ab′)2, Fv or scFv.

Embodiment 138. The method of any one of embodiments 1 to 137, wherein the antibody is a fully human antibody.

Embodiment 139. The method of any one of embodiments 1 to 138, wherein the antibody or antigen binding fragment thereof is recombinantly produced.

Embodiment 140. The method of any one of embodiments 1 to 139, wherein the ADC has the following structure:

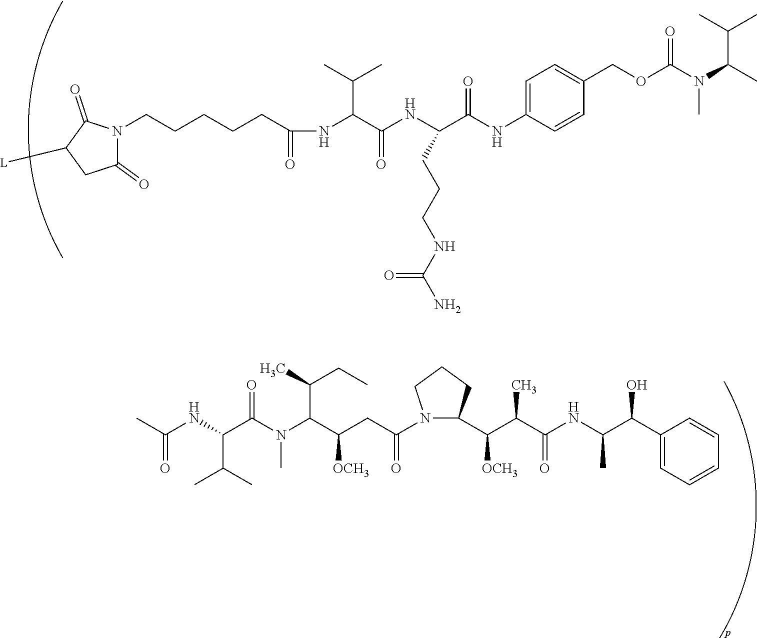

wherein L− represents the antibody or antigen binding fragment thereof and p is from 1 to 10.

Embodiment 141. The method of embodiment 140, wherein p is from 2 to 8.

Embodiment 142. The method of embodiment 140 or 141, wherein p is from 3 to 5.

Embodiment 143. The method of any one of embodiments 1 to 139, wherein the antibody or antigen binding fragment is conjugated to each unit of MMAE via a linker.

Embodiment 144. The method of embodiment 143, wherein the linker is an enzyme-cleavable linker, and wherein the linker forms a bond with a sulfur atom of the antibody or antigen binding fragment thereof.

Embodiment 145. The method of embodiment 143 or 144, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

Embodiment 146. The method of embodiment 145, wherein the stretcher unit has the structure of Formula (1) below; the amino acid unit is valine-citrulline; and the spacer unit is a PAB group comprising the structure of Formula (2) below:

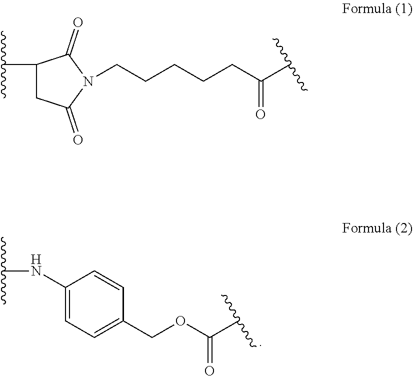 [SMILES: *C(=O)CCCCCN1C(=O)CC(*)C1=O ; *Nc1ccc(COC(*)=O)cc1]

Embodiment 147. The method of embodiment 145 or 146, wherein the stretcher unit forms a bond with a sulfur atom of the antibody or antigen binding fragment thereof; and wherein the spacer unit is linked to MMAE via a carbamate group.

Embodiment 148. The method of any one of embodiments 1 to 139 and 143 to 147, wherein the ADC comprises from 1 to 20 units of MMAE per antibody or antigen binding fragment thereof.

Embodiment 149. The method of any one of embodiments 1 to 139 and 143 to 148, wherein the ADC comprises from 1 to 10 units of MMAE per antibody or antigen binding fragment thereof.

Embodiment 150. The method of any one of embodiments 1 to 139 and 143 to 149, wherein the ADC comprises from 2 to 8 units of MMAE per antibody or antigen binding fragment thereof.

Embodiment 151. The method of any one of embodiments 1 to 139 and 143 to 150, wherein the ADC comprises from 3 to 5 units of MMAE per antibody or antigen binding fragment thereof.

Embodiment 152. The method of any one of embodiments 1, 5, 9, 13, and 17 to 151, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

Embodiment 153. The method of any one of embodiments 1, 5, 9, 13, and 17 to 152, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

Embodiment 154. The method of any one of embodiments embodiments 1, 5, 9, 13, and 17 to 153, wherein the ADC is administered at a dose of about 1 mg/kg of the subject's body weight.

Embodiment 155. The method of any one of embodiments embodiments 1, 5, 9, 13, and 17 to 153, wherein the ADC is administered at a dose of about 1.25 mg/kg of the subject's body weight.

Embodiment 156. The method of any one of embodiments 2 to 4, 6 to 8, 10 to 12, 14 to 151, wherein the first dose of the ADC is a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

Embodiment 157. The method of embodiment 156, wherein the first dose of the ADC is a dose of about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

Embodiment 158. The method of embodiment 156 or 157, wherein the first dose of ADC is a dose of about 1 mg/kg of the subject's body weight.

Embodiment 159. The method of embodiment 156 or 157, wherein the first dose of ADC is a dose of about 1.25 mg/kg of the subject's body weight.

Embodiment 160. The method of any one of embodiments 156 to 159, wherein the second dose of the ADC is lower than the first dose by about 0.1 mg/kg to about 1 mg/kg of the subject's body weight.

Embodiment 161. The method of any one of embodiments 156 to 160, wherein the second dose of the ADC is lower than the first dose by about 0.1 mg/kg, about 0.2 mg/kg, about 0.25 mg/kg, about 0.3 mg/kg, about 0.4 mg/kg, about 0.5 mg/kg, about 0.6 mg/kg, about 0.7 mg/kg, about 0.75 mg/kg, about 0.8 mg/kg, about 0.9 mg/kg, or about 1 mg/kg of the subject's body weight.

Embodiment 162. The method of any one of embodiments 156 to 161, wherein the second dose of the ADC is lower than the first dose by about 0.25 mg/kg of the subject's body weight.

Embodiment 163. The method of any one of embodiments 156 to 161, wherein the second dose of the ADC is lower than the first dose by about 0.5 mg/kg of the subject's body weight.

Embodiment 164. The method of any one of embodiments 156 to 161, wherein the second dose of the ADC is lower than the first dose by about 0.75 mg/kg of the subject's body weight.

Embodiment 165. The method of any one of embodiments 156 to 161, wherein the second dose of the ADC is lower than the first dose by about 1.0 mg/kg of the subject's body weight.

Embodiment 166. The method of any one of embodiments 156 to 165, wherein the second dose of the ADC is a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, or about 2.25 mg/kg of the subject's body weight.

Embodiment 167. The method of any one of embodiments 156 to 166, wherein the second dose of the ADC is identical to the first dose of the ADC.

Embodiment 168. The method of any one of embodiments 1 to 166, wherein the ADC is administered by an intravenous (IV) injection or infusion.

Embodiment 169. The method of any one of embodiments 1 to 168, wherein the ADC is administered by an IV injection or infusion three times every four-week cycle.

Embodiment 170. The method of any one of embodiments 1 to 169, wherein the ADC is administered by an IV injection or infusion on Days 1, 8 and 15 of every four-week cycle.

Embodiment 171. The method of any one of embodiments 1 to 170, wherein the ADC is administered by an IV injection or infusion over about 30 minutes three times every four-week cycle.

Embodiment 172. The method of any one of embodiments 1 to 171, wherein the ADC is administered by an IV injection or infusion over about 30 minutes on Days 1, 8 and 15 of every four-week cycle.

Embodiment 173. The method of any one of embodiments 1 to 172, wherein the ADC is formulated in a pharmaceutical composition comprising L-histidine, polysorbate-20 (TWEEN-20), and trehalose dehydrate.

Embodiment 174. The method of any one of embodiments 1 to 173, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 175. The method of any one of embodiments 1 to 173, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 176. The method of any one of embodiments 1 to 175, wherein the cancer is bladder cancer, urothelial cancer, gastric cancer, esophageal cancer, head cancer, neck cancer, squamous non-small cell lung cancer (NSCLC), non-squamous NSCLC, breast cancer, ovarian cancer, cervical cancer, biliary cancer and cholangiocarcinomas, pancreatic cancer, squamous cell carcinoma of the vulva and penis, prostate adenocarcinoma, or endometrial carcinoma.

Embodiment 177. The method of any one of embodiments 1 to 176, wherein the cancer is locally advanced cancer.

Embodiment 178. The method of any one of embodiments 1 to 176, wherein the cancer is metastatic cancer.

Embodiment 179. The method of any one of embodiments 176 to 178, wherein the breast cancer is ER negative, PR negative, and HER2 negative (ER−/PR−/HER2−) breast cancer.

Embodiment 180. The method of any one of embodiments 176 to 179, wherein the breast cancer is hormone receptor positive and human epidermal growth factor receptor 2 negative (HR+/HER2−) breast cancer.

Embodiment 181. The method of any one of embodiments 176 to 178, wherein the urothelial cancer is papillary urothelial carcinoma or flat urothelial carcinoma.

Embodiment 182. The method of any one of embodiments 176 to 178, wherein the bladder cancer is non-muscle-invasive bladder cancer (NMIBC) or muscle-invasive bladder cancer.

Embodiment 183. The method of embodiment 182, wherein the muscle-invasive bladder cancer is squamous cell carcinoma, adenocarcinoma, small cell carcinoma, or sarcoma.

### 4. BRIEF DESCRIPTION OF THE DRAWINGS

FIGS. 1A-E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2A depicts intracellular accumulation of MMAE. Intracellular free MMAE was measured in T-24 parental and T-24 cells expressing nectin-4 (clone 1A9) after 24 hours of treatment with an anti-nectin-4 ADC (enfortumab vedotin or AGS-22C3E). BLLQ means below lower limit of quantification. FIG. 2B depicts co-localization of anti-nectin-4 ADC (enfortumab vedotin, panels i, iv; green) with the lysosome (LAMP1, panels ii, iv; red) and Hoescht DNA stain (panels iii, iv; blue). The anti-nectin-4 ADC (enfortumab vedotin or EV) internalizes and co-localizes with LAMP1, a lysosomal marker. White arrows or merged yellow staining show the areas where enfortumab vedotin is colocalized with LAMP1 vesicles. Briefly, T-24 cells expressing nectin-4 were treated with enfortumab vedotin (EV) for 48 hours and stained as indicated. FIG. 2C depicts the cytotoxicity of the anti-nectin-4 ADC (enfortumab vedotin or AGS-22C3E) treatment. Enfortumab vedotin directly killed the T-24 Nectin-4 cells while the parental T-24 cell line lacking Nectin-4 is insensitive to enfortumab vedotin (AGS-22C3E). FIG. 2D depicts caspase 3/7 induction in response to the anti-nectin-4 ADC (AGS-22C3E) treatment. The anti-nectin-4 ADC induced caspase 3/7 in UM-UC-3 Nectin-4 cells but not in the parental UM-UC-3 cell line lacking Nectin-4.

FIG. 3A depicts bystander cell killing of antigen negative cancer cells (GFP positive) through a targeted delivery of drug to antigen positive cells (GFP negative). Q1 (EV killed Nectin-4⁺ cells); Q2 (bystander effect killed Nectin-4⁻ cells); Q3 (GFP⁺, Nectin-4⁻ live cells); Q4 (GFP⁻, Nectin-4⁺ live cells). In FIG. 3A, AnnexinV is a marker of cell death and GFP is green fluorescent protein. Cells used in FIG. 3A are 1:1 ratio of UM-UC-3 expressing GFP to UM-UC-3 expressing Nectin-4. FIGS. 3B and 3C depict cell viability of antigen negative cancer cells in response to the treatment of an anti-nectin-4 ADC (AGS-22C3E) or the control treatment (non-binding ADC) in UM-UC-3 cells (3B) or T-24 cells (3C). In FIGS. 3B and 3C, The percentage of viable cells in Q3 from FIG. 3A representing the Nectin-4 negative population was determined after 168 hours of treatment in a 1:1 co-culture with varying concentrations of enfortumab vedotin or non-binding ADC control for the UM-UC-3 and T-24 bladder cells. Cells in FIG. 3B are admix 1:1 (168 hrs) of UM-UC-3 expressing human nectin-4 (clone 1D11): UM-UC-3 expressing GFP. Cells in FIG. 3C are admix 1:1 (168 hrs) of T-24 expressing human nectin-4 (clone 1A9): T-24 expressing GFP.

FIG. 4A is a cartoon representation of the release of ATP and HMGB1 by cells treated with an anti-nectin-4 ADC and the effects of the released ATP and HMGB1 on macrophages. FIG. 4B is a cartoon representation of the release of ATP and HMGB1 by cells treated with an anti-nectin-4 ADC, the activation of the immune cells by the ATP and HMGB1, and the potential immunogenic cell death or immunogenic cell killing by the activated immune cells. FIG. 4C depicts ATP release in control T-24 cells and T-24 cells expressing nectin-4 after various treatments as indicated. FIG. 4D depicts ATP release in control UM-UC-3 cells and UM-UC-3 cells expressing nectin-4 after various treatments as indicated. FIG. 4E depicts HMGB1 release in control T-24 cells and T-24 cells expressing nectin-4 after various treatments as indicated. FIGS. 4F and 4G depict cell surface calreticulin (4F) and HSP70 (4G), respectively, in cells upon various treatments as indicated. Increase in ICD cell surface markers such as calreticulin or HSP70 can be detected on T-24-Nectin-4 cells upon treatment with an anti-nectin-4 ADC (1 ug/mL) or MMAE (100 nM) compared with untreated or treatment with the control hIgG-MMAE (1 mg/mL). In FIGS. 4C to 4G, EV and AGS-22C3E indicate the same anti-nectin-4 ADC.

FIGS. 5A-5C depict the general study designs. Briefly, the T-24 bladder cells expressing human nectin-4 were implanted into nude mice and passaged via trocar, allowed to reach approximately 200 mm³ tumor volume, and subsequently treated with a single intraperitoneal (IP) dose of an anti-nectin-4 ADC (AGS-22C3E) (3 mg/kg) or a control non-binding ADC (hIgG1-MMAE(4)) (3 mg/kg) with 5 animals per treatment group. FIG. 5A depicts the time course of the tumor volume of tumor from T-24 cells expressing human nectin-4 implanted in mice after treatment with an anti-nectin-4 ADC (AGS-22C3E) or a non-binding ADC control. FIG. 5B depicts nectin-4 staining of the tumors under each treatment and follow-up immunogenic cell death (ICD) studies with this model. Briefly, tumors from each treatment shown were collected 5 days post treatment for downstream analysis by RNA-seq, flow cytometry, immunohistochemistry (IHC), and Luminex. FIG. 5C depicts RNA-seq differential gene expression analyses, which indicate that EV treated cells produce gene signatures consistent with microtubule disruption, ER stress, and immunogenic cell death. RNA gene signatures from 1267 differentially regulated genes were used to identify signatures that went up or down between the EV treatment vs untreated samples (n=7). The p-value is calculated using the Wilcoxon test.

FIG. 6A depicts IHC staining of tumors showing enrichment of immune cell infiltration by increased F4/80 and CD11C staining in response to treatment by an anti-nectin-4 ADC (AGS-22C3E) compared to untreated or a non-binding ADC control. FIGS. 6B and 6C depict staining of tumors for immune infiltration by determining the percentage of F4/80 (6B) or CD11C (6C) positive cells using semi-quantitive immunohistochemistry. Statistical analysis was performed using an unpaired t test. In FIGS. 6A-6C, tumors from the T-24 Nectin-4 (clone 1A9) xenograft were collected at Day 5 post treatment as indicated and divided for downstream analysis by IHC or flow cytometry. In FIGS. 6B-6C, p-value indicators are: ***<0.001; **<0.01; *<0.05.

FIG. 7A depicts RNA-seq gene transcripts analyses showing upregulation of human HLA/MHC and immune regulated genes in tumors treated with the anti-nectin-4 ADC (AGS-22C3E) compared to the untreated or tumors treated with a non-binding control. RNA-seq gene transcripts identified MHC class I and the transporter TAP2 genes upregulated upon treatment with enfortumab vedotin compared to untreated or non-binding ADC. Upregulation of MHC genes can allow neo-antigens to be presented where MHC class I genes activate CD8 to prime the adaptive immune response. Statistical analysis was performed using an unpaired t test. P-value indicators are: ***<0.001; **<0.01; *<0.05. FIG. 7A shows that human HLA/MHC and immune regulated genes are elevated upon treatment with EV. FIG. 7B depicts RNA-seq gene transcripts analyses showing upregulation of interferon and immune activation transcriptional regulators in tumors treated with the anti-nectin-4 ADC (AGS-22C3E) compared to the untreated or tumors treated with a non-binding control. RNA-seq gene transcripts identified interferon and immune activation transcriptional regulators from the human transcriptome to be upregulated upon treatment with enfortumab vedotin (AGS-22C3E) compared to untreated or non-binding ADC. Statistical analysis was performed using an unpaired t test. P-value indicators are: ***<0.001; **<0.01; *<0.05. FIG. 7C depicts exemplary MHC class I regulation.

FIG. 8A depicts RNA-seq gene transcripts analyses showing upregulation of MHC class II gene in tumors treated with the anti-nectin-4 ADC (AGS-22C3E) compared to the untreated or tumors treated with a non-binding control (hIgG1-MMAE(4)). RNA-seq gene transcripts identified MHC class II genes upregulated upon treatment with enfortumab vedotin compared to untreated or non-binding ADC. Upregulation of MHC genes can allow neo-antigens to be presented where MHC class II genes activate CD4 T-cells to prime the adaptive immune response. Statistical analysis was performed using an unpaired t test. P-value indicators are: ***<0.001; **<0.01; *<0.05. FIG. 8B depicts RNA-seq gene transcripts analyses showing upregulation of mouse MHC class II genes in tumors treated with the anti-nectin-4 ADC (AGS-22C3E) compared to the untreated or tumors treated with a non-binding control (hIgG1-MMAE(4)). RNA-seq gene transcripts identified MHC class II genes from the mouse transcriptome to be upregulated upon treatment with enfortumab vedotin compared to untreated or non-binding ADC. Upregulation of MHC genes can allow neo-antigens to be presented where MHC class II genes activate CD4 cells to prime the adaptive immune response. Statistical analysis was performed using an unpaired t test. P-value indicators are: ****<0.0001, ***<0.001; **<0.01; *<0.05. FIG. 8C depicts RNA-seq gene transcripts analyses showing upregulation of mouse MHC class III genes in tumors treated with the anti-nectin-4 ADC (AGS-22C3E) compared to the untreated or tumors treated with a non-binding control (hIgG1-MMAE(4)). RNA-seq gene transcripts identified MHC class III genes from the mouse transcriptome to be upregulated upon treatment with enfortumab vedotin compared to untreated or non-binding ADC. P-value indicators are: ***<0.001; **<0.01. FIG. 8D depicts activation of macrophages and stimulation of the cytokine release in EV-treated (AGS-22C3E-treated) cells. T-24 Nectin-4 (clone1A9) cells were treated with drugs as indicated for 24 hours. Cell debris material was collected and incubated with macrophages from PBMCs. Macrophages were collected and stained for activation markers by flow cytometry such as cell surface expression of MHC-II. Cytokine profiling was performed using Luminex Human Cytokine array.

FIG. 9A depicts anti-nectin-4 ADC-mediated disruption of microtubules and subsequent endoplasmic reticulum (ER) stress. T-24 Nectin-4 (clone1A9) cells were treated with an anti-nectin-4 ADC (AGS-22C3E) for 48 hours and stained with β-tubulin for microtubules and DAPI, a nuclear DNA stain. FIGS. 9B and 9C depict activation of phospho-INK in response to the treatment with an anti-nectin-4 ADC (AGS-22C3E). In FIG. 9B, western blots show an increase in phospho-JNK over a period of 48 hours upon treatment with an anti-nectin-4 ADC (AGS-22C3E) at 1 ug/mL). In FIG. 9C, Phosphorylation of JNK is observed in treatment with an anti-nectin-4 ADC (AGS-22C3E) and MMAE but absent in untreated or non-binding ADC control.

FIGS. 10A-10C depict increased murine cytokines within tumors (n=7 per treatment group) upon treatment by an anti-nectin-4 ADC as measured by Luminex. FIG. 10A depicts changes in T cell stimulators tumors treated with an anti-nectin-4 ADC (AGS-22C3E) or a control non-binding ADC comparing with untreated tumors. T cell stimulators are significantly upregulated upon AGS-22C3E treatment. FIG. 10B depicts changes in macrophage/innate stimulators in tumors treated with an anti-nectin-4 ADC (AGS-22C3E) or a control non-binding ADC comparing with untreated tumors. Macrophage or innate stimulators such as IL-1a and M-CSF are elevated upon AGS-22C3E treatment. FIG. 10C depicts changes in chemoattractants in tumors treated with an anti-nectin-4 ADC (AGS-22C3E) or a control non-binding ADC comparing with untreated tumors. Chemoattractant levels secreted by immune cells are elevated upon treatment with AGS-22C3E. Analysis of the RNA-seq data aligns with findings from the Luminex cytokine analysis where upregulation of mouse immune cytokines such as MIP1a and MIP1β were elevated in EV treated samples. Indicators for statistical analysis by t-test: ** p<0.005, * p<0.05, n.s. not significant.

FIG. 11A depicts RNA-seq gene transcripts analyses showing upregulation of human interferon and immune activation transcriptional regulators in tumors treated with the anti-nectin-4 ADC (AGS-22C3E) compared to the untreated or tumors treated with a non-binding control. RNA-seq gene transcripts identified interferon and immune activation transcriptional regulators from the human transcriptome to be upregulated upon treatment with enfortumab vedotin (AGS-22C3E) compared to untreated or non-binding ADC. The transcriptional regulatory factors shown are known to promote MHC class II gene expression. Statistical analysis was performed using an unpaired t test. P-value indicators are: ***<0.001; **<0.01; *<0.05. FIGS. 11B and 11C depict exemplary MHC class II regulation.

FIG. 12 depicts RNA-seq gene transcripts analyses showing upregulation of mouse interferon and immune activation transcriptional regulators in tumors treated with the anti-nectin-4 ADC (AGS-22C3E) compared to the untreated or tumors treated with a non-binding control. RNA-seq gene transcripts identified interferon and immune activation transcriptional regulators from the mouse transcriptome to be upregulated upon treatment with enfortumab vedotin compared to untreated or non-binding ADC. The transcriptional regulatory factors shown are known to promote MHC class II gene expression. Statistical analysis was performed using an unpaired t test. P-value indicators are: ****<0.0001, ***<0.001; **<0.01; *<0.05.

FIG. 13 depicts changes in certain innate Toll-like receptors or siglec1 as indicated in tumors in response to anti-nectin-4 ADC (AGS-22C3E) or a control non-binding ADC treatment (hIgG1-MMAE(4)). P-value indicators are: ***<0.001; **<0.01; *<0.05.

FIG. 14 depicts RNA-seq gene transcripts analyses showing upregulation of human and mouse interleukin receptors in tumors treated with the anti-nectin-4 ADC (AGS-22C3E) compared to the untreated or tumors treated with a non-binding control. RNA-seq gene transcripts identified interleukin receptor family genes from the mouse and human transcriptome to be upregulated upon treatment with enfortumab vedotin compared to untreated or non-binding ADC. Therapeutic agents against these upregulated interleukin receptors can be combined with EV as potential combination therapies. Statistical analysis was performed using an unpaired t test. p-value; ****<0.0001, ***<0.001; **<0.01; *<0.05.

FIGS. 15A and 15B depict RNA-seq gene transcripts analyses showing upregulation of human B7 family (FIG. 15A) and Ig superfamily (FIG. 15B) in tumors treated with the anti-nectin-4 ADC (AGS-22C3E) compared to the untreated or tumors treated with a non-binding control. RNA-seq gene transcripts identified B7 family genes (FIG. 15A) and Ig superfamily genes (FIG. 15B) from the human transcriptome to be upregulated upon treatment with enfortumab vedotin compared to untreated or non-binding ADC. Therapeutic agents against these upregulated B7 family members (FIG. 15A) and Ig superfamily members (FIG. 15B) can be combined with EV as potential combination therapies. CD276 (B7H3) and VTCN1 (B7H4) belong to the family of immune regulatory ligands. PVRIG, PVRL2 (Nectin-2), and TIGIT belong to the Nectin or Polio virus receptor family. LAG3 (CD223) is a member of the Ig superfamily. Statistical analysis was performed using an unpaired t test. p-value; ****<0.0001, ***<0.001; **<0.01; *<0.05; ns, not significant. Ig stands for immunoglobulin.

FIGS. 16A-16C depict RNA-seq gene transcripts analyses showing upregulation of mouse receptor tyrosine kinases (FIG. 16A), mouse IFN receptors (FIG. 16B) and human and mouse TNF family receptors (FIG. 16C) in tumors treated with the anti-nectin-4 ADC (AGS-22C3E) compared to the untreated or tumors treated with a non-binding control. RNA-seq gene transcripts identified receptor tyrosine kinase genes (FIG. 16A), IFN receptor family genes (FIG. 16B) and TNF family receptor genes (FIG. 16C) from the transcriptome to be upregulated upon treatment with enfortumab vedotin compared to untreated or non-binding ADC. Therapeutic agents against these upregulated receptor tyrosine kinases (FIG. 16A), IFN receptors (FIG. 16B) and TNF family receptors (FIG. 16C) can be combined with EV as potential combination therapies. Receptor tyrosine kinases upregulated upon enfortumab vedotin treatment include Csf1r, Pdgfrb, Tek/Tie2, and Flt3. Members of the TNF family of receptors of either mouse immune or human cancer genes were upregulated upon enfortumab vedotin treatment. Statistical analysis was performed using an unpaired t test. p-value; ****<0.0001, ***<0.001; **<0.01; *<0.05.

FIGS. 17A and 17B depict RNA-seq gene transcripts analyses showing upregulation of inhibitory immunoreceptors (FIG. 17A) and metabolic enzymes (FIG. 17B) in tumors treated with the anti-nectin-4 ADC (AGS-22C3E) compared to the untreated or tumors treated with a non-binding control. RNA-seq gene transcripts identified inhibitory immunoreceptor genes (FIG. 17A) and metabolic enzyme genes (FIG. 17B) from the transcriptome to be upregulated upon treatment with enfortumab vedotin compared to untreated or non-binding ADC. Therapeutic agents against these upregulated inhibitory immunoreceptors (FIG. 17A) and metabolic enzymes (FIG. 17B) can be combined with EV as potential combination therapies. Statistical analysis was performed using an unpaired t test. P-value indicators are ****<0.0001, ***<0.001; **<0.01; *<0.05; ns, not significant.

FIG. 18 depicts changes in ER stress genes in tumors treated with an anti-nectin-4 ADC (AGS-22C3E) or a control non-binding ADC (hIgG1-MMAE(4)) comparing with untreated tumors. RNA-seq gene transcripts identified genes associated with the GO positive regulation of response to endoplasmic reticulum stress (GO: 1902237) to be upregulated upon treatment with an anti-nectin-4 ADC (AGS-22C3E) compared to untreated or non-binding ADC. Statistical analysis was performed using an unpaired t test. P-value indicators are: ****<0.0001, ***<0.001; **<0.01; *<0.05.

FIG. 19A depicts changes in expression of Rho GTPase genes in tumors treated with an anti-nectin-4 ADC (AGS-22C3E) or a control non-binding ADC (hIgG1-MMAE(4)) comparing with untreated tumors. Disruption of microtubules can activate or suppress genes associated with the actin cytoskeleton to support the cell structure. Rho GTPases are known to regulate the actin cytoskeleton and changes are observed in these genes with anti-nectin-4 ADC (AGS-22C3E) treatment. FIG. 19B depicts changes in expression of Rho GTPase regulators in tumors treated with an anti-nectin-4 ADC (AGS-22C3E) or a control non-binding ADC (hIgG1-MMAE(4)) comparing with untreated tumors. FIG. 19C depicts changes in GTPase related kinase gene expression in tumors treated with an anti-nectin-4 ADC (AGS-22C3E) or a control non-binding ADC (hIgG1-MMAE(4)) comparing with untreated tumors. In FIGS. 19A-19C, statistical analysis was performed using an unpaired t test and P-value indicators are: ****<0.0001, ***<0.001; **<0.01; *<0.05.

FIG. 20 depicts changes in GO positive autophagy regulator genes (GO positive regulation of autophagy (GO: 0010508)) genes in tumors treated with an anti-nectin-4 ADC (AGS-22C3E) or a control non-binding ADC (hIgG1-MMAE(4)) comparing with untreated tumors. RNA-seq gene transcripts identified genes associated with the GO positive regulation of autophagy (GO: 0010508) to be upregulated upon treatment with enfortumab vedotin (AGS-22C3E) compared to untreated or non-binding ADC. Statistical analysis was performed using an unpaired t test and p-value indicators are: ****<0.0001, ***<0.001; **<0.01; *<0.05.

FIG. 21A depicts changes in ER/Mitochondria ATPase genes in tumors treated with an anti-nectin-4 ADC (AGS-22C3E) or a control non-binding ADC (hIgG1-MMAE(4)) comparing with untreated tumors. FIG. 21B depicts changes in cell death genes in tumors treated with an anti-nectin-4 ADC (AGS-22C3E) or a control non-binding ADC (hIgG1-MMAE(4)) treatment comparing with untreated tumors. FIG. 21C depicts changes in mitotic arrest genes in tumors treated with an anti-nectin-4 ADC (AGS-22C3E) or a control non-binding ADC (hIgG1-MMAE(4)) treatment comparing with untreated tumors. RNA-seq gene transcripts identified genes associated with the GO Mitotic cell cycle arrest (GO: 0071850) to be upregulated upon treatment with enfortumab vedotin compared to untreated or non-binding ADC. In FIGS. 21A-21C, statistical analysis was performed using an unpaired t test and p-value indicators are: ****<0.0001, ***<0.001; **<0.01; *<0.05.

FIG. 22A depicts Volcano Plot of human gene expression in untreated and anti-nectin-4 ADC (enfortumab vedotin or EV) treated tumors. FIG. 22B depicts the results of RNA-seq analysis comparing gene expression of no treatment, treatment with an anti-nectin-4 ADC (AGS-22C3E), and treatment with a control non-binding ADC (hIgG1-MMAE(4)). Upper panel depicts 736 human genes relevant to ER stress and microtubule formation. Lower panel depicts 539 mouse genes relevant to immune cell populations and inflammatory response. Colored bars on the right indicate the treatment and the changes. FIG. 22C depicts the biological processes that are changed upon anti-nectin-4 ADC (AGS-22C3E) treatment comparing with untreated in the human transcriptome.

In all figures TPM stands for total reads per million as further described below. In all figures, pg/ml stands for picograms per milliliter. For all the figures, transcripts from mouse (immune and microenvironment) and human (cancer cells) are determined as described in Section 6.1 (Example 1).

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies can be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen can be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions can include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen can be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and can be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” can be measured by assays known in the art, for example by a binding assay. The K_{D} can be measured in a RIA, for example, performed with the Fab version of an antibody of interest and its antigen (Chen et al., 1999, J. Mol Biol 293:865-81). The K_{D} or K_{D} value can also be measured by using biolayer interferometry (BLI) or surface plasmon resonance (SPR) assays by Octet®, using, for example, a Octet®QK384 system, or by Biacore®, using, for example, a Biacore®TM-2000 or a Biacore®TM-3000. An “on-rate” or “rate of association” or “association rate” or “kₒₙ” can also be determined with the same biolayer interferometry (BLI) or surface plasmon resonance (SPR) techniques described above using, for example, the Octet®QK384, the Biacore®TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, can not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that can be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure can be prepared by the hybridoma methodology first described by Kohler et al., 1975, Nature 256:495, or can be made using recombinant DNA methods in bacterial or eukaryotic animal or plant cells (see, e.g., U.S. Pat. No. 4,816,567). The “monoclonal antibodies” can also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain can be referred to as “VH.” The variable region of the light chain can be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence can contain fewer or additional amino acids corresponding to a shortening of, or insertion into, an FR or CDR of the variable domain. For example, a heavy chain variable domain can include a single amino acid insert (residue 52a according to Kabat) after residue 52 and three inserted residues (e.g., residues 82a, 82b, and 82c, etc. according to Kabat) after residue 82. The Kabat numbering of residues can be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below Table 1.

TABLE 1

| | Kabat | AbM | Chothia | Contact | IMGT |
|---|---|---|---|---|---|
| CDR-L1 | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105-L117 |
| CDR-H1 | H31--H35B | H26--H35B | H26--H32 . . . 34 | H30--H35B | H27--H38 |
| | (Kabat | | | | |
| | Numbering) | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H52--H56 | H47--H58 | H56--H65 |
| CDR-H3 | H95--H102 | H95--H102 | H95--H102 | H93--H101 | H105-H117 |

The boundaries of a given CDR can vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions can comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region can contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region can be removed, for example, during production or purification of the antibody, or by recombinantly engineering the nucleic acid encoding a heavy chain of the antibody. Accordingly, a composition of intact antibodies can comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure can be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise noted, the term “alkyl” refers to a saturated straight or branched hydrocarbon comprising from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Alkyl groups, whether alone or as part of another group, can be optionally substituted with one or more groups, preferably 1 to 3 groups (and any additional substituents selected from halogen), including, but not limited to, -halogen, —O—(C1-C8 alkyl), —O—(C2-C8 alkenyl), —O—(C2-C8 alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains comprising from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1 butynyl. Alkenyl and alkynyl groups, whether alone or as part of another group, can be optionally substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkyenl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical comprising from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH═CHCH₂—).

Unless otherwise noted, the term “alkynylene” refers to an optionally substituted alkylene group containing at least one carbon-carbon triple bond. Exemplary alkynylene groups include, for example, acetylene (—C≡C—), propargyl (—CH₂C≡C—), and 4-pentynyl (—CH₂CH₂CH₂C≡CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO₂, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group.

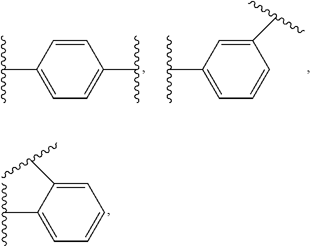

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure. Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960). Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl. Preferred “heterocycle” groups include, but are not limited to, benzofuranyl, benzothiophenyl, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; position 4 of a morpholine; and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo [4,5], [5,5], [5,6] or [6,6] system, or 9 or 10 ring atoms arranged as a bicyclo [5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms. Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and -cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

When a particular group is “substituted”, that group can have one or more substituents, preferably from one to five substituents, more preferably from one to three substituents, most preferably from one to two substituents, independently selected from the list of substituents. The group can, however, generally have any number of substituents selected from halogen. Groups that are substituted are so indicated. It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Protective groups as used herein refer to groups which selectively block, either temporarily or permanently, one reactive site in a multifunctional compound. Suitable hydroxy-protecting groups for use in the present invention are pharmaceutically acceptable and may or may not need to be cleaved from the parent compound after administration to a subject in order for the compound to be active. Cleavage is through normal metabolic processes within the body. Hydroxy protecting groups are well-known in the art, see, Protective Groups in Organic Synthesis by T. W. Greene and P. G. M. Wuts (John Wiley & sons, 3^{rd} Edition) incorporated herein by reference in its entirety and for all purposes and include, for example, ether (e.g., alkyl ethers and silyl ethers including, for example, dialkylsilylether, trialkylsilylether, dialkylalkoxysilylether), ester, carbonate, carbamates, sulfonate, and phosphate protecting groups. Examples of hydroxy protecting groups include, but are not limited to, methyl ether; methoxymethyl ether, methylthiomethyl ether, (phenyldimethylsilyl)methoxymethyl ether, benzyloxymethyl ether, p-methoxybenzyloxymethyl ether, p-nitrobenzyloxymethyl ether, o-nitrobenzyloxymethyl ether, (4-methoxyphenoxy)methyl ether, guaiacolmethyl ether, t-butoxymethyl ether, 4-pentenyloxymethyl ether, siloxymethyl ether, 2-methoxyethoxymethyl ether, 2,2,2-trichloroethoxymethyl ether, bis(2-chloroethoxy)methyl ether, 2-(trimethylsilyl)ethoxymethyl ether, menthoxymethyl ether, tetrahydropyranyl ether, 1-methoxycylcohexyl ether, 4-methoxytetrahydrothiopyranyl ether, 4-methoxytetrahydrothiopyranyl ether S,S-Dioxide, 1-[(2-choro-4-methyl)phenyl]-4-methoxypiperidin-4-yl ether, 1-(2-fluorophneyl)-4-methoxypiperidin-4-yl ether, 1,4-dioxan-2-yl ether, tetrahydrofuranyl ether, tetrahydrothiofuranyl ether; substituted ethyl ethers such as 1-ethoxyethyl ether, 1-(2-chloroethoxy)ethyl ether, 1-[2-(trimethylsilyl)ethoxy]ethyl ether, 1-methyl-1-methoxyethyl ether, 1-methyl-1-benzyloxyethyl ether, 1-methyl-1-benzyloxy-2-fluoroethyl ether, 1-methyl-1phenoxyethyl ether, 2-trimethylsilyl ether, t-butyl ether, allyl ether, propargyl ethers, p-chlorophenyl ether, p-methoxyphenyl ether, benzyl ether, p-methoxybenzyl ether 3,4-dimethoxybenzyl ether, trimethylsilyl ether, triethylsilyl ether, tripropylsilylether, dimethylisopropylsilyl ether, diethylisopropylsilyl ether, dimethylhexylsilyl ether, t-butyldimethylsilyl ether, diphenylmethylsilyl ether, benzoylformate ester, acetate ester, chloroacetate ester, dichloroacetate ester, trichloroacetate ester, trifluoroacetate ester, methoxyacetate ester, triphneylmethoxyacetate ester, phenylacetate ester, benzoate ester, alkyl methyl carbonate, alkyl 9-fluorenylmethyl carbonate, alkyl ethyl carbonate, alkyl 2,2,2, -trichloroethyl carbonate, 1,1, -dimethyl-2,2,2-trichloroethyl carbonate, alkylsulfonate, methanesulfonate, benzylsulfonate, tosylate, methylene acetal, ethylidene acetal, and t-butylmethylidene ketal. Preferred protecting groups are represented by the formulas —R^{a}, —Si(R^{a})(R^{a})(R^{a}), —C(O)R^{a}, —C(O)OR^{a}, —C(O)NH(R^{a}), —S(O)₂R^{a}, —S(O)₂OH, P(O)(OH)₂, and —P(O)(OH)OR^{a}, wherein R^{a} is C₁-C₂₀ alkyl, C₂-C₂₀ alkenyl, C₂-C₂₀ alkynyl, —C₁-C₂₀ alkylene(carbocycle), —C₂-C₂₀ alkenylene(carbocycle), —C₂-C₂₀ alkynylene(carbocycle), —C₆-C₁₀ aryl, —C₁-C₂₀ alkylene(aryl), —C₂-C₂₀ alkenylene(aryl), —C₂-C₂₀ alkynylene(aryl), —C₁-C₂₀ alkylene(heterocycle), —C₂-C₂₀ alkenylene(heterocycle), or —C₂-C₂₀ alkynylene(heterocycle) wherein said alkyl, alkenyl, alkynyl, alkylene, alkenylene, alkynylene, aryl, carbocycle, and heterocycle radicals whether alone or as part of another group are optionally substituted.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

The term “compound” refers to and encompasses the chemical compound itself as well as, whether explicitly stated or not, and unless the context makes clear that the following are to be excluded: amorphous and crystalline forms of the compound, including polymorphic forms, where these forms can be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer can be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions can be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules can be the same or different. In some instances, reference made herein to a compound of the invention will include an explicit reference to one or of the above forms, e.g., salts and/or solvates; however, this reference is for emphasis only, and is not to be construed as excluding other of the above forms as identified above.

As used herein, the term “conservative substitution” refers to substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and can be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid
substitution matrix (block substitution matrix). The higher the
value, the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, Md. (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and)(BLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the)(BLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of)(BLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies can also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” can be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating can be determined by assessing whether there has been a decrease, alleviation and/or mitigation of one or more symptoms associated with the underlying disorder such that an improvement is observed with the patient, despite that the patient can still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

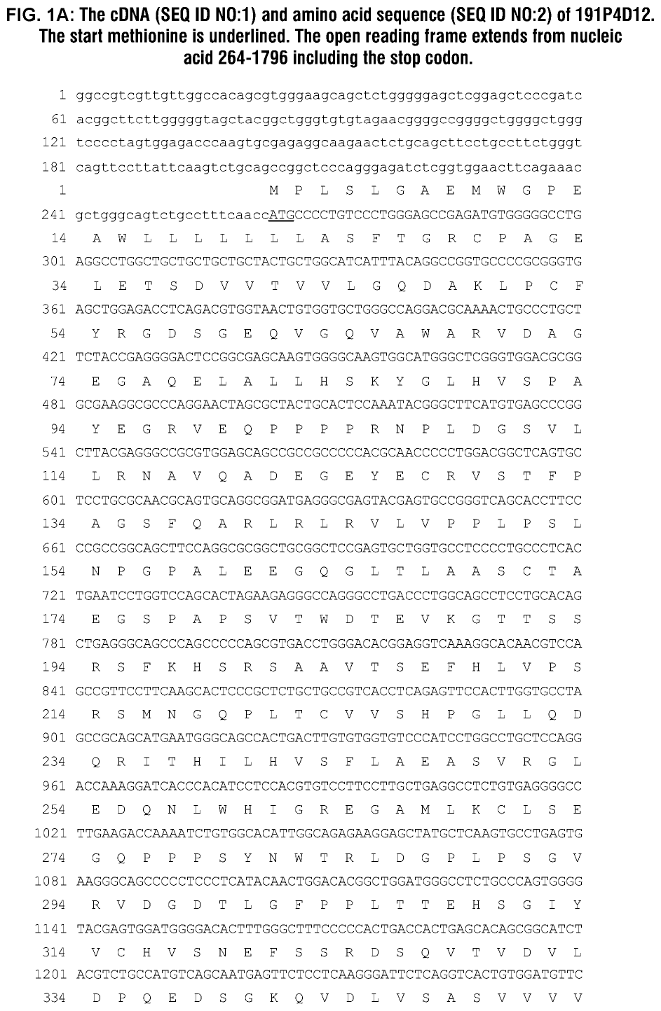

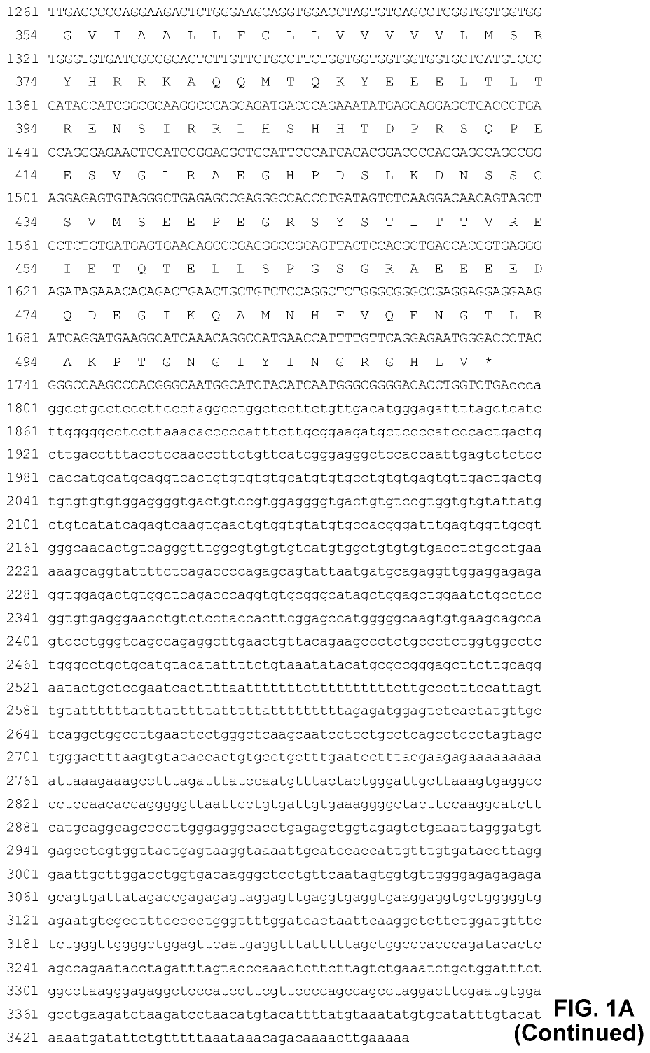

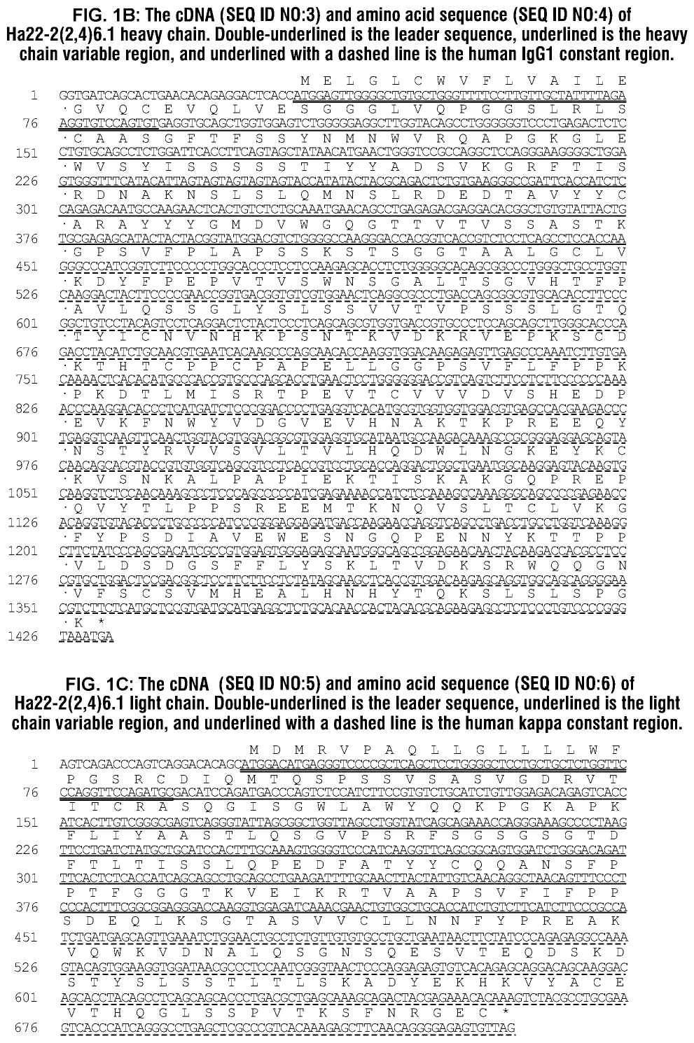

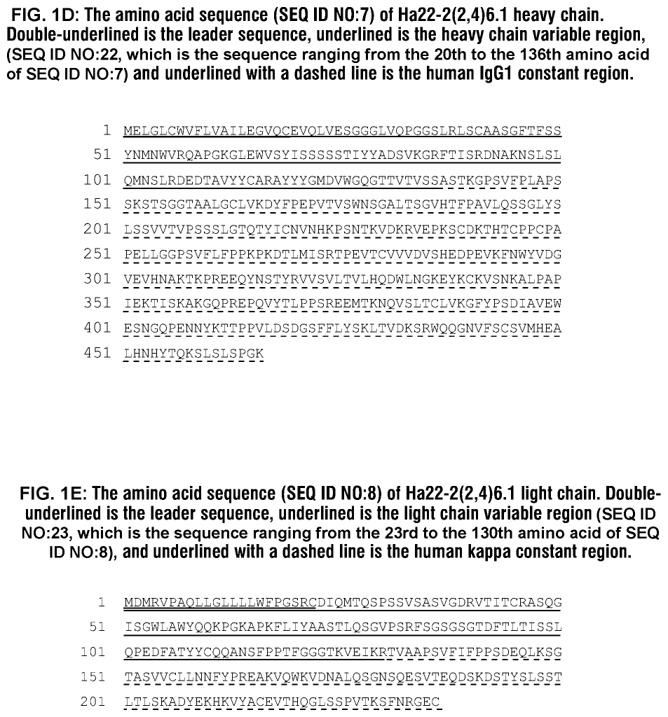

The term “variant” refers to a molecule that exhibits a variation from a described type or norm, such as a protein that has one or more different amino acid residues in the corresponding position(s) of a specifically described protein (e.g. the 191P4D12 protein shown in FIG. 1.) An analog is an example of a variant protein. Splice isoforms and single nucleotides polymorphisms (SNPs) are further examples of variants.

The “191P4D12 proteins” and/or “191P4D12 related proteins” of the invention include those specifically identified herein (see, FIG. 1), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the invention, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids.

As used herein, the term “ADC Marker genes” refers to both ADC Set I Marker genes and ADC Set II Marker genes, each as defined herein.

As used herein, the term “ADC Set I Marker genes” refers to any set or subset of the follow group of genes: MHC signature genes, TLR family genes, interleukin receptor family genes, immune checkpoint receptor genes, receptor tyrosin kinase genes, IFN receptor family genes, TNF family receptor genes, inhibitory immunoreceptor genes, and/or metabolic enzyme genes, each as defined herein, in any combination or permutation.

As used herein, the term “major histocompatibility complex (MHC) signature genes” (“MHC signature genes”) is intended to mean genes that have the following two attributes: (1) whose expression level correlates, either positively or negatively, with the level of MHC protein at the surface of the cells, and (2) either (a) whose expression products are components of the MHC or (b) which or the expression products of which regulate the expression level of any of the components of MHC. MHC signature genes include “MHC class genes” and “MHC regulator genes” as described herein.

As used herein, the term “MHC class genes” is intended to mean genes whose expression products are components of the MHC. MHC class genes include “MHC class I genes,” the expression products of which are components of MHC class I, and “MHC class II genes,” the expression products of which are components of MHC class II. MHC class genes also include “MHC class III genes,” the expression products of which are members of MHC class III. MHC class I, MHC class II, and MHC class III have been intensively studied and the components of each are well known. Examples of MHC class genes include MHC class I, MHC class II, and MHC class III genes described in Wieczorek M et al., Front Immunol. 2017; 8: 292; Handunnetthi L et al., Genes Immun. 11(2): 99-112 (2010 March); Neefjes J et al., Nature Reviews Immunology 11:823-836 (2011); Rock K et al., Trends Immunol. 2016 November; 37(11): 724-737; Carlini F et al., PLoS One. 2016; 11(10): e0163570; Takashi Shiina et al., Journal of Human Genetics (2009) 54, 15-39; Doxiadis G et al., Mol. Biol. Evol. 29(12):3843-3853 (2012); Gruen, J R, et al., Frontiers in Bioscience. 6 (3): D960-172; and C Yung Yu et al., Immunol Today. 2000 July; 21(7):320-8, all of which are herein incorporated in their entirety by reference.

The “MHC class I” is an antigen- or peptide-presenting protein complex that includes peptide-binding (or peptide-presenting) subunits, which bind to sequences of amino acids for antigen presentation, and molecules aiding antigen-processing or peptide presentation (such as Transporter associated with antigen processing (TAP) and tapasin). The peptide-binding subunits of MHC class I include two chains, a single heavy α-chain (HC or α-chain) and a membrane-proximal immunoglobulin (Ig) domain supporting the peptide-binding unit (also known as β chain, β2 microglobulin (β2m or B2M)). The MHC class I α-chain has a transmembrane domain (transmembrane helix) anchoring the α-chain of MHC class I in the membrane. In human, the α-chain of MHC class I is known as members of the human leukocyte antigen (HLA), and is encoded by HLA gene loci including HLA-A, HLA-B, HLA-C, HLA-E, HLA-F, HLA-G and HLA-H. The HLA-A, HLA-B, HLA-C, HLA-E, HLA-F, HLA-G and HLA-H gene loci in human as well as the counterpart MHC class I α-chain in other species are highly polymorphic. The term “MHC class I genes” includes all natural gene variants for above described components of MHC class I, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. The term “MHC class I genes” also encompasses “full-length,” unprocessed genes as well as any form of MHC class I genes that results from processing in the cell. Examples of the MHC class I genes encompass genes of any vertebrate source, including mammals such as primates (e.g., humans and cynomolgus monkeys (cynos)), dogs, and rodents (e.g., mice and rats), unless otherwise indicated. In some specific examples, MHC class I genes include HLA-A, HLA-B, HLA-C, HLA-E, HLA-F, HLA-G, HLA-H, Transporter 2, ATP binding cassette subfamily B member (TAP2), and/or tapasin. Other examples of MHC class I genes include such genes disclosed in Wieczorek M et al., Front Immunol. 2017; 8: 292; Handunnetthi L et al., Genes Immun. 11(2): 99-112 (2010 March); Neefjes J et al., Nature Reviews Immunology 11:823-836 (2011); Rock K et al., Trends Immunol. 2016 November; 37(11): 724-737; Carlini F et al., PLoS One. 2016; 11(10): e0163570; and Takashi Shiina et al., Journal of Human Genetics (2009) 54, 15-39; Doxiadis G et al., Mol. Biol. Evol. 29(12):3843-3853 (2012); all of which are herein incorporated in their entirety by reference.

The “MHC class II” is an antigen- or peptide-presenting protein complex that includes peptide-binding (or peptide-presenting) subunits (e.g. HLA-DQ, HLA-DR, and HLA-DP), which bind to sequences of amino acids for antigen presentation, and proteins assisting antigen loading onto MHC class II's peptide-binding proteins (e.g. HLA-DM, Ii, and HLA-DO). The peptide-binding subunits of MHC class II include two chains, an α-chain and a β-chain, each having a membrane-proximal immunoglobulin (Ig) domain supporting the peptide-binding unit. In human, MHC class II is known as members of human leukocyte antigen. Human MHC class II gene loci (e.g. HLA-DQ, HLA-DR, and HLA-DP) as well as the counterpart MHC class II genes in other species are highly polymorphic. The term “MHC class II genes” includes all natural gene variants for above described components of MHC class II, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. The term “MHC class II genes” also encompasses “full-length,” unprocessed genes as well as any form of MHC class II genes that results from processing in the cell. Examples of the MHC class II genes encompass genes of any vertebrate source, including mammals such as primates (e.g., humans and cynomolgus monkeys (cynos)), dogs, and rodents (e.g., mice and rats), unless otherwise indicated. In some specific examples, MHC class II genes include HLA-DRA, HLA-DRB, HLA-DRB, HLA-DQA1, HLA-DQB, HLA-DPA, HLA-DPB, HLA-DMA, HLA-DMB, HLA-DOA, and/or HLA-DOB. Certain MHC class II genes can be further classified by its gene location. For Example, HLA-DRB includes HLA-DRB1, HLA-DRB3, HLA-DRB4, and HLA-DRB5; HLA-DQA includes HLA-DQA1 and HLA-DQA2; HLA-DQB includes HLA-DQB1 and HLA-DQB2; HLA-DPA includes HLA-DPA1; and HLA-DPB includes HLA-DPB1. Other examples of MHC class II genes include such genes disclosed in Wieczorek M et al., Front Immunol. 2017; 8: 292; Handunnetthi L et al., Genes Immun. 11(2): 99-112 (2010 March); Neefjes J et al., Nature Reviews Immunology 11:823-836 (2011); Rock K et al., Trends Immunol. 2016 November; 37(11): 724-737; Carlini F et al., PLoS One. 2016; 11(10): e0163570; and Takashi Shiina et al., Journal of Human Genetics (2009) 54, 15-39; Doxiadis Get at, Mol. Biol. Evol. 29(12):3843-3853 (2012); all of which are herein incorporated in their entirety by reference.

The “MHC class III genes” refers to a cluster of genes found between MHC class I and MHC class II genes on the human chromosome 6 (which region on chromosome 6 is referred to as MHC class III region). As used herein, the term “MHC class III genes” also includes genes that are located in the telomeric end of the MHC class III region and that appear to be involved in both global and specific inflammatory responses, which genes are also known in some literature as genes of the MHC class VI or inflammatory region. The term “MHC class III genes” includes all natural gene variants for WIC class III genes, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. The term “MHC class III genes” also encompasses “full-length,” unprocessed genes as well as any form of MHC class III genes that results from processing in the cell. Examples of the MHC class III genes encompass genes of any vertebrate source, including mammals such as primates (e.g., humans and cynomolgus monkeys (cynos)), dogs, and rodents (e.g., mice and rats), unless otherwise indicated. Examples of MHC class III genes include complement components C₂, C₄, and factor B. Additional specific examples of WIC class III genes include Lst1, Ltb, Aif1, and/or TNF. Other examples of MHC class III genes include such genes disclosed in Gruen, J R, et al., Frontiers in Bioscience. 6 (3): D960-172; and C Yung Yu et al., Immunol Today. 2000 July; 21(7):320-8, both of which are herein incorporated in their entirety by reference.

As used herein, the term “MHC regulator genes” is intended to mean genes that have the following two attributes: (1) whose expression level correlates, either positively or negatively, with the expression level of MHC class genes, and (2) which or the expression products of which regulate the expression level of the WIC class genes. WIC regulator genes include genes that play a role or whose expression products play a role in the signaling pathway that controls the expression level of WIC class genes. The MHC regulators produced by the WIC regulator genes can increase, turn on, or speed up the expression of the WIC class genes, the folding of the protein subunits of the MHC, or the transportation of the WIC. Examples of WIC regulator genes include: genes for transcription factors that regulate the expression of MHC class genes; genes for molecules that regulate the location, stability, or activation of the transcription factors regulating WIC class genes; genes for the molecules of the signaling cascades whose activation results in increased MHC levels; and/or cis- or trans-regulatory elements of the MHC class genes. The term “MHC regulator genes” includes all natural gene variants for the MHC regulator genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. The term “MHC regulator genes” also encompasses “full-length,” unprocessed genes as well as any form of MHC regulator genes that results from processing in the cell. Examples of the MHC regulator genes encompass genes of any vertebrate source, including mammals such as primates (e.g., humans and cynomolgus monkeys (cynos)), dogs, and rodents (e.g., mice and rats), unless otherwise indicated. Specific examples of MHC regulator genes include interferon regulatory factor 7 (IRF7) gene, nuclear factor kappa-light-chain-enhancer of activated B cells (NF-κB) family genes, signal transducer and activator of transcription (STAT) family genes, and/or indoleamine 2,3-dioxygenase 1 (IDO1). Other examples of MHC regulator genes include IRF7 gene, nuclear factor kappa B subunit 2 (NFKB2), RELA, STAT2, and/or IDO1.

As used herein, the term “NF-κB family genes” is intended to mean genes for NF-κB transcription factors as shown in the following Table 4 for mammals and the corresponding orthologs and paralogs in non-mammalian species, including all natural gene variants for NF-κB transcription factors, such as polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the NF-κB family genes encompass genes of any vertebrate source, including mammals such as primates (e.g., humans and cynomolgus monkeys (cynos)), dogs, and rodents (e.g., mice and rats), unless otherwise indicated. Examples of NF-κB family genes also include those listed in Table 4 and such genes disclosed in Cesidio Giuliani C et al., Front. Endocrinol. 9: 471 (2018); Zhang Q, et al., Cell 168:37-57 (2017); Napetschnig J, et al., Annu Rev Biophys. 42:443-68 (2013); Hinz M, et al., EMBO Rep. 15:46-61 (2013); Hayden T H, et al., Genes Dev. 26:203-34 (2012); all of which are herein incorporated in their entirety by reference.

TABLE 4
The NF-κB family members in mammals.

| Protein | Precursor | Gene |
|---|---|---|
| p50 subunit | p105 | NFkB1 (or |
| | | NFKB1) |
| p52 subunit | p100 | NFkB2 (or |
| | | NFKB2) |
| p65 subunit (RelA) | None | RELA |
| c-Rel | None | REL |
| RelB | None | RELB |

As used herein, the term “interferon regulatory factor genes” and “IRF genes” are used interchangeably to refer to genes whose expression product forms a family of 9 transcription factors (IRF1-9) that share significant homology within their N-terminal DNA-binding domain (DBD) of ˜120 amino acids, which DBD forms a helix-loop-helix motif that recognizes specific DNA sequences similar to the interferon stimulated response element (ISRE). IRF genes include all natural gene variants for IRF genes, such as polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of IRF genes include IRF1, IRF2, IRF3, IRF4, IRF5, IRF6, IRF7, IRF8, and IRF9 in human and their equivalents in other mammals such as primates (e.g., cynomolgus monkeys (cynos)), dogs, and rodents (e.g., mice and rats), the corresponding orthologs or paralogs in other non-mammalian species, and such genes disclosed in Caroline A. Jefferies, Frontiers in Immunology 10: Article 325 (2019), which is herein incorporated in its entirety by reference.

As used herein, the term “nuclear transcription factor Y genes” and “NFY genes” are used interchangeably to refer to genes whose expression product forms the nuclear transcription factor Y complex, which has three different subunits, NFYA, NFYB and NFYC. The 3-subunits NFY complex binds to CCAAT boxes in promoters of its target genes. NFY genes include all natural gene variants for MFY genes, such as polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of NFY genes include NFYA, NFYB and NFYC in human and their equivalents in other mammals such as primates (e.g., cynomolgus monkeys (cynos)), dogs, and rodents (e.g., mice and rats), the corresponding orthologs or paralogs in other non-mammalian species, and such genes disclosed in Luong Linh Ly, et al., Am J Cancer Res. 3(4): 339-346 (2013), which is herein incorporated in its entirety by reference.

As used herein, the term “STAT family genes” is intended to mean the genes for the signal transducers and activators of transcription (STAT) proteins that include STAT1, STAT2, STAT3, STAT4, STAT5a, STAT5b and STAT6 in mammals and the corresponding orthologs or paralogs in other non-mammalian species. STAT family genes include all natural gene variants for STAT family genes, such as polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of STAT family genes include STAT1, STAT2, STAT3, STAT4, STAT5a, STAT5b and STAT6 in mammals such as primates (e.g., humans and cynomolgus monkeys (cynos)), dogs, and rodents (e.g., mice and rats), the corresponding orthologs or paralogs in other non-mammalian species, and such genes disclosed in Levy D E, et al., Nat Rev Mol Cell Biol. 3:651-(2002); and Mitchell T et al., Immunology 114(3): 301-312 (2005 March); both of which are herein incorporated in their entirety by reference.

As used herein, the term “GTPase related kinase genes” is intended to mean

As used herein, the term “ADC Set II Marker genes” is intended to mean genes that have both attributes of: (1) which are not ADC Set I Marker genes and (2) whose expression correlates with the increase in immunogenic cell death (ICD). “Immunogenic cell death” refers to regulated cell death in which immunocompetent hosts activate an adaptive immune response against dead cell-associated antigens and cause cell death. For example, ICD includes an immunologically unique type of regulated cell death that enables, rather than suppresses, T cell-driven immune responses that are specific for antigens derived from the dying cells. Other examples of ICD include such genes disclosed in Bezu L et al., Front Immunol. 6: 187 (2015); Vanmeerbeek I et al., Oncoimmunology 9(1):1703449 (2020 Jan. 9); Pol J et al, Oncoimmunology. 4(4):e1008866 (2015 Mar. 2), all of which are herein incorporated in their entirety by reference. Examples of ADC Set II Marker genes include genes whose expression products play a role in ICD but which are not ADC Set I Marker genes. Other examples ADC Set II Marker genes include any set or subset of the follow group of genes: the ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and/or GTPase related kinase genes, each as defined herein, in any combination or permutation. Additional examples of ADC Set II Marker genes are described in WO 2019/183438 or US20190290775A1, both of which are herein incorporated in their entirety by reference.

As used herein, the term “gene expression” or “expression of a gene” is intended to mean the levels of expression and/or pattern of expression of a gene in a biological sample, such as immune cells, cancer cells, a population of immune cells, a population of cancer cells, cancer tissues, or other tissues. The term “gene expression” or “expression of a gene” can be used herein in the absolute sense, e.g. the absolute levels of the gene expression product (such as number of molecules of the gene expression product), or in the relative or comparative sense, e.g. relative to one or more reference genes. When the term “gene expression” or “expression of a gene” is used to mean relative or comparative gene expression, the reference genes can be a different gene (e.g. housekeeping gene), or the same genes but at a different time point or from a different biological sample (e.g. expression of the same gene but without treatment or with only control treatment). “Gene expression” or “expression of a gene” is determined by the levels of the expression product, such as the level of the transcribed mRNA product of the gene or the level of the protein product encoded by the gene.

As used herein, the term “increase,” when used in the context of expression of a target gene with respect to that of a reference gene, is intended to mean a higher level of the expression product of the target gene compared with the reference gene. For example, an increase of the expression of a MHC signature gene in the subject after administration of a ADC compared to the expression of the MHC signature gene in the subject before the administration of the ADC would mean that the level of the expression product of the MHC signature gene in the subject after ADC administration is higher than the level of expression of the MHC signature gene in the subject before the administration of the ADC. Examples of the increase of the gene expression disclosed herein include an increase of 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, or more when compared with the reference gene. Other examples of the increase of the gene expression disclosed herein also include an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 fold or more when compared with the reference gene.

“ER stress genes” refer to any genes that have both attributes of: (1) whose expression correlates with the increase in ICD and (2) which express at higher level as a result of a stress acting at the endoplasmic reticulum (ER), which stress often results from the accumulation of unfolded or misfolded proteins in the ER lumen. Examples of ER stress genes include genes whose expression products signal to other cells, e.g. immune cells, the occurrence of the stress acting on the ER and genes whose expression products are involved in the unfolded protein response (UPR), including inositol-requiring protein 1 (IRE1), PKR-like endoplasmic reticulum kinase (PERK), and activating transcription factor (ATF)-6. The term “ER stress genes” includes all natural gene variants for the ER stress genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the ER stress genes encompass genes of any vertebrate source, including mammals such as primates (e.g., humans and cynomolgus monkeys (cynos)), dogs, and rodents (e.g., mice and rats), unless otherwise indicated. Examples of ER stress genes also include such genes disclosed in Malhi H et al., J Hepatol. 54(4): 795-809 (2011 April); Daisuke Ariyasu et al., Int J Mol Sci. 18(2): 382 (2017 Fe); Jonathan H. Lin et al., Annu Rev Pathol. 3: 399-425(2008); Stefania Lenna et al., Arthritis Rheum. 65(5): 1357-1366 (2013 May); Dan Lindholm et al., Front. Cell Dev. Biol., 5: 48 (May 2017); all of which are herein incorporated in their entirety by reference. Other specific examples of ER stress genes include XBP-1S, ERP29, TRAF2, c-JUN, BCL2L11, BCAP31, SERINC3, DAP2IP, ERN1, ATF6, NCK2, PPP1R15A, UBQLN2, BAG6, and BOK. Further examples of ER stress genes include genes listed in the Gene Ontology (GO) positive regulation of response to endoplasmic reticulum stress (GO: 1902237) and genes listed in the GO response to endoplasmic reticulum stress (GO: 0034976), which can be found in various databases such as geneontology.org or amigo.geneontology.org with the GO ID or the GO name. Additional examples of ER stress genes are described in WO 2019/183438, which is herein incorporated in its entirety by reference.

“ER/mitochondria ATPase genes,” or short as “ER ATPase genes” or “mitochondria ATPase genes,” refer to any genes that have both attributes of: (1) whose expression correlates with the increase in ICD and (2) whose expression products are ATPases (e.g. ATP synthase and/or ATP hydrolase) in ER or mitochondria. The term “ER/mitochondria ATPase genes” includes all natural gene variants for the ER/mitochondria ATPase genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the ER/mitochondria ATPase genes encompass genes of any vertebrate source, including mammals such as primates (e.g., humans and cynomolgus monkeys (cynos)), dogs, and rodents (e.g., mice and rats), unless otherwise indicated. Examples of ER/mitochondria ATPase genes also include such genes disclosed in Maria R. Depaoli et al., Biological Reviews 94(2): 610-628 (2019); An I. Jonckheere et al., Journal of Inherited Metabolic Disease 35:211-225 (2012); Alain Dautant et al., Front Physiol. 9: 329— (2018); all of which are herein incorporated in their entirety by reference. Other specific examples of ER/mitochondria ATPase genes include ATP2A3, MT-ATP6, and/or MT-ATP8.

“Cell death genes” refer to any genes that have both attributes of: (1) whose expression correlates with the increase in ICD and (2) whose expression products play a role in programmed cell death (“apoptosis”). The term “cell death genes” includes all natural gene variants for the cell death genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the cell death genes encompass genes of any vertebrate source, including mammals such as primates (e.g., humans and cynomolgus monkeys (cynos)), dogs, and rodents (e.g., mice and rats), unless otherwise indicated. Examples of cell death genes also include such genes disclosed in Lorenzo Galluzzi et al., Cell Death & Differentiation 25:486-541 (2018) (including such genes disclosed in Sections of “Intrinsic apoptosis” and “Extrinsic apoptosis”), which is herein incorporated in its entirety by reference. Other specific examples of cell death genes include Bax, BCL2L1, BCL2L11, and BOK.

“T cell stimulator genes” refer to any genes that have both attributes of: (1) whose expression correlates with the increase in ICD and (2) whose expression products play a role in stimulating T cells during adaptive immune response. The term “T cell stimulator genes” includes all natural gene variants for the T cell stimulator genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the T cell stimulator genes encompass genes of any vertebrate source, including mammals such as primates (e.g., humans and cynomolgus monkeys (cynos)), dogs, and rodents (e.g., mice and rats), unless otherwise indicated. Examples of T cell stimulator genes also include such genes disclosed in Ryuma Tokunaga, et al., Cancer Treat Rev. 63: 40-47 (2018 February); Anu Sharma et al., Chapter 77—Immunotherapy of Cancer in Clinical Immunology (Fifth Edition) Principles and Practice 2019, Pages 1033-1048.el; all of which are herein incorporated in their entirety by reference. Other specific examples of T cell stimulator genes include MIG (CXCL9) and/or IP10 (CXCL10).

“Macrophage/innate immunity stimulator genes,” or short as “macrophage stimulator genes” or “innate immunity stimulator genes,” refer to any genes that have both attributes of: (1) whose expression correlates with the increase in ICD and (2) whose expression products play a role in stimulating macrophage or innate immunity during adaptive immune response. The term “macrophage/innate immunity stimulator genes” includes all natural gene variants for the macrophage/innate immunity stimulator genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the macrophage/innate immunity stimulator genes encompass genes of any vertebrate source, including mammals such as primates (e.g., humans and cynomolgus monkeys (cynos)), dogs, and rodents (e.g., mice and rats), unless otherwise indicated. Examples of macrophage/innate immunity stimulator genes also include such genes disclosed in Vijay Kumar, Chapter of Macrophages: The Potent Immunoregulatory Innate Immune Cells in Macrophage Activation—Biology and Disease Edited by Khalid Hussain Bhat (2019); Nelson C Di Paolo et al., Nat Immunol. 17(8): 906-913 (2016 Jul. 19); David M. Mosser et al., Nat Rev Immunol. 8(12): 958-969 (2008 December); Düwell P et al., Hematol Oncol Clin North Am. 33(2):215-231 (2019 April); all of which are herein incorporated in their entirety by reference. Other specific examples of macrophage/innate immunity stimulator genes include IL-1α and/or M-CSF (CSF).

“Chemoattractant genes” refer to any genes that have both attributes of: (1) whose expression correlates with the increase in ICD and (2) whose expression products induce movement of immune cells in the direction towards the higher concentration of the expression products. The term “chemoattractant genes” includes all natural gene variants for the chemoattractant genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the chemoattractant genes encompass genes of any vertebrate source, including mammals such as primates (e.g., humans and cynomolgus monkeys (cynos)), dogs, and rodents (e.g., mice and rats), unless otherwise indicated. Examples of chemoattractant genes also include such genes disclosed in Jonathon W. Homeister et al., Section of “Chemoattractants, Cytokines, and Chemokines” of Chapter 83—Immunologic Mechanisms of Vasculitis in Seldin and Giebisch's The Kidney (Fifth Edition) Physiology & Pathophysiology 1-2 Pages 2817-2846 (2013); Chao Shi and Eric G. Pamer Nat Rev Immunol. 11(11): 762-774 (2011 Oct. 10); both of which are herein incorporated in their entirety by reference. Other specific examples of chemoattractant genes include Eotaxin (CCL11), MIP1α, MIP1β, and/or MCP1.

“Toll-like receptor family genes” refer to genes encoding the toll-like receptors (TLRs), including 10 members (TLR1-TLR10) in human, 12 (TLR1-TLR9, TLR11-TLR13) in mouse, and the orthologs and paralogs in other species. The term “Toll-like receptor family genes” includes all natural gene variants for the TLR family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of TLRs and TLR genes are further disclosed in Takumi Kawasaki and Taro Kawai Front. Immunol. 5:461 (25 Sep. 2014), which is incorporated herein in its entirety by reference. Other specific examples of TLR family genes include TLR7, TLR8, and TLR9.

“Rho GTPase genes” refers to any genes that have both attributes of: (1) whose expression correlates with the increase in ICD and (2) whose expression products are a family of small GTP-binding proteins involved in cell cytoskeleton organization, cell migration and signaling of cell migration. The term “Rho GTPase genes” includes all natural gene variants for the Rho GTPase genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the Rho GTPase genes encompass genes of any vertebrate source, including mammals such as primates (e.g., humans and cynomolgus monkeys (cynos)), dogs, and rodents (e.g., mice and rats), unless otherwise indicated. Examples of Rho GTPase genes also include such genes disclosed in Raquel B. Haga and Anne J. Ridley, Small GTPases. 7(4): 207-221 (2016 October-December); Sandrine Etienne-Manneville and Alan Hall, Nature 420:629-635 (2002); both of which are herein incorporated in their entirety by reference. Other specific examples of Rho GTPase genes include RhoB, RhoF, and/or RhoG.

“Rho GTPase regulator genes” refer to any genes that have both attributes of: (1) whose expression correlates with the increase in ICD and (2) whose expression products regulate the activity, location, concentration, conformation, or function of Rho GTPase. Examples of Rho GTPase regulator genes include genes whose expression products are guanine nucleotide dissociation inhibitors (GDIs), GTPase-activating proteins (GAPs), and/or guanine nucleotide exchange factors (GEFs). The term “Rho GTPase regulator genes” includes all natural gene variants for the Rho GTPase regulator genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the Rho GTPase regulator genes also encompass genes of any vertebrate source, including mammals such as primates (e.g., humans and cynomolgus monkeys (cynos)), dogs, and rodents (e.g., mice and rats), unless otherwise indicated. Some specific examples of Rho GTPase regulator genes include such genes disclosed in Raquel B. Haga and Anne J. Ridley, Small GTPases. 7(4): 207-221 (2016 October-December); Sandrine Etienne-Manneville and Alan Hall, Nature 420:629-635 (2002); both of which are herein incorporated in their entirety by reference. Other specific examples of Rho GTPase regulator genes include DAP2IP, ARHGEF18, ARHGEF5, and/or RASAL1.

“Mitotic arrest genes” refer to any genes that have the attributes of: (1) whose expression correlates with the increase in ICD, and (2) whose expression products play a role in the process in which the mitotic cell cycle is halted during one of the normal phases (G1, S, G2, and M). Examples of mitotic arrest genes include genes listed in the Gene Ontology (GO) Mitotic Cell Cycle Arrest (GO: 0071850), which can be found in various databases such as geneontology.org or amigo.geneontology.org with the GO ID or the GO name. The term “mitotic arrest genes” includes all natural gene variants for the mitotic arrest genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the mitotic arrest genes also encompass genes of any vertebrate source, including mammals such as primates (e.g., humans and cynomolgus monkeys (cynos)), dogs, and rodents (e.g., mice and rats), unless otherwise indicated. Specific examples of mitotic arrest genes include such genes disclosed in Hirofumi Harashima et al., Trends in Cell Biology, 23(7):345-356 (July, 2013); Vermeulen K et al., Cell Prolif. 36(3):131-49 (2003 June); Schafer K A, Vet Pathol. 35(6):461-78 (1998 November), which are herein incorporated in their entireties by reference. Other specific examples of mitotic arrest genes include CCND1, CDKN1A, GADD45B, E4F1, CDC14B, and DAPK1.

“RFX transcription factor family genes” refers to genes whose expression products are members of the Regulatory Factor binding to the X-box transcription factors. In human, the RFX transcription factor family genes encompasses RFX1, RFX2, RFX3, RFX4, RFX5, RFX6, RFX7, RFXAP, RFXANK, and RFX8. The term “RFX transcription factor family genes” encompasses all the paralogs and orthologs of the metazoan genomes that corresponds to the human RFX1-8, for example, C. elegans possesses one, Drosophila has two, mammals have eight, fishes have nine RFX genes. The term “RFX transcription factor family genes” includes all natural gene variants for the RFX transcription factor family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the RFX transcription factor family genes also encompass genes of any vertebrate source, including mammals such as primates (e.g., humans and cynomolgus monkeys (cynos)), dogs, and rodents (e.g., mice and rats), unless otherwise indicated. Specific examples of RFX transcription factor family genes include such genes disclosed in Debora Sugiaman-Trapman et al., BMC Genomics 19: Article number 181 (2018); Syed Aftab et al., BMC Evolutionary Biology 8:Article number: 226 (2008), both of which are herein incorporated in their entireties by reference.

“Siglec family genes” refers to genes whose expression products are members of a family of immune regulatory receptors that are sialic acid-binding immunoglobulin-type lectins. In human, the siglec family genes encompasses siglec-1, siglec-2, siglec-3, siglec-4, siglec-5, siglec-6, siglec-7, siglec-8, siglec-9, siglec-10, siglec-11, siglec-12, siglec-13, siglec-14, siglec-15, and siglec-16. The term “siglec family genes” encompasses all the paralogs and orthologs of the metazoan genomes that corresponds to the human siglec1-16, for example. The term “siglec family genes” includes all natural gene variants for the siglec family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the siglec family genes also encompass genes of any vertebrate source, including mammals such as primates (e.g., humans and cynomolgus monkeys (cynos)), dogs, and rodents (e.g., mice and rats), unless otherwise indicated. Specific examples of RFX transcription factor family genes include such genes disclosed in Kim F. Bornhöfft et al., Developmental & Comparative Immunology, 86:219-231 (September 2018,), which is herein incorporated in its entirety by reference.

“GO positive autophagy regulator genes” refer to any genes that have the attributes of: (1) whose expression correlates with the increase in ICD, and (2) whose expression products play a role in the process that activates, maintains or increases the rate of autophagy (autophagy is the process in which cells digest parts of their own cytoplasm). Examples of GO positive autophagy regulator genes are listed in the Gene Ontology (GO) positive regulation of autophagy (GO: 0010508), which can be found in various databases such as geneontology.org or amigo.geneontology.org with the GO ID or the GO name. The term “GO positive autophagy regulator genes” includes all natural gene variants for the GO positive autophagy regulator genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the GO positive autophagy regulator genes also encompass genes of any vertebrate source, including mammals such as primates (e.g., humans and cynomolgus monkeys (cynos)), dogs, and rodents (e.g., mice and rats), unless otherwise indicated. Specific examples of GO positive autophagy regulator genes include such genes disclosed in Congcong He et al., Annu Rev Genet. 43: 67-93 (2009); Chiara Di Malta et al., Front. Cell Dev. Biol. 7:114 (2019); Jens Füllgrabe et al., Journal of Cell Science 129: 3059-3066 (2016); Ying Yang et al., Cell Death & Differentiation 27:858-871(2020), all of which are herein incorporated in their entireties by reference. Other specific examples of GO positive autophagy regulator genes include BCL2L11, ROCK1, TSC1, TSC2, BAG3, MFN2, RIPK1, RIPK4, HDAC6, STK11, ULK1, FOXO1, FOXO3, and/or MUL1.

“GTPase related kinase genes” refer to any genes that have the attributes of: (1) whose expression correlates with the increase in ICD, (2) whose expression products are kinases, and (3) whose expression products have functions related to the function of the GTPases. Examples of GTPase related kinase genes include ROCK1 and/or PAK4.

“Interleukin receptor family genes” refer to any genes that have the attributes of: (1) whose expression correlates with the increase in ICD, and (2) whose expression products function as receptors to a group of cytokines known as interleukins (ILs), e.g. these expression products are cell surface proteins that bind interleukins and trigger intracellular changes influencing the behavior of cells. The term “interleukin receptor family genes” includes all natural gene variants for the interleukin receptor family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the interleukin receptor family genes also encompass genes of any vertebrate source, including mammals such as primates (e.g., humans and cynomolgus monkeys (cynos)), dogs, and rodents (e.g., mice and rats), unless otherwise indicated. Specific examples of the interleukin receptor family genes include such genes encoding receptors for IL1-40. Other specific examples of the interleukin receptor family genes include IL2RA, IL2RB, IL2RG, IL21R, IL27R, IL1RN, IL17RA, IL3RA, IL1R1, IL17RC, IL20RA, and/or IL22RA1.

“Immune checkpoint receptor genes” refer to any genes that have the attributes of: (1) whose expression correlates with the increase in ICD, (2) whose expression products are immune checkpoint proteins, and (3) whose expression products are also receptors of a ligand. Without being limited by a particular theory, immune checkpoint proteins are proteins involved in a series of immunomodulatory pathways that are (1) inhibitory or co-inhibitory, e.g. pathways that keep immune responses in check (such as negatively regulating T cell activation or function), or (2) stimulatory or co-stimulatory, e.g. pathways that enhance the body's immune response against pathogens (such as promoting T cell activation or function). The term “immune checkpoint receptor genes” includes all natural gene variants for the immune checkpoint receptor genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the immune checkpoint receptor genes also encompass genes of any vertebrate source, including mammals such as primates (e.g., humans and cynomolgus monkeys (cynos)), dogs, and rodents (e.g., mice and rats), unless otherwise indicated. Some examples of immune checkpoint receptor genes include those described in Qin S. et al., Molecular Cancer 18:Article number: 155 (2019); Darvin P. et al., Experimental & Molecular Medicine 50:1-11 (2018); Linhares A. et al., 9: Article 1909 (2018), all of which are herein incorporated in their entireties by reference. Other specific examples of the immune checkpoint receptor genes include B7 family genes and/or Ig superfamily genes.

“B7 family genes” refer to any genes that have the attributes of: (1) whose expression correlates with the increase in ICD, and (2) whose expression products are the B7 family of immune-regulatory ligands, including B7-1, B7-2, B7-H1, B7-DC, B7-H2, B7-H3 (also known as CD276), B7-H4 (also known as VTCN1), B7-H5, BTNL2, B7-H6, and B7-H7, for example as described in Yongbo Zhao et al., Frontiers in Immunology, 11: Article 458 (2020); Mary Collins et al., Genome Biol. 6(6): 223 (2005), both of which are herein incorporated in their entireties by reference. The term “B7 family genes” includes all natural gene variants for the B7 family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the B7 family genes also encompass genes of any vertebrate source, including mammals such as primates (e.g., humans and cynomolgus monkeys (cynos)), dogs, and rodents (e.g., mice and rats), unless otherwise indicated. Other specific examples of the B7 family genes include VTCN1 and/or CD276.

“Ig superfamily genes” refer to any genes that have the attributes of: (1) whose expression correlates with the increase in ICD, and (2) whose expression products have a domain known as an immunoglobulin domain or immunoglobulin fold, which is a shared structural features with immunoglobulins (also known as antibodies). The term “Ig superfamily genes” includes all natural gene variants for the Ig superfamily genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the Ig superfamily genes also encompass genes of any vertebrate source, including mammals such as primates (e.g., humans and cynomolgus monkeys (cynos)), dogs, and rodents (e.g., mice and rats), unless otherwise indicated. Other specific examples of the Ig superfamily genes include nectin family genes and/or LAG3.

“Nectin family genes” refer to any genes that have the attributes of: (1) whose expression correlates with the increase in ICD, and (2) whose expression products consist of a family of immunoglobulin superfamily members (nectin-1, nectin-2 (also known as PVRL2), nectin-3, nectin-4, NECL-1, NECL-2, NECL-3, NECL-4, NECL-5, as described in Yoshimi Takai et al., Nature Reviews Molecular Cell Biology 9: 603-615(2008), herein incorporated in its entirety by reference) and their binding receptors/ligands (such as PVRIG and TIGIT, as described in Beatriz Sanchez-Correa et al., Cancers (Basel). 11(6): 877 (2019 June)). The term “nectin family genes” includes all natural gene variants for the nectin family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the nectin family genes also encompass genes of any vertebrate source, including mammals such as primates (e.g., humans and cynomolgus monkeys (cynos)), dogs, and rodents (e.g., mice and rats), unless otherwise indicated. Other specific examples of the nectin family genes include PVRIG, PVRL2, and/or TIGIT.

“Receptor tyrosin kinase genes” refer to any genes that have the attributes of: (1) whose expression correlates with the increase in ICD, and (2) whose expression products are a family of cell-surface receptors transducing signals across the cell membrane and share the common structure feature of an extracellular ligand binding domain, a single transmembrane helix, a cytoplasmic region containing the protein tyrosine kinase activity (occasionally split into two domains by an insertion, termed the kinase insertion). The term “receptor tyrosin kinase genes” includes all natural gene variants for the receptor tyrosin kinase genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the receptor tyrosin kinase genes also encompass genes of any vertebrate source, including mammals such as primates (e.g., humans and cynomolgus monkeys (cynos)), dogs, and rodents (e.g., mice and rats), unless otherwise indicated. Other specific examples of the receptor tyrosin kinase genes include CSF1R, PDGFRB, TEK/TIE2, and/or FLT3.

“TNF family receptor genes” refer to any genes that have the attributes of: (1) whose expression correlates with the increase in ICD, and (2) whose expression products are type 1 transmembrane proteins that adopt elongated structures by a scaffold of disulfide bridges, which are about 40 amino acid pseudorepeats (“cysteine-rich domains”) typically defined by 3 intrachain disulfides among 6 highly conserved cysteines and are the hallmark of the TNFR superfamily. The term “TNF family receptor genes” includes all natural gene variants for the TNF family receptor genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the TNF family receptor genes also encompass genes of any vertebrate source, including mammals such as primates (e.g., humans and cynomolgus monkeys (cynos)), dogs, and rodents (e.g., mice and rats), unless otherwise indicated. Additional examples of TNF family receptor genes include those described in Richard M. Locksley et al., Cell 104(4):487-501 (2001); and Thomas Hehlgans and Klaus Pfeffer Immunology. 115(1): 1-20 (2005 May), both of which are herein incorporated in their entireties by reference. Other specific examples of the TNF family receptor genes include CD40, TNFRSF1A, TNFRSF21, and/or TNFRSF1B.

“IFN receptor family genes” refer to any genes that have the attributes of: (1) whose expression correlates with the increase in ICD, and (2) whose expression products are receptors for interferons (IFNs), including for example receptors for type I (α, β κ and ω), type II (γ), and type III (λ) interferons. The term “IFN receptor family genes” includes all natural gene variants for the IFN receptor family genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the IFN receptor family genes also encompass genes of any vertebrate source, including mammals such as primates (e.g., humans and cynomolgus monkeys (cynos)), dogs, and rodents (e.g., mice and rats), unless otherwise indicated. Additional examples of IFN receptor family genes include those described in Jacob Piehler et al Immunol Rev. 250(1): 317-334 (2012 November); Daniel S. Green et al., The Journal of Biological Chemistry, 292: 13925-13933 (2017); and Nicole A. de Weerd, et al., The Journal of Biological Chemistry 282, 20053-20057 (2007), all of which are herein incorporated in their entireties by reference. Other specific examples of the IFN receptor family genes include IFNAR1 and/or IFNAR2.

“Inhibitory immunoreceptor genes” refer to any genes that have the attributes of: (1) whose expression correlates with the increase in ICD, and (2) whose expression products function as immune-checkpoint molecule, e.g. suppresses T-cell activation or other immune response. The term “inhibitory immunoreceptor genes” includes all natural gene variants for the inhibitory immunoreceptor genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the inhibitory immunoreceptor genes also encompass genes of any vertebrate source, including mammals such as primates (e.g., humans and cynomolgus monkeys (cynos)), dogs, and rodents (e.g., mice and rats), unless otherwise indicated. Additional examples of inhibitory immunoreceptor genes include those described in Annika De Sousa Linhares et al Front. Immunol. 9: Article 1909 (31 Aug. 2018); Shiang Qin et al., Molecular Cancer 18: Article 155 (2019), all of which are herein incorporated in their entireties by reference. Other specific examples of the inhibitory immunoreceptor genes include TIM3 and/or VSIR.

“Metabolic enzyme genes” refer to any genes that have the attributes of: (1) whose expression correlates with the increase in ICD, and (2) whose expression products are enzymes in the metabolic pathways in a cell or an organism. The term “metabolic enzyme genes” includes all natural gene variants for the metabolic enzyme genes described herein, including polymorphic variants or allelic variants, (e.g., SNP variants); recombination variants; truncation variants; intron- or exon-skipping variants; intro- or exon-deletion variants; insertional variants (e.g. the insertion of one or more nucleotide or the insertion of a transposable genetic element); splice variants; fragments; and derivatives. Examples of the metabolic enzyme genes also encompass genes of any vertebrate source, including mammals such as primates (e.g., humans and cynomolgus monkeys (cynos)), dogs, and rodents (e.g., mice and rats), unless otherwise indicated. Additional examples of metabolic enzyme genes include those described in Natalya N. Pavlova et al Cell Metab 23(1): 27-47 (2016 January); Metabolism of Cancer Cells and Immune Cells in the Tumor Microenvironment, edited by Yongsheng Li and Bo Zhu, Frontiers in Immunology (a collection of related articles published from 2017 to 2019), all of which are herein incorporated in their entireties by reference. Other specific examples of the metabolic enzyme genes include IDO1, TDO2, EIF2AK2, ACSS1, and ACSS2.

The types of genes provided herein are not mutually exclusive, some genes can belong to more than types.

As used herein, the term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies can also be conjugated to an anti-cancer pro-drug activating enzyme capable of converting the pro-drug to an active form of any of the cytotoxic agent described herein.

As used herein, the term “further conditioned on,” when used in connection between a method step and a condition, is intended to mean that the condition must be satisfied before the method step can be carried out. For example, the expression “step A is further conditioned on condition C” would mean that condition C must occur or be satisfied before step A can be carried out. If execution of step A already requires condition B, then the expression “step A is further conditioned on condition C” would mean that both conditions B and C must occur or be satisfied, before step A can be carried out.

As used herein, the term “ARHGEF18” refers to “Rho/Rac Guanine Nucleotide Exchange Factor 18,” also known as “Rho Guanine Nucleotide Exchange Factor (GEF) 18,” “Septin-associated RhoGEF,” or “114 KDa Rho-Specific Guanine Nucleotide Exchange Factor,” in Uniprot or GenBank database. The term “ARHGEF18” encompasses the ARHGEF18 polypeptides, the ARHGEF18 RNA transcripts, and the ARHGEF18 genes. The term “ARHGEF18 gene” refers to genes encoding ARHGEF18 polypeptides. ARHGEF18 is expressed in various cells and tissues including the pancreas and kidney, among others. Examples of ARHGEF18 genes encompass any such native gene from any vertebrate source, including mammals such as primates (e.g., humans and chimpanzees), cows, dogs, and rodents (e.g. mice and rats), unless otherwise indicated. In certain embodiments, the ARHGEF18 gene includes all natural variants of ARHGEF18 gene, including allelic variants (e.g., SNP variants) and mutations. NCBI Reference Sequence NG_047135 provides an exemplary human ARHGEF18 nucleic acid sequence. In certain embodiments, ARHGEF18 gene expression is determined by the amounts of its mRNA transcripts. In certain embodiments, the mRNA transcripts are splice variants, fragments or derivatives of all native and natural variants of transcripts of the ARHGEF18 gene. NCBI Reference Sequences NM_001367823.1, NM_015318.4, NM_001130955.2, NM_001367824.1, XM_005272464.4, XM_006722706.3, XM_011527835.2, XM_011527836.2, XM_011527837.2, XM_011527838.3, XM_011527839.2, XM_011527840.2 and XM_011527841.2 provide exemplary human ARHGEF18 mRNA transcript sequences. The ARHGEF18 polypeptide acts as a guanine exchange factor (GEF) for Rho GTPases. The ARHGEF18 polypeptide plays a central role in the formation of actin stress fibers and other cytoskeletal rearrangements. Examples of ARHGEF18 polypeptides include any such native polypeptide from any vertebrate source as described above. In certain embodiments, ARHGEF18 gene expression is determined by the amounts of the ARHGEF18 polypeptides expressed from ARHGEF18 genes. In certain embodiments, the ARHGEF18 polypeptide includes all polypeptides encoded by the natural variants of the ARHGEF18 genes and transcripts thereof, including natural allelic variants (e.g., SNP variants); splice variants; fragments; and derivatives. The ARHGEF18 polypeptide of the present disclosure also encompasses “full-length,” unprocessed ARHGEF18 polypeptide as well as any form of ARHGEF18 polypeptide that results from processing in the cell. In some embodiments, NCBI Reference Sequences NP_001354752.1, NP_056133.2, NP_001124427.2, NP_001354753.1, XP_005272521.1, XP_006722769.1, XP_011526137.1, XP_011526138.1, XP_011526139.1, XP_011526140.1, XP_011526141.1, XP_011526142.1 and XP_011526143.1 provide exemplary ARHGEF18 polypeptide sequences. The database sequences referred to in this paragraph are all incorporated in their entirety by reference.

As used herein, the term “ARHGEF5” refers to “Rho Guanine Nucleotide Exchange Factor 5,” also known as “Transforming Immortalized Mammary Oncogene,” “Oncogene TIM,” “Ephexin-3,” “p60 TIM,” or “Guanine Nucleotide Regulatory Protein TIM,” in Uniprot or GenBank database. The term “ARHGEF5” encompasses the ARHGEF5 polypeptides, the ARHGEF5 RNA transcripts, and the ARHGEF5 genes. The term “ARHGEF5 gene” refers to genes encoding ARHGEF5 polypeptides. ARHGEF5 is expressed in various cells and tissues including liver, skin, and spleen, among others. Examples of ARHGEF5 gene encompass any such native gene from any vertebrate source, including mammals such as primates (e.g., humans and chimpanzees), cows, dogs, and rodents (e.g. mice and rats), unless otherwise indicated. In certain embodiments, the ARHGEF5 gene includes all natural variants of ARHGEF5 gene, including allelic variants (e.g., SNP variants) and mutations. NCBI Reference Sequence NC_000007.14 range 144355396 . . . 144380632 provides an exemplary human ARHGEF5 nucleic acid sequence. In certain embodiments, ARHGEF5 gene expression is determined by the amounts of the its mRNA transcripts. In certain embodiments, the mRNA transcripts include splice variants, fragments or derivatives of all native and natural variants of transcripts of the ARHGEF5 gene. NCBI Reference Sequences NM_005435.4 and XM_017012623.2 provide exemplary human ARHGEF5 mRNA transcript sequences. The ARHGEF5 polypeptide activates Rho GTPases and is involved in control of cytoskeletal organization. Examples of ARHGEF5 polypeptides include any such native polypeptide from any vertebrate source as described above. In certain embodiments, ARHGEF5 gene expression is determined by the amounts of ARHGEF5 polypeptides expressed from the ARHGEF5 gene. In certain embodiments, the ARHGEF5 polypeptide includes all polypeptides encoded by natural variants of the ARHGEF5 genes and transcripts thereof, including allelic variants (e.g., SNP variants); splice variants; fragments; and derivatives. ARHGEF5 polypeptide of the present disclosure also encompasses “full-length,” unprocessed ARHGEF5 polypeptide as well as any form of ARHGEF5 polypeptide that results from processing in the cell. In some embodiments, NCBI Reference Sequences NP_005426.2 and XP_016868112.1 provide exemplary human ARHGEF5 polypeptide sequences.

As used herein, the term “ATP2A3” refers to “ATPase Sarcoplasmic/Endoplasmic Reticulum Ca2+Transporting 3,” also known as “Sarcoplasmic/Endoplasmic Reticulum Calcium ATPase 3” or “Calcium Pump 3,” in Uniprot or GenBank database. The term “ATP2A3” encompasses the ATP2A3 polypeptides, the ATP2A3 RNA transcripts, and the ATP2A3 genes. The term “ATP2A3 gene” refers to genes encoding ATP2A3 polypeptides. ATP2A3 is expressed various cells and tissues including in blood, kidney, and heart among others. Examples of ATP2A3 genes encompass any such native gene from any vertebrate source, including mammals such as primates (e.g., humans and chimpanzees), cows, dogs, and rodents (e.g. mice and rats), unless otherwise indicated. In certain embodiments, the ATP2A3 genes include all natural variants of ATP2A3 genes, including allelic variants (e.g., SNP variants) and mutations. NCBI Reference Sequence NG_029041 provides an exemplary human ATP2A3 nucleic acid sequence. In certain embodiments, ATP2A3 gene expression is determined by the amounts of ATP2A3 mRNA transcripts. In certain embodiments, the mRNA transcripts are splice variants, fragments or derivatives of all native and natural variants of the transcripts of ATP2A3 genes. NCBI Reference Sequences NM_005173.4, NM_174953.3, NM_174954.3, NM_174955.3, NM_174956.2, NM_174957.3, NM_174958.3, XM_011523881.2, XM_011523882.2, XM_011523884.3, XM_011523885.1, XM_011523888.2, XM_011523889.1, XM_011523892.2, XM_017024692.1 and XM_017024693.2 provide exemplary human ATP2A3 mRNA transcript sequences. The ATP2A3 polypeptide catalyzes hydrolysis of ATP coupled with the transport of calcium. Examples of ATP2A3 polypeptides include any such native polypeptide from any vertebrate source as described above. In certain embodiments, ATP2A3 gene expression is determined by the amounts of ATP2A3 polypeptides expressed from the ATP2A3 gene. In certain embodiments, the ATP2A3 polypeptide includes all polypeptides encoded by the natural variants of ATP2A3 genes and transcripts thereof, including allelic variants (e.g., SNP variants); splice variants; fragments; and derivatives. ATP2A3 polypeptide of the present disclosure also encompasses “full-length,” unprocessed ATP2A3 polypeptide as well as any form of ATP2A3 polypeptide that results from processing in the cell. In some embodiments, NCBI Reference Sequences NP_005164.2, NP_777613.1, NP_777614.1, NP_777615.1, NP_777616.1, NP_777617.1, NP_777618.1, XP_011522183.1, XP_011522184.1, XP_011522186.1, XP_011522187.1, XP_011522190.1, XP_011522191.1, XP_011522194.1, XP_016880181.1 and XP_016880182.1 provide exemplary human ATP2A3 polypeptide sequences.

As used herein, the term “Bax” refers to “BCL2 Associated X, Apoptosis Regulator,” also known as“BCL2 Associated X Protein, Regulatory Subunit 52” or “Apoptosis Regulator BAX,” in Uniprot or GenBank database. Bax is expressed in various cells and tissues including blood, bone marrow, nervous system, among others. The term “Bax” encompasses the Bax polypeptides, the Bax RNA transcripts, and the Bax genes. The term “Bax gene” refers to genes encoding Bax polypeptides. Examples of Bax encompass any such native genes from any vertebrate source, including mammals such as primates (e.g., humans and chimpanzees), cows, dogs, and rodents (e.g. mice and rats), unless otherwise indicated. In certain embodiments, the term “Bax gene” includes all natural variants of Bax genes, including allelic variants (e.g., SNP variants) and mutations. NCBI Reference Sequence NG_012191 provides an exemplary human Bax nucleic acid sequence. In certain embodiments, Bax gene expression is determined by the amounts of mRNA transcripts. In certain embodiments, the mRNA transcripts are splice variants, fragments or derivatives of all native and natural variants of the transcripts of Bax gene. NCBI Reference Sequences NM_138761.4, NM_004324.4, NM_138763.4, NM_138764.5, NM_001291428.2, NM_001291429.2, NM_001291430.1, NM_001291431.2, NR 027882.2 and XM_017027077.1 provide exemplary human Bax mRNA transcript sequences. The Bax polypeptide is involved in mitochondrial apoptosis. Under stress conditions, Bax undergoes a conformational change, causing the mitochondrial membrane to translocate and release cytochrome c and caspase 3 activation. Examples of Bax polypeptides include any such native polypeptide from any vertebrate source as described above, unless otherwise indicated. In certain embodiments, Bax gene expression is determined by the amounts of Bax polypeptides expressed from the Bax genes. In certain embodiments, a Bax polypeptide includes all polypeptides encoded by the natural variants of Bax genes and transcripts thereof, including allelic variants (e.g., SNP variants); splice variants; fragments; and derivatives. A Bax polypeptide of the present disclosure also encompasses “full-length,” unprocessed Bax polypeptide as well as any form of Bax polypeptide that results from processing in the cell. In some embodiments, NCBI Reference Sequences NP_612815.1, NP_001182.1, NP_001304848.1, NP_001304849.1, NP_001304850.1, NP_001309168.1, NP_001309169.1, NP_001309171.1 and XP_011527266.1 provide exemplary human Bax polypeptide sequences.

As used herein, the term “BCL2L1” refers to “BCL2 Like 1,” also known as “Protein Phosphatase 1, Regulatory Subunit 52” or “Apoptosis Regulator Bcl-X,” in Uniprot or GenBank database. The term “BCL2L1” encompasses the BCL2L1 polypeptides, the BCL2L1 RNA transcripts, and the BCL2L1 genes. BCL2L1 is expressed in various cells and tissues including blood, bone marrow, lymph node, spleen and thyroid, among others. The term “BCL2L1 gene” refers to genes encoding BCL2L1 polypeptides. Examples of BCL2L1 genes encompass any such native gene from any vertebrate source, including mammals such as primates (e.g., humans and chimpanzees), cows, dogs, and rodents (e.g. mice and rats), unless otherwise indicated. In certain embodiments, BCL2L1 genes include all natural variants of BCL2L1, including allelic variants (e.g., SNP variants) and mutations. NCBI Reference Sequence NG_029002 provides an exemplary human BCL2L1 nucleic acid sequence. In certain embodiments, BCL2L1 gene expression is determined by the amounts of the mRNA transcripts. In certain embodiments, the mRNA transcripts are splice variants, fragments or derivatives of all native and natural variants of the transcripts of BCL2L1 genes. NCBI Reference Sequences NM_138578.3, NM_001191.4, NM_001317919.2, NM_001317920.2, NM_001317921.2, NM_001322239.2, NM_001322240.2, NM_001322242.2, NR 134257.1, XM_011528964.2, XM_017027993.1, XR_936599.3 and XR_001754364.2 provide exemplary human BCL2L1 mRNA transcript sequences. The BCL2L1 polypeptide forms hetero- or homodimers to act as anti- or pro-apoptotic regulators. The BCL2L1 polypeptide is located at the outer mitochondrial membrane and also acts as a regulator of G2 checkpoint and progression during mitosis. Examples of BCL2L1 polypeptides include any such native polypeptide from any vertebrate source as described above. In certain embodiments, BCL2L1 gene expression is determined by the amounts of the BCL2L1 polypeptides expressed from the BCL2L1 genes. In certain embodiments, the BCL2L1 polypeptide includes all polypeptides encoded by the natural variants of BCL2L1 genes and transcripts thereof, including allelic variants (e.g., SNP variants); splice variants; fragments; and derivatives. The BCL2L1 polypeptide of the present disclosure also encompasses “full-length,” unprocessed BCL2L1 polypeptide as well as any form of BCL2L1 polypeptide that results from processing in the cell. In some embodiments, NCBI Reference Sequences NP_612815.1, NP_001182.1, NP_001304848.1, NP_001304849.1, NP_001304850.1, NP_001309168.1, NP_001309169.1, NP_001309171.1, XP_011527266.1 and XP_016883482.1 provide exemplary human BCL2L1 polypeptide sequences.

As used herein, the term “CCND1” refers to “Cyclin D1,” also known as “B-Cell Lymphoma 1 Protein” or “G1/S-Specific Cyclin-D1,” in Uniprot or GenBank database. The term “CCND1” encompasses the CCND1 polypeptides, the CCND1 RNA transcripts, and the CCND1 genes. CCND1 is expressed in various cells and tissues including the thyroid gland, lymph node, blood and bone marrow, among others. The term “CCND1 gene” refers to genes encoding CCND1 polypeptides. Examples of CCND1 genes encompass any such native gene from any vertebrate source, including mammals such as primates (e.g., humans and chimpanzees), cows, dogs, and rodents (e.g. mice and rats), unless otherwise indicated. In certain embodiments, the CCND1 genes include all natural variants of CCND1, including allelic variants (e.g., SNP variants) and mutations. NCBI Reference Sequence NG_007375 provides an exemplary human CCND1 nucleic acid sequence. In certain embodiments, CCND1 gene expression is determined by the amounts of mRNA transcripts of CCND1. In certain embodiments, the mRNA transcripts are splice variants, fragments or derivatives of all native and natural variants of the transcripts of the CCND1 genes. NCBI Reference Sequence NM_053056.3 provides an exemplary human CCND1 mRNA transcript sequence. The CCND1 polypeptide functions as cell cycle regulator during G(1)/S transition. Examples of CCND1 polypeptides include any such native polypeptide from any vertebrate source as described above, unless otherwise indicated. In certain embodiments, CCND1 gene expression is determined by the amounts of the CCND1 polypeptides expressed from the CCND1 gene. In certain embodiments, the CCND1 polypeptide includes all polypeptides encoded by the natural variants of CCND1 genes and transcripts thereof, including allelic variants (e.g., SNP variants); splice variants; fragments; and derivatives. The CCND1 polypeptide of the present disclosure also encompasses “full-length,” unprocessed CCND1 polypeptide as well as any form of CCND1 polypeptide that results from processing in the cell. In some embodiments, NCBI Reference Sequence NP_444284.1 provides an exemplary human CCND1 polypeptide sequence.

As used herein, the term “c-JUN” or “c-Jun” refers to “Jun Proto-Oncogene, AP-1 Transcription Factor Subunit,” also known as “V-Jun Avian Sarcoma Virus 17 Oncogene Homolog” or “Transcription Factor AP-1,” in Uniprot or GenBank database. The term “c-JUN” encompasses the c-JUN polypeptides, the c-JUN RNA transcripts, and the c-JUN genes. C-JUN is expressed in various cells and tissues including the thyroid gland, lymph node, blood and bone marrow, among others. The term “c-JUN gene” refers to genes encoding c-JUN polypeptides. Examples of c-JUN genes encompass any such native gene from any vertebrate source, including mammals such as primates (e.g., humans and chimpanzees), cows, and rodents (e.g. mice and rats), unless otherwise indicated. In certain embodiments, the c-JUN genes include all natural variants of c-JUN, including allelic variants (e.g., SNP variants) and mutations. NCBI Reference Sequence NG_047027 provides an exemplary human c-JUN nucleic acid sequence. In certain embodiments, c-JUN gene expression is determined by the amounts of the mRNA transcripts. In certain embodiments, the mRNA transcripts are splice variants, fragments or derivatives of all transcripts of the native and natural variants of c-JUN genes. NCBI Reference Sequence NM_002228.4 provides an exemplary human c-JUN mRNA transcript sequence. The c-JUN polypeptide functions as a transcription factor, binding to DNA sequences to regulate expression of the target gene. Examples of c-JUN polypeptides include any such native polypeptide from any vertebrate source as described above. In certain embodiments, c-JUN gene expression is determined by the amounts of the c-JUN polypeptides expressed from the c-JUN genes. In certain embodiments, the c-JUN polypeptide includes all polypeptides encoded by the natural variants of c-JUN genes and transcripts thereof, including allelic variants (e.g., SNP variants); splice variants; fragments; and derivatives. The c-JUN polypeptide of the present disclosure also encompasses “full-length,” unprocessed c-JUN polypeptide as well as any form of c-JUN polypeptide that results from processing in the cell. In some embodiments, NCBI Reference Sequence NP_002219.1 provides an exemplary human c-JUN polypeptide sequence.

As used herein, the terms “DAB2IP” and “DAP2IP” are used interchangeably to refer to “DAB2 Interacting Protein,” also known as “Disabled Homolog 2-Interacting Protein” or “DOC-2/DAB2 Interactive Protein,” in Uniprot or GenBank database. The term “DAB2IP” encompasses the DAB2IP polypeptides, the DAB2IP RNA transcripts, and the DAB2IP genes. DAB2IP is expressed in various cells and tissues including endothelial and vascular smooth muscle cells, among others. The term “DAB2IP gene” refers to genes encoding DAB2IP polypeptides. Examples of DAB2IP genes encompass any such native genes from any vertebrate source, including mammals such as primates (e.g., humans and chimpanzees), cows, and rodents (e.g. mice and rats), unless otherwise indicated. In certain embodiments, the DAB2IP genes include all natural variants of DAB2IP, including allelic variants (e.g., SNP variants) and mutations. NCBI Reference Sequence NC_000009.12 range 121566883 . . . 121785530 provides an exemplary human DAB2IP nucleic acid sequence. In certain embodiments, DAB2IP gene expression is determined by the amounts of the mRNA transcripts. In certain embodiments, the mRNA transcripts are splice variants, fragments or derivatives of all native and natural variants of the transcripts of the DAB2IP genes. NCBI Reference Sequences NM_032552.3, NM_138709.2, XM_005251719.4, XM_005251721.1, XM_011518264.3, XM_011518265.3, XM_011518266.2, XM_011518267.2, XM_011518270.2, XM_011518271.2, XM_017014298.2, XM_017014299.1, XM_017014300.1, XM_024447417.1 and XM_024447418.1 provide exemplary human DAB2IP mRNA transcript sequences. The DAB2IP polypeptide functions as a scaffold protein to promote signaling pathways. Such signaling pathways include those involved in the innate immune response, inflammation, cell growth inhibition, apoptosis, cell survival, angiogenesis, cell migration and maturation. Examples of DAB2IP polypeptides include any such native polypeptide from any vertebrate source as described above. In certain embodiments, DAB2IP gene expression is determined by the amounts of the DAB2IP polypeptides expressed from the DAB2IP genes. In certain embodiments, the DAB2IP polypeptide includes all polypeptides encoded by the natural variants of DAB2IP genes and transcripts thereof, including allelic variants (e.g., SNP variants); splice variants; fragments; and derivatives. The DAB2IP polypeptide of the present disclosure also encompasses “full-length,” unprocessed DAB2IP polypeptide as well as any form of DAB2IP polypeptide that results from processing in the cell. In some embodiments, NCBI Reference Sequences NP_115941.2, NP_619723.1, XP_005251776.1, XP_005251778.1, XP_011516566.1, XP_011516567.1, XP_011516568.1, XP_011516569.1, XP_011516572.1, XP_011516573.1, XP_016869787.1, XP_016869788.1, XP_016869789.1, XP_024303185.1 and XP_024303186.1 provide exemplary human DAB2IP polypeptide sequences.

As used herein, the terms “Eotaxin,” “CCL11,” and “Eotaxin (CCL11)” are used interchangeably to refer to “C—C Motif Chemokine Ligand 11,” also known as “Small Inducible Cytokine Subfamily A (Cys-Cys), Member 11 (Eotaxin)” or “Chemokine (C—C Motif) Ligand 11,” in Uniprot or GenBank database. The term “CCL11” encompasses the CCL11 polypeptides, the CCL11 RNA transcripts, and the CCL11 genes. The term “CCL11 gene” refers to genes encoding CCL11 polypeptides. CCL11 is expressed in various cells and tissues including lung, intestine, blood and skin, among others. Examples of CCL11 genes encompass any such native genes from any vertebrate source, including mammals such as primates (e.g., humans and chimpanzees), cows, and rodents (e.g. mice and rats), unless otherwise indicated. In certain embodiments, the CCL11 genes include all natural variants of CCL11, including allelic variants (e.g., SNP variants) and mutations. NCBI Reference Sequence NG_012212 provides an exemplary human CCL11 nucleic acid sequence. In certain embodiments, CCL11 gene expression is determined by the amounts of the mRNA transcripts. In certain embodiments, the mRNA transcripts are splice variants, fragments or derivatives of all native and natural variants of the transcripts of the CCL11 genes. NCBI Reference Sequence NM_002986 provides an exemplary human CCL11 mRNA transcript sequence. The CCL11 polypeptide is a chemokine that participates in immunoregulatory and inflammatory processes. The CCL11 polypeptide displays chemotactic activity for eosinophils. Examples of CCL11 polypeptides include any such native polypeptide from any vertebrate source as described above. In certain embodiments, CCL11 gene expression is determined by the amounts of the CCL11 polypeptides expressed from the CCL11 genes. In certain embodiments, the CCL11 polypeptide includes all polypeptides encoded by the natural variants of the CCL11 genes and transcripts thereof, including allelic variants (e.g., SNP variants); splice variants; fragments; and derivatives. The CCL11 polypeptide of the present disclosure also encompasses “full-length,” unprocessed CCL11 polypeptide as well as any form of the CCL11 polypeptide that results from processing in the cell. In some embodiments, NCBI Reference Sequence NP_002977 provides an exemplary human CCL11 polypeptide sequence.

As used herein, the term “ERP29” refers to “Endoplasmic Reticulum Protein 29,” also known as “Protein Disulfide Isomerase Family A, Member 9” or “Endoplasmic Reticulum Resident Protein 28,” in Uniprot or GenBank database. The term “ERP29” encompasses the ERP29 polypeptides, the ERP29 RNA transcripts, and the ERP29 genes. The term “ERP29 gene” refers to genes encoding ERP29 polypeptides. ERP29 is expressed in various cells and tissues including the lymph node, thyroid gland, spleen, blood, among others. Examples of ERP29 genes encompass any such native genes from any vertebrate source, including mammals such as primates (e.g., humans and chimpanzees), cows, dogs and rodents (e.g. mice and rats), unless otherwise indicated. In certain embodiments, the term “ERP29 gene” include all natural variants of ERP29 genes, including allelic variants (e.g., SNP variants) and mutations. NCBI Reference Sequence NC_000012.12 range 112013340 . . . 112023449 provides an exemplary human ERP29 nucleic acid sequence. In certain embodiments, ERP29 gene expression is determined by the amounts of the mRNA transcripts. In certain embodiments, the mRNA transcripts are splice variants, fragments or derivatives of all native and natural variants of the transcripts of the ERP29 genes. NCBI Reference Sequences NM_006817.4, NM_001034025.1 and XM_017018720.1 provide exemplary human ERP29 mRNA transcript sequences. The ERP29 polypeptide is a member of the disulfide isomerase (PDI) protein family although it lacks an active motif. It primarily functions by localizing the to the endoplasmic reticulum lumen, where it processes and folds secretory proteins. Examples of ERP29 polypeptides include any such native polypeptides from any vertebrate source as described above. In certain embodiments, ERP29 gene expression is determined by the amounts of the ERP29 polypeptides expressed from the ERP29 genes. In certain embodiments, an ERP29 polypeptide includes all polypeptides encoded by natural variants of ERP29 genes and transcripts thereof, including allelic variants (e.g., SNP variants); splice variants; fragments; and derivatives. The ERP29 polypeptide of the present disclosure also encompasses “full-length,” unprocessed ERP29 polypeptide as well as any form of ERP29 polypeptide that results from processing in the cell. In some embodiments, NCBI Reference Sequences NP_006808.1, NP_001029197.1 and XP_016874209.1 provide exemplary human ERP29 polypeptide sequences.

As used herein, the term “HLA-A” refers to “human leukocyte antigen-A,” also known as “major histocompatibility complex, class I, A” or “leukocyte antigen class I A,” and belongs to the HLA Class I heavy chain paralogues. The term “HLA-A” encompasses the HLA-A polypeptides, the HLA-A RNA transcripts, and the HLA-A genes. The term “HLA-A gene” refers to genes encoding HLA-A polypeptides. HLA-A is expressed in almost all cells including bone marrow-derived stem cells, among others. In certain embodiments, the HLA-A gene contains 8 exons with sequence variations in the exons 2 and 3 that dictate peptide binding specificity. Examples of HLA-A genes encompass any such native gene in human. In certain embodiments, the HLA-A genes include all natural variants of HLA-A, including allelic variants (e.g., SNP variants) and mutations. NCBI Reference Sequence NG_029217.2 provides an exemplary human HLA-A nucleic acid sequence. In certain embodiments, HLA-A expression is determined by the amount of the mRNA transcripts. In certain embodiments, the mRNA transcripts are splice variants, fragments or derivatives of all native and natural variants of HLA-A. NCBI Reference Sequences NM_001242758.1 and NM_002116.8 provide exemplary human HLA-A mRNA transcript sequences. The HLA-A polypeptide is involved in the immune system and functions by presenting antigens to immune cells. In certain embodiments, the HLA-A expression is determined by the amounts of the HLA-A polypeptides expressed from the HLA-A genes. Examples of HLA-A polypeptides include any such native polypeptides in human. In certain embodiments, the HLA-A polypeptide includes all polypeptides encoded by natural variants of the HLA-A genes and transcripts thereof, including allelic variants (e.g., SNP variants); splice variants; fragments; and derivatives. The HLA-A polypeptide of the present disclosure also encompasses “full-length,” unprocessed HLA-A polypeptide as well as any form of HLA-A polypeptide that results from processing in the cell. In some embodiments, NCBI Reference Sequences NP_001229687.1 and NP_002107.3 provide exemplary human HLA-A polypeptide sequences.

As used herein, the term “HLA-B” refers to “Major Histocompatibility Complex, Class I, B,” also known as “HLA Class I Histocompatibility Antigen, B Alpha Chain” or “HLAB,” and belongs to the HLA Class I heavy chain paralogues. The term “HLA-B” encompasses the HLA-B polypeptides, the HLA-B RNA transcripts, and the HLA-B genes. The term “HLA-B gene” refers to genes encoding HLA-B polypeptides. HLA-B is expressed in various cells and tissues including T Helper cells and thymocytes, among others. Examples of HLA-B genes encompass any such native genes in human. In certain embodiments, the HLA-B genes include all natural variants of HLA-B, including allelic variants (e.g., SNP variants) and mutations. NCBI Reference Sequence NG_023187 provides an exemplary human HLA-B nucleic acid sequence. In certain embodiments, HLA-B expression is determined by the amounts of the mRNA transcripts. In certain embodiments, the mRNA transcripts include splice variants, fragments or derivatives of all native and natural variants of HLA-B. NCBI Reference Sequence NM_005514.8 provides an exemplary human HLA-B mRNA transcript sequence. The HLA-B polypeptide is involved in the immune system and functions by presenting antigens to immune cells. In certain embodiments, the HLA-B expression is determined by the amounts of the HLA-B polypeptides expressed from the HLA-B genes. Examples of the HLA-B polypeptides include any such native polypeptide in human. In certain embodiments, the HLA-B polypeptides include all polypeptides encoded by the natural variants of HLA-B genes and transcripts thereof, including allelic variants (e.g., SNP variants); splice variants; fragments; and derivatives. The HLA-B polypeptide of the present disclosure also encompasses “full-length,” unprocessed HLA-B polypeptide as well as any form of HLA-B polypeptide that results from processing in the cell. In some embodiments, NCBI Reference Sequence NP_005505.2 provides an exemplary human HLA-B polypeptide sequence.

As used herein, the term “HLA-C” refers to “Major Histocompatibility Complex, Class I, C,” also known as “HLA Class I Histocompatibility Antigen, C Alpha Chain” or “HLAC,” and belongs to the HLA Class I heavy chain paralogues. The term “HLA-C” encompasses the HLA-C polypeptides, the HLA-C RNA transcripts, and the HLA-C genes. The term “HLA-C gene” refers to genes encoding HLA-C polypeptides. HLA-C is expressed in various cells and tissues including granulocytes and thymocytes, among others. Examples of HLA-C genes encompass any such native gene in human. In certain embodiments, the HLA-C genes include all natural variants of HLA-C, including allelic variants (e.g., SNP variants) and mutations. NCBI Reference Sequence NG_029422 provides an exemplary human HLA-C nucleic acid sequence. In certain embodiments, HLA-C expression is determined by the amount of the mRNA transcripts. In certain embodiments, the mRNA transcripts include splice variants, fragments or derivatives of all native and natural variants of the transcripts of the HLA-C genes. NCBI Reference Sequences NM_001243042.1 and NM_002117.6 provide exemplary human HLA-C mRNA transcript sequences. The HLA-C polypeptide is involved in the immune system and functions by presenting antigens to immune cells. In certain embodiments, the HLA-C expression is determined by the amounts of the HLA-C polypeptides expressed from the HLA-C genes. Examples of HLA-C polypeptides include any such native polypeptides in human. In certain embodiments, the HLA-C polypeptides include all polypeptides encoded by the natural variants of HLA-C genes and transcripts thereof, including allelic variants (e.g., SNP variants); splice variants; fragments; and derivatives. An HLA-C polypeptide of the present disclosure also encompasses “full-length,” unprocessed HLA-C polypeptide as well as any form of HLA-C polypeptide that results from processing in the cell. In some embodiments, NCBI Reference Sequences NP_001229971.1 and NP_002108.4 provide exemplary human HLA-C polypeptide sequences.

As used herein, the term “HLA-E” refers to “Major Histocompatibility Complex, Class I, E,” also known as “HLA Class I Histocompatibility Antigen, Alpha Chain E” or “MHC Class I Antigen E,” and belongs to the HLA Class I heavy chain paralogues. The term “HLA-E” encompasses the HLA-E polypeptides, the HLA-E RNA transcripts, and the HLA-E genes. The term “HLA-E gene” refers to genes encoding HLA-E polypeptides. HLA-E is expressed in various cells and tissues including T Helper cells and thymocytes, among others. Examples of HLA-E genes encompass any such native gene in human. In certain embodiments, the HLA-E genes include all natural variants of HLA-E genes, including allelic variants (e.g., SNP variants) and mutations. NCBI Reference Sequence NC_000006.12 range 30489508 . . . 30494194 provides an exemplary human HLA-E nucleic acid sequence. In certain embodiments, HLA-E expression is determined by the amounts of the mRNA transcripts. In certain embodiments, the mRNA transcripts include splice variants, fragments or derivatives of all native and natural variants of HLA-E. NCBI Reference Sequences NM_005516.6, XM_017010807.1, XM_017010808.1 and XM_017010809.2 provide exemplary human HLA-E mRNA transcript sequences. The HLA-E polypeptide is involved in immune self-nonself discrimination. In certain embodiments, the HLA-E expression is determined by the amounts of the HLA-E polypeptides expressed from the HLA-E genes. Examples of HLA-E polypeptides include any such native polypeptides in human. In certain embodiments, the HLA-E polypeptides include all polypeptides encoded by the natural variants of HLA-E genes and transcripts thereof, including allelic variants (e.g., SNP variants); splice variants; fragments; and derivatives. The HLA-E polypeptides of the present disclosure also encompass “full-length,” unprocessed HLA-E polypeptide as well as any form of HLA-E polypeptide that results from processing in the cell. In some embodiments, NCBI Reference Sequences NP_005507.3, XP_016866296.1, XP_016866297.1 and XP_016866298.1 provide exemplary human HLA-E polypeptide sequences.

As used herein, the term “HLA-F” refers to “Major Histocompatibility Complex, Class I, F,” also known as “HLA Class I Histocompatibility Antigen, Alpha Chain F” or “MHC Class I Antigen F,” and belongs to the HLA Class I heavy chain paralogues. The term “HLA-F” encompasses the HLA-F polypeptides, the HLA-F RNA transcripts, and the HLA-F genes. The term “HLA-F gene” refers to genes encoding HLA-F polypeptides. HLA-F is expressed in various cells and tissues including thymocytes and CD8 T cells, among others. Examples of HLA-F genes encompass any such native genes in human. In certain embodiments, the term includes all natural variants of HLA-F genes, including allelic variants (e.g., SNP variants) and mutations. NCBI Reference Sequence NG_012009 provides an exemplary human HLA-F nucleic acid sequence. In certain embodiments, HLA-F expression is determined by the amounts of the mRNA transcripts. In certain embodiments, the mRNA transcripts are splice variants, fragments or derivatives of all native and natural variants of the transcripts of the HLA-F genes. NCBI Reference Sequences NM_001098479.2, NM_018950.2, NM_001098478.2, XM_011514564.1, XM_017010810.1, XM_017010811.1, XM_017010813.1, XM_017010814.1, XM_017010815.1, XR_001743373.1, XR_001743374.1 and XR_001743376.1 provide exemplary human HLA-F mRNA transcript sequences. The HLA-F polypeptide is involved in immune surveillance, immune tolerance and inflammation. In certain embodiments, the HLA-F expression is determined by the amounts of the HLA-F polypeptides expressed from the HLA-F genes. Examples of HLA-F polypeptides include any such native polypeptides in human. In certain embodiments, the HLA-F polypeptide includes all polypeptides encoded by the natural variants of HLA-F genes and transcripts thereof, including allelic variants (e.g., SNP variants); splice variants; fragments; and derivatives. The HLA-F polypeptides of the present disclosure also encompass “full-length,” unprocessed HLA-F polypeptide as well as any form of HLA-F polypeptide that results from processing in the cell. In some embodiments, NCBI Reference Sequences NP_001091949.1, NP_061823.2, NP_001091948.1, XP_011512866.1, XP_016866299.1, XP_016866300.1, XP_016866301.1, XP_016866302.1, XP_016866303.1 and XP_016866304.1 provide exemplary human HLA-F polypeptide sequences.

As used herein, the term “HLA-DMA” refers to “Major Histocompatibility Complex, Class II, DM Alpha,” also known as “HLA Class II Histocompatibility Antigen, DM Alpha Chain” or “Really Interesting New Gene 6 Protein,” and belongs to the HLA Class II alpha chain paralogues. The term “HLA-DMA” encompasses the HLA-DMA polypeptides, the HLA-DMA RNA transcripts, and the HLA-DMA genes. The term “HLA-DMA gene” refers to genes encoding HLA-DMA polypeptides. HLA-DMA is expressed in various cells and tissues including intracellular vesicles, among others. Examples of HLA-DMA genes encompass any such native genes in human. In certain embodiments, the term includes all natural variants of HLA-DMA genes, including allelic variants (e.g., SNP variants) and mutations. NCBI Reference Sequence NG_012006 and GenBank Gene ID: 3108 provide exemplary human HLA-DMA nucleic acid sequences. In certain embodiments, HLA-DMA gene expression is determined by the amounts of the mRNA transcripts. In certain embodiments, the mRNA transcripts are splice variants, fragments or derivatives of all native and natural variants of the transcripts of the HLA-DMA genes. NCBI Reference Sequence NM_006120.4 provides an exemplary human HLA-DMA mRNA transcript sequence. The HLA-DMA polypeptide a transmembrane polypeptide forming a heterodimer with a beta chain (DMB). The HLA-DMA is involved in peptide loading of MHC by catalyzing the release of class II-associated invariant chain peptide (CLIP). Examples of the HLA-DMA polypeptides include any such native polypeptides in human. In certain embodiments, the HLA-DMA expression is determined by the amounts of the HLA-DMA polypeptides expressed from the HLA-DMA genes. In certain embodiments, the HLA-DMA polypeptide includes all polypeptides encoded by the natural variants of the HLA-DMA genes and transcripts thereof, including allelic variants (e.g., SNP variants); splice variants; fragments; and derivatives. The HLA-DMA polypeptides of the present disclosure also encompass “full-length,” unprocessed HLA-DMA polypeptide as well as any form of HLA-DMA polypeptide that results from processing in the cell. In some embodiments, NCBI Reference Sequence NP_006111 provides an exemplary human HLA-DMA polypeptide sequence.

As used herein, the term “HLA-DMB” refers to “Major Histocompatibility Complex, Class II, DM beta,” also known as “HLA Class II Histocompatibility Antigen, DM beta Chain” or “Really interesting new gene 7 protein,” and belongs to the HLA Class II beta chain paralogues. The term “HLA-DMB” encompasses the HLA-DMB polypeptides, the HLA-DMB RNA transcripts, and the HLA-DMB genes. The term “HLA-DMB gene” refers to genes encoding HLA-DMB polypeptides. HLA-DMB is expressed in various cells and tissues including intracellular vesicles, among others. Examples of HLA-DMB genes encompass any such native genes in human. In certain embodiments, the term includes all natural variants of HLA-DMB genes, including allelic variants (e.g., SNP variants) and mutations. NCBI Reference Sequence NC_000006 (NC_000006.12 ranges 32934636 . . . 32941028, complement) and GenBank Gene ID: 3109 provide exemplary human HLA-DMB nucleic acid sequences. In certain embodiments, HLA-DMB gene expression is determined by amounts of the mRNA transcripts. In certain embodiments, the mRNA transcripts are splice variants, fragments or derivatives of all native and natural variants of the transcripts of the HLA-DMB genes. NCBI Reference Sequence NM_002118.5 provides an exemplary human HLA-DMB mRNA transcript sequence. HLA-DMB polypeptide is a transmembrane polypeptide, forming a heterodimer with an alpha chain (DMA). HLA-DMB is involved in peptide loading of MHC by catalyzing the release of class II-associated invariant chain peptide (CLIP). Examples of HLA-DMB polypeptides include any such native polypeptides in human. In certain embodiments, HLA-DMB expression is determined by the amounts of the HLA-DMB polypeptides expressed from the HLA-DMB genes. In certain embodiments, the HLA-DMB polypeptide includes all polypeptides encoded by the natural variants of HLA-DMB genes and transcripts thereof, including allelic variants (e.g., SNP variants); splice variants; fragments; and derivatives. The HLA-DMB polypeptide of the present disclosure also encompasses “full-length,” unprocessed HLA-DMB polypeptide as well as any form of HLA-DMB polypeptide that results from processing in the cell. In some embodiments, NCBI Reference Sequence NP_002109.2 provides an exemplary human HLA-DMB polypeptide sequence.

As used herein, the term “HLA-DRB1” refers to “Major Histocompatibility Complex, Class II, DR Beta 1,” also known as “Major Histocompatibility Complex, Class II, DR Beta 1 Precursor” or “HLA Class II Histocompatibility Antigen, DR-1 Beta Chain,” and belongs to the HLA Class II beta chain paralogues. The term “HLA-DRB1” encompasses the HLA-DRB1 polypeptides, the HLA-DRB1 RNA transcripts, and the HLA-DRB1 genes. The term “HLA-DRB1 gene” refers to genes encoding HLA-DRB1 polypeptides. HLA-DRB1 is expressed in various cells and tissues including the lung and the lymph node, among others. Examples of HLA-DRB1 genes encompass any such native genes in human. In certain embodiments, the HLA-DRB1 genes include all natural variants of HLA-DRB1 genes, including allelic variants (e.g., SNP variants) and mutations. NCBI Reference Sequence NG_029921 provides an exemplary human HLA-DRB1 nucleic acid sequence. In certain embodiments, HLA-DRB1 gene expression is determined by the amounts of the mRNA transcripts. In certain embodiments, the mRNA transcripts are splice variants, fragments or derivatives of all native and natural variants of the transcripts of HLA-DRB1. In certain embodiments, NCBI Reference Sequences NM_001243965.1, NM_002124.3, NM_001359193.1, NM_001359194.1, XM_024452553.1, XM_024452554.1, XR_002958969.1 and XR_002958970.1 provide exemplary human HLA-DRB1 mRNA transcript sequences. HLA-DRB1 polypeptide is involved in the immune system and participates in antigen presentation. Examples of HLA-DRB1 polypeptides include any such native polypeptides in human. In certain embodiments, HLA-DRB1 gene expression is determined by the amounts HLA-DRB1 polypeptides expressed from the HLA-DRB1 genes. In certain embodiments, the HLA-DRB1 polypeptides include all polypeptides encoded by the natural variants of HLA-DRB1 genes and transcripts thereof, including allelic variants (e.g., SNP variants); splice variants; fragments; and derivatives. The HLA-DRB1 polypeptide of the present disclosure also encompasses “full-length,” unprocessed HLA-DRB1 polypeptide as well as any form of HLA-DRB1 polypeptide that results from processing in the cell. In some embodiments, NCBI Reference Sequences NP_001230894.1, NP_002115.2, NP_001346122.1, NP_001346123.1, XP_024308321.1 and XP_024308322.1 provide exemplary human HLA-DRB1 polypeptide sequences.

As used herein, the term “HLA-DRA” refers to “Major Histocompatibility Complex, Class II, DR Alpha,” also known as “HLA Class II Histocompatibility Antigen, DR Alpha Chain” or “MHC Class II Antigen DRA,” and belongs to the HLA Class II alpha chain paralogues. The term “HLA-DRA” encompasses the HLA-DRA polypeptides, the HLA-DRA RNA transcripts, and the HLA-DRA genes. The term “HLA-DRA gene” refers to genes encoding HLA-DRA polypeptides. HLA-DRA is expressed in various cells and tissues including plasmacytoid dendritic cells and T Helper cells, among others. Examples of HLA-DRA genes encompass any such native genes in human. In certain embodiments, the HLA-DRA genes include all natural variants of HLA-DRA genes, including allelic variants (e.g., SNP variants) and mutations. NCBI Reference Sequence NC_000006.12, range 32439887 . . . 32445046, provides an exemplary human HLA-DRA nucleic acid sequence. In certain embodiments, HLA-DRA gene expression is determined by the amounts of the mRNA transcripts. In certain embodiments, the mRNA transcripts are splice variants, fragments or derivatives of all native and natural variants of the transcripts of the HLA-DRA genes. NCBI Reference Sequence NM_019111.5 provides an exemplary human HLA-DRA mRNA transcript sequence. HLA-DRA is involved in the immune system and participates in antigen presentation. Examples of HLA-DRA polypeptides include any such native polypeptides in human. In certain embodiments, HLA-DRA gene expression is determined by the amounts of the HLA-DRA polypeptides expressed from HLA-DRA genes. In certain embodiments, a HLA-DRA polypeptide includes all polypeptides encoded by the natural variants of HLA-DRA genes and transcripts thereof, including allelic variants (e.g., SNP variants); splice variants; fragments; and derivatives. The HLA-DRA polypeptide of the present disclosure also encompasses “full-length,” unprocessed HLA-DRA polypeptide as well as any form of HLA-DRA polypeptide that results from processing in the cell. NCBI Reference Sequence NP_061984.2 provides an exemplary human HLA-DRA polypeptide sequence.

As used herein, the term “HLA-DPA1” refers to “Major Histocompatibility Complex, Class II, DP Alpha 1,” also known as “HLA Class II Histocompatibility Antigen, DP Alpha 1 Chain” or “MHC Class II DP3-Alpha,” and belongs to the HLA Class II alpha chain paralogues. The term “HLA-DPA1” encompasses the HLA-DPA1 polypeptides, the HLA-DPA1 RNA transcripts, and the HLA-DPA1 genes. The term “HLA-DPA1 gene” refers to genes encoding HLA-DPA1 polypeptides. HLA-DPA1 is expressed in various cells and tissues including plasmacytoid dendritic cells, T helper cells, and B lymphocytes, among others. Examples of HLA-DPA1 genes encompass any such native genes in human. In certain embodiments, the HLA-DPA1 genes include all natural variants of HLA-DPA1 genes, including allelic variants (e.g., SNP variants) and mutations. NCBI Reference Sequence NG_033241 provides an exemplary human HLA-DPA1 nucleic acid sequence. In certain embodiments, HLA-DPA1 gene expression is determined by the amounts of the mRNA transcripts. In certain embodiments, the mRNA transcripts are splice variants, fragments or derivatives of all native and natural variants of transcripts of the HLA-DPA1 gene. NCBI Reference Sequences NM_033554.3, NM_001242524.2 and NM_001242525.2 provide exemplary human HLA-DPA1 mRNA transcript sequences. HLA-DPA1 is involved in the immune system and participates in antigen presentation. Examples of HLA-DPA1 polypeptides include any such native polypeptides in human. In certain embodiments, HLA-DPA1 gene expression is determined by the amounts of the HLA-DPA1 polypeptides expressed from the HLA-DPA1 genes. In certain embodiments, the HLA-DPA1 polypeptides include all polypeptides encoded by the natural variants of HLA-DPA1 genes and transcripts thereof, including allelic variants (e.g., SNP variants); splice variants; fragments; and derivatives. The HLA-DPA1 polypeptides of the present disclosure also encompass “full-length,” unprocessed HLA-DPA1 polypeptide as well as any form of HLA-DPA1 polypeptide that results from processing in the cell. NCBI Reference Sequences NP_291032.2, NP_001229453.1 and NP_001229454.1 provide exemplary human HLA-DPA1 polypeptide sequences.

As used herein, the term “IL-1α” refers to “Interleukin 1 Alpha,” also known as “Hematopoietin-1” or “Pro-Interleukin-1-Alpha,” in Uniprot or GenBank database. The term “IL-1α” encompasses the IL-1α polypeptides, the IL-1α RNA transcripts, and the IL-1α genes. The term “IL-1α gene” refers to genes encoding IL-1α polypeptides. IL-1α is expressed in various cells and tissues including lung, skin, blood and bone marrow, among others. Examples of IL-1α genes encompass any such native genes from any vertebrate source, including mammals such as primates (e.g., humans and chimpanzees), cows, dogs and rodents (e.g. mice and rats), unless otherwise indicated. In certain embodiments, the IL-1α genes include all natural variants of IL-1α genes, including allelic variants (e.g., SNP variants) and mutations. NCBI Reference Sequence NG_008850 provides an exemplary human IL-1α nucleic acid sequence. In certain embodiments, IL-1α gene expression is determined by the amounts of the mRNA transcripts. In certain embodiments, the mRNA transcripts are splice variants, fragments or derivatives of all native and natural variants of the transcripts of the IL-1α genes. NCBI Reference Sequences NM_000575.5 and NM_001371554.1 provide exemplary human IL-1α mRNA transcript sequences. Examples of IL-1α polypeptides include any such native polypeptides from any vertebrate source as described above. In certain embodiments, IL-1α gene expression is determined by the amounts of the IL-1α polypeptides expressed from IL-1α genes. In certain embodiments, the IL-1α polypeptides include all polypeptides encoded by natural variants of IL-1α genes and transcripts thereof, including allelic variants (e.g., SNP variants); splice variants; fragments; and derivatives. The IL-1α polypeptides of the present disclosure also encompass “full-length,” unprocessed IL-1α polypeptide as well as any form of IL-1α polypeptide that results from processing in the cell. NCBI Reference Sequences NP_000566.3 and NP_001358483.1 provide exemplary human IL-1α polypeptide sequences.

As used herein, the terms “IP10 (CXCL10),” “IP10” and “CXCL10” are used interchangeably to refer to “C—X—C Motif Chemokine Ligand 10,” also known as “Small Inducible Cytokine Subfamily B (Cys-X-Cys), Member 10” or “10 KDa Interferon Gamma-Induced Protein,” in Uniprot or GenBank database. The term “IP10” encompasses the IP10 polypeptides, the IP10 RNA transcripts, and the IP10 genes. The term “IP10 gene” refers to genes encoding IP10 polypeptides. IP10 is expressed in various cells and tissues including skin, blood, the lymph node and spleen, among others. Examples of IP10 genes encompass any such native genes from any vertebrate source, including mammals such as primates (e.g., humans and chimpanzees), cows, dogs and rodents (e.g. mice and rats), unless otherwise indicated. In certain embodiments, the IP10 genes include all natural variants of IP10 genes, including allelic variants (e.g., SNP variants) and mutations. NCBI Reference Sequence NC_000004.12 (range 76021118 . . . 76023497, complement) provides an exemplary human IP10 nucleic acid sequence. In certain embodiments, IP10 gene expression is determined by the amounts of the mRNA transcripts. In certain embodiments, the mRNA transcripts are splice variants, fragments or derivatives of all native and natural variants of the transcripts of the IP10 genes. NCBI Reference Sequence NM_001565.4 provides an exemplary human IP10 mRNA transcript sequence. Examples of IP10 polypeptides include any such native polypeptides from any vertebrate source as described above. In certain embodiments, IP10 gene expression is determined by the amounts of the IP10 polypeptides expressed from the IP10 genes. In certain embodiments, the IP10 polypeptides include all polypeptides encoded by the natural variants of IP10 genes and transcripts thereof, including allelic variants (e.g., SNP variants); splice variants; fragments; and derivatives. The IP10 polypeptides of the present disclosure also encompass “full-length,” unprocessed IP10 polypeptide as well as any form of IP10 polypeptide that results from processing in the cell. NCBI Reference Sequence NP_001556.2 provides exemplary human IP10 polypeptide sequences.

As used herein, the term “interferon regulatory factor 7” or “IRF7” refers to “Interferon Regulatory Factor 7G Isoform,” also known as “Interferon Regulatory Factor-7H” or “IRF-7H,” in Uniprot or GenBank database. The term “IRF7” encompasses the IRF7 polypeptides, the IRF7 RNA transcripts, and the IRF7 genes. The term “IRF7 gene” refers to genes encoding IRF7 polypeptides. The IRF7 gene is expressed in various cells and tissues including spleen, thymus and peripheral blood leukocytes, among others. Examples of IRF7 genes encompass any such native genes from any vertebrate source, including mammals such as primates (e.g., humans and chimpanzees), dogs, cow, chicken and rodents (e.g., mice and rats), unless otherwise indicated. In certain embodiments, the term “IRF7 gene” includes all natural variants of IRF7 genes, including allelic variants (e.g., SNP variants) and mutations. NCBI Reference Sequence NG_029106 provides an exemplary human IRF7 nucleic acid sequence. In certain embodiments, IRF7 gene expression is determined by the amounts of the mRNA transcripts. IRF7 gene encodes various transcript variants. In certain embodiments, the mRNA transcripts are splice variants, fragments or derivatives of all native and natural variants of the transcripts of the IRF7 genes. NCBI Reference Sequences NM_001572.5, NM_004029.4, NM_004031.4, XM_005252907.3, XM_005252909.3, XM_011520066.3 and XM_017017674.1 provide exemplary human IRF7 mRNA transcript sequences. Examples of IRF7 polypeptides include any such native polypeptides from any vertebrate source, as described above. In certain embodiments, IRF7 gene expression is determined by the amounts of the IRF7 polypeptides expressed from the IRF7 genes. In certain embodiments, the IRF7 polypeptides include all polypeptides encoded by the natural variants of IRF7 genes and transcripts thereof, including allelic variants (e.g., SNP variants); splice variants; fragments; and derivatives. The IRF7 polypeptides of the present disclosure also encompass “full-length,” unprocessed IRF7 polypeptide as well as any form of IRF7 polypeptide that results from processing in the cell. NCBI Reference Sequences NP_001563.2, NP_004020.1, NP_004022.2, XP_005252964.1, XP_005252966.1, XP_011518368.1 and XP_016873163.1 provide exemplary human IRF7 polypeptide sequences.

As used herein, the term “MCP1” refers to “Monocyte chemoattractant protein-1,” also known as “C—C Motif Chemokine Ligand 2,” “Monocyte Chemotactic And Activating Factor,” “Monocyte chemotactic protein 1,” “Small-inducible cytokine A2,” or “Monocyte Secretory Protein JE,” in Uniprot or GenBank database. The term “MCP1” encompasses the MCP1 polypeptides, the MCP1 RNA transcripts, and the MCP1 genes. The term “MCP1 gene” refers to genes encoding MCP1 polypeptides. MCP1 is expressed in various cells and tissues including lymph node, blood, spleen, bone marrow, among others. Examples of MCP1 genes encompass any such native genes from any vertebrate source, including mammals such as primates (e.g., humans and chimpanzees), cows, dogs and rodents (e.g. mice and rats), unless otherwise indicated. In certain embodiments, the term “MCP1 gene” includes all natural variants of MCP1 genes, including allelic variants (e.g., SNP variants) and mutations. NCBI Reference Sequence NG_012123 provides an exemplary human MCP1 nucleic acid sequence. In certain embodiments, MCP1 gene expression is determined by the amounts of the mRNA transcripts. In certain embodiments, the mRNA transcripts are splice variants, fragments or derivatives of all native and natural variants of transcripts of the MCP1. NCBI Reference Sequence NM_002982 provides an exemplary human MCP1 mRNA transcript sequence. The MCP1 polypeptide is involved in immunoregulatory and inflammatory processes and functions as a cytokine with chemotactic activity for monocytes and basophils. Examples of MCP1 polypeptides include any such native polypeptides from any vertebrate source as described above. In certain embodiments, MCP1 gene expression is determined by the amounts of the MCP1 polypeptides expressed from the MCP1 genes. In certain embodiments, the MCP1 polypeptides include all polypeptides encoded by the natural variants of the MCP1 genes and transcripts thereof, including allelic variants (e.g., SNP variants); splice variants; fragments; and derivatives. The MCP1 polypeptides of the present disclosure also encompass “full-length,” unprocessed MCP1 polypeptide as well as any form of MCP1 polypeptide that results from processing in the cell. NCBI Reference Sequence NP_002973 provides an exemplary human MCP1 polypeptide sequence.

As used herein, the terms “M-CSF (CSF),” “M-CSF”, and “CSF” are used interchangeably to refer to “macrophage colony-stimulating factor,” which include three different M-CSF isoforms including M-CSF1, M-CSF2 and M-CSF3.

As used herein, the term “M-CSF1” refer to “Colony Stimulating Factor 1,” also known as “Colony Stimulating Factor 1 (Macrophage)” or “Macrophage Colony-Stimulating Factor 1,” in Uniprot or GenBank database. The term “M-CSF1” encompasses the M-CSF1 polypeptides, the M-CSF1 RNA transcripts, and the M-CSF1 genes. The term “M-CSF1 gene” refers to genes encoding M-CSF1 polypeptides. M-CSF1 is expressed in various cells and tissues including fibroblasts, lymph nodes, endothelial cells and epithelial cells, among others. Examples of M-CSF1 genes encompass any such native genes from any vertebrate source, including mammals such as primates (e.g., humans and chimpanzees), cows, dogs and rodents (e.g. mice and rats), unless otherwise indicated. NCBI Reference Sequence NG_030008 provides an exemplary human M-CSF1 nucleic acid sequence. In certain embodiments, M-CSF1 gene expression is determined by the amounts of the mRNA transcripts. In certain embodiments, the mRNA transcripts are splice variants, fragments or derivatives of all native and natural variants of the transcripts of the M-CSF1. NCBI Reference Sequences NM_000757.6, NM_172210.3, NM_172211.4, NM_172211.4 and XM_017000369.1 provide exemplary human M-CSF1 mRNA transcript sequences. M-CSF1 polypeptide regulates production, differentiation and function of macrophages. Examples of M-CSF1 polypeptides include any such native polypeptides from any vertebrate source as described above. In certain embodiments, M-CSF1 gene expression is determined by the amounts of the M-CSF1 polypeptides expressed from the M-CSF1 genes. In certain embodiments, the M-CSF1 polypeptides include all polypeptides encoded by the natural variants of M-CSF1 genes and transcripts thereof, including allelic variants (e.g., SNP variants); splice variants; fragments; and derivatives. The M-CSF1 polypeptides of the present disclosure also encompass “full-length,” unprocessed M-CSF1 polypeptide as well as any form of M-CSF1 polypeptide that results from processing in the cell. NCBI Reference Sequence NP_000748.4, NP_757349.2, NP_757350.2, NP_757351.2 and XP_016855858.1 provide exemplary human M-CSF1 polypeptide sequences.

As used herein, the term “M-CSF2” refers to “Colony Stimulating Factor 2,” “Sargramostim,” also known as “Colony Stimulating Factor 2 (Granulocyte-Macrophage),” in Uniprot or GenBank database. The term “M-CSF2” encompasses the M-CSF2 polypeptides, the M-CSF2 RNA transcripts, and the M-CSF2 genes. The term “M-CSF2 gene” refers to genes encoding M-CSF2 polypeptides. M-CSF2 is expressed in bone marrow, spleen, and lymph node among others. Examples of M-CSF2 genes encompass any such native genes from any vertebrate source, including mammals such as primates (e.g., humans and chimpanzees), cows, dogs and rodents (e.g. mice and rats), unless otherwise indicated. NCBI Reference Sequence NG_033024 provides an exemplary human M-CSF2 nucleic acid sequence. In certain embodiments, M-CSF2 gene expression is determined by the amounts of the mRNA transcripts. In certain embodiments, the mRNA transcripts are splice variants, fragments or derivatives of all native and natural variants of the transcripts of the M-CSF2 genes. NCBI Reference Sequence NM_000758.4 provides an exemplary human M-CSF2 mRNA transcript. M-CSF2 polypeptide regulates the production, differentiation and function of granulocytes and macrophages. Examples of M-CSF2 polypeptides include any such native polypeptides from any vertebrate source as described above. In certain embodiments, M-CSF2 gene expression is determined by the amounts of the M-CSF2 polypeptides expressed from the M-CSF2 genes. In certain embodiments, the M-CSF2 polypeptide includes all polypeptides encoded by the natural variants of M-CSF2 genes and transcripts thereof, including allelic variants (e.g., SNP variants); splice variants; fragments; and derivatives. The M-CSF2 polypeptides of the present disclosure also encompass “full-length,” unprocessed M-CSF2 polypeptide as well as any form of M-CSF2 polypeptide that results from processing in the cell. NCBI Reference Sequence NP_000749.2 provides an exemplary human M-CSF2 polypeptide sequence.

As used herein, the term “M-CSF3” gene refers to “Colony Stimulating Factor 3,” also known as “Pluripoietin,” in Uniprot or GenBank database. The term “M-CSF3” encompasses the M-CSF3 polypeptides, the M-CSF3 RNA transcripts, and the M-CSF3 genes. The term “M-CSF3 gene” refers to genes encoding M-CSF3 polypeptides. M-CSF3 is expressed in bone marrow, spleen, lymph node, among others. M-CSF3 regulates the production, differentiation and function of granulocytes. Examples of M-CSF3 genes encompass any such native genes from any vertebrate source, including mammals such as primates (e.g., humans and chimpanzees), cows, dogs and rodents (e.g. mice and rats), unless otherwise indicated. NCBI Reference Sequence NC_000017.11 range 40015440 . . . 40017813 provides an exemplary human M-CSF3 nucleic acid sequence. In certain embodiments, M-CSF3 gene expression is determined by the amounts of the mRNA transcripts. In certain embodiments, the mRNA transcripts are splice variants, fragments or derivatives of all native and natural variants of the transcripts of the M-CSF3 genes. NCBI Reference Sequences NM_172219.3, NM_000759.4, NM_172220.3, NM_001178147.2 and NR 033662.2 provide exemplary human M-CSF3 mRNA transcript. Examples of M-CSF3 polypeptides include any such native polypeptides from any vertebrate source as described above. In certain embodiments, M-CSF3 gene expression is determined by the amounts of the M-CSF3 polypeptides expressed from the M-CSF3 genes. In certain embodiments, the M-CSF3 polypeptides include all polypeptides encoded by the natural variants of M-CSF3 genes and transcripts thereof, including allelic variants (e.g., SNP variants); splice variants; fragments; and derivatives. The M-CSF3 polypeptides of the present disclosure also encompass “full-length,” unprocessed M-CSF polypeptide as well as any form of M-CSF polypeptide that results from processing in the cell. NCBI Reference Sequences NP_757373.1, NP_000750.1, NP_757374.2 and NP_001171618.1 provide exemplary human M-CSF3 polypeptide sequences.

As used herein, the terms “MIG (CXCL9),” “MIG” and “CXCL9” are used interchangeably to refer to “C—X—C Motif Chemokine Ligand 9,” also known as “Monokine Induced By Interferon-Gamma,” or “Chemokine (C—X—C Motif) Ligand 9,” in Uniprot or GenBank database. The term “MIG” encompasses the MIG polypeptides, the MIG RNA transcripts, and the MIG genes. The term “MIG gene” refers to genes encoding MIG polypeptides. MIG is expressed in various cells and tissues including the spleen, the lymph node and blood, among others. Examples of MIG genes encompass any such native genes from any vertebrate source, including mammals such as primates (e.g., humans and chimpanzees), cows, dogs and mice, unless otherwise indicated. In certain embodiments, the term “MIG gene” includes all natural variants of MIG genes, including allelic variants (e.g., SNP variants) and mutations. NCBI Gene ID 4283 provides an exemplary human MIG nucleic acid sequence. In certain embodiments, MIG gene expression is determined by the amounts of the mRNA transcripts. In certain embodiments, the mRNA transcripts are splice variants, fragments or derivatives of all native and natural variants of the transcripts of MIG genes. NCBI Reference Sequence NM_002416.3 provides an exemplary human MIG mRNA transcript sequence. The MIG polypeptide plays a role in T cell trafficking and immune and inflammatory responses. Examples of MIG polypeptides include any such native polypeptides from any vertebrate source as described above, unless otherwise indicated. In certain embodiments, MIG gene expression is determined by the amounts of the MIG polypeptides expressed from the MIG genes. In certain embodiments, the MIG polypeptides include all polypeptides encoded by the natural variants of the MIG genes and transcripts thereof, including allelic variants (e.g., SNP variants); splice variants; fragments; and derivatives. The MIG polypeptides of the present disclosure also encompass “full-length,” unprocessed MIG polypeptide as well as any form of MIG polypeptide that results from processing in the cell. NCBI Reference Sequence NP_002407 provides an exemplary human MIG polypeptide sequence.

As used herein, the term “MIP1α” refers to “Macrophage Inflammatory Protein 1-Alpha,” also known as “C—C Motif Chemokine Ligand 3,” or “Small Inducible Cytokine A3,” “Tonsillar lymphocyte LD78 alpha protein,” in Uniprot or GenBank database. The term “MIP1α” encompasses the MIP1α polypeptides, the MIP1α RNA transcripts, and the MIP1α genes. The term “MIP1α gene” refers to genes encoding MIP1α polypeptides. MIP1α is expressed in various cells and tissues including bone marrow, spleen, the lymph node and blood, among others. Examples of MIP1α genes encompass any such native genes from any vertebrate source, including mammals such as primates (e.g., humans and chimpanzees), cows and dogs, unless otherwise indicated. In certain embodiments, the term “MIP1α gene” includes all natural variants of MIP1α genes, including allelic variants (e.g., SNP variants) and mutations. NCBI Reference Sequence NG_027730 provides an exemplary human MIP1α nucleic acid sequence. In certain embodiments, MIP1α gene expression is determined by the amounts of the mRNA transcripts. In certain embodiments, the mRNA transcripts are splice variants, fragments or derivatives of all native and natural variants of the transcripts of the MIP1α genes. NCBI Reference Sequence NM_002983 provides an exemplary human MIP1α mRNA transcript sequence. The MIP1α polypeptide plays a role in inflammatory responses by binding CCR1, CCR4 and CCR5 receptors. Examples of MIP1α polypeptides include any such native polypeptides from any vertebrate source as described above. In certain embodiments, MIP1α gene expression is determined by the amounts of the MIP1α polypeptides expressed from the MIP1α genes. In certain embodiments, the MIP1α polypeptides include all polypeptides encoded by the natural variants of MIP1α genes and transcripts thereof, including allelic variants (e.g., SNP variants); splice variants; fragments; and derivatives. The MIP1α polypeptides of the present disclosure also encompass “full-length,” unprocessed MIP1α polypeptide as well as any form of MIP1α polypeptide that results from processing in the cell. NCBI Reference Sequence NP_002974 provides an exemplary human MIP1α polypeptide sequence.

As used herein, the term “MIP1β” refers to “Macrophage Inflammatory Protein 1-Beta,” also known as “C—C Motif Chemokine Ligand 4,” “Small Inducible Cytokine A4” or “lymphocyte activation gene 1 protein,” in Uniprot or GenBank database. The term “MIP1β” encompasses the MIP1β polypeptides, the MIP1β RNA transcripts, and the MIP1β genes. The term “MIP1β gene” refers to genes encoding MIP1β polypeptides. MIP1β is expressed in various cells and tissues including bone marrow, spleen, the lymph node and blood, among others. Examples of MIP1β genes encompass any such native genes from any vertebrate source, including mammals such as primates (e.g., humans and chimpanzees), cows, dogs and rodents (e.g. mice and rats), unless otherwise indicated. In certain embodiments, the term “MIP1β gene” includes all natural variants of MIP1β genes, including allelic variants (e.g., SNP variants) and mutations. NCBI Reference Sequence NG_033066 provides an exemplary human MIP1β nucleic acid sequence. In certain embodiments, MIP1β gene expression is determined by the amounts of the mRNA transcripts. In certain embodiments, the mRNA transcripts are splice variants, fragments or derivatives of all native and natural variants of the transcripts of the MIP1β genes. NCBI Reference Sequence NM_002984.4 provides an exemplary human MIP1β mRNA transcript sequence. MIP1β polypeptide is a monokine with chemokinetic and inflammatory functions. Examples of MIP1β polypeptides include any such native polypeptides from any vertebrate source as described above. In certain embodiments, MIP1β gene expression is determined by the amounts of the MIP1β polypeptide expressed from the MIP1β genes. In certain embodiments, the MIP1β polypeptides include all polypeptides encoded by the natural variants of MIP1β genes and transcripts thereof, including allelic variants (e.g., SNP variants); splice variants; fragments; and derivatives. The MIP1β polypeptides of the present disclosure also encompass “full-length,” unprocessed MIP1α polypeptide as well as any form of MIP1β polypeptide that results from processing in the cell. NCBI Reference Sequence NP_002975.1 provides an exemplary human MIP1β polypeptide sequence.

As used herein, the term “MT-ATP6” refers to “Mitochondrially Encoded ATP Synthase Membrane Subunit 6,” also known as “MTATP6,” “ATPASE6” or “ATP6,” in Uniprot or GenBank database. The term “MT-ATP6” encompasses the MT-ATP6 polypeptides, the MT-ATP6 RNA transcripts, and the MT-ATP6 genes. The term “MT-ATP6 gene” refers to genes encoding MT-ATP6 polypeptides. MT-ATP6 is expressed in various cells and tissues including the thyroid, lymph node, bone marrow and adrenal gland, among others. Examples of MT-ATP6 genes encompass any such native genes from any vertebrate source, including mammals such as primates (e.g., humans and chimpanzees), mice, chicken and lizards, unless otherwise indicated. In certain embodiments, the term “MT-ATP6 gene” includes all natural variants of MT-ATP6 genes, including allelic variants (e.g., SNP variants) and mutations. NCBI Reference Sequence NC_012920.1 range 8527 . . . 9207 provides an exemplary human MT-ATP6 nucleic acid sequence. In certain embodiments, MT-ATP6 gene expression is determined by the amounts of the mRNA transcripts. In certain embodiments, the mRNA transcripts are splice variants, fragments or derivatives of all native and natural variants of the transcripts of the MT-ATP6 genes. In certain embodiments, MT-ATP6 gene expression is determined by the amounts of the MT-ATP6 polypeptide expressed from the MT-ATP6 genes. MT-ATP6 polypeptide acts as a mitochondrial membrane ATP synthase, which produces ATP from ADP. Examples of MT-ATP6 polypeptides include any such native polypeptides from any vertebrate source as described above. In certain embodiments, the MT-ATP6 polypeptides include all polypeptides encoded by the natural variants of MT-ATP8 genes and transcripts thereof, including allelic variants (e.g., SNP variants); splice variants; fragments; and derivatives. The MT-ATP6 polypeptides of the present disclosure also encompass “full-length,” unprocessed MT-ATP6 polypeptide as well as any form of MT-ATP6 polypeptide that results from processing in the cell. NCBI Reference Sequence YP_003024031.1 provides an exemplary human MT-ATP8 polypeptide sequence.

As used herein, the term “MT-ATP8” refers to “Mitochondrially Encoded ATP Synthase Membrane Subunit 8,” also known as “MTATP8,” “ATASE8 or “ATP8,” in Uniprot or GenBank database. The term “MT-ATP8” encompasses the MT-ATP8 polypeptides, the MT-ATP8 RNA transcripts, and the MT-ATP8 genes. The term “MT-ATP8 gene” refers to genes encoding MT-ATP8 polypeptides. MT-ATP8 is expressed in various cells and tissues including the thyroid, lymph node, bone marrow and adrenal gland, among others. Examples of MT-ATP8 genes encompass any such native genes from any vertebrate source, including mammals such as primates (e.g., humans and chimpanzees), unless otherwise indicated. In certain embodiments, the term “MT-ATP8 gene” includes all natural variants of MT-ATP8 genes, including allelic variants (e.g., SNP variants) and mutations. NCBI Reference Sequence NC_012920.1 range 8366 . . . 8572 provides an exemplary human MT-ATP8 nucleic acid sequence. In certain embodiments, MT-ATP8 gene expression is determined by the amounts of the mRNA transcripts. In certain embodiments, the mRNA transcripts are splice variants, fragments or derivatives of all native and natural variants of the transcripts of the MT-ATP8 genes. In certain embodiments, MT-ATP8 gene expression is determined by the amounts of the MT-ATP8 polypeptides expressed from the MT-ATP8 genes. MT-ATP8 polypeptide is a mitochondrial membrane ATP synthase that produces ATP from ADP. Examples of MT-ATP8 polypeptides include any such native polypeptides from any vertebrate source as described above. In certain embodiments, the MT-ATP8 polypeptides include all polypeptides encoded by the natural variants of MT-ATP8 genes and transcripts thereof, including allelic variants (e.g., SNP variants); splice variants; fragments; and derivatives. The MT-ATP8 polypeptides of the present disclosure also encompass “full-length,” unprocessed MT-ATP8 polypeptide as well as any form of MT-ATP8 polypeptide that results from processing in the cell. NCBI Reference Sequence YP_003024030.1 provides an exemplary human MT-ATP8 polypeptide sequence.

As used herein, the term “NFKB1” refers to “Nuclear Factor Kappa B Subunit 1,” also known as “Nuclear Factor Of Kappa Light Polypeptide Gene Enhancer In B-Cells 1” or “Nuclear Factor NF-Kappa-B P105 Subunit,” in Uniprot or GenBank database. The term “NFKB1” encompasses the NFKB1 polypeptides, the NFKB1 RNA transcripts, and the NFKB1 genes. The term “NFKB1 gene” refers to genes encoding NFKB1 polypeptides. NFKB1 is expressed in nearly all cell types including hematopoietic bone marrow, peripheral blood mononuclear cells and the lymph node, among others. Examples of NFKB1 genes encompass any such native genes from any vertebrate source, including mammals such as primates (e.g., humans and chimpanzees), cows, dogs and rodents (e.g. mice and rats), unless otherwise indicated. In certain embodiments, the term “NFKB1 gene” includes all natural variants of NFKB1 genes, including allelic variants (e.g., SNP variants) and mutations. NCBI Reference Sequence NG_050628 provides an exemplary human NFKB1 nucleic acid sequence. In certain embodiments, NFKB1 gene expression is determined by the amounts of the mRNA transcripts. In certain embodiments, the mRNA transcripts are splice variants, fragments or derivatives of all native and natural variants of the transcripts of the NFKB1 genes. NCBI Reference Sequences NM_003998.4, NM_001165412.2, NM_001319226.2, XM_011532006.2, XM_024454067.1, XM_024454068.1 and XM_024454069.1 provide exemplary human NFKB1 mRNA transcript sequences. NFKB1 polypeptide is present in nearly all cell types and is activated in response to many stimuli including inflammation, immune activation, differentiation and cell growth, among other biological processes. Examples of NFKB1 polypeptides include any such native polypeptides from any vertebrate source as described above. In certain embodiments, NFKB1 gene expression is determined by the amounts of the NFKB1 polypeptides expressed from the NFKB1 genes. In certain embodiments, the NFKB1 polypeptides include all polypeptides encoded by the natural variants of NFKB1 genes and transcripts thereof, including allelic variants (e.g., SNP variants); splice variants; fragments; and derivatives. The NFKB1 polypeptides of the present disclosure also encompass “full-length,” unprocessed NFKB1 polypeptide as well as any form of NFKB1 polypeptide that results from processing in the cell. NCBI Reference Sequences NP_003989.2, NP_001158884.1, NP_001306155.1, XP_011530308.1, XP_024309835.1, XP_024309836.1 and XP_024309837.1 provide exemplary human NFKB1 polypeptide sequences.

As used herein, the term “NFKB2” refers to “Nuclear Factor Kappa B Subunit 2,” also known as “Nuclear Factor Of Kappa Light Polypeptide Gene Enhancer In B-Cells 2 (P49/P100)” or “Lymphocyte Translocation Chromosome 10 Protein,” in Uniprot or GenBank database. The term “NFKB2” encompasses the NFKB2 polypeptides, the NFKB2 RNA transcripts, and the NFKB2 genes. The term “NFKB2 gene” refers to genes encoding NFKB2 polypeptides. NFKB2 is expressed in various cells and tissues including hematopoietic bone marrow, peripheral blood mononuclear cells and the lymph node, among others. Examples of NFKB2 genes encompass any such native genes from any vertebrate source, including mammals such as primates (e.g., humans and chimpanzees), cows, dogs and rodents (e.g. mice and rats), unless otherwise indicated. In certain embodiments, the term “NFKB2 gene” includes all natural variants of NFKB2 genes, including allelic variants (e.g., SNP variants) and mutations. NCBI Reference Sequence NG_033874 provides an exemplary human NFKB2 nucleic acid sequence. In certain embodiments, NFKB2 gene expression is determined by the amounts of the mRNA transcripts. In certain embodiments, the mRNA transcripts are splice variants, fragments or derivatives of all native and natural variants of the transcripts of the NFKB2 genes. NCBI Reference Sequences NM_001322934.2, NM_002502.6, NM_001077494.3, NM_001261403.3, NM_001288724.1, NM_001322935.1, XM_011539830.3, XM_011539831.2, XM_017016278.1, XM_024448026.1 and XM_024448027.1 provide exemplary human NFKB2 mRNA transcript sequences. NFKB2 polypeptide is a transcription factor with dual functions including cytoplasmic retention of NFKB complex proteins and p52 cotranslational processing. Examples of NFKB2 polypeptides include any such native polypeptides from any vertebrate source as described above. In certain embodiments, NFKB2 gene expression is determined by the amounts of the NFKB2 polypeptides expressed from the NFKB2 genes. In certain embodiments, the NFKB2 polypeptides include all polypeptides encoded by the natural variants of the NFKB2 genes and transcripts thereof, including allelic variants (e.g., SNP variants); splice variants; fragments; and derivatives. The NFKB2 polypeptides of the present disclosure also encompass “full-length,” unprocessed NFKB2 polypeptide as well as any form of NFKB2 polypeptide that results from processing in the cell. NCBI Reference Sequences NP_001309863.1, NP_002493.3, NP_001070962.1, NP_001248332.1, NP_001275653.1, NP_001309864.1, XP_011538132.1, XP_011538133.1, XP_016871767.1, XP_024303794.1 and XP_024303795.1 provide exemplary human NFKB2 polypeptide sequences.

As used herein, the term “RELA” refers to “RELA Proto-Oncogene, NF-κB Subunit,” also known as “Nuclear factor NF-kappa-B p65 subunit,” “Transcription factor p65,” “NFKB3,” “Nuclear Factor Of Kappa Light Polypeptide Gene Enhancer In B-Cells 3” or “V-Rel Avian Reticuloendotheliosis Viral Oncogene Homolog A,” in Uniprot or GenBank database. The term “RELA” encompasses the RELA polypeptides, the RELA RNA transcripts, and the RELA genes. The term “RELA gene” refers to genes encoding RELA polypeptides. RELA is expressed in various cells and tissues including peripheral blood cells, the lymph node and spleen, among others. Examples of RELA genes encompass any such native genes from any vertebrate source, including mammals such as primates (e.g., humans and chimpanzees), cows, dogs and rodents (e.g. mice and rats), unless otherwise indicated. In certain embodiments, the term “RELA gene” includes all natural variants of RELA genes, including allelic variants (e.g., SNP variants) and mutations. NCBI Reference Sequence NG_029971 provides an exemplary human RELA nucleic acid sequence. In certain embodiments, RELA gene expression is determined by the amounts of the mRNA transcripts. In certain embodiments, the mRNA transcripts are splice variants, fragments or derivatives of all native and natural variants of transcripts of the RELA genes. NCBI Reference Sequences NM_021975.4, NM_001145138.2, NM_001243984.2, NM_001243985.1, XM_011545206.2 and XM_011545207.2 provide exemplary human RELA mRNA transcript sequences. Examples of RELA polypeptides include any such native polypeptides from any vertebrate source as described above, unless otherwise indicated. In certain embodiments, RELA gene expression is determined by the amounts of the RELA polypeptides expressed from the RELA genes. In certain embodiments, the RELA polypeptides include all polypeptides encoded by the natural variants of RELA genes and transcripts thereof, including allelic variants (e.g., SNP variants); splice variants; fragments; and derivatives. The RELA polypeptides of the present disclosure also encompass “full-length,” unprocessed RELA polypeptide as well as any form of RELA polypeptide that results from processing in the cell. NCBI Reference Sequences NP_068810.3, NP_001138610.1, NP_001230913.1, NP_001230914.1, XP_011543508.1 and XP_011543509.1 provide exemplary human RELA polypeptide sequences.

As used herein, the term “RELB” refers to “RELB Proto-Oncogene, NF-κB Subunit,” also known as “V-Rel Avian Reticuloendotheliosis Viral Oncogene Homolog B,” “Nuclear Factor Of Kappa Light Polypeptide Gene Enhancer In B-Cells 3,” or “Transcription Factor RelB,” in Uniprot or GenBank database. The term “RELB” encompasses the RELB polypeptides, the RELB RNA transcripts, and the RELB genes. The term “RELB gene” refers to genes encoding RELB polypeptides. RELB is expressed in various cells and tissues including whole blood, B lymphocytes and peripheral mononuclear cells, among others. Examples of RELB genes encompass any such native genes from any vertebrate source, including mammals such as primates (e.g., humans and chimpanzees), cows, dogs and rodents (e.g. mice and rats), unless otherwise indicated. In certain embodiments, the term “RELB gene” includes all natural variants of RELB genes, including allelic variants (e.g., SNP variants) and mutations. NCBI Reference Sequence NC_000019.10 range 45001449 . . . 45038194 provides exemplary human RELB nucleic acid sequences. In certain embodiments, RELB gene expression is determined by the amounts of the mRNA transcripts. In certain embodiments, the mRNA transcripts are splice variants, fragments or derivatives of all native and natural variants of the transcripts of the RELB genes. NCBI Reference Sequences NM_006509.4, XM_005259127.3, and XM_005259128.2 provide exemplary human RELB mRNA transcript sequences. Examples of RELB polypeptides include any such native polypeptides from any vertebrate source as described above. In certain embodiments, RELB gene expression is determined by the amounts of the RELB polypeptides expressed from the RELB genes. In certain embodiments, the RELB polypeptides includes all polypeptides encoded by the natural variants of RELB genes and transcripts thereof, including allelic variants (e.g., SNP variants); splice variants; fragments; and derivatives. The RELB polypeptides of the present disclosure also encompass “full-length,” unprocessed RELB polypeptide as well as any form of RELB polypeptide that results from processing in the cell. NCBI Reference Sequences NP_006500.2, XP_005259184.1 and XP_005259185.1 provide exemplary human RELB polypeptide sequences.

As used herein, the term “REL” refers to “REL Proto-Oncogene, NF-κB Subunit,” also known as “V-Rel Avian Reticuloendotheliosis Viral Oncogene Homolog” or “Proto-Oncogene C-Rel,” in Uniprot or GenBank database. The term “REL” encompasses the REL polypeptides, the REL RNA transcripts, and the REL genes. The term “REL gene” refers to genes encoding REL polypeptides. REL is expressed in various cells and tissues including B cells, monocytes and peripheral blood mononuclear cells, among others. Examples of REL genes encompass any such native genes from any vertebrate source, including mammals such as primates (e.g., humans and chimpanzees), cows, dogs and rodents (e.g. mice and rats), unless otherwise indicated. In certain embodiments, the term “REL gene” includes all natural variants of REL genes, including allelic variants (e.g., SNP variants) and mutations. NCBI Reference Sequence NC_000002.12 range 60881521 . . . 60931612 provides an exemplary human REL nucleic acid sequence. In certain embodiments, REL gene expression is determined by the amounts of the mRNA transcripts. In certain embodiments, the mRNA transcripts are splice variants, fragments or derivatives of all native and natural variants of the transcripts of the REL genes. NCBI Reference Sequences NM_001291746.2, NM_002908.4, XM_011533010.3 and XM_017004627.2 provide exemplary human REL mRNA transcript sequences. Examples of REL polypeptides include any such native polypeptide from any vertebrate source as described above. In certain embodiments, REL gene expression is determined by the amounts of the REL polypeptides expressed from the REL genes. In certain embodiments, the REL polypeptides include all polypeptides encoded by the natural variants of REL genes and transcripts thereof, including allelic variants (e.g., SNP variants); splice variants; fragments; and derivatives. The REL polypeptides of the present disclosure also encompass “full-length,” unprocessed REL polypeptide as well as any form of REL polypeptide that results from processing in the cell. NCBI Reference Sequences NP_001278675.1, NP_002899.1, XP_011531312.1 and XP_016860116.1 provide exemplary human REL polypeptide sequences.

As used herein, the term “BASAL 1” refers to “RasGAP-Activating-Like Protein 1”, also known as “RAS Protein Activator Like 1,” or “Ras GTPase-Activating-Like Protein,” in Uniprot or GenBank database. The term “RASAL1” encompasses the RASAL1 polypeptides, the RASAL1 RNA transcripts, and the RASAL1 genes. The term “RASAL1 gene” refers to genes encoding RASAL1 polypeptides. RASAL1 is expressed in various cells and tissues including the thyroid and adrenal medulla. Examples of RASAL1 genes encompass any such native genes from any vertebrate source, including mammals such as primates (e.g., humans and chimpanzees), dogs, cow, chicken, reptiles (e.g. lizards and frogs), and rodents (e.g., mice and rats), unless otherwise indicated. In certain embodiments, the term “RASAL1 gene” includes all natural variants of RASAL1 genes, including allelic variants (e.g., SNP variants) and mutations. NCBI Reference Sequence NG_047089 provides an exemplary human RASAL1 nucleic acid sequence. In certain embodiments, RASAL1 gene expression is determined by the amounts of the mRNA transcripts. RASAL1 gene encodes various transcript variants. In certain embodiments, the mRNA transcripts are splice variants, fragments or derivatives of all native and natural variants of the transcripts of the RASAL1 genes. NCBI Reference Sequences NM_001301202.1, NM_004658.2, NM_001193520.1, NM_001193521.1, XM_005253950.4, XM_006719641.3, XM_006719642.3, XM_011538852.2, XM_011538853.2, XM_011538854.2, XM_017020028.1, XM_017020029.1, XM_017020030.1, XM_017020031.1, XR_001748902.1, XR_001748903.1 and XR_002957386.1 provide exemplary human RASAL1 mRNA transcript sequences. The RASAL1 polypeptide is a member of the GAP1 family of GTPase-activating proteins. RASAL1 polypeptide suppresses RAS function allowing for control of proliferation and differentiation. Examples of RASAL1 polypeptides include any such native polypeptides from any vertebrate source as described above. In certain embodiments, RASAL1 gene expression is determined by the amounts of the RASAL1 polypeptides expressed from the RASAL1 genes. In certain embodiments, the RASAL1 polypeptides includes all polypeptides encoded by the natural variants of the RASAL1 genes and transcripts thereof, including allelic variants (e.g., SNP variants); splice variants; fragments; and derivatives. The RASAL1 polypeptides of the present disclosure also encompass “full-length,” unprocessed RASAL1 polypeptide as well as any form of RASAL1 polypeptide that results from processing in the cell. NCBI Reference Sequences NP_001288131.1, NP_004649.2, NP_001180449.1, NP_001180450.1, XP_005254007.1, XP_006719704.1, XP_006719705.1, XP_011537154.1, XP_011537155.1, XP_011537156.1, XP_016875517.1, XP_016875518.1, XP_016875519.1 and XP_016875520.1 provide exemplary human RASAL1 polypeptide sequences.

As used herein, the term “RhoB” refers to “Ras Homolog Family Member B,” also known as “Rho-Related GTP-Binding Protein RhoB,” or “Ras Homolog Gene Family, Member B,” in Uniprot or GenBank database. The term “RhoB” encompasses the RhoB polypeptides, the RhoB RNA transcripts, and the RhoB genes. The term “RhoB gene” refers to genes encoding RhoB polypeptides. RhoB is expressed in various cells and tissues including nervous system, blood and the spleen, among others. Examples of RhoB genes encompass any such native genes from any vertebrate source, including mammals such as primates (e.g., humans and chimpanzees), cows, dogs, and rats, unless otherwise indicated. In certain embodiments, the term “RhoB gene” includes all natural variants of RhoB genes, including allelic variants (e.g., SNP variants) and mutations. NCBI Reference Sequence NC_000002.12 range 20447074 . . . 20449440 provides an exemplary human RhoB nucleic acid sequence. In certain embodiments, RhoB gene expression is determined by the amounts of the mRNA transcripts. In certain embodiments, the mRNA transcripts are splice variants, fragments or derivatives of all native and natural variants of the transcripts of the RhoB genes. NCBI Reference Sequence NM_004040.4 provides an exemplary human RhoB mRNA transcript sequence. Examples of RhoB polypeptides include any such native polypeptides from any vertebrate source as described above. In certain embodiments, RhoB gene expression is determined by the amounts of the RhoB polypeptides expressed from the RhoB genes. In certain embodiments, the RhoB polypeptide includes all polypeptides encoded by the natural variants of the RhoB genes and transcripts thereof, including allelic variants (e.g., SNP variants); splice variants; fragments; and derivatives. The RhoB polypeptides of the present disclosure also encompass “full-length,” unprocessed RhoB polypeptide as well as any form of RhoB polypeptide that results from processing in the cell. NCBI Reference Sequence NP_004031.1 provides an exemplary human RhoB polypeptide sequence.

As used herein, the term “RhoF” refers to “Rho-related GTP-binding protein RhoF,” also known as “Ras Homolog Family Member F, Filopodia Associated,” “Rho In Filopodia” or “Ras Homolog Gene Family, Member F (In Filopodia),” in Uniprot or GenBank database. The term “RhoF” encompasses the RhoF polypeptides, the RhoF RNA transcripts, and the RhoF genes. The term “RhoF gene” refers to genes encoding RhoF polypeptides. RhoF is expressed in various cells and tissues including intestine, lung and pancreas, among others. Examples of RhoF genes encompass any such native genes from any vertebrate source, including mammals such as primates (e.g., humans and chimpanzees), cows, dogs, and rats, unless otherwise indicated. In certain embodiments, the term “RhoF gene” includes all natural variants of RhoF genes, including allelic variants (e.g., SNP variants) and mutations. NCBI Reference Sequence NC_000012.12 (range 121777754 . . . 121793688, complement) provides an exemplary human RhoF nucleic acid sequence. In certain embodiments, RhoF gene expression is determined by the amounts of the mRNA transcripts. In certain embodiments, the mRNA transcripts are splice variants, fragments or derivatives of all native and natural variants of the transcripts of the RhoF genes. NCBI Reference Sequence NM_019034.3 provides an exemplary human RhoF mRNA transcript sequence. The RhoF polypeptide is involved in formation of thin, actin-rich surface projections, known as filopodia. Examples of RhoF polypeptides include any such native polypeptides from any vertebrate source as described above. In certain embodiments, RhoF gene expression is determined by the amounts of the RhoF polypeptides expressed from the RhoF genes. In certain embodiments, the RhoF polypeptides include all polypeptides encoded by the natural variants of the RhoF genes and transcripts thereof, including allelic variants (e.g., SNP variants); splice variants; fragments; and derivatives. The RhoF polypeptides of the present disclosure also encompass “full-length,” unprocessed RhoF polypeptide as well as any form of RhoF polypeptide that results from processing in the cell. Reference Sequence NP_061907.2 provides an exemplary human RhoF polypeptide sequence.

As used herein, the term “RhoG” refers to “Rho-Related GTP-Binding Protein RhoG,” also known as “Ras Homolog Family Member G,” “Ras Homolog Gene Family, Member G (Rho G),” in Uniprot or GenBank database. The term “RhoG” encompasses the RhoG polypeptides, the RhoG RNA transcripts, and the RhoG genes. The term “RhoG gene” refers to genes encoding RhoG polypeptides. RhoG is expressed in various cells and tissues including the neutrophil, T lymphocyte and peripheral blood mononuclear cells, among others. Examples of RhoG genes encompass any such native genes from any vertebrate source, including mammals such as primates (e.g., humans and chimpanzees), cows, dogs, and rodents (e.g. mice and rats), unless otherwise indicated. In certain embodiments, the term “RhoG gene” includes all natural variants of RhoG genes, including allelic variants (e.g., SNP variants) and mutations. NCBI Reference Sequence NC_000011.10 (range 3826978 . . . 3840959, complement) provides an exemplary human RhoG nucleic acid sequence. In certain embodiments, RhoG gene expression is determined by the amounts of the mRNA transcripts. In certain embodiments, the mRNA transcripts are splice variants, fragments or derivatives of all native and natural variants of the transcripts of the RhoG genes. NCBI Reference Sequences NM_001665.4, XM_005252916.2 and XM_017017719.1 provide exemplary human RhoG mRNA transcript sequences. The RhoG polypeptide is involved in formation of membrane ruffles during micropinocytosis and plays a role in cell migration. Examples of RhoG polypeptides include any such native polypeptides from any vertebrate source as described above. In certain embodiments, RhoG gene expression is determined by the amounts of the RhoG polypeptides expressed from the RhoG genes. In certain embodiments, the RhoG polypeptides include all polypeptides encoded by the natural variants of RhoG genes and transcripts thereof, including allelic variants (e.g., SNP variants); splice variants; fragments; and derivatives. The RhoG polypeptides of the present disclosure also encompass “full-length,” unprocessed RhoG polypeptide as well as any form of RhoG polypeptide that results from processing in the cell. NCBI Reference Sequences NP_001656.2, XP_005252973.1, and XP_016873208.1 provide exemplary human RhoG polypeptide sequence.

As used herein, the term “STAT1” refers to “Signal Transducer And Activator Of Transcription 1,” also known as “Transcription Factor ISGF-3 Components P91/P84” or “Signal Transducer And Activator Of Transcription 1-Alpha/Beta,” in Uniprot or GenBank database. The term “STAT1” encompasses the STAT1 polypeptides, the STAT1 RNA transcripts, and the STAT1 genes. The term “STAT1 gene” refers to genes encoding STAT1 polypeptides. STAT1 is expressed in various cells and tissues including T helper cells, T-cytotoxic cells, lymph node and spleen, among others. Examples of STAT1 genes encompass any such native genes from any vertebrate source, including mammals such as primates (e.g., humans and chimpanzees), cows, dogs, and rodents (e.g. mice and rats), unless otherwise indicated. In certain embodiments, the term “STAT1 gene” includes all natural variants of STAT1 genes, including allelic variants (e.g., SNP variants) and mutations. NCBI Reference Sequence NG_008294 provides an exemplary human STAT1 nucleic acid sequence. In certain embodiments, STAT1 gene expression is determined by the amounts of the mRNA transcripts. In certain embodiments, the mRNA transcripts are splice variants, fragments or derivatives of all native and natural variants of the transcripts of the STAT1 genes. NCBI Reference Sequences NM_007315.4, NM_139266.2, XM_006712718.1, XM_017004783.2, XR_001738914.2 and XR_001738915.2 provide exemplary human STAT1 mRNA transcript sequences. Examples of STAT1 polypeptides include any such native polypeptides from any vertebrate source as described above. In certain embodiments, STAT1 gene expression is determined by the amounts of the STAT1 polypeptides expressed from the STAT1 genes. In certain embodiments, the STAT1 polypeptides include all polypeptides encoded by the natural variants of the STAT1 genes and transcripts thereof, including allelic variants (e.g., SNP variants); splice variants; fragments; and derivatives. The STAT1 polypeptides of the present disclosure also encompass “full-length,” unprocessed STAT1 polypeptide as well as any form of STAT1 polypeptide that results from processing in the cell. NCBI Reference Sequences NP_009330.1, NP_644671.1, XP_006712781.1, and XP_016860272.1 provide exemplary human STAT1 polypeptide sequences.

As used herein, the term “STAT2” refers to “Signal Transducer And Activator Of Transcription 2,” also known as “Signal Transducer And Activator Of Transcription 2, 113 kDa” or “P113,” in Uniprot or GenBank database. The term “STAT2” encompasses the STAT2 polypeptides, the STAT2 RNA transcripts, and the STAT2 genes. The term “STAT2 gene” refers to genes encoding STAT2 polypeptides. STAT2 is expressed in various cells and tissues including monocytes, bone marrow stromal cells, peripheral blood mononuclear cells and the lymph node, among others. Examples of STAT2 genes encompass any such native genes from any vertebrate source, including mammals such as primates (e.g., humans and chimpanzees), cows, dogs, and rodents (e.g. mice and rats), unless otherwise indicated. In certain embodiments, the term “STAT2 gene” includes all natural variants of STAT2 genes, including allelic variants (e.g., SNP variants) and mutations. NCBI Reference Sequence NG_046314 provides an exemplary human STAT2 nucleic acid sequence. In certain embodiments, STAT2 gene expression is determined by the amounts of the mRNA transcripts. In certain embodiments, the mRNA transcripts are splice variants, fragments or derivatives of all native and natural variants of the transcripts of the STAT2 genes. NCBI Reference Sequences NM_005419.4, NM_198332.2, XM_011538697.2, XM_011538698.3, XM_011538699.3, XM_011538700.2, XM_017019904.2, XR_245953.3, XR_001748856.1, XR_001748857.1, XR_001748858.2, XR_002957375.1 and XR_002957376.1 provide exemplary human STAT2 mRNA transcript sequences. Examples of STAT2 polypeptides include any such native polypeptides from any vertebrate source as described above. In certain embodiments, STAT2 gene expression is determined by the amounts of the STAT2 polypeptides expressed from the STAT2 genes. In certain embodiments, the STAT2 polypeptide includes all polypeptides encoded by the natural variants of the STAT2 genes and transcripts thereof, including allelic variants (e.g., SNP variants); splice variants; fragments; and derivatives. The STAT2 polypeptides of the present disclosure also encompass “full-length,” unprocessed STAT2 polypeptide as well as any form of STAT2 polypeptide that results from processing in the cell. NCBI Reference Sequences NP_005410.1, NP_938146.1, XP_011536999.1, XP_011537000.1, XP_011537001.1, XP_011537002.1 and XP_016875393.1 provide exemplary human STAT2 polypeptide sequences.

As used herein, the term “STAT3” refers to “Signal Transducer And Activator Of Transcription 3,” also known as “Acute-Phase Response Factor” or “APRF,” in Uniprot or GenBank database. The term “STAT3” encompasses the STAT3 polypeptides, the STAT3 RNA transcripts, and the STAT3 genes. The term “STAT3 gene” refers to genes encoding STAT3 polypeptides. STAT3 is expressed in various cells and tissues including bone marrow and lymph node, among others. Examples of STAT3 genes encompass any such native genes from any vertebrate source, including mammals such as primates (e.g., humans and chimpanzees), cows, dogs, and rodents (e.g. mice and rats), unless otherwise indicated. In certain embodiments, the term “STAT3 gene” includes all natural variants of STAT3 genes, including allelic variants (e.g., SNP variants) and mutations. NCBI Reference Sequence NG_007370 provides an exemplary human STAT3 nucleic acid sequence. In certain embodiments, STAT3 gene expression is determined by the amounts of the mRNA transcripts. In certain embodiments, the mRNA transcripts are splice variants, fragments or derivatives of all native and natural variants of the transcripts of the STAT3 genes. NCBI Reference Sequences NM_139276.3, NM_003150.4, NM_213662.2, NM_001369512.1, NM_001369513.1, NM_001369514.1, NM_001369516.1, NM_001369517.1, NM_001369518.1, NM_001369519.1, NM_001369520.1, XM_017024973.2 and XM_024450896.1 provide exemplary human STAT3 mRNA transcript sequences. Examples of STAT3 polypeptides include any such native polypeptides from any vertebrate source as described above. In certain embodiments, STAT3 gene expression is determined by the amounts of the STAT3 polypeptides expressed from the STAT3 genes. In certain embodiments, the STAT3 polypeptides include all polypeptides encoded by the natural variants of the STAT3 genes and transcripts thereof, including allelic variants (e.g., SNP variants); splice variants; fragments; and derivatives. The STAT3 polypeptides of the present disclosure also encompass “full-length,” unprocessed STAT3 polypeptide as well as any form of STAT3 polypeptide that results from processing in the cell. NCBI Reference Sequences NP_644805.1, NP_003141.2, NP_998827.1, NP_001356441.1, NP_001356442.1, NP_001356443.1, NP_001356445.1, NP_001356446.1, NP_001356447.1, NP_001356448.1, NP_001356449.1, XP_016880462.1 and XP_024306664.1 provide exemplary human STAT3 polypeptide sequences.

As used herein, the term “STAT4” refers to “Signal Transducer And Activator Of Transcription 4,” in Uniprot or GenBank database. The term “STAT4” encompasses the STAT4 polypeptides, the STAT4 RNA transcripts, and the STAT4 genes. The term “STAT4 gene” refers to genes encoding STAT4 polypeptides. STAT4 is expressed in various cells and tissues including conventional dendritic cells, pancreatic duct cells and peripheral blood mononuclear cells, among others. Examples of STAT4 genes encompass any such native genes from any vertebrate source, including mammals such as primates (e.g., humans and chimpanzees), cows, dogs, and rodents (e.g. mice and rats), unless otherwise indicated. In certain embodiments, the term “STAT4 gene” includes all natural variants of STAT4 genes, including allelic variants (e.g., SNP variants) and mutations. NCBI Reference Sequence NG_012852 provides an exemplary human STAT4 nucleic acid sequence. In certain embodiments, STAT4 gene expression is determined by the amounts of the mRNA transcripts. In certain embodiments, the mRNA transcripts are splice variants, fragments or derivatives of all native and natural variants of the transcripts of the STAT4 genes. NCBI Reference Sequences NM_003151.4, NM_001243835.2, XM_006712719.3, XM_011511705.2 and XM_017004784.2 provide exemplary human STAT4 mRNA transcript sequences. Examples of STAT4 polypeptides include any such native polypeptides from any vertebrate source as described above. In certain embodiments, STAT4 gene expression is determined by the amount of the STAT4 polypeptides expressed from the STAT4 genes. In certain embodiments, the STAT4 polypeptide includes all polypeptides encoded by the natural variants of the STAT4 genes and transcripts thereof, including allelic variants (e.g., SNP variants); splice variants; fragments; and derivatives. The STAT4 polypeptides of the present disclosure also encompass “full-length,” unprocessed STAT4 polypeptide as well as any form of STAT4 polypeptide that results from processing in the cell. NCBI Reference Sequences NP_003142.1, NP_001230764.1, XP_006712782.1, XP_011510007.1 and XP_016860273.1 provide exemplary human STAT4 polypeptide sequences.

As used herein, the term “STAT5” refers to “STAT5A,” “STAT5B,” or both “STAT5A” and “STAT5B.” As used herein, the term “STAT5A” refers to “Signal Transducer And Activator Of Transcription 5A,” in Uniprot or GenBank database. The term “STAT5A” encompasses the STAT5A polypeptides, the STAT5A RNA transcripts, and the STAT5A genes. The term “STAT5A gene” refers to genes encoding STAT5A polypeptides. STAT5A is expressed in various cells and tissues including erythroblasts, peripheral blood mononuclear cells and T lymphocyte, among others. Examples of STAT5A genes encompass any such native genes from any vertebrate source, including mammals such as primates (e.g., humans and chimpanzees), cows, dogs, and rodents (e.g. mice and rats), unless otherwise indicated. In certain embodiments, the term “STAT5A gene” includes all natural variants of STAT5A genes, including allelic variants (e.g., SNP variants) and mutations. NCBI Reference Sequence NC_000017.11 range 42287547 . . . 42311943 provides an exemplary human STAT5A nucleic acid sequence. In certain embodiments, STAT5A gene expression is determined by the amounts of the mRNA transcripts. In certain embodiments, the mRNA transcripts are splice variants, fragments or derivatives of all native and natural variants of the transcripts of the STAT5A genes. NCBI Reference Sequences NM_001288718.1, NM_003152.3, NM_001288719.1, NM_001288720.1 and XM_005257624.3 provide exemplary human STAT5A mRNA transcript sequences. Examples of STAT5A polypeptides include any such native polypeptides from any vertebrate source as described above. In certain embodiments, STAT5A gene expression is determined by the amounts of the STAT5A polypeptides expressed from the STAT5A genes. In certain embodiments, the STAT5A polypeptides include all polypeptides encoded by the natural variants of the STAT5A genes and transcripts thereof, including allelic variants (e.g., SNP variants); splice variants; fragments; and derivatives. The STAT5A polypeptides of the present disclosure also encompass “full-length,” unprocessed STAT5A polypeptide as well as any form of STAT5A polypeptide that results from processing in the cell. NCBI Reference Sequences NP_001275647.1, NP_003143.2, NP_001275648.1, NP_001275649.1 and XP_005257681.1 provide exemplary human STAT5A polypeptide sequences.

As used herein, the term “STAT5B” refers to “Signal Transducer And Activator Of Transcription 5B,” also known as “Transcription Factor STAT5B,” in Uniprot or GenBank database. The term “STAT5B” encompasses the STAT5B polypeptides, the STAT5B RNA transcripts, and the STAT5B genes. The term “STAT5B gene” refers to genes encoding STAT5B polypeptides. STAT5B is expressed in various cells and tissues including peripheral blood mononuclear cells, CD8 T cells and the lymph node, among others. Examples of STAT5B genes encompass any such native genes from any vertebrate source, including mammals such as primates (e.g., humans and chimpanzees), cows, dogs, and rodents (e.g. mice and rats), unless otherwise indicated. In certain embodiments, the term “STAT5B gene” includes all natural variants of STAT5B genes, including allelic variants (e.g., SNP variants) and mutations. NCBI Reference Sequence NG_007271 provides an exemplary human STAT5B nucleic acid sequence. In certain embodiments, STAT5B gene expression is determined by the amounts of the mRNA transcripts. In certain embodiments, the mRNA transcripts are splice variants, fragments or derivatives of all native and natural variants of the transcripts of the STAT5B genes. NCBI Reference Sequence NM_012448.4, XM_005257626.4, XM_017024977.1, XM_024450897.1 and XM_024450898.1 provide exemplary human STAT5B mRNA transcript sequences. Examples of STAT5B polypeptides include any such native polypeptides from any vertebrate source as described above. In certain embodiments, STAT5B gene expression is determined by the amounts of the STAT5B polypeptides expressed from the STAT5B. In certain embodiments, the STAT5B polypeptide includes all polypeptides encoded by the natural variants of the STAT5B genes and transcripts thereof, including allelic variants (e.g., SNP variants); splice variants; fragments; and derivatives. The STAT5B polypeptides of the present disclosure also encompass “full-length,” unprocessed STAT5B polypeptide as well as any form of STAT5B polypeptide that results from processing in the cell. NCBI Reference Sequences NP_036580.2, XP_005257683.1, XP_016880466.1, XP_024306665.1 and XP_024306666.1 provide exemplary human STAT5B polypeptide sequences.

As used herein, the term “STAT6” refers to “Signal Transducer And Activator Of Transcription 6,” also known as “Signal Transducer And Activator Of Transcription 6, Interleukin-4 Induced,” “IL-4 STAT,” or “Transcription Factor IL-4 STAT,” in Uniprot or GenBank database. The term “STAT6” encompasses the STAT6 polypeptides, the STAT6 RNA transcripts, and the STAT6 genes. The term “STAT6 gene” refers to genes encoding STAT6 polypeptides. STAT6 is expressed in various cells and tissues including whole blood, the lymph node and the spleen, among others. Examples of STAT6 genes encompass any such native genes from any vertebrate source, including mammals such as primates (e.g., humans and chimpanzees), cows, dogs, and rodents (e.g. mice and rats), unless otherwise indicated. In certain embodiments, the term “STAT6 gene” includes all natural variants of STAT6 genes, including allelic variants (e.g., SNP variants) and mutations. NCBI Reference Sequence NG_021272 provides an exemplary human STAT6 nucleic acid sequence. In certain embodiments, STAT6 gene expression is determined by the amounts of the mRNA transcripts. In certain embodiments, the mRNA transcripts are splice variants, fragments or derivatives of all native and natural variants of the transcripts of the STAT6 genes. NCBI Reference Sequences NM_003153.5, NM_001178078.2, NM_001178079.2, NM_001178080.2, NM_001178081.2, NR 033659.2, XM_011538703.3, XM_011538704.3, XM_011538705.3, XM_011538707.3 and XM_011538708.3 provide exemplary human STAT6 mRNA transcript sequences. Examples of STAT6 polypeptides include any such native polypeptides from any vertebrate source as described above. In certain embodiments, STAT6 gene expression is determined by the amounts of the STAT6 polypeptides expressed from the STAT6 genes. In certain embodiments, the STAT6 polypeptides include all polypeptides encoded by the natural variants of the STAT6 genes and transcripts thereof, including allelic variants (e.g., SNP variants); splice variants; fragments; and derivatives. The STAT6 polypeptides of the present disclosure also encompass “full-length,” unprocessed STAT6 polypeptide as well as any form of STAT6 polypeptide that results from processing in the cell. NCBI Reference Sequences NP_003144.3, NP_001171549.1, NP_001171550.1, NP_001171551.1, NP_001171552.1, XP_011537005.1, XP_011537006.1, XP_011537007.1, XP_011537009.1 and XP_011537010.1 provide exemplary human STAT6 polypeptide sequences.

As used herein, the term “TAP2” refers to “Transporter 2, ATP binding cassette subfamily B member,” also known as “antigen peptide transporter 2,” “ATP-binding cassette, sub-family B (MDR/TAP), member 3,” “Peptide Transporter Involved In Antigen Processing 2” or “Really Interesting New Gene 11 Protein,” in Uniprot or GenBank database. The term “TAP2” encompasses the TAP2 polypeptides, the TAP2 RNA transcripts, and the TAP2 genes. The term “TAP2 gene” refers to genes encoding TAP2 polypeptides. TAP2 is expressed in various cells and tissues including peripheral blood mononuclear cells, among others. Examples of TAP2 genes encompass any such native genes from any vertebrate source, including mammals such as primates (e.g., humans and chimpanzees), dogs, cow, chicken, reptiles (e.g. lizard), and rodents (e.g., mice and rats), unless otherwise indicated. In certain embodiments, the term “TAP2 gene” includes all natural variants of TAP2 genes, including allelic variants (e.g., SNP variants) and mutations. NCBI Reference Sequence NG_009793 provides an exemplary human TAP2 nucleic acid sequence. In certain embodiments, TAP2 gene expression is determined by the amounts of the mRNA transcripts. TAP2 gene encodes various transcript variants. In certain embodiments, the mRNA transcripts are splice variants, fragments or derivatives of all native and natural variants of the transcripts of the TAP2 genes. NCBI Reference Sequences NM_001290043.2, NM_000544.3 and NM_018833.2 provide exemplary human TAP2 mRNA transcript sequences. Examples of TAP2 polypeptides include any such native polypeptides from any vertebrate source as described above. In certain embodiments, TAP2 gene expression is determined by the amounts of the TAP2 polypeptides expressed from the TAP2 genes. In certain embodiments, the TAP2 polypeptides include all polypeptides encoded by the natural variants of the TAP2 genes and transcripts thereof, including allelic variants (e.g., SNP variants); splice variants; fragments; and derivatives. The TAP2 polypeptides of the present disclosure also encompass “full-length,” unprocessed TAP2 polypeptide as well as any form of TAP2 polypeptide that results from processing in the cell. NCBI Reference Sequences NP_001276972.1, NP_000535.3, and NP_061313.2 provide exemplary human TAP2 polypeptide sequences.

As used herein, the term “TLR7” refers to “Toll Like Receptor 7,” also known as “Toll-Like Receptor 7” or “Toll-Like Receptor 7-Like,” in Uniprot or GenBank database. The term “TLR7” encompasses the TLR7 polypeptides, the TLR7 RNA transcripts, and the TLR7 genes. The term “TLR7 gene” refers to genes encoding TLR7 polypeptides. TLR7 is expressed in various cells and tissues including plasmacytoid dendritic cells and podocytes, among others. Examples of TLR7 genes encompass any such native genes from any vertebrate source, including mammals such as primates (e.g., humans and chimpanzees), cows, dogs, and rodents (e.g. mice and rats), unless otherwise indicated. In certain embodiments, the term “TLR7 gene” includes all natural variants of TLR7 genes, including allelic variants (e.g., SNP variants) and mutations. NCBI Reference Sequence NG_012569 provides an exemplary human TLR7 nucleic acid sequence. In certain embodiments, TLR7 gene expression is determined by the amounts of the mRNA transcripts. In certain embodiments, the mRNA transcripts are splice variants, fragments or derivatives of all native and natural variants of the transcripts of the TLR7 genes. NCBI Reference Sequence NM_016562.4 provides an exemplary human TLR7 mRNA transcript sequences. Examples of TLR7 polypeptides include any such native polypeptides from any vertebrate source as described above. In certain embodiments, TLR7 gene expression is determined by the amounts of the TLR7 polypeptides expressed from the TLR7 genes. In certain embodiments, the TLR7 polypeptides include all polypeptides encoded by the natural variants of the TLR7 genes and transcripts thereof, including allelic variants (e.g., SNP variants); splice variants; fragments; and derivatives. The TLR7 polypeptides of the present disclosure also encompass “full-length,” unprocessed TLR7 polypeptide as well as any form of TLR7 polypeptide that results from processing in the cell. NCBI Reference Sequence NP_057646.1 provides an exemplary human TLR7 polypeptide sequence.

As used herein, the term “TLR8” refers to “Toll Like Receptor 8,” also known as “Toll-Like Receptor 8” or “CD288 Antigen,” in Uniprot or GenBank database. The term “TLR8” encompasses the TLR8 polypeptides, the TLR8 RNA transcripts, and the TLR8 genes. The term “TLR8 gene” refers to genes encoding TLR8 polypeptides. TLR8 is expressed in various cells and tissues including monocytes and B lymphocytes, among others. Examples of TLR8 genes encompass any such native genes from any vertebrate source, including mammals such as primates (e.g., humans and chimpanzees), cows, dogs, and rodents (e.g. mice and rats), unless otherwise indicated. In certain embodiments, the term “TLR8 gene” includes all natural variants of TLR8 genes, including allelic variants (e.g., SNP variants) and mutations. NCBI Reference Sequence NG_012882 provides an exemplary human TLR8 nucleic acid sequence. In certain embodiments, TLR8 gene expression is determined by the amounts of the mRNA transcripts. In certain embodiments, the mRNA transcripts are splice variants, fragments or derivatives of all native and natural variants of the transcripts of the TLR8 genes. NCBI Reference Sequences NM_138636.5, NM_016610.4, XM_011545529.1 and XM_011545530.2 provide exemplary human TLR8 mRNA transcript sequences. Examples of TLR8 polypeptides include any such native polypeptides from any vertebrate source as described above. In certain embodiments, TLR8 gene expression is determined by the amounts of the TLR8 polypeptides expressed from the TLR8 gene. In certain embodiments, the TLR8 polypeptides include all polypeptides encoded by the natural variants of the TLR8 genes and transcripts thereof, including allelic variants (e.g., SNP variants); splice variants; fragments; and derivatives. The TLR8 polypeptides of the present disclosure also encompass “full-length,” unprocessed TLR8 polypeptide as well as any form of TLR8 polypeptide that results from processing in the cell. NCBI Reference Sequences NP_619542.1, NP_057694.2, XP_011543831.1 and XP_011543832.1 provide exemplary human TLR8 polypeptide sequence.

As used herein, the term “TLR9” refers to “Toll Like Receptor 9,” also known as “Toll-Like Receptor 9” or “CD289 Antigen,” in Uniprot or GenBank database. The term “TLR9” encompasses the TLR9 polypeptides, the TLR9 RNA transcripts, and the TLR9 genes. The term “TLR9 gene” refers to genes encoding TLR9 polypeptides. TLR9 is expressed in various cells and tissues including the B lymphocyte, adipocyte and spleen, among others. Examples of TLR9 genes encompass any such native genes from any vertebrate source, including mammals such as primates (e.g., humans and chimpanzees), cows, dogs, and rodents (e.g. mice and rats), unless otherwise indicated. In certain embodiments, the term “TLR9 gene” includes all natural variants of TLR9 genes, including allelic variants (e.g., SNP variants) and mutations. NCBI Reference Sequence NG_033933 provides an exemplary human TLR9 nucleic acid sequence. In certain embodiments, TLR9 gene expression is determined by the amounts of the mRNA transcripts. In certain embodiments, the mRNA transcripts are splice variants, fragments or derivatives of all native and natural variants of the transcripts of the TLR9 genes. NCBI Reference Sequence NM_017442.3 provides an exemplary human TLR9 mRNA transcript sequence. Examples of TLR9 polypeptides include any such native polypeptides from any vertebrate source as described above. In certain embodiments, TLR9 gene expression is determined by the amounts of the TLR9 polypeptides expressed from the TLR genes. In certain embodiments, the TLR9 polypeptides include all polypeptides encoded by the natural variants of the TLR9 genes and transcripts thereof, including allelic variants (e.g., SNP variants); splice variants; fragments; and derivatives. The TLR9 polypeptides of the present disclosure also encompass “full-length,” unprocessed TLR9 polypeptide as well as any form of TLR9 polypeptide that results from processing in the cell. NCBI Reference Sequence NP_059138.1 provides an exemplary human TLR9 polypeptide sequence.

As used herein, the term “TRAF2” refers to “TNF Receptor Associated Factor 2,” also known as “RING-Type E3 Ubiquitin Transferase TRAF2,” “Tumor necrosis factor type 2 receptor-associated protein 3,” or “E3 Ubiquitin-Protein Ligase TRAF2,” in Uniprot or GenBank database. The term “TRAF2” encompasses the TRAF2 polypeptides, the TRAF2 RNA transcripts, and the TRAF2 genes. The term “TRAF2 gene” refers to genes encoding TRAF2 polypeptides. TRAF2 is expressed in various cells and tissues including epithelial cells, muscle, heart and liver, among others. Examples of TRAF2 genes encompass any such native genes from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TRAF2 gene” includes all natural variants of the TRAF2 genes, including allelic variants (e.g., SNP variants) and mutations. NCBI Gene ID 7186 provides an exemplary human TRAF2 nucleic acid sequence. In certain embodiments, TRAF2 gene expression is determined by the amounts of the mRNA transcripts. TRAF2 gene encodes various transcript variants. In certain embodiments, the mRNA transcripts are splice variants, fragments or derivatives of all native and natural variants of the transcripts of TRAF2. NCBI Reference Sequences NM_021138.4, XM_011518974.2, XM_011518976.3 and XM_011518977.2 provide exemplary human TRAF2 mRNA transcript sequences. Examples of TRAF2 polypeptides include any such native polypeptides from any vertebrate source as described above. In certain embodiments, TRAF2 gene expression is determined by the amounts of the TRAF2 polypeptides. In certain embodiments, the TRAF2 polypeptides include all polypeptides encoded by the natural variants of the TRAF2 genes and transcripts thereof, including allelic variants (e.g., SNP variants); splice variants; fragments; and derivatives. The TRAF2 polypeptides of the present disclosure also encompass “full-length,” unprocessed TRAF2 polypeptide as well as any form of TRAF2 polypeptide that results from processing in the cell. NCBI Reference Sequences NP_066961.2, XP_011517276.1, XP_011517278.1, and XP_011517279.1 provide exemplary human TRAF2 polypeptide sequences.

As used herein, the term “XBP-1” refers to “X-Box Binding Protein 1,” also known as “Tax-Responsive Element-Binding Protein 5” or “X-Box-Binding Protein 1,” in Uniprot or GenBank database. The term “XBP-1” encompasses the XBP-1 polypeptides, the XBP-1 RNA transcripts, and the XBP-1 genes. The term “XBP-1 gene” refers to genes encoding XBP-1 polypeptides. XBP-1 is expressed in various cells and tissues including plasma and endothelial cells, among others. Examples of XBP-1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “XBP-1 gene” includes all natural variants of the XBP-1 genes, including allelic variants (e.g., SNP variants) and mutations. NCBI Reference Sequence NG_012266.1 provides an exemplary human XBP-1 nucleic acid sequence. In certain embodiments, XBP-1 gene expression is determined by the amounts of the mRNA transcripts. XBP-1 gene encodes various transcript variants. In certain embodiments, the mRNA transcripts are splice variants, fragments or derivatives of all native and natural variants of the transcripts of the XBP-1 genes. NCBI Reference Sequences NM_001079539.1 and NM_005080.3 provide exemplary human XBP-1 mRNA transcript sequences. Examples of XBP-1 polypeptides include any such native polypeptides from any vertebrate source as described above. In certain embodiments, XBP-1 gene expression is determined by the amounts of the XBP-1 polypeptides. In certain embodiments, the XBP-1 polypeptides include all polypeptides encoded by the natural variants of the XBP-1 genes and transcripts thereof, including allelic variants (e.g., SNP variants); splice variants; fragments; and derivatives. The XBP-1 polypeptides of the present disclosure also encompass “full-length,” unprocessed XBP-1 polypeptide as well as any form of XBP-1 polypeptide that results from processing in the cell. NCBI Reference Sequences NP_001073007.1 and NP_005071.2 provide exemplary human XBP-1 polypeptide sequences. “XBP-1S” refers to the spliced form of XBP-1 that is a transcription factor and a marker of ER stress and the corresponding polypeptide. Examples of XBP-1S include human XBP-1S corresponding to Ensembl entry ID ENST00000216037.10 or equivalents thereof in other species. “XBP-1L” refers to the long form of spliced XBP-1 that is a transcription repressor and the corresponding polypeptide. Examples of XBP-1L include human XBP-1L corresponding to to Ensembl entry ID ENST00000344347.5 or equivalents thereof in other species.

As used herein, the term “RFX1” refers to “regulatory factor X, 1,” also known as “MHC class II regulatory factor RFX1” or “transcription factor RFX1,” in Uniprot or GenBank database. The term “RFX1” encompasses the RFX1 polypeptides, the RFX1 RNA transcripts, and the RFX1 genes. The term “RFX1 gene” refers to genes encoding RFX1 polypeptides. RFX1 is expressed in various cells and tissues including plasma and endothelial cells, among others. Examples of RFX1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RFX1 gene” includes all natural variants of the RFX1 genes, including allelic variants (e.g., SNP variants) and mutations. NCBI Gene ID: 5989 and NCBI Reference Sequence NC_000019.10 (range 13961530 . . . 14007514, complement) provide exemplary human RFX1 nucleic acid sequences. In certain embodiments, RFX1 gene expression is determined by the amounts of the mRNA transcripts. RFX1 gene encodes various transcript variants. In certain embodiments, the mRNA transcripts are splice variants, fragments or derivatives of all native and natural variants of the transcripts of the RFX1 genes. NCBI Reference Sequences NM_002918.5, XM_011528170.2, XM_011528167.2, XM_011528168.2, XM_011528165.2, XM_011528169.2, and XM_011528166.2 provide exemplary human RFX1 mRNA transcript sequences. Examples of RFX1 polypeptides include any such native polypeptides from any vertebrate source as described above. In certain embodiments, RFX1 gene expression is determined by the amounts of the RFX1 polypeptides. In certain embodiments, the RFX1 polypeptides include all polypeptides encoded by the natural variants of the RFX1 genes and transcripts thereof, including allelic variants (e.g., SNP variants); splice variants; fragments; and derivatives. The RFX1 polypeptides of the present disclosure also encompass “full-length,” unprocessed RFX1 polypeptide as well as any form of RFX1 polypeptide that results from processing in the cell. NCBI Reference Sequences NP_002909.4, XP_011526472.1, XP_011526469.1, XP_011526470.1, XP_011526467.1, XP_011526471.1, and XP_011526468.1 provide exemplary human RFX1 polypeptide sequences.

As used herein, the term “RFX5” refers to “Regulatory factor X 5,” also known as “DNA-binding protein RFX5,” in Uniprot or GenBank database. The term “RFX5” encompasses the RFX5 polypeptides, the RFX5 RNA transcripts, and the RFX5 genes. The term “RFX5 gene” refers to genes encoding RFX5 polypeptides. RFX5 is expressed in various cells and tissues including plasma and endothelial cells, among others. Examples of RFX5 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RFX5 gene” includes all natural variants of the RFX5 genes, including allelic variants (e.g., SNP variants) and mutations. NCBI Gene ID: 5993 and NCBI Reference Sequence NC_000001.11 (range 151340640 . . . 151347319, complement) provide exemplary human RFX5 nucleic acid sequences. In certain embodiments, RFX5 gene expression is determined by the amounts of the mRNA transcripts. RFX5 gene encodes various transcript variants. In certain embodiments, the mRNA transcripts are splice variants, fragments or derivatives of all native and natural variants of the transcripts of the RFX5 genes. NCBI Reference Sequences NM_000449.4, NM_001025603.2, and NM_001379412.1 provide exemplary human RFX5 mRNA transcript sequences. Examples of RFX5 polypeptides include any such native polypeptides from any vertebrate source as described above. In certain embodiments, RFX5 gene expression is determined by the amounts of the RFX5 polypeptides. In certain embodiments, the RFX5 polypeptides include all polypeptides encoded by the natural variants of the RFX5 genes and transcripts thereof, including allelic variants (e.g., SNP variants); splice variants; fragments; and derivatives. The RFX5 polypeptides of the present disclosure also encompass “full-length,” unprocessed RFX5 polypeptide as well as any form of RFX5 polypeptide that results from processing in the cell. NCBI Reference Sequences NP_000440.1, NP_001020774.1, and NP_001366341.1 provide exemplary human RFX5 polypeptide sequences.

As used herein, the term “RFX7” refers to “Regulatory factor X 7,” also known as “DNA-binding protein RFX7,” or “Regulatory factor X domain-containing protein 2,” in Uniprot or GenBank database. The term “RFX7” encompasses the RFX7 polypeptides, the RFX7 RNA transcripts, and the RFX7 genes. The term “RFX7 gene” refers to genes encoding RFX7 polypeptides. RFX7 is expressed in various cells and tissues including plasma and endothelial cells, among others. Examples of RFX7 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RFX7 gene” includes all natural variants of the RFX7 genes, including allelic variants (e.g., SNP variants) and mutations. NCBI Gene ID: 64864 and NCBI Reference Sequence NC_000015.10 (range 56087280 . . . 56247654, complement) provide exemplary human RFX7 nucleic acid sequences. In certain embodiments, RFX7 gene expression is determined by the amounts of the mRNA transcripts. RFX7 gene encodes various transcript variants. In certain embodiments, the mRNA transcripts are splice variants, fragments or derivatives of all native and natural variants of the transcripts of the RFX7 genes. NCBI Reference Sequences NM_001368073.2, NM_001368074.1, NM_001370561.1, and NM_001370554.1 provide exemplary human RFX7 mRNA transcript sequences. Examples of RFX7 polypeptides include any such native polypeptides from any vertebrate source as described above. In certain embodiments, RFX7 gene expression is determined by the amounts of the RFX7 polypeptides. In certain embodiments, the RFX7 polypeptides include all polypeptides encoded by the natural variants of the RFX7 genes and transcripts thereof, including allelic variants (e.g., SNP variants); splice variants; fragments; and derivatives. The RFX7 polypeptides of the present disclosure also encompass “full-length,” unprocessed RFX7 polypeptide as well as any form of RFX7 polypeptide that results from processing in the cell. NCBI Reference Sequences NP_001355002.1, NP_001355003.1, NP_001357490.1, and NP_001357483.1 provide exemplary human RFX7 polypeptide sequences.

As used herein, the term “CTCF” refers to “transcriptional repressor CTCF,” also known as “11-zinc finger protein,” “CCCTC-binding factor,” or “CTCFL paralog,” in Uniprot or GenBank database. The term “CTCF” encompasses the CTCF polypeptides, the CTCF RNA transcripts, and the CTCF genes. The term “CTCF gene” refers to genes encoding CTCF polypeptides. CTCF is expressed in various cells and tissues including plasma and endothelial cells, among others. Examples of CTCF genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CTCF gene” includes all natural variants of the CTCF genes, including allelic variants (e.g., SNP variants) and mutations. NCBI Gene ID: 10664 and NCBI Reference Sequence NC_000016.10 (range 67562526 . . . 67639185) provide exemplary human CTCF nucleic acid sequences. In certain embodiments, CTCF gene expression is determined by the amounts of the mRNA transcripts. CTCF gene encodes various transcript variants. In certain embodiments, the mRNA transcripts are splice variants, fragments or derivatives of all native and natural variants of the transcripts of the CTCF genes. NCBI Reference Sequences NM_001191022.2, NM_001363916.1, and NM_006565.4 provide exemplary human CTCF mRNA transcript sequences. Examples of CTCF polypeptides include any such native polypeptides from any vertebrate source as described above. In certain embodiments, CTCF gene expression is determined by the amounts of the CTCF polypeptides. In certain embodiments, the CTCF polypeptides include all polypeptides encoded by the natural variants of the CTCF genes and transcripts thereof, including allelic variants (e.g., SNP variants); splice variants; fragments; and derivatives. The CTCF polypeptides of the present disclosure also encompass “full-length,” unprocessed CTCF polypeptide as well as any form of CTCF polypeptide that results from processing in the cell. NCBI Reference Sequences NP_001177951.1, NP_001350845.1, and NP_006556.1 provide exemplary human CTCF polypeptide sequences.

As used herein, the term “CIITA” refers to “class II major histocompatibility complex transactivator,” also known as “MHC class II transactivator,” in Uniprot or GenBank database. The term “CIITA” encompasses the CIITA polypeptides, the CIITA RNA transcripts, and the CIITA genes. The term “CIITA gene” refers to genes encoding CIITA polypeptides. CIITA is expressed in various cells and tissues including plasma and endothelial cells, among others. Examples of CIITA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CIITA gene” includes all natural variants of the CIITA genes, including allelic variants (e.g., SNP variants) and mutations. NCBI Gene ID: 4261 and NCBI Reference Sequence NC_000016.10 (range 10866208 . . . 10941562) provide exemplary human CIITA nucleic acid sequences. In certain embodiments, CIITA gene expression is determined by the amounts of the mRNA transcripts. CIITA gene encodes various transcript variants. In certain embodiments, the mRNA transcripts are splice variants, fragments or derivatives of all native and natural variants of the transcripts of the CIITA genes. NCBI Reference Sequences NM_000246.3, NM_001286402.1, and NM_001286403.2 provide exemplary human CIITA mRNA transcript sequences. Examples of CIITA polypeptides include any such native polypeptides from any vertebrate source as described above. In certain embodiments, CIITA gene expression is determined by the amounts of the CIITA polypeptides. In certain embodiments, the CIITA polypeptides include all polypeptides encoded by the natural variants of the CIITA genes and transcripts thereof, including allelic variants (e.g., SNP variants); splice variants; fragments; and derivatives. The CIITA polypeptides of the present disclosure also encompass “full-length,” unprocessed CIITA polypeptide as well as any form of CIITA polypeptide that results from processing in the cell. NCBI Reference Sequences NP_000237.2, NP_001273331.1, and NP_001273332.1 provide exemplary human CIITA polypeptide sequences.

As used herein, the term “BCL2L11” refers to “bcl-2-like protein 11,” also known as “Bcl2-interacting mediator of cell death,” in Uniprot or GenBank database. The term “BCL2L11” encompasses the BCL2L11 polypeptides, the BCL2L11 RNA transcripts, and the BCL2L11 genes. The term “BCL2L11 gene” refers to genes encoding BCL2L11 polypeptides. BCL2L11 is expressed in various cells and tissues including plasma and endothelial cells, among others. Examples of BCL2L11 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BCL2L11 gene” includes all natural variants of the BCL2L11 genes, including allelic variants (e.g., SNP variants) and mutations. NCBI Gene ID: 10018 and NCBI Reference Sequence NC_000002.12 (range 111120914 . . . 111168445) provide exemplary human BCL2L11 nucleic acid sequences. In certain embodiments, BCL2L11 gene expression is determined by the amounts of the mRNA transcripts. BCL2L11 gene encodes various transcript variants. In certain embodiments, the mRNA transcripts are splice variants, fragments or derivatives of all native and natural variants of the transcripts of the BCL2L11 genes. NCBI Reference Sequences NM_001204106.2, NM_001204107.1, and NM_001204108.1 provide exemplary human BCL2L11 mRNA transcript sequences. Examples of BCL2L11 polypeptides include any such native polypeptides from any vertebrate source as described above. In certain embodiments, BCL2L11 gene expression is determined by the amounts of the BCL2L11 polypeptides. In certain embodiments, the BCL2L11 polypeptides include all polypeptides encoded by the natural variants of the BCL2L11 genes and transcripts thereof, including allelic variants (e.g., SNP variants); splice variants; fragments; and derivatives. The BCL2L11 polypeptides of the present disclosure also encompass “full-length,” unprocessed BCL2L11 polypeptide as well as any form of BCL2L11 polypeptide that results from processing in the cell. NCBI Reference Sequences NP_001191035.1, NP_001191036.1, and NP_001191037.1 provide exemplary human BCL2L11 polypeptide sequences.

As used herein, the term “BCAP31” refers to “B-cell receptor-associated protein 31,” also known as “6C₆-AG tumor-associated antigen,” in Uniprot or GenBank database. The term “BCAP31” encompasses the BCAP31 polypeptides, the BCAP31 RNA transcripts, and the BCAP31 genes. The term “BCAP31 gene” refers to genes encoding BCAP31 polypeptides. BCAP31 is expressed in various cells and tissues including plasma and endothelial cells, among others. Examples of BCAP31 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BCAP31 gene” includes all natural variants of the BCAP31 genes, including allelic variants (e.g., SNP variants) and mutations. NCBI Gene ID: 10134 and NCBI Reference Sequence NC_000023.11 (range 153700492 . . . 153724746, complement) provide exemplary human BCAP31 nucleic acid sequences. In certain embodiments, BCAP31 gene expression is determined by the amounts of the mRNA transcripts. BCAP31 gene encodes various transcript variants. In certain embodiments, the mRNA transcripts are splice variants, fragments or derivatives of all native and natural variants of the transcripts of the BCAP31 genes. NCBI Reference Sequences NM_001139441.1, NM_001139457.2, and NM_001256447.2 provide exemplary human BCAP31 mRNA transcript sequences. Examples of BCAP31 polypeptides include any such native polypeptides from any vertebrate source as described above. In certain embodiments, BCAP31 gene expression is determined by the amounts of the BCAP31 polypeptides. In certain embodiments, the BCAP31 polypeptides include all polypeptides encoded by the natural variants of the BCAP31 genes and transcripts thereof, including allelic variants (e.g., SNP variants); splice variants; fragments; and derivatives. The BCAP31 polypeptides of the present disclosure also encompass “full-length,” unprocessed BCAP31 polypeptide as well as any form of BCAP31 polypeptide that results from processing in the cell. NCBI Reference Sequences NP_001132913.1, NP_001132929.1, and NP_001243376.1 provide exemplary human BCAP31 polypeptide sequences.

As used herein, the term “SERINC3” refers to “serine incorporator 3,” also known as “tumor differentially expressed protein 1,” in Uniprot or GenBank database. The term “SERINC3” encompasses the SERINC3 polypeptides, the SERINC3 RNA transcripts, and the SERINC3 genes. The term “SERINC3 gene” refers to genes encoding SERINC3 polypeptides. SERINC3 is expressed in various cells and tissues including plasma and endothelial cells, among others. Examples of SERINC3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “SERINC3 gene” includes all natural variants of the SERINC3 genes, including allelic variants (e.g., SNP variants) and mutations. NCBI Gene ID: 10955 and NCBI Reference Sequence NC_000020.11 (range 44496221 . . . 44522116, complement) provide exemplary human SERINC3 nucleic acid sequences. In certain embodiments, SERINC3 gene expression is determined by the amounts of the mRNA transcripts. SERINC3 gene encodes various transcript variants. In certain embodiments, the mRNA transcripts are splice variants, fragments or derivatives of all native and natural variants of the transcripts of the SERINC3 genes. NCBI Reference Sequences NM_006811.4, and NM_198941.2 provide exemplary human SERINC3 mRNA transcript sequences. Examples of SERINC3 polypeptides include any such native polypeptides from any vertebrate source as described above. In certain embodiments, SERINC3 gene expression is determined by the amounts of the SERINC3 polypeptides. In certain embodiments, the SERINC3 polypeptides include all polypeptides encoded by the natural variants of the SERINC3 genes and transcripts thereof, including allelic variants (e.g., SNP variants); splice variants; fragments; and derivatives. The SERINC3 polypeptides of the present disclosure also encompass “full-length,” unprocessed SERINC3 polypeptide as well as any form of SERINC3 polypeptide that results from processing in the cell. NCBI Reference Sequences NP_006802.1, and NP_945179.1 provide exemplary human SERINC3 polypeptide sequences.

As used herein, the term “ERN1” refers to “Serine/threonine-protein kinase/endoribonuclease IRE1,” also known as “endoplasmic reticulum to nucleus signaling 1,” or “Inositol-requiring protein 1,” in Uniprot or GenBank database. The term “ERN1” encompasses the ERN1 polypeptides, the ERN1 RNA transcripts, and the ERN1 genes. The term “ERN1 gene” refers to genes encoding ERN1 polypeptides. ERN1 is expressed in various cells and tissues including plasma and endothelial cells, among others. Examples of ERN1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ERN1 gene” includes all natural variants of the ERN1 genes, including allelic variants (e.g., SNP variants) and mutations. NCBI Gene ID: 2081 and NCBI Reference Sequence NC_000017.11 (range 64039142 . . . 64132469, complement) provide exemplary human ERN1 nucleic acid sequences. In certain embodiments, ERN1 gene expression is determined by the amounts of the mRNA transcripts. ERN1 gene encodes various transcript variants. In certain embodiments, the mRNA transcripts are splice variants, fragments or derivatives of all native and natural variants of the transcripts of the ERN1 genes. NCBI Reference Sequences NM_001433.5, XM_017024347.2, and XM_017024348.2 provide exemplary human ERN1 mRNA transcript sequences. Examples of ERN1 polypeptides include any such native polypeptides from any vertebrate source as described above. In certain embodiments, ERN1 gene expression is determined by the amounts of the ERN1 polypeptides. In certain embodiments, the ERN1 polypeptides include all polypeptides encoded by the natural variants of the ERN1 genes and transcripts thereof, including allelic variants (e.g., SNP variants); splice variants; fragments; and derivatives. The ERN1 polypeptides of the present disclosure also encompass “full-length,” unprocessed ERN1 polypeptide as well as any form of ERN1 polypeptide that results from processing in the cell. NCBI Reference Sequences NP_001424.3, XP_016879836.1, and XP_016879837.1 provide exemplary human ERN1 polypeptide sequences.

As used herein, the term “ATF6” refers to “cyclic AMP-dependent transcription factor ATF-6 alpha,” also known as “Activating transcription factor 6 alpha,” or “cAMP-dependent transcription factor ATF-6 alpha,” in Uniprot or GenBank database. The term “ATF6” encompasses the ATF6 polypeptides, the ATF6 RNA transcripts, and the ATF6 genes. The term “ATF6 gene” refers to genes encoding ATF6 polypeptides. ATF6 is expressed in various cells and tissues including plasma and endothelial cells, among others. Examples of ATF6 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ATF6 gene” includes all natural variants of the ATF6 genes, including allelic variants (e.g., SNP variants) and mutations. NCBI Gene ID: 22926 and NCBI Reference Sequence NC_000001.11 (range 161766320 . . . 161964070) provide exemplary human ATF6 nucleic acid sequences. In certain embodiments, ATF6 gene expression is determined by the amounts of the mRNA transcripts. ATF6 gene encodes various transcript variants. In certain embodiments, the mRNA transcripts are splice variants, fragments or derivatives of all native and natural variants of the transcripts of the ATF6 genes. NCBI Reference Sequences NM_007348.4, XM_011509308.1, and XM_011509309.1 provide exemplary human ATF6 mRNA transcript sequences. Examples of ATF6 polypeptides include any such native polypeptides from any vertebrate source as described above. In certain embodiments, ATF6 gene expression is determined by the amounts of the ATF6 polypeptides. In certain embodiments, the ATF6 polypeptides include all polypeptides encoded by the natural variants of the ATF6 genes and transcripts thereof, including allelic variants (e.g., SNP variants); splice variants; fragments; and derivatives. The ATF6 polypeptides of the present disclosure also encompass “full-length,” unprocessed ATF6 polypeptide as well as any form of ATF6 polypeptide that results from processing in the cell, and any ATF6 polypeptide localized or re-localized anywhere in the cell. NCBI Reference Sequences NP_031374.2, XP_011507610.1, and XP_011507611.1 provide exemplary human ATF6 polypeptide sequences.

As used herein, the term “NCK2” refers to “NCK adaptor protein 2,” also known as “cytoplasmic protein NCK2,” “SH2/SH3 adaptor protein NCK-beta,” or “growth factor receptor-bound protein 4,” in Uniprot or GenBank database. The term “NCK2” encompasses the NCK2 polypeptides, the NCK2 RNA transcripts, and the NCK2 genes. The term “NCK2 gene” refers to genes encoding NCK2 polypeptides. NCK2 is expressed in various cells and tissues including plasma and endothelial cells, among others. Examples of NCK2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “NCK2 gene” includes all natural variants of the NCK2 genes, including allelic variants (e.g., SNP variants) and mutations. NCBI Gene ID: 8440 and NCBI Reference Sequence NC_000002.12 (range 105744649 . . . 105894274) provide exemplary human NCK2 nucleic acid sequences. In certain embodiments, NCK2 gene expression is determined by the amounts of the mRNA transcripts. NCK2 gene encodes various transcript variants. In certain embodiments, the mRNA transcripts are splice variants, fragments or derivatives of all native and natural variants of the transcripts of the NCK2 genes. NCBI Reference Sequences NM_001004720.3, NM_001004722.3, and NM_003581.5 provide exemplary human NCK2 mRNA transcript sequences. Examples of NCK2 polypeptides include any such native polypeptides from any vertebrate source as described above. In certain embodiments, NCK2 gene expression is determined by the amounts of the NCK2 polypeptides. In certain embodiments, the NCK2 polypeptides include all polypeptides encoded by the natural variants of the NCK2 genes and transcripts thereof, including allelic variants (e.g., SNP variants); splice variants; fragments; and derivatives. The NCK2 polypeptides of the present disclosure also encompass “full-length,” unprocessed NCK2 polypeptide as well as any form of NCK2 polypeptide that results from processing in the cell. NCBI Reference Sequences NP_001004720.1, NP_001004722.1, and NP_003572.2 provide exemplary human NCK2 polypeptide sequences.

As used herein, the term “PPP1R15A” refers to “protein phosphatase 1 regulatory subunit 15A,” also known as “Growth arrest and DNA damage-inducible protein GADD34,” or “myeloid differentiation primary response protein MyD116 homolog,” in Uniprot or GenBank database. The term “PPP1R15A” encompasses the PPP1R15A polypeptides, the PPP1R15A RNA transcripts, and the PPP1R15A genes. The term “PPP1R15A gene” refers to genes encoding PPP1R15A polypeptides. PPP1R15A is expressed in various cells and tissues including plasma and endothelial cells, among others. Examples of PPP1R15A genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “PPP1R15A gene” includes all natural variants of the PPP1R15A genes, including allelic variants (e.g., SNP variants) and mutations. NCBI Gene ID: 23645 and NCBI Reference Sequence NC_000019.10 (range 48872392 . . . 48876062) provide exemplary human PPP1R15A nucleic acid sequences. In certain embodiments, PPP1R15A gene expression is determined by the amounts of the mRNA transcripts. PPP1R15A gene encodes various transcript variants. In certain embodiments, the mRNA transcripts are splice variants, fragments or derivatives of all native and natural variants of the transcripts of the PPP1R15A genes. NCBI Reference Sequence NM_014330.3 provides an exemplary human PPP1R15A mRNA transcript sequence. Examples of PPP1R15A polypeptides include any such native polypeptides from any vertebrate source as described above. In certain embodiments, PPP1R15A gene expression is determined by the amounts of the PPP1R15A polypeptides. In certain embodiments, the PPP1R15A polypeptides include all polypeptides encoded by the natural variants of the PPP1R15A genes and transcripts thereof, including allelic variants (e.g., SNP variants); splice variants; fragments; and derivatives. The PPP1R15A polypeptides of the present disclosure also encompass “full-length,” unprocessed PPP1R15A polypeptide as well as any form of PPP1R15A polypeptide that results from processing in the cell. NCBI Reference Sequence NP_055145.3 provides an exemplary human PPP1R15A polypeptide sequence.

As used herein, the term “UBQLN2” refers to “Ubiquilin-2,” also known as “ubiquitin-like product Chap1/Dsk2,” or “Protein linking IAP with cytoskeleton 2,” in Uniprot or GenBank database. The term “UBQLN2” encompasses the UBQLN2 polypeptides, the UBQLN2 RNA transcripts, and the UBQLN2 genes. The term “UBQLN2 gene” refers to genes encoding UBQLN2 polypeptides. UBQLN2 is expressed in various cells and tissues including plasma and endothelial cells, among others. Examples of UBQLN2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “UBQLN2 gene” includes all natural variants of the UBQLN2 genes, including allelic variants (e.g., SNP variants) and mutations. NCBI Gene ID: 29978 and NCBI Reference Sequence NC_000023.11 (range 56563627 . . . 56567868) provide exemplary human UBQLN2 nucleic acid sequences. In certain embodiments, UBQLN2 gene expression is determined by the amounts of the mRNA transcripts. UBQLN2 gene encodes various transcript variants. In certain embodiments, the mRNA transcripts are splice variants, fragments or derivatives of all native and natural variants of the transcripts of the UBQLN2 genes. NCBI Reference Sequence NM_013444.4 provides an exemplary human UBQLN2 mRNA transcript sequence. Examples of UBQLN2 polypeptides include any such native polypeptides from any vertebrate source as described above. In certain embodiments, UBQLN2 gene expression is determined by the amounts of the UBQLN2 polypeptides. In certain embodiments, the UBQLN2 polypeptides include all polypeptides encoded by the natural variants of the UBQLN2 genes and transcripts thereof, including allelic variants (e.g., SNP variants); splice variants; fragments; and derivatives. The UBQLN2 polypeptides of the present disclosure also encompass “full-length,” unprocessed UBQLN2 polypeptide as well as any form of UBQLN2 polypeptide that results from processing in the cell. NCBI Reference Sequence NP_038472.2 provides an exemplary human UBQLN2 polypeptide sequence.

As used herein, the term “BAG6” refers to “large proline-rich protein BAG6,” also known as “BCL2-associated athanogene 6,” or “BAG family molecular chaperone regulator 6,” in Uniprot or GenBank database. The term “BAG6” encompasses the BAG6 polypeptides, the BAG6 RNA transcripts, and the BAG6 genes. The term “BAG6 gene” refers to genes encoding BAG6 polypeptides. BAG6 is expressed in various cells and tissues including plasma and endothelial cells, among others. Examples of BAG6 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BAG6 gene” includes all natural variants of the BAG6 genes, including allelic variants (e.g., SNP variants) and mutations. NCBI Gene ID: 7917 and NCBI Reference Sequence NC_000006.12 (range 31639028 . . . 31660900, complement) provide exemplary human BAG6 nucleic acid sequences. In certain embodiments, BAG6 gene expression is determined by the amounts of the mRNA transcripts. BAG6 gene encodes various transcript variants. In certain embodiments, the mRNA transcripts are splice variants, fragments or derivatives of all native and natural variants of the transcripts of the BAG6 genes. NCBI Reference Sequences NM_001098534.2, NM_001199697.1, and NM_001199698.1 provide exemplary human BAG6 mRNA transcript sequences. Examples of BAG6 polypeptides include any such native polypeptides from any vertebrate source as described above. In certain embodiments, BAG6 gene expression is determined by the amounts of the BAG6 polypeptides. In certain embodiments, the BAG6 polypeptides include all polypeptides encoded by the natural variants of the BAG6 genes and transcripts thereof, including allelic variants (e.g., SNP variants); splice variants; fragments; and derivatives. The BAG6 polypeptides of the present disclosure also encompass “full-length,” unprocessed BAG6 polypeptide as well as any form of BAG6 polypeptide that results from processing in the cell. NCBI Reference Sequences NP_001092004.1, NP_001186626.1, and NP_001186627.1 provide exemplary human BAG6 polypeptide sequences.

As used herein, the term “BOK” refers to “bcl-2-related ovarian killer protein,” also known as “Bcl-2-like protein 9,” in Uniprot or GenBank database. The term “BOK” encompasses the BOK polypeptides, the BOK RNA transcripts, and the BOK genes. The term “BOK gene” refers to genes encoding BOK polypeptides. BOK is expressed in various cells and tissues including plasma and endothelial cells, among others. Examples of BOK genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BOK gene” includes all natural variants of the BOK genes, including allelic variants (e.g., SNP variants) and mutations. NCBI Gene ID: 666 and NCBI Reference Sequence NC_000002.12 (range 241558745 . . . 241574131) provide exemplary human BOK nucleic acid sequences. In certain embodiments, BOK gene expression is determined by the amounts of the mRNA transcripts. BOK gene encodes various transcript variants. In certain embodiments, the mRNA transcripts are splice variants, fragments or derivatives of all native and natural variants of the transcripts of the BOK genes. NCBI Reference Sequences NM_032515.5, XM_017004775.1, and XM_011511697.3 provide exemplary human BOK mRNA transcript sequences. Examples of BOK polypeptides include any such native polypeptides from any vertebrate source as described above. In certain embodiments, BOK gene expression is determined by the amounts of the BOK polypeptides. In certain embodiments, the BOK polypeptides include all polypeptides encoded by the natural variants of the BOK genes and transcripts thereof, including allelic variants (e.g., SNP variants); splice variants; fragments; and derivatives. The BOK polypeptides of the present disclosure also encompass “full-length,” unprocessed BOK polypeptide as well as any form of BOK polypeptide that results from processing in the cell. NCBI Reference Sequences NP_115904.1, XP_016860264.1, and XP_011509999.1 provide exemplary human BOK polypeptide sequences.

As used herein, the term “ROCK1” refers to “Rho associated coiled-coil containing protein kinase 1,” also known as “Bcl-2-like protein 9,” or “renal carcinoma antigen NY-REN-35,” in Uniprot or GenBank database. The term “ROCK1” encompasses the ROCK1 polypeptides, the ROCK1 RNA transcripts, and the ROCK1 genes. The term “ROCK1 gene” refers to genes encoding ROCK1 polypeptides. ROCK1 is expressed in various cells and tissues including plasma and endothelial cells, among others. Examples of ROCK1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ROCK1 gene” includes all natural variants of the ROCK1 genes, including allelic variants (e.g., SNP variants) and mutations. NCBI Gene ID: 6093 and NCBI Reference Sequence NC_000018.10 (range 20946906 . . . 21111813, complement) provide exemplary human ROCK1 nucleic acid sequences. In certain embodiments, ROCK1 gene expression is determined by the amounts of the mRNA transcripts. ROCK1 gene encodes various transcript variants. In certain embodiments, the mRNA transcripts are splice variants, fragments or derivatives of all native and natural variants of the transcripts of the ROCK1 genes. NCBI Reference Sequence NM_005406.3 provides an exemplary human ROCK1 mRNA transcript sequence. Examples of ROCK1 polypeptides include any such native polypeptides from any vertebrate source as described above. In certain embodiments, ROCK1 gene expression is determined by the amounts of the ROCK1 polypeptides. In certain embodiments, the ROCK1 polypeptides include all polypeptides encoded by the natural variants of the ROCK1 genes and transcripts thereof, including allelic variants (e.g., SNP variants); splice variants; fragments; and derivatives. The ROCK1 polypeptides of the present disclosure also encompass “full-length,” unprocessed ROCK1 polypeptide as well as any form of ROCK1 polypeptide that results from processing in the cell. NCBI Reference Sequence NP_005397.1 provides an exemplary human ROCK1 polypeptide sequence.

As used herein, the term “CDKN1A” refers to “cyclin-dependent kinase inhibitor 1,” also known as “CDK-interacting protein 1,” or “melanoma differentiation associated protein 6,” in Uniprot or GenBank database. The term “CDKN1A” encompasses the CDKN1A polypeptides, the CDKN1A RNA transcripts, and the CDKN1A genes. The term “CDKN1A gene” refers to genes encoding CDKN1A polypeptides. CDKN1A is expressed in various cells and tissues including plasma and endothelial cells, among others. Examples of CDKN1A genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CDKN1A gene” includes all natural variants of the CDKN1A genes, including allelic variants (e.g., SNP variants) and mutations. NCBI Gene ID: 1026 and NCBI Reference Sequence NC_000006.12 (range 36676463 . . . 36687332) provide exemplary human CDKN1A nucleic acid sequences. In certain embodiments, CDKN1A gene expression is determined by the amounts of the mRNA transcripts. CDKN1A gene encodes various transcript variants. In certain embodiments, the mRNA transcripts are splice variants, fragments or derivatives of all native and natural variants of the transcripts of the CDKN1A genes. NCBI Reference Sequences NM_000389.5, NM_001220777.2, and NM_001220778.2 provide exemplary human CDKN1A mRNA transcript sequences. Examples of CDKN1A polypeptides include any such native polypeptides from any vertebrate source as described above. In certain embodiments, CDKN1A gene expression is determined by the amounts of the CDKN1A polypeptides. In certain embodiments, the CDKN1A polypeptides include all polypeptides encoded by the natural variants of the CDKN1A genes and transcripts thereof, including allelic variants (e.g., SNP variants); splice variants; fragments; and derivatives. The CDKN1A polypeptides of the present disclosure also encompass “full-length,” unprocessed CDKN1A polypeptide as well as any form of CDKN1A polypeptide that results from processing in the cell. NCBI Reference Sequences NP_000380.1, NP_001207706.1, and NP_001207707.1 provide exemplary human CDKN1A polypeptide sequences.

As used herein, the term “GADD45B” refers to “growth arrest and DNA damage inducible beta,” also known as “Myeloid differentiation primary response protein MyD118,” or “negative growth regulatory protein MyD118,” in Uniprot or GenBank database. The term “GADD45B” encompasses the GADD45B polypeptides, the GADD45B RNA transcripts, and the GADD45B genes. The term “GADD45B gene” refers to genes encoding GADD45B polypeptides. GADD45B is expressed in various cells and tissues including plasma and endothelial cells, among others. Examples of GADD45B genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “GADD45B gene” includes all natural variants of the GADD45B genes, including allelic variants (e.g., SNP variants) and mutations. NCBI Gene ID: 4616 and NCBI Reference Sequence NC_000019.10 (range 2476127 . . . 2478259) provide exemplary human GADD45B nucleic acid sequences. In certain embodiments, GADD45B gene expression is determined by the amounts of the mRNA transcripts. GADD45B gene encodes various transcript variants. In certain embodiments, the mRNA transcripts are splice variants, fragments or derivatives of all native and natural variants of the transcripts of the GADD45B genes. NCBI Reference Sequences NM_015675.4 and XM_017026822.1 provide exemplary human GADD45B mRNA transcript sequences. Examples of GADD45B polypeptides include any such native polypeptides from any vertebrate source as described above. In certain embodiments, GADD45B gene expression is determined by the amounts of the GADD45B polypeptides. In certain embodiments, the GADD45B polypeptides include all polypeptides encoded by the natural variants of the GADD45B genes and transcripts thereof, including allelic variants (e.g., SNP variants); splice variants; fragments; and derivatives. The GADD45B polypeptides of the present disclosure also encompass “full-length,” unprocessed GADD45B polypeptide as well as any form of GADD45B polypeptide that results from processing in the cell. NCBI Reference Sequences NP_056490.2 and XP_016882311.1 provide exemplary human GADD45B polypeptide sequences.

As used herein, the term “E4F1” refers to “E4F transcription factor 1,” also known as “transcription factor E4F1,” “Putative E3 ubiquitin-protein ligase E4F1,” or “RING-type E3 ubiquitin transferase E4F1,” in Uniprot or GenBank database. The term “E4F1” encompasses the E4F1 polypeptides, the E4F1 RNA transcripts, and the E4F1 genes. The term “E4F1 gene” refers to genes encoding E4F1 polypeptides. E4F1 is expressed in various cells and tissues including plasma and endothelial cells, among others. Examples of E4F1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “E4F1 gene” includes all natural variants of the E4F1 genes, including allelic variants (e.g., SNP variants) and mutations. NCBI Gene ID: 1877 and NCBI Reference Sequence NC_000016.10 (range 2223488 . . . 2235742) provide exemplary human E4F1 nucleic acid sequences. In certain embodiments, E4F1 gene expression is determined by the amounts of the mRNA transcripts. E4F1 gene encodes various transcript variants. In certain embodiments, the mRNA transcripts are splice variants, fragments or derivatives of all native and natural variants of the transcripts of the E4F1 genes. NCBI Reference Sequences NM_001288776.1, NM_001288778.1, and NM_004424.5 provide exemplary human E4F1 mRNA transcript sequences. Examples of E4F1 polypeptides include any such native polypeptides from any vertebrate source as described above. In certain embodiments, E4F1 gene expression is determined by the amounts of the E4F1 polypeptides. In certain embodiments, the E4F1 polypeptides include all polypeptides encoded by the natural variants of the E4F1 genes and transcripts thereof, including allelic variants (e.g., SNP variants); splice variants; fragments; and derivatives. The E4F1 polypeptides of the present disclosure also encompass “full-length,” unprocessed E4F1 polypeptide as well as any form of E4F1 polypeptide that results from processing in the cell. NCBI Reference Sequences NP_001275705.1, NP_001275707.1, and NP_004415.4 provide exemplary human E4F1 polypeptide sequences.

As used herein, the term “CDC14B” refers to “dual specificity protein phosphatase CDC14B,” also known as “cell division cycle 14B,” or “CDC14 cell division cycle 14 homolog B,” in Uniprot or GenBank database. The term “CDC14B” encompasses the CDC14B polypeptides, the CDC14B RNA transcripts, and the CDC14B genes. The term “CDC14B gene” refers to genes encoding CDC14B polypeptides. CDC14B is expressed in various cells and tissues including plasma and endothelial cells, among others. Examples of CDC14B genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CDC14B gene” includes all natural variants of the CDC14B genes, including allelic variants (e.g., SNP variants) and mutations. NCBI Gene ID: 8555 and NCBI Reference Sequence NC_000009.12 (range 96492743 . . . 96619843, complement) provide exemplary human CDC14B nucleic acid sequences. In certain embodiments, CDC14B gene expression is determined by the amounts of the mRNA transcripts. CDC14B gene encodes various transcript variants. In certain embodiments, the mRNA transcripts are splice variants, fragments or derivatives of all native and natural variants of the transcripts of the CDC14B genes. NCBI Reference Sequences NM_001077181.3, NM_001351567.2, and NM_001351568.2 provide exemplary human CDC14B mRNA transcript sequences. Examples of CDC14B polypeptides include any such native polypeptides from any vertebrate source as described above. In certain embodiments, CDC14B gene expression is determined by the amounts of the CDC14B polypeptides. In certain embodiments, the CDC14B polypeptides include all polypeptides encoded by the natural variants of the CDC14B genes and transcripts thereof, including allelic variants (e.g., SNP variants); splice variants; fragments; and derivatives. The CDC14B polypeptides of the present disclosure also encompass “full-length,” unprocessed CDC14B polypeptide as well as any form of CDC14B polypeptide that results from processing in the cell. NCBI Reference Sequences NP_001070649.1, NP_001338496.1, and NP_001338497.1 provide exemplary human CDC14B polypeptide sequences.

As used herein, the term “DAPK1” refers to “death associated protein kinase 1,” also known as “DAP kinase 1,” in Uniprot or GenBank database. The term “DAPK1” encompasses the DAPK1 polypeptides, the DAPK1 RNA transcripts, and the DAPK1 genes. The term “DAPK1 gene” refers to genes encoding DAPK1 polypeptides. DAPK1 is expressed in various cells and tissues including plasma and endothelial cells, among others. Examples of DAPK1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “DAPK1 gene” includes all natural variants of the DAPK1 genes, including allelic variants (e.g., SNP variants) and mutations. NCBI Gene ID: 1612 and NCBI Reference Sequence NC_000009.12 (range 87497228 . . . 87708634) provide exemplary human DAPK1 nucleic acid sequences. In certain embodiments, DAPK1 gene expression is determined by the amounts of the mRNA transcripts. DAPK1 gene encodes various transcript variants. In certain embodiments, the mRNA transcripts are splice variants, fragments or derivatives of all native and natural variants of the transcripts of the DAPK1 genes. NCBI Reference Sequences NM_001288729.1, NM_001288730.2, and NM_001288731.2 provide exemplary human DAPK1 mRNA transcript sequences. Examples of DAPK1 polypeptides include any such native polypeptides from any vertebrate source as described above. In certain embodiments, DAPK1 gene expression is determined by the amounts of the DAPK1 polypeptides. In certain embodiments, the DAPK1 polypeptides include all polypeptides encoded by the natural variants of the DAPK1 genes and transcripts thereof, including allelic variants (e.g., SNP variants); splice variants; fragments; and derivatives. The DAPK1 polypeptides of the present disclosure also encompass “full-length,” unprocessed DAPK1 polypeptide as well as any form of DAPK1 polypeptide that results from processing in the cell. NCBI Reference Sequences NP_001275659.1, NP_001275660.1, and NP_001275658.1 provide exemplary human DAPK1 polypeptide sequences.

As used herein, the term “TSC1” refers to “TSC complex subunit 1,” also known as “tuberous sclerosis 1 protein,” or “Hamartin,” in Uniprot or GenBank database. The term “TSC1” encompasses the TSC1 polypeptides, the TSC1 RNA transcripts, and the TSC1 genes. The term “TSC1 gene” refers to genes encoding TSC1 polypeptides. TSC1 is expressed in various cells and tissues including plasma and endothelial cells, among others. Examples of TSC1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TSC1 gene” includes all natural variants of the TSC1 genes, including allelic variants (e.g., SNP variants) and mutations. NCBI Gene ID: 7248 and NCBI Reference Sequence NC_000009.12 (range 132891349 . . . 132945269, complement) provide exemplary human TSC1 nucleic acid sequences. In certain embodiments, TSC1 gene expression is determined by the amounts of the mRNA transcripts. TSC1 gene encodes various transcript variants. In certain embodiments, the mRNA transcripts are splice variants, fragments or derivatives of all native and natural variants of the transcripts of the TSC1 genes. NCBI Reference Sequences NM_000368.5, NM_001162426.2, and NM_001162427.2 provide exemplary human TSC1 mRNA transcript sequences. Examples of TSC1 polypeptides include any such native polypeptides from any vertebrate source as described above. In certain embodiments, TSC1 gene expression is determined by the amounts of the TSC1 polypeptides. In certain embodiments, the TSC1 polypeptides include all polypeptides encoded by the natural variants of the TSC1 genes and transcripts thereof, including allelic variants (e.g., SNP variants); splice variants; fragments; and derivatives. The TSC1 polypeptides of the present disclosure also encompass “full-length,” unprocessed TSC1 polypeptide as well as any form of TSC1 polypeptide that results from processing in the cell. NCBI Reference Sequences NP_000359.1, NP_001155898.1, and NP_001155899.1 provide exemplary human TSC1 polypeptide sequences.

As used herein, the term “TSC2” refers to “TSC complex subunit 2,” also known as “Tuberous sclerosis 2 protein,” or “Tuberin,” in Uniprot or GenBank database. The term “TSC2” encompasses the TSC2 polypeptides, the TSC2 RNA transcripts, and the TSC2 genes. The term “TSC2 gene” refers to genes encoding TSC2 polypeptides. TSC2 is expressed in various cells and tissues including plasma and endothelial cells, among others. Examples of TSC2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TSC2 gene” includes all natural variants of the TSC2 genes, including allelic variants (e.g., SNP variants) and mutations. NCBI Gene ID: 7249 and NCBI Reference Sequence NC_000016.10 (range 2047804 . . . 2089491) provide exemplary human TSC2 nucleic acid sequences. In certain embodiments, TSC2 gene expression is determined by the amounts of the mRNA transcripts. TSC2 gene encodes various transcript variants. In certain embodiments, the mRNA transcripts are splice variants, fragments or derivatives of all native and natural variants of the transcripts of the TSC2 genes. NCBI Reference Sequences NM_000548.5, NM_001077183.2, and NM_001114382.2 provide exemplary human TSC2 mRNA transcript sequences. Examples of TSC2 polypeptides include any such native polypeptides from any vertebrate source as described above. In certain embodiments, TSC2 gene expression is determined by the amounts of the TSC2 polypeptides. In certain embodiments, the TSC2 polypeptides include all polypeptides encoded by the natural variants of the TSC2 genes and transcripts thereof, including allelic variants (e.g., SNP variants); splice variants; fragments; and derivatives. The TSC2 polypeptides of the present disclosure also encompass “full-length,” unprocessed TSC2 polypeptide as well as any form of TSC2 polypeptide that results from processing in the cell. NCBI Reference Sequences NP_000539.2, NP_001070651.1, and NP_001107854.1 provide exemplary human TSC2 polypeptide sequences.

As used herein, the term “BAG3” refers to “BAG cochaperone 3,” also known as “BAG family molecular chaperone regulator 3,” “docking protein CAIR-1,” or “BCL2 associated athanogene 3,” in Uniprot or GenBank database. The term “BAG3” encompasses the BAG3 polypeptides, the BAG3 RNA transcripts, and the BAG3 genes. The term “BAG3 gene” refers to genes encoding BAG3 polypeptides. BAG3 is expressed in various cells and tissues including plasma and endothelial cells, among others. Examples of BAG3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BAG3 gene” includes all natural variants of the BAG3 genes, including allelic variants (e.g., SNP variants) and mutations. NCBI Gene ID: 9531 and NCBI Reference Sequence NC_000010.11 (range 119651380 . . . 119677819) provide exemplary human BAG3 nucleic acid sequences. In certain embodiments, BAG3 gene expression is determined by the amounts of the mRNA transcripts. BAG3 gene encodes various transcript variants. In certain embodiments, the mRNA transcripts are splice variants, fragments or derivatives of all native and natural variants of the transcripts of the BAG3 genes. NCBI Reference Sequences NM_004281.4 and XM_005270287.2 provide exemplary human BAG3 mRNA transcript sequences. Examples of BAG3 polypeptides include any such native polypeptides from any vertebrate source as described above. In certain embodiments, BAG3 gene expression is determined by the amounts of the BAG3 polypeptides. In certain embodiments, the BAG3 polypeptides include all polypeptides encoded by the natural variants of the BAG3 genes and transcripts thereof, including allelic variants (e.g., SNP variants); splice variants; fragments; and derivatives. The BAG3 polypeptides of the present disclosure also encompass “full-length,” unprocessed BAG3 polypeptide as well as any form of BAG3 polypeptide that results from processing in the cell. NCBI Reference Sequences NP_004272.2 and XP_005270344.1 provide exemplary human BAG3 polypeptide sequences.

As used herein, the term “MFN2” refers to “mitofusin 2,” also known as “Transmembrane GTPase MFN2,” “hyperplasia suppressor,” or “mitofusin-2,” in Uniprot or GenBank database. The term “MFN2” encompasses the MFN2 polypeptides, the MFN2 RNA transcripts, and the MFN2 genes. The term “MFN2 gene” refers to genes encoding MFN2 polypeptides. MFN2 is expressed in various cells and tissues including plasma and endothelial cells, among others. Examples of MFN2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “MFN2 gene” includes all natural variants of the MFN2 genes, including allelic variants (e.g., SNP variants) and mutations. NCBI Gene ID: 9927 and NCBI Reference Sequence NC_000001.11 (range 11980181 . . . 12013515) provide exemplary human MFN2 nucleic acid sequences. In certain embodiments, MFN2 gene expression is determined by the amounts of the mRNA transcripts. MFN2 gene encodes various transcript variants. In certain embodiments, the mRNA transcripts are splice variants, fragments or derivatives of all native and natural variants of the transcripts of the MFN2 genes. NCBI Reference Sequences NM_001127660.1, NM_014874.4, and XM_005263548.3 provide exemplary human MFN2 mRNA transcript sequences. Examples of MFN2 polypeptides include any such native polypeptides from any vertebrate source as described above. In certain embodiments, MFN2 gene expression is determined by the amounts of the MFN2 polypeptides. In certain embodiments, the MFN2 polypeptides include all polypeptides encoded by the natural variants of the MFN2 genes and transcripts thereof, including allelic variants (e.g., SNP variants); splice variants; fragments; and derivatives. The MFN2 polypeptides of the present disclosure also encompass “full-length,” unprocessed MFN2 polypeptide as well as any form of MFN2 polypeptide that results from processing in the cell. NCBI Reference Sequences NP_001121132.1, NP_055689.1, and XP_005263605.1 provide exemplary human MFN2 polypeptide sequences.

As used herein, the term “RIPK1” refers to “receptor interacting serine/threonine-protein kinase 1,” also known as “receptor-interacting protein 1,” “cell death protein RIP,” or “receptor interacting serine/threonine kinase 1,” in Uniprot or GenBank database. The term “RIPK1” encompasses the RIPK1 polypeptides, the RIPK1 RNA transcripts, and the RIPK1 genes. The term “RIPK1 gene” refers to genes encoding RIPK1 polypeptides. RIPK1 is expressed in various cells and tissues including plasma and endothelial cells, among others. Examples of RIPK1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RIPK1 gene” includes all natural variants of the RIPK1 genes, including allelic variants (e.g., SNP variants) and mutations. NCBI Gene ID: 8737 and NCBI Reference Sequence NC_000006.12 (range 3063967 . . . 3115187) provide exemplary human RIPK1 nucleic acid sequences. In certain embodiments, RIPK1 gene expression is determined by the amounts of the mRNA transcripts. RIPK1 gene encodes various transcript variants. In certain embodiments, the mRNA transcripts are splice variants, fragments or derivatives of all native and natural variants of the transcripts of the RIPK1 genes. NCBI Reference Sequences NM_001317061.3, NM_001354930.2, and NM_001354931.2 provide exemplary human RIPK1 mRNA transcript sequences. Examples of RIPK1 polypeptides include any such native polypeptides from any vertebrate source as described above. In certain embodiments, RIPK1 gene expression is determined by the amounts of the RIPK1 polypeptides. In certain embodiments, the RIPK1 polypeptides include all polypeptides encoded by the natural variants of the RIPK1 genes and transcripts thereof, including allelic variants (e.g., SNP variants); splice variants; fragments; and derivatives. The RIPK1 polypeptides of the present disclosure also encompass “full-length,” unprocessed RIPK1 polypeptide as well as any form of RIPK1 polypeptide that results from processing in the cell. NCBI Reference Sequences NP_001303990.1, NP_001341859.1, and NP_001341860.1 provide exemplary human RIPK1 polypeptide sequences.

As used herein, the term “RIPK4” refers to “receptor interacting serine/threonine-protein kinase 4,” also known as “Ankyrin repeat domain-containing protein 3,” “PKC-deRa-interacting protein kinase,” or “receptor interacting serine/threonine kinase 4,” in Uniprot or GenBank database. The term “RIPK4” encompasses the RIPK4 polypeptides, the RIPK4 RNA transcripts, and the RIPK4 genes. The term “RIPK4 gene” refers to genes encoding RIPK4 polypeptides. RIPK4 is expressed in various cells and tissues including plasma and endothelial cells, among others. Examples of RIPK4 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RIPK4 gene” includes all natural variants of the RIPK4 genes, including allelic variants (e.g., SNP variants) and mutations. NCBI Gene ID: 54101 and NCBI Reference Sequence NC_000021.9 (range 41739373 . . . 41767052, complement) provide exemplary human RIPK4 nucleic acid sequences. In certain embodiments, RIPK4 gene expression is determined by the amounts of the mRNA transcripts. RIPK4 gene encodes various transcript variants. In certain embodiments, the mRNA transcripts are splice variants, fragments or derivatives of all native and natural variants of the transcripts of the RIPK4 genes. NCBI Reference Sequence NM_020639.3 provides an exemplary human RIPK4 mRNA transcript sequence. Examples of RIPK4 polypeptides include any such native polypeptides from any vertebrate source as described above. In certain embodiments, RIPK4 gene expression is determined by the amounts of the RIPK4 polypeptides. In certain embodiments, the RIPK4 polypeptides include all polypeptides encoded by the natural variants of the RIPK4 genes and transcripts thereof, including allelic variants (e.g., SNP variants); splice variants; fragments; and derivatives. The RIPK4 polypeptides of the present disclosure also encompass “full-length,” unprocessed RIPK4 polypeptide as well as any form of RIPK4 polypeptide that results from processing in the cell. NCBI Reference Sequence NP_065690.2 provides an exemplary human RIPK4 polypeptide sequence.

As used herein, the term “HDAC6” refers to “histone deacetylase 6,” also known as “Tubulin-lysine deacetylase HDAC6,” or “protein phosphatase 1, regulatory subunit 90,” in Uniprot or GenBank database. The term “HDAC6” encompasses the HDAC6 polypeptides, the HDAC6 RNA transcripts, and the HDAC6 genes. The term “HDAC6 gene” refers to genes encoding HDAC6 polypeptides. HDAC6 is expressed in various cells and tissues including plasma and endothelial cells, among others. Examples of HDAC6 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “HDAC6 gene” includes all natural variants of the HDAC6 genes, including allelic variants (e.g., SNP variants) and mutations. NCBI Gene ID: 10013 and NCBI Reference Sequence NC_000023.11 (range 48801398 . . . 48824982) provide exemplary human HDAC6 nucleic acid sequences. In certain embodiments, HDAC6 gene expression is determined by the amounts of the mRNA transcripts. HDAC6 gene encodes various transcript variants. In certain embodiments, the mRNA transcripts are splice variants, fragments or derivatives of all native and natural variants of the transcripts of the HDAC6 genes. NCBI Reference Sequences NM_001321225.2, NM_001321226.2, and NM_001321227.2 provide exemplary human HDAC6 mRNA transcript sequences. Examples of HDAC6 polypeptides include any such native polypeptides from any vertebrate source as described above. In certain embodiments, HDAC6 gene expression is determined by the amounts of the HDAC6 polypeptides. In certain embodiments, the HDAC6 polypeptides include all polypeptides encoded by the natural variants of the HDAC6 genes and transcripts thereof, including allelic variants (e.g., SNP variants); splice variants; fragments; and derivatives. The HDAC6 polypeptides of the present disclosure also encompass “full-length,” unprocessed HDAC6 polypeptide as well as any form of HDAC6 polypeptide that results from processing in the cell. NCBI Reference Sequences NP_001308154.1, NP_001308155.1, and NP_001308156.1 provide exemplary human HDAC6 polypeptide sequences.

As used herein, the term “STK11” refers to “serine/threonine kinase 11,” also known as “serine/threonine-protein kinase STK11,” or “renal carcinoma antigen NY-REN-19,” in Uniprot or GenBank database. The term “STK11” encompasses the STK11 polypeptides, the STK11 RNA transcripts, and the STK11 genes. The term “STK11 gene” refers to genes encoding STK11 polypeptides. STK11 is expressed in various cells and tissues including plasma and endothelial cells, among others. Examples of STK11 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “STK11 gene” includes all natural variants of the STK11 genes, including allelic variants (e.g., SNP variants) and mutations. NCBI Gene ID: 6794 and NCBI Reference Sequence NC_000019.10 (range 1205778 . . . 1228431) provide exemplary human STK11 nucleic acid sequences. In certain embodiments, STK11 gene expression is determined by the amounts of the mRNA transcripts. STK11 gene encodes various transcript variants. In certain embodiments, the mRNA transcripts are splice variants, fragments or derivatives of all native and natural variants of the transcripts of the STK11 genes. NCBI Reference Sequence NM_000455.5 provides an exemplary human STK11 mRNA transcript sequence. Examples of STK11 polypeptides include any such native polypeptides from any vertebrate source as described above. In certain embodiments, STK11 gene expression is determined by the amounts of the STK11 polypeptides. In certain embodiments, the STK11 polypeptides include all polypeptides encoded by the natural variants of the STK11 genes and transcripts thereof, including allelic variants (e.g., SNP variants); splice variants; fragments; and derivatives. The STK11 polypeptides of the present disclosure also encompass “full-length,” unprocessed STK11 polypeptide as well as any form of STK11 polypeptide that results from processing in the cell. NCBI Reference Sequence NP_000446.1 provides an exemplary human STK11 polypeptide sequence.

As used herein, the term “ULK1” refers to “unc-51 like autophagy activating kinase 1,” also known as “serine/threonine-protein kinase ULK1,” or “Autophagy-related protein 1 homolog,” in Uniprot or GenBank database. The term “ULK1” encompasses the ULK1 polypeptides, the ULK1 RNA transcripts, and the ULK1 genes. The term “ULK1 gene” refers to genes encoding ULK1 polypeptides. ULK1 is expressed in various cells and tissues including plasma and endothelial cells, among others. Examples of ULK1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ULK1 gene” includes all natural variants of the ULK1 genes, including allelic variants (e.g., SNP variants) and mutations. NCBI Gene ID: 8408 and NCBI Reference Sequence NC_000012.12 (range 131894622 . . . 131923150) provide exemplary human ULK1 nucleic acid sequences. In certain embodiments, ULK1 gene expression is determined by the amounts of the mRNA transcripts. ULK1 gene encodes various transcript variants. In certain embodiments, the mRNA transcripts are splice variants, fragments or derivatives of all native and natural variants of the transcripts of the ULK1 genes. NCBI Reference Sequences NM_003565.4, XM_011538798.3, and XM_011538799.2 provide exemplary human ULK1 mRNA transcript sequences. Examples of ULK1 polypeptides include any such native polypeptides from any vertebrate source as described above. In certain embodiments, ULK1 gene expression is determined by the amounts of the ULK1 polypeptides. In certain embodiments, the ULK1 polypeptides include all polypeptides encoded by the natural variants of the ULK1 genes and transcripts thereof, including allelic variants (e.g., SNP variants); splice variants; fragments; and derivatives. The ULK1 polypeptides of the present disclosure also encompass “full-length,” unprocessed ULK1 polypeptide as well as any form of ULK1 polypeptide that results from processing in the cell. NCBI Reference Sequences NP_003556.2, XP_011537100.1, and XP_011537101.1 provide exemplary human ULK1 polypeptide sequences.

As used herein, the term “FOXO1” refers to “forkhead box 01,” also known as “forkhead box protein 01,” or “Forkhead box protein 01A,” in Uniprot or GenBank database. The term “FOXO1” encompasses the FOXO1 polypeptides, the FOXO1 RNA transcripts, and the FOXO1 genes. The term “FOXO1 gene” refers to genes encoding FOXO1 polypeptides. FOXO1 is expressed in various cells and tissues including plasma and endothelial cells, among others. Examples of FOXO1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “FOXO1 gene” includes all natural variants of the FOXO1 genes, including allelic variants (e.g., SNP variants) and mutations. NCBI Gene ID: 2308 and NCBI Reference Sequence NC_000013.11 (range 40555667 . . . 40666641, complement) provide exemplary human FOXO1 nucleic acid sequences. In certain embodiments, FOXO1 gene expression is determined by the amounts of the mRNA transcripts. FOXO1 gene encodes various transcript variants. In certain embodiments, the mRNA transcripts are splice variants, fragments or derivatives of all native and natural variants of the transcripts of the FOXO1 genes. NCBI Reference Sequences NM_002015.4, XM_011535008.2, and XM_011535010.2 provide exemplary human FOXO1 mRNA transcript sequences. Examples of FOXO1 polypeptides include any such native polypeptides from any vertebrate source as described above. In certain embodiments, FOXO1 gene expression is determined by the amounts of the FOXO1 polypeptides. In certain embodiments, the FOXO1 polypeptides include all polypeptides encoded by the natural variants of the FOXO1 genes and transcripts thereof, including allelic variants (e.g., SNP variants); splice variants; fragments; and derivatives. The FOXO1 polypeptides of the present disclosure also encompass “full-length,” unprocessed FOXO1 polypeptide as well as any form of FOXO1 polypeptide that results from processing in the cell. NCBI Reference Sequences NP_002006.2, XP_011533310.1, and XP_011533312.1 provide exemplary human FOXO1 polypeptide sequences.

As used herein, the term “FOXO3” refers to “forkhead box 03,” also known as “forkhead box protein 03,” or “forkhead in rhabdomyosarcoma-like 1,” in Uniprot or GenBank database. The term “FOXO3” encompasses the FOXO3 polypeptides, the FOXO3 RNA transcripts, and the FOXO3 genes. The term “FOXO3 gene” refers to genes encoding FOXO3 polypeptides. FOXO3 is expressed in various cells and tissues including plasma and endothelial cells, among others. Examples of FOXO3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “FOXO3 gene” includes all natural variants of the FOXO3 genes, including allelic variants (e.g., SNP variants) and mutations. NCBI Gene ID: 2309 and NCBI Reference Sequence NC_000006.12 (range 108559825 . . . 108684774) provide exemplary human FOXO3 nucleic acid sequences. In certain embodiments, FOXO3 gene expression is determined by the amounts of the mRNA transcripts. FOXO3 gene encodes various transcript variants. In certain embodiments, the mRNA transcripts are splice variants, fragments or derivatives of all native and natural variants of the transcripts of the FOXO3 genes. NCBI Reference Sequences NM_001455.4, NM_201559.3, and XM_005266867.4 provide exemplary human FOXO3 mRNA transcript sequences. Examples of FOXO3 polypeptides include any such native polypeptides from any vertebrate source as described above. In certain embodiments, FOXO3 gene expression is determined by the amounts of the FOXO3 polypeptides. In certain embodiments, the FOXO3 polypeptides include all polypeptides encoded by the natural variants of the FOXO3 genes and transcripts thereof, including allelic variants (e.g., SNP variants); splice variants; fragments; and derivatives. The FOXO3 polypeptides of the present disclosure also encompass “full-length,” unprocessed FOXO3 polypeptide as well as any form of FOXO3 polypeptide that results from processing in the cell. NCBI Reference Sequences NP_001446.1, NP_963853.1, and XP_005266924.1 provide exemplary human FOXO3 polypeptide sequences.

As used herein, the term “MUL1” refers to “mitochondrial E3 ubiquitin protein ligase 1,” also known as “Mitochondrial ubiquitin ligase activator of NFKB 1,” or “E3 ubiquitin-protein ligase MUL1,” in Uniprot or GenBank database. The term “MUL1” encompasses the MUL1 polypeptides, the MUL1 RNA transcripts, and the MUL1 genes. The term “MUL1 gene” refers to genes encoding MUL1 polypeptides. MUL1 is expressed in various cells and tissues including plasma and endothelial cells, among others. Examples of MUL1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “MUL1 gene” includes all natural variants of the MUL1 genes, including allelic variants (e.g., SNP variants) and mutations. NCBI Gene ID: 79594 and NCBI Reference Sequence NC_000001.11 (range 20499448 . . . 20508483, complement) provide exemplary human MUL1 nucleic acid sequences. In certain embodiments, MUL1 gene expression is determined by the amounts of the mRNA transcripts. MUL1 gene encodes various transcript variants. In certain embodiments, the mRNA transcripts are splice variants, fragments or derivatives of all native and natural variants of the transcripts of the MUL1 genes. NCBI Reference Sequences NM_024544.3 and XM_011542137.2 provide exemplary human MUL1 mRNA transcript sequences. Examples of MUL1 polypeptides include any such native polypeptides from any vertebrate source as described above. In certain embodiments, MUL1 gene expression is determined by the amounts of the MUL1 polypeptides. In certain embodiments, the MUL1 polypeptides include all polypeptides encoded by the natural variants of the MUL1 genes and transcripts thereof, including allelic variants (e.g., SNP variants); splice variants; fragments; and derivatives. The MUL1 polypeptides of the present disclosure also encompass “full-length,” unprocessed MUL1 polypeptide as well as any form of MUL1 polypeptide that results from processing in the cell. NCBI Reference Sequences NP_078820.2, and XP_011540439.1 provide exemplary human MUL1 polypeptide sequences.

As used herein, the term “HLA-DPB1” refers to “major histocompatibility complex, class II, DP beta 1,” also known as “HLA class II histocompatibility antigen, DP (W4) beta chain,” or “MHC class II antigen DPB1,” in Uniprot or GenBank database. The term “HLA-DPB 1” encompasses the HLA-DPB1 polypeptides, the HLA-DPB1 RNA transcripts, and the HLA-DPB1 genes. The term “HLA-DPB1 gene” refers to genes encoding HLA-DPB1 polypeptides. HLA-DPB1 is expressed in various cells and tissues including plasma and endothelial cells, among others. Examples of HLA-DPB1 genes encompass any such native gene in human. In certain embodiments, the term “HLA-DPB1 gene” includes all natural variants of the HLA-DPB1 genes, including allelic variants (e.g., SNP variants) and mutations. NCBI Gene ID: 3115 and NCBI Reference Sequence NC_000006.12 (range 33075990 . . . 33089696) provide exemplary human HLA-DPB1 nucleic acid sequences. In certain embodiments, HLA-DPB1 gene expression is determined by the amounts of the mRNA transcripts. HLA-DPB1 gene encodes various transcript variants. In certain embodiments, the mRNA transcripts are splice variants, fragments or derivatives of all native and natural variants of the transcripts of the HLA-DPB1 genes. NCBI Reference Sequence NM_002121.6 provides an exemplary human HLA-DPB1 mRNA transcript sequence. Examples of HLA-DPB1 polypeptides include any such native polypeptides in human. In certain embodiments, HLA-DPB 1 gene expression is determined by the amounts of the HLA-DPB1 polypeptides. In certain embodiments, the HLA-DPB1 polypeptides include all polypeptides encoded by the natural variants of the HLA-DPB1 genes and transcripts thereof, including allelic variants (e.g., SNP variants); splice variants; fragments; and derivatives. The HLA-DPB1 polypeptides of the present disclosure also encompass “full-length,” unprocessed HLA-DPB1 polypeptide as well as any form of HLA-DPB1 polypeptide that results from processing in the cell. NCBI Reference Sequence NP_002112.3 provides an exemplary human HLA-DPB1 polypeptide sequence.

As used herein, the term “EDEM2” refers to “ER degradation enhancing alpha-mannosidase like protein 2,” also known as “ER degradation-enhancing alpha-mannosidase-like protein 2,” or “ER degradation-enhancing-mannosidase-like protein 2,” in Uniprot or GenBank database. The term “EDEM2” encompasses the EDEM2 polypeptides, the EDEM2 RNA transcripts, and the EDEM2 genes. The term “EDEM2 gene” refers to genes encoding EDEM2 polypeptides. EDEM2 is expressed in various cells and tissues including plasma and endothelial cells, among others. Examples of EDEM2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “EDEM2 gene” includes all natural variants of the EDEM2 genes, including allelic variants (e.g., SNP variants) and mutations. NCBI Gene ID: 55741 and NCBI Reference Sequence NC_000020.11 (range 35115364 . . . 35147336, complement) provide exemplary human EDEM2 nucleic acid sequences. In certain embodiments, EDEM2 gene expression is determined by the amounts of the mRNA transcripts. EDEM2 gene encodes various transcript variants. In certain embodiments, the mRNA transcripts are splice variants, fragments or derivatives of all native and natural variants of the transcripts of the EDEM2 genes. NCBI Reference Sequences NM_001145025.2 and NM_018217.3 provide exemplary human EDEM2 mRNA transcript sequences. Examples of EDEM2 polypeptides include any such native polypeptides from any vertebrate source as described above. In certain embodiments, EDEM2 gene expression is determined by the amounts of the EDEM2 polypeptides. In certain embodiments, the EDEM2 polypeptides include all polypeptides encoded by the natural variants of the EDEM2 genes and transcripts thereof, including allelic variants (e.g., SNP variants); splice variants; fragments; and derivatives. The EDEM2 polypeptides of the present disclosure also encompass “full-length,” unprocessed EDEM2 polypeptide as well as any form of EDEM2 polypeptide that results from processing in the cell. NCBI Reference Sequences NP_001138497.1 and NP_060687.2 provide exemplary human EDEM2 polypeptide sequences.

As used herein, the term “FAS” refers to “Fas cell surface death receptor,” also known as “tumor necrosis factor receptor superfamily member 6,” or “apoptosis-mediating surface antigen FAS,” in Uniprot or GenBank database. The term “FAS” encompasses the FAS polypeptides, the FAS RNA transcripts, and the FAS genes. The term “FAS gene” refers to genes encoding FAS polypeptides. FAS is expressed in various cells and tissues including plasma and endothelial cells, among others. Examples of FAS genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “FAS gene” includes all natural variants of the FAS genes, including allelic variants (e.g., SNP variants) and mutations. NCBI Gene ID: 355 and NCBI Reference Sequence NC_000010.11 (range 88968429 . . . 89017059) provide exemplary human FAS nucleic acid sequences. In certain embodiments, FAS gene expression is determined by the amounts of the mRNA transcripts. FAS gene encodes various transcript variants. In certain embodiments, the mRNA transcripts are splice variants, fragments or derivatives of all native and natural variants of the transcripts of the FAS genes. NCBI Reference Sequences NM_000043.6, NM_001320619.2, and NM_152871.4 provide exemplary human FAS mRNA transcript sequences. Examples of FAS polypeptides include any such native polypeptides from any vertebrate source as described above. In certain embodiments, FAS gene expression is determined by the amounts of the FAS polypeptides. In certain embodiments, the FAS polypeptides include all polypeptides encoded by the natural variants of the FAS genes and transcripts thereof, including allelic variants (e.g., SNP variants); splice variants; fragments; and derivatives. The FAS polypeptides of the present disclosure also encompass “full-length,” unprocessed FAS polypeptide as well as any form of FAS polypeptide that results from processing in the cell. NCBI Reference Sequences NP_000034.1, NP_001307548.1, and NP_690610.1 provide exemplary human FAS polypeptide sequences.

As used herein, the term “TLR3” refers to “toll like receptor 3,” also known as “CD283,” in Uniprot or GenBank database. The term “TLR3” encompasses the TLR3 polypeptides, the TLR3 RNA transcripts, and the TLR3 genes. The term “TLR3 gene” refers to genes encoding TLR3 polypeptides. TLR3 is expressed in various cells and tissues including plasma and endothelial cells, among others. Examples of TLR3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TLR3 gene” includes all natural variants of the TLR3 genes, including allelic variants (e.g., SNP variants) and mutations. NCBI Gene ID: 7098 and NCBI Reference Sequence NC_000004.12 (range 186069156 . . . 186088073) provide exemplary human TLR3 nucleic acid sequences. In certain embodiments, TLR3 gene expression is determined by the amounts of the mRNA transcripts. TLR3 gene encodes various transcript variants. In certain embodiments, the mRNA transcripts are splice variants, fragments or derivatives of all native and natural variants of the transcripts of the TLR3 genes. NCBI Reference Sequence NM_003265.3 provides an exemplary human TLR3 mRNA transcript sequence. Examples of TLR3 polypeptides include any such native polypeptides from any vertebrate source as described above. In certain embodiments, TLR3 gene expression is determined by the amounts of the TLR3 polypeptides. In certain embodiments, the TLR3 polypeptides include all polypeptides encoded by the natural variants of the TLR3 genes and transcripts thereof, including allelic variants (e.g., SNP variants); splice variants; fragments; and derivatives. The TLR3 polypeptides of the present disclosure also encompass “full-length,” unprocessed TLR3 polypeptide as well as any form of TLR3 polypeptide that results from processing in the cell. NCBI Reference Sequence NP_003256.1 provides an exemplary human TLR3 polypeptide sequence.

As used herein, the term “CDC42” refers to “cell division cycle 42,” also known as “Cell division control protein 42 homolog,” or “G25K GTP-binding protein,” in Uniprot or GenBank database. The term “CDC42” encompasses the CDC42 polypeptides, the CDC42 RNA transcripts, and the CDC42 genes. The term “CDC42 gene” refers to genes encoding CDC42 polypeptides. CDC42 is expressed in various cells and tissues including plasma and endothelial cells, among others. Examples of CDC42 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CDC42 gene” includes all natural variants of the CDC42 genes, including allelic variants (e.g., SNP variants) and mutations. NCBI Gene ID: 998 and NCBI Reference Sequence NC_000001.11 (range 22052709 . . . 22101360) provide exemplary human CDC42 nucleic acid sequences. In certain embodiments, CDC42 gene expression is determined by the amounts of the mRNA transcripts. CDC42 gene encodes various transcript variants. In certain embodiments, the mRNA transcripts are splice variants, fragments or derivatives of all native and natural variants of the transcripts of the CDC42 genes. NCBI Reference Sequence NM_001039802.2 provides an exemplary human CDC42 mRNA transcript sequence. Examples of CDC42 polypeptides include any such native polypeptides from any vertebrate source as described above. In certain embodiments, CDC42 gene expression is determined by the amounts of the CDC42 polypeptides. In certain embodiments, the CDC42 polypeptides include all polypeptides encoded by the natural variants of the CDC42 genes and transcripts thereof, including allelic variants (e.g., SNP variants); splice variants; fragments; and derivatives. The CDC42 polypeptides of the present disclosure also encompass “full-length,” unprocessed CDC42 polypeptide as well as any form of CDC42 polypeptide that results from processing in the cell. NCBI Reference Sequence NP_001034891.1 provides an exemplary human CDC42 polypeptide sequence.

As used herein, the term “RhoA” refers to “ras homolog family member A,” also known as “transforming protein RhoA,” in Uniprot or GenBank database. The term “RhoA” encompasses the RhoA polypeptides, the RhoA RNA transcripts, and the RhoA genes. The term “RhoA gene” refers to genes encoding RhoA polypeptides. RhoA is expressed in various cells and tissues including plasma and endothelial cells, among others. Examples of RhoA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RhoA gene” includes all natural variants of the RhoA genes, including allelic variants (e.g., SNP variants) and mutations. NCBI Gene ID: 387 and NCBI Reference Sequence NC_000003.12 (range 49359145 . . . 49411976, complement) provide exemplary human RhoA nucleic acid sequences. In certain embodiments, RhoA gene expression is determined by the amounts of the mRNA transcripts. RhoA gene encodes various transcript variants. In certain embodiments, the mRNA transcripts are splice variants, fragments or derivatives of all native and natural variants of the transcripts of the RhoA genes. NCBI Reference Sequences NM_001313941.2, NM_001313943.2, and NM_001313944.2 provide exemplary human RhoA mRNA transcript sequences. Examples of RhoA polypeptides include any such native polypeptides from any vertebrate source as described above. In certain embodiments, RhoA gene expression is determined by the amounts of the RhoA polypeptides. In certain embodiments, the RhoA polypeptides include all polypeptides encoded by the natural variants of the RhoA genes and transcripts thereof, including allelic variants (e.g., SNP variants); splice variants; fragments; and derivatives. The RhoA polypeptides of the present disclosure also encompass “full-length,” unprocessed RhoA polypeptide as well as any form of RhoA polypeptide that results from processing in the cell. NCBI Reference Sequences NP_001300870.1, NP_001300872.1, and NP_001300873.1 provide exemplary human RhoA polypeptide sequences.

As used herein, the term “RhoC” refers to “ras homolog family member C,” also known as “rho-related GTP-binding protein RhoC,” or “rho cDNA clone 9,” in Uniprot or GenBank database. The term “RhoC” encompasses the RhoC polypeptides, the RhoC RNA transcripts, and the RhoC genes. The term “RhoC gene” refers to genes encoding RhoC polypeptides. RhoC is expressed in various cells and tissues including plasma and endothelial cells, among others. Examples of RhoC genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RhoC gene” includes all natural variants of the RhoC genes, including allelic variants (e.g., SNP variants) and mutations. NCBI Gene ID: 389 and NCBI Reference Sequence NC_000001.11 (range 112701127 . . . 112707403, complement) provide exemplary human RhoC nucleic acid sequences. In certain embodiments, RhoC gene expression is determined by the amounts of the mRNA transcripts. RhoC gene encodes various transcript variants. In certain embodiments, the mRNA transcripts are splice variants, fragments or derivatives of all native and natural variants of the transcripts of the RhoC genes. NCBI Reference Sequences NM_001042678.1, NM_001042679.1, and NM_175744.5 provide exemplary human RhoC mRNA transcript sequences. Examples of RhoC polypeptides include any such native polypeptides from any vertebrate source as described above. In certain embodiments, RhoC gene expression is determined by the amounts of the RhoC polypeptides. In certain embodiments, the RhoC polypeptides include all polypeptides encoded by the natural variants of the RhoC genes and transcripts thereof, including allelic variants (e.g., SNP variants); splice variants; fragments; and derivatives. The RhoC polypeptides of the present disclosure also encompass “full-length,” unprocessed RhoC polypeptide as well as any form of RhoC polypeptide that results from processing in the cell. NCBI Reference Sequences NP_001036143.1, NP_001036144.1, and NP_786886.1 provide exemplary human RhoC polypeptide sequences.

As used herein, the term “DDIAS” refers to “DNA damage induced apoptosis suppressor,” also known as “DNA damage-induced apoptosis suppressor protein,” or “Nitric oxide-inducible gene protein,” in Uniprot or GenBank database. The term “DDIAS” encompasses the DDIAS polypeptides, the DDIAS RNA transcripts, and the DDIAS genes. The term “DDIAS gene” refers to genes encoding DDIAS polypeptides. DDIAS is expressed in various cells and tissues including plasma and endothelial cells, among others. Examples of DDIAS genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “DDIAS gene” includes all natural variants of the DDIAS genes, including allelic variants (e.g., SNP variants) and mutations. NCBI Gene ID: 220042 and NCBI Reference Sequence NC_000011.10 (range 82901735 . . . 82934659) provide exemplary human DDIAS nucleic acid sequences. In certain embodiments, DDIAS gene expression is determined by the amounts of the mRNA transcripts. DDIAS gene encodes various transcript variants. In certain embodiments, the mRNA transcripts are splice variants, fragments or derivatives of all native and natural variants of the transcripts of the DDIAS genes. NCBI Reference Sequences NM_001363481.2, NM_145018.4, and XM_024448400.1 provide exemplary human DDIAS mRNA transcript sequences. Examples of DDIAS polypeptides include any such native polypeptides from any vertebrate source as described above. In certain embodiments, DDIAS gene expression is determined by the amounts of the DDIAS polypeptides. In certain embodiments, the DDIAS polypeptides include all polypeptides encoded by the natural variants of the DDIAS genes and transcripts thereof, including allelic variants (e.g., SNP variants); splice variants; fragments; and derivatives. The DDIAS polypeptides of the present disclosure also encompass “full-length,” unprocessed DDIAS polypeptide as well as any form of DDIAS polypeptide that results from processing in the cell. NCBI Reference Sequences NP_001350410.1, NP_659455.3, and XP_024304168.1 provide exemplary human DDIAS polypeptide sequences.

As used herein, the term “CDK1” refers to “cyclin dependent kinase 1,” also known as “cell division control protein 2 homolog,” “p34 protein kinase,” or “cell division protein kinase 1,” in Uniprot or GenBank database. The term “CDK1” encompasses the CDK1 polypeptides, the CDK1 RNA transcripts, and the CDK1 genes. The term “CDK1 gene” refers to genes encoding CDK1 polypeptides. CDK1 is expressed in various cells and tissues including plasma and endothelial cells, among others. Examples of CDK1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CDK1 gene” includes all natural variants of the CDK1 genes, including allelic variants (e.g., SNP variants) and mutations. NCBI Gene ID: 983 and NCBI Reference Sequence NC_000010.11 (range 60778331 . . . 60794852) provide exemplary human CDK1 nucleic acid sequences. In certain embodiments, CDK1 gene expression is determined by the amounts of the mRNA transcripts. CDK1 gene encodes various transcript variants. In certain embodiments, the mRNA transcripts are splice variants, fragments or derivatives of all native and natural variants of the transcripts of the CDK1 genes. NCBI Reference Sequences NM_001170406.1, NM_001170407.1, and NM_001320918.1 provide exemplary human CDK1 mRNA transcript sequences. Examples of CDK1 polypeptides include any such native polypeptides from any vertebrate source as described above. In certain embodiments, CDK1 gene expression is determined by the amounts of the CDK1 polypeptides. In certain embodiments, the CDK1 polypeptides include all polypeptides encoded by the natural variants of the CDK1 genes and transcripts thereof, including allelic variants (e.g., SNP variants); splice variants; fragments; and derivatives. The CDK1 polypeptides of the present disclosure also encompass “full-length,” unprocessed CDK1 polypeptide as well as any form of CDK1 polypeptide that results from processing in the cell. NCBI Reference Sequences NP_001163877.1, NP_001163878.1, and NP_001307847.1 provide exemplary human CDK1 polypeptide sequences.

As used herein, the term “BNIP3” refers to “BCL2 interacting protein 3,” also known as “BCL2/adenovirus E1B 19 kDa protein-interacting protein 3,” in Uniprot or GenBank database. The term “BNIP3” encompasses the BNIP3 polypeptides, the BNIP3 RNA transcripts, and the BNIP3 genes. The term “BNIP3 gene” refers to genes encoding BNIP3 polypeptides. BNIP3 is expressed in various cells and tissues including plasma and endothelial cells, among others. Examples of BNIP3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BNIP3 gene” includes all natural variants of the BNIP3 genes, including allelic variants (e.g., SNP variants) and mutations. NCBI Gene ID: 664 and NCBI Reference Sequence NC_000010.11 (range 31967683 . . . 131982013, complement) provide exemplary human BNIP3 nucleic acid sequences. In certain embodiments, BNIP3 gene expression is determined by the amounts of the mRNA transcripts. BNIP3 gene encodes various transcript variants. In certain embodiments, the mRNA transcripts are splice variants, fragments or derivatives of all native and natural variants of the transcripts of the BNIP3 genes. NCBI Reference Sequence NM_004052.3 provides an exemplary human BNIP3 mRNA transcript sequence. Examples of BNIP3 polypeptides include any such native polypeptides from any vertebrate source as described above. In certain embodiments, BNIP3 gene expression is determined by the amounts of the BNIP3 polypeptides. In certain embodiments, the BNIP3 polypeptides include all polypeptides encoded by the natural variants of the BNIP3 genes and transcripts thereof, including allelic variants (e.g., SNP variants); splice variants; fragments; and derivatives. The BNIP3 polypeptides of the present disclosure also encompass “full-length,” unprocessed BNIP3 polypeptide as well as any form of BNIP3 polypeptide that results from processing in the cell. NCBI Reference Sequence NP_004043.3 provides an exemplary human BNIP3 polypeptide sequence.

As used herein, the term “BNIP3L” refers to “BCL2 interacting protein 3 like,” also known as “BCL2/adenovirus E1B 19 kDa protein-interacting protein 3-like,” “BCL2/adenovirus E1B 19 kDa protein-interacting protein 3A,” “Adenovirus E1B19K-binding protein B5,” or “NIP3-like protein X,” in Uniprot or GenBank database. The term “BNIP3L” encompasses the BNIP3L polypeptides, the BNIP3L RNA transcripts, and the BNIP3L genes. The term “BNIP3L gene” refers to genes encoding BNIP3L polypeptides. BNIP3L is expressed in various cells and tissues including plasma and endothelial cells, among others. Examples of BNIP3L genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “BNIP3L gene” includes all natural variants of the BNIP3L genes, including allelic variants (e.g., SNP variants) and mutations. NCBI Gene ID: 665 and NCBI Reference Sequence NC_000008.11 (range 26383054 . . . 26413127) provide exemplary human BNIP3L nucleic acid sequences. In certain embodiments, BNIP3L gene expression is determined by the amounts of the mRNA transcripts. BNIP3L gene encodes various transcript variants. In certain embodiments, the mRNA transcripts are splice variants, fragments or derivatives of all native and natural variants of the transcripts of the BNIP3L genes. NCBI Reference Sequences NM_001330491.2 and NM_004331.3 provide exemplary human BNIP3L mRNA transcript sequences. Examples of BNIP3L polypeptides include any such native polypeptides from any vertebrate source as described above. In certain embodiments, BNIP3L gene expression is determined by the amounts of the BNIP3L polypeptides. In certain embodiments, the BNIP3L polypeptides include all polypeptides encoded by the natural variants of the BNIP3L genes and transcripts thereof, including allelic variants (e.g., SNP variants); splice variants; fragments; and derivatives. The BNIP3L polypeptides of the present disclosure also encompass “full-length,” unprocessed BNIP3L polypeptide as well as any form of BNIP3L polypeptide that results from processing in the cell. NCBI Reference Sequences NP_001317420.1 and NP_004322.1 provide exemplary human BNIP3L polypeptide sequences.

As used herein, the term “IL2RA” refers to “interleukin 2 receptor subunit alpha,” also known as “IL-2 receptor subunit alpha,” “TAC antigen,” “CD25,” or “IL-2R subunit alpha,” in Uniprot or GenBank database. The term “IL2RA” encompasses the IL2RA polypeptides, the IL2RA RNA transcripts, and the IL2RA genes. The term “IL2RA gene” refers to genes encoding IL2RA polypeptides. IL2RA is expressed in various cells and tissues including plasma and endothelial cells, among others. Examples of IL2RA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL2RA gene” includes all natural variants of the IL2RA genes, including allelic variants (e.g., SNP variants) and mutations. NCBI Gene ID: 3559 and NCBI Reference Sequence NC_000010.11 (range 6010689 . . . 6062367, complement) provide exemplary human IL2RA nucleic acid sequences. In certain embodiments, IL2RA gene expression is determined by the amounts of the mRNA transcripts. IL2RA gene encodes various transcript variants. In certain embodiments, the mRNA transcripts are splice variants, fragments or derivatives of all native and natural variants of the transcripts of the IL2RA genes. NCBI Reference Sequences NM_000417.3, NM_001308242.2, and NM_001308243.2 provide exemplary human IL2RA mRNA transcript sequences. Examples of IL2RA polypeptides include any such native polypeptides from any vertebrate source as described above. In certain embodiments, IL2RA gene expression is determined by the amounts of the IL2RA polypeptides. In certain embodiments, the IL2RA polypeptides include all polypeptides encoded by the natural variants of the IL2RA genes and transcripts thereof, including allelic variants (e.g., SNP variants); splice variants; fragments; and derivatives. The IL2RA polypeptides of the present disclosure also encompass “full-length,” unprocessed IL2RA polypeptide as well as any form of IL2RA polypeptide that results from processing in the cell. NCBI Reference Sequences NP_000408.1, NP_001295171.1, and NP_001295172.1 provide exemplary human IL2RA polypeptide sequences.

As used herein, the term “IL2RB” refers to “interleukin 2 receptor subunit beta,” also known as “IL-2 receptor subunit beta,” “interleukin-15 receptor subunit beta,” “CD122,” or “High affinity IL-2 receptor subunit beta,” in Uniprot or GenBank database. The term “IL2RB” encompasses the IL2RB polypeptides, the IL2RB RNA transcripts, and the IL2RB genes. The term “IL2RB gene” refers to genes encoding IL2RB polypeptides. IL2RB is expressed in various cells and tissues including plasma and endothelial cells, among others. Examples of IL2RB genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL2RB gene” includes all natural variants of the IL2RB genes, including allelic variants (e.g., SNP variants) and mutations. NCBI Gene ID: 3560 and NCBI Reference Sequence NC_000022.11 (range 37125838 . . . 37175118, complement) provide exemplary human IL2RB nucleic acid sequences. In certain embodiments, IL2RB gene expression is determined by the amounts of the mRNA transcripts. IL2RB gene encodes various transcript variants. In certain embodiments, the mRNA transcripts are splice variants, fragments or derivatives of all native and natural variants of the transcripts of the IL2RB genes. NCBI Reference Sequences NM_000878.5, NM_001346222.1, and NM_001346223.2 provide exemplary human IL2RB mRNA transcript sequences. Examples of IL2RB polypeptides include any such native polypeptides from any vertebrate source as described above. In certain embodiments, IL2RB gene expression is determined by the amounts of the IL2RB polypeptides. In certain embodiments, the IL2RB polypeptides include all polypeptides encoded by the natural variants of the IL2RB genes and transcripts thereof, including allelic variants (e.g., SNP variants); splice variants; fragments; and derivatives. The IL2RB polypeptides of the present disclosure also encompass “full-length,” unprocessed IL2RB polypeptide as well as any form of IL2RB polypeptide that results from processing in the cell. NCBI Reference Sequences NP_000869.1, NP_001333151.1, and NP_001333152.1 provide exemplary human IL2RB polypeptide sequences.

As used herein, the term “IL2RG” refers to “interleukin 2 receptor subunit gamma,” also known as “cytokine receptor common subunit gamma,” “IL-2 receptor subunit gamma,” “CD132,” or “gammaC,” in Uniprot or GenBank database. The term “IL2RG” encompasses the IL2RG polypeptides, the IL2RG RNA transcripts, and the IL2RG genes. The term “IL2RG gene” refers to genes encoding IL2RG polypeptides. IL2RG is expressed in various cells and tissues including plasma and endothelial cells, among others. Examples of IL2RG genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL2RG gene” includes all natural variants of the IL2RG genes, including allelic variants (e.g., SNP variants) and mutations. NCBI Gene ID: 3561 and NCBI Reference Sequence NC_000023.11 (range 71107404 . . . 71111577, complement) provide exemplary human IL2RG nucleic acid sequences. In certain embodiments, IL2RG gene expression is determined by the amounts of the mRNA transcripts. IL2RG gene encodes various transcript variants. In certain embodiments, the mRNA transcripts are splice variants, fragments or derivatives of all native and natural variants of the transcripts of the IL2RG genes. NCBI Reference Sequences NM_000206.3 and AB102797 provide exemplary human IL2RG mRNA transcript sequences. Examples of IL2RG polypeptides include any such native polypeptides from any vertebrate source as described above. In certain embodiments, IL2RG gene expression is determined by the amounts of the IL2RG polypeptides. In certain embodiments, the IL2RG polypeptides include all polypeptides encoded by the natural variants of the IL2RG genes and transcripts thereof, including allelic variants (e.g., SNP variants); splice variants; fragments; and derivatives. The IL2RG polypeptides of the present disclosure also encompass “full-length,” unprocessed IL2RG polypeptide as well as any form of IL2RG polypeptide that results from processing in the cell. NCBI Reference Sequences NP_000197.1 and BAD89388.1 provide exemplary human IL2RG polypeptide sequences.

As used herein, the term “IL21R” refers to “interleukin 21 receptor,” also known as “interleukin-21 receptor,” “IL-21 receptor,” “CD360,” or “novel interleukin receptor,” in Uniprot or GenBank database. The term “IL21R” encompasses the IL21R polypeptides, the IL21R RNA transcripts, and the IL21R genes. The term “IL21R gene” refers to genes encoding IL21R polypeptides. IL21R is expressed in various cells and tissues including plasma and endothelial cells, among others. Examples of IL21R genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL21R gene” includes all natural variants of the IL21R genes, including allelic variants (e.g., SNP variants) and mutations. NCBI Gene ID: 50615 and NCBI Reference Sequence NC_000016.10 (range 27402162 . . . 27452043) provide exemplary human IL21R nucleic acid sequences. In certain embodiments, IL21R gene expression is determined by the amounts of the mRNA transcripts. IL21R gene encodes various transcript variants. In certain embodiments, the mRNA transcripts are splice variants, fragments or derivatives of all native and natural variants of the transcripts of the IL21R genes. NCBI Reference Sequences NM_021798.4, NM_181078.3, and NM_181079.5 provide exemplary human IL21R mRNA transcript sequences. Examples of IL21R polypeptides include any such native polypeptides from any vertebrate source as described above. In certain embodiments, IL21R gene expression is determined by the amounts of the IL21R polypeptides. In certain embodiments, the IL21R polypeptides include all polypeptides encoded by the natural variants of the IL21R genes and transcripts thereof, including allelic variants (e.g., SNP variants); splice variants; fragments; and derivatives. The IL21R polypeptides of the present disclosure also encompass “full-length,” unprocessed IL21R polypeptide as well as any form of IL21R polypeptide that results from processing in the cell. NCBI Reference Sequences NP_068570.1, NP_851564.1, and NP_851565.4 provide exemplary human IL21R polypeptide sequences.

As used herein, the term “IL21R” refers to “interleukin 27 receptor subunit alpha,” also known as “IL-27 receptor subunit alpha,” “cytokine receptor WSX-1,” “cytokine receptor-like 1,” or “type I T-cell cytokine receptor,” in Uniprot or GenBank database. The term “IL21R” encompasses the IL21R polypeptides, the IL21R RNA transcripts, and the IL21R genes. The term “IL21R gene” refers to genes encoding IL21R polypeptides. IL21R is expressed in various cells and tissues including plasma and endothelial cells, among others. Examples of IL21R genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL21R gene” includes all natural variants of the IL21R genes, including allelic variants (e.g., SNP variants) and mutations. NCBI Gene ID: 9466 and NCBI Reference Sequence NC_000019.10 (range 14031762 . . . 14053218) provide exemplary human IL21R nucleic acid sequences. In certain embodiments, IL21R gene expression is determined by the amounts of the mRNA transcripts. IL21R gene encodes various transcript variants. In certain embodiments, the mRNA transcripts are splice variants, fragments or derivatives of all native and natural variants of the transcripts of the IL21R genes. NCBI Reference Sequences NM_004843.4 and BC028003 provide exemplary human IL21R mRNA transcript sequences. Examples of IL21R polypeptides include any such native polypeptides from any vertebrate source as described above. In certain embodiments, IL21R gene expression is determined by the amounts of the IL21R polypeptides. In certain embodiments, the IL21R polypeptides include all polypeptides encoded by the natural variants of the IL21R genes and transcripts thereof, including allelic variants (e.g., SNP variants); splice variants; fragments; and derivatives. The IL21R polypeptides of the present disclosure also encompass “full-length,” unprocessed IL21R polypeptide as well as any form of IL21R polypeptide that results from processing in the cell. NCBI Reference Sequences NP_004834.1 and AAH28003 provide exemplary human IL21R polypeptide sequences.

As used herein, the term “IL1RN” refers to “interleukin 1 receptor antagonist protein,” also known as “interleukin 1 receptor antagonist,” “IL1 inhibitor,” “IL-1ra,” or “intracellular interleukin-1 receptor antagonist (icIL-1ra),” in Uniprot or GenBank database. The term “IL1RN” encompasses the IL1RN polypeptides, the IL1RN RNA transcripts, and the IL1RN genes. The term “IL1RN gene” refers to genes encoding IL1RN polypeptides. IL1RN is expressed in various cells and tissues including plasma and endothelial cells, among others. Examples of IL1RN genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL1RN gene” includes all natural variants of the IL1RN genes, including allelic variants (e.g., SNP variants) and mutations. NCBI Gene ID: 3557 and NCBI Reference Sequence NC_000002.12 (range 113099365 . . . 113134016) provide exemplary human IL1RN nucleic acid sequences. In certain embodiments, IL1RN gene expression is determined by the amounts of the mRNA transcripts. IL1RN gene encodes various transcript variants. In certain embodiments, the mRNA transcripts are splice variants, fragments or derivatives of all native and natural variants of the transcripts of the IL1RN genes. NCBI Reference Sequences NM_000577.5, NM_001318914.2, NM_173841.3, and NM_173842.3 provide exemplary human IL1RN mRNA transcript sequences. Examples of IL1RN polypeptides include any such native polypeptides from any vertebrate source as described above. In certain embodiments, IL1RN gene expression is determined by the amounts of the IL1RN polypeptides. In certain embodiments, the IL1RN polypeptides include all polypeptides encoded by the natural variants of the IL1RN genes and transcripts thereof, including allelic variants (e.g., SNP variants); splice variants; fragments; and derivatives. The IL1RN polypeptides of the present disclosure also encompass “full-length,” unprocessed IL1RN polypeptide as well as any form of IL1RN polypeptide that results from processing in the cell. NCBI Reference Sequences NP_000568.1, NP_001305843.1, NP_776213.1, and NP_776214.1 provide exemplary human IL1RN polypeptide sequences.

As used herein, the term “IL17RA” refers to “interleukin 17 receptor A,” also known as “IL-17 receptor A,” “IL-17RA,” “CD217,” or “CDw217,” in Uniprot or GenBank database. The term “IL17RA” encompasses the IL17RA polypeptides, the IL17RA RNA transcripts, and the IL17RA genes. The term “IL17RA gene” refers to genes encoding IL17RA polypeptides. IL17RA is expressed in various cells and tissues including plasma and endothelial cells, among others. Examples of IL17RA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL17RA gene” includes all natural variants of the IL17RA genes, including allelic variants (e.g., SNP variants) and mutations. NCBI Gene ID: 23765 and NCBI Reference Sequence NC_000022.11 (range 17084959 . . . 17115694) provide exemplary human IL17RA nucleic acid sequences. In certain embodiments, IL17RA gene expression is determined by the amounts of the mRNA transcripts. IL17RA gene encodes various transcript variants. In certain embodiments, the mRNA transcripts are splice variants, fragments or derivatives of all native and natural variants of the transcripts of the IL17RA genes. NCBI Reference Sequences NM_001289905.1 and NM_014339.7 provide exemplary human IL17RA mRNA transcript sequences. Examples of IL17RA polypeptides include any such native polypeptides from any vertebrate source as described above. In certain embodiments, IL17RA gene expression is determined by the amounts of the IL17RA polypeptides. In certain embodiments, the IL17RA polypeptides include all polypeptides encoded by the natural variants of the IL17RA genes and transcripts thereof, including allelic variants (e.g., SNP variants); splice variants; fragments; and derivatives. The IL17RA polypeptides of the present disclosure also encompass “full-length,” unprocessed IL17RA polypeptide as well as any form of IL17RA polypeptide that results from processing in the cell. NCBI Reference Sequences NP_001276834.1 and NP_055154.3 provide exemplary human IL17RA polypeptide sequences.

As used herein, the term “IL3RA” refers to “interleukin-3 receptor subunit alpha,” also known as “IL-3 receptor subunit alpha,” “IL-3R subunit alpha,” “CD123,” or “IL-3R-alpha,” in Uniprot or GenBank database. The term “IL3RA” encompasses the IL3RA polypeptides, the IL3RA RNA transcripts, and the IL3RA genes. The term “IL3RA gene” refers to genes encoding IL3RA polypeptides. IL3RA is expressed in various cells and tissues including plasma and endothelial cells, among others. Examples of IL3RA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL3RA gene” includes all natural variants of the IL3RA genes, including allelic variants (e.g., SNP variants) and mutations. NCBI Gene ID: 3563, NCBI Reference Sequence NC_000023.11 (range 1336574 . . . 1382689), and NC_000024.10 (range 1336574 . . . 1382689) provide exemplary human IL3RA nucleic acid sequences. In certain embodiments, IL3RA gene expression is determined by the amounts of the mRNA transcripts. IL3RA gene encodes various transcript variants. In certain embodiments, the mRNA transcripts are splice variants, fragments or derivatives of all native and natural variants of the transcripts of the IL3RA genes. NCBI Reference Sequences NM_001267713.1, NM_002183.4, and XM_005274431.5 provide exemplary human IL3RA mRNA transcript sequences. Examples of IL3RA polypeptides include any such native polypeptides from any vertebrate source as described above. In certain embodiments, IL3RA gene expression is determined by the amounts of the IL3RA polypeptides. In certain embodiments, the IL3RA polypeptides include all polypeptides encoded by the natural variants of the IL3RA genes and transcripts thereof, including allelic variants (e.g., SNP variants); splice variants; fragments; and derivatives. The IL3RA polypeptides of the present disclosure also encompass “full-length,” unprocessed IL3RA polypeptide as well as any form of IL3RA polypeptide that results from processing in the cell. NCBI Reference Sequences NP_001254642.1, NP_002174.1, and XP_005274488.1 provide exemplary human IL3RA polypeptide sequences.

As used herein, the term “IL1R1” refers to “interleukin 1 receptor type 1,” also known as “Interleukin-1 receptor alpha,” “IL-1RT-1,” “CD121a,” or “CD121 antigen-like family member A,” in Uniprot or GenBank database. The term “IL1R1” encompasses the IL1R1 polypeptides, the IL1R1 RNA transcripts, and the IL1R1 genes. The term “IL1R1 gene” refers to genes encoding IL1R1 polypeptides. IL1R1 is expressed in various cells and tissues including plasma and endothelial cells, among others. Examples of IL1R1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL1R1 gene” includes all natural variants of the IL1R1 genes, including allelic variants (e.g., SNP variants) and mutations. NCBI Gene ID: 3554 and NCBI Reference Sequence NC_000002.12 (range 102069638 . . . 102179874) provide exemplary human IL1R1 nucleic acid sequences. In certain embodiments, IL1R1 gene expression is determined by the amounts of the mRNA transcripts. IL1R1 gene encodes various transcript variants. In certain embodiments, the mRNA transcripts are splice variants, fragments or derivatives of all native and natural variants of the transcripts of the IL1R1 genes. NCBI Reference Sequences NM_000877.4, NM_001288706.2, NM_001320980.2, NM_001320981.2, NM_001320982.2, NM_001320983.1, NM_001320984.1, NM_001320985.1, M 001320986.2, and NM_001320978.2 provide exemplary human IL1R1 mRNA transcript sequences. Examples of IL1R1 polypeptides include any such native polypeptides from any vertebrate source as described above. In certain embodiments, IL1R1 gene expression is determined by the amounts of the IL1R1 polypeptides. In certain embodiments, the IL1R1 polypeptides include all polypeptides encoded by the natural variants of the IL1R1 genes and transcripts thereof, including allelic variants (e.g., SNP variants); splice variants; fragments; and derivatives. The IL1R1 polypeptides of the present disclosure also encompass “full-length,” unprocessed IL1R1 polypeptide as well as any form of IL1R1 polypeptide that results from processing in the cell. NCBI Reference Sequences NP_000868.1, NP_001275635.1, NP_001307909.1, NP_001307910.1, NP_001307911.1, NP_001307912.1, NP_001307913.1, NP_001307914.1, NP_001307915.1, and NP_001307907.1 provide exemplary human IL1R1 polypeptide sequences.

As used herein, the term “IL17RC” refers to “interleukin-17 receptor C,” also known as “IL-17 receptor C,” “interleukin-17 receptor homolog (IL17Rhom),” “Interleukin-17 receptor-like protein (IL-17RL),” or “ZcytoR14,” in Uniprot or GenBank database. The term “IL17RC” encompasses the IL17RC polypeptides, the IL17RC RNA transcripts, and the IL17RC genes. The term “IL17RC gene” refers to genes encoding IL17RC polypeptides. IL17RC is expressed in various cells and tissues including plasma and endothelial cells, among others. Examples of IL17RC genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL17RC gene” includes all natural variants of the IL17RC genes, including allelic variants (e.g., SNP variants) and mutations. NCBI Gene ID: 84818 and NCBI Reference Sequence NC_000003.12 (range 9917074 . . . 9933627) provide exemplary human IL17RC nucleic acid sequences. In certain embodiments, IL17RC gene expression is determined by the amounts of the mRNA transcripts. IL17RC gene encodes various transcript variants. In certain embodiments, the mRNA transcripts are splice variants, fragments or derivatives of all native and natural variants of the transcripts of the IL17RC genes. NCBI Reference Sequences NM_001203263.2, NM_001203264.1, NM_001203265.2, NM_001367278.1, NM_001367279.1, NM_001367280.1, NM_032732.6, NM_153460.4, and NM_153461.4 provide exemplary human IL17RC mRNA transcript sequences. Examples of IL17RC polypeptides include any such native polypeptides from any vertebrate source as described above. In certain embodiments, IL17RC gene expression is determined by the amounts of the IL17RC polypeptides. In certain embodiments, the IL17RC polypeptides include all polypeptides encoded by the natural variants of the IL17RC genes and transcripts thereof, including allelic variants (e.g., SNP variants); splice variants; fragments; and derivatives. The IL17RC polypeptides of the present disclosure also encompass “full-length,” unprocessed IL17RC polypeptide as well as any form of IL17RC polypeptide that results from processing in the cell. NCBI Reference Sequences NP_001190192.2, NP_001190193.1, NP_001190194.2, NP_001354207.1, NP_001354208.1, NP_001354209.1, NP_116121.3, NP_703190.2, and NP_703191.2 provide exemplary human IL17RC polypeptide sequences.

As used herein, the term “IL20RA” refers to “interleukin 20 receptor subunit alpha,” also known as “IL-20 receptor subunit alpha,” “cytokine receptor family 2 member 8,” “class II cytokine receptor ZCYTOR7,” or “cytokine receptor class-II member 8,” in Uniprot or GenBank database. The term “IL20RA” encompasses the IL20RA polypeptides, the IL20RA RNA transcripts, and the IL20RA genes. The term “IL20RA gene” refers to genes encoding IL20RA polypeptides. IL20RA is expressed in various cells and tissues including plasma and endothelial cells, among others. Examples of IL20RA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL20RA gene” includes all natural variants of the IL20RA genes, including allelic variants (e.g., SNP variants) and mutations. NCBI Gene ID: 53832 and NCBI Reference Sequence NC_000006.12 (range 136999971 . . . 137045180, complement) provide exemplary human IL20RA nucleic acid sequences. In certain embodiments, IL20RA gene expression is determined by the amounts of the mRNA transcripts. IL20RA gene encodes various transcript variants. In certain embodiments, the mRNA transcripts are splice variants, fragments or derivatives of all native and natural variants of the transcripts of the IL20RA genes. NCBI Reference Sequences NM_001278722.1, NM_001278723.1, NM_001278724.2, and NM_014432.3 provide exemplary human IL20RA mRNA transcript sequences. Examples of IL20RA polypeptides include any such native polypeptides from any vertebrate source as described above. In certain embodiments, IL20RA gene expression is determined by the amounts of the IL20RA polypeptides. In certain embodiments, the IL20RA polypeptides include all polypeptides encoded by the natural variants of the IL20RA genes and transcripts thereof, including allelic variants (e.g., SNP variants); splice variants; fragments; and derivatives. The IL20RA polypeptides of the present disclosure also encompass “full-length,” unprocessed IL20RA polypeptide as well as any form of IL20RA polypeptide that results from processing in the cell. NCBI Reference Sequences NP_001265651.1, NP_001265652.1, NP_001265653.2, and NP_055247.3 provide exemplary human IL20RA polypeptide sequences.

As used herein, the term “IL22RA1” refers to “interleukin 22 receptor subunit alpha 1,” also known as “IL-22 receptor subunit alpha-1,” “cytokine receptor family 2 member 9,” “cytokine receptor class-II member 9,” or “zcytoR11,” in Uniprot or GenBank database. The term “IL22RA1” encompasses the IL22RA1 polypeptides, the IL22RA1 RNA transcripts, and the IL22RA1 genes. The term “IL22RA1 gene” refers to genes encoding IL22RA1 polypeptides. IL22RA1 is expressed in various cells and tissues including plasma and endothelial cells, among others. Examples of IL22RA1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IL22RA1 gene” includes all natural variants of the IL22RA1 genes, including allelic variants (e.g., SNP variants) and mutations. NCBI Gene ID: 58985 and NCBI Reference Sequence NC_000001.11 (range 24119771 . . . 24143179, complement) provide exemplary human IL22RA1 nucleic acid sequences. In certain embodiments, IL22RA1 gene expression is determined by the amounts of the mRNA transcripts. IL22RA1 gene encodes various transcript variants. In certain embodiments, the mRNA transcripts are splice variants, fragments or derivatives of all native and natural variants of the transcripts of the IL22RA1 genes. NCBI Reference Sequences NM_021258.4 and XM_011541882.1 provide exemplary human IL22RA1 mRNA transcript sequences. Examples of IL22RA1 polypeptides include any such native polypeptides from any vertebrate source as described above. In certain embodiments, IL22RA1 gene expression is determined by the amounts of the IL22RA1 polypeptides. In certain embodiments, the IL22RA1 polypeptides include all polypeptides encoded by the natural variants of the IL22RA1 genes and transcripts thereof, including allelic variants (e.g., SNP variants); splice variants; fragments; and derivatives. The IL22RA1 polypeptides of the present disclosure also encompass “full-length,” unprocessed IL22RA1 polypeptide as well as any form of IL22RA1 polypeptide that results from processing in the cell. NCBI Reference Sequences NP_067081.2 and XP_011540184.1 provide exemplary human IL22RA1 polypeptide sequences.

As used herein, the term “VTCN1” refers to “V-set domain containing T cell activation inhibitor 1,” also known as “V-set domain-containing T-cell activation inhibitor 1,” “B7 family member, H4,” “B7 homolog 4,” or “immune costimulatory protein B7-H4,” in Uniprot or GenBank database. The term “VTCN1” encompasses the VTCN1 polypeptides, the VTCN1 RNA transcripts, and the VTCN1 genes. The term “VTCN1 gene” refers to genes encoding VTCN1 polypeptides. VTCN1 is expressed in various cells and tissues including plasma and endothelial cells, among others. Examples of VTCN1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “VTCN1 gene” includes all natural variants of the VTCN1 genes, including allelic variants (e.g., SNP variants) and mutations. NCBI Gene ID: 79679 and NCBI Reference Sequence NC_000001.11 (range 117143587 . . . 117210985, complement) provide exemplary human VTCN1 nucleic acid sequences. In certain embodiments, VTCN1 gene expression is determined by the amounts of the mRNA transcripts. VTCN1 gene encodes various transcript variants. In certain embodiments, the mRNA transcripts are splice variants, fragments or derivatives of all native and natural variants of the transcripts of the VTCN1 genes. NCBI Reference Sequences NM_001253849.1, NM_001253850.1, and NM_024626.4 provide exemplary human VTCN1 mRNA transcript sequences. Examples of VTCN1 polypeptides include any such native polypeptides from any vertebrate source as described above. In certain embodiments, VTCN1 gene expression is determined by the amounts of the VTCN1 polypeptides. In certain embodiments, the VTCN1 polypeptides include all polypeptides encoded by the natural variants of the VTCN1 genes and transcripts thereof, including allelic variants (e.g., SNP variants); splice variants; fragments; and derivatives. The VTCN1 polypeptides of the present disclosure also encompass “full-length,” unprocessed VTCN1 polypeptide as well as any form of VTCN1 polypeptide that results from processing in the cell. NCBI Reference Sequences NP_001240778.1, NP_001240779.1, and NP_078902.2 provide exemplary human VTCN1 polypeptide sequences.

As used herein, the term “CD276” refers to “CD276 antigen,” also known as “CD276 molecule,” “B7 homolog 3,” “4Ig-B7-H3,” or “costimulatory molecule,” in Uniprot or GenBank database. The term “CD276” encompasses the CD276 polypeptides, the CD276 RNA transcripts, and the CD276 genes. The term “CD276 gene” refers to genes encoding CD276 polypeptides. CD276 is expressed in various cells and tissues including plasma and endothelial cells, among others. Examples of CD276 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CD276 gene” includes all natural variants of the CD276 genes, including allelic variants (e.g., SNP variants) and mutations. NCBI Gene ID: 80381 and NCBI Reference Sequence NC_000015.10 (range 73683966 . . . 73714518) provide exemplary human CD276 nucleic acid sequences. In certain embodiments, CD276 gene expression is determined by the amounts of the mRNA transcripts. CD276 gene encodes various transcript variants. In certain embodiments, the mRNA transcripts are splice variants, fragments or derivatives of all native and natural variants of the transcripts of the CD276 genes. NCBI Reference Sequences NM_001024736.2, NM_001329628.2, NM_001329629.2, and NM_025240.2 provide exemplary human CD276 mRNA transcript sequences. Examples of CD276 polypeptides include any such native polypeptides from any vertebrate source as described above. In certain embodiments, CD276 gene expression is determined by the amounts of the CD276 polypeptides. In certain embodiments, the CD276 polypeptides include all polypeptides encoded by the natural variants of the CD276 genes and transcripts thereof, including allelic variants (e.g., SNP variants); splice variants; fragments; and derivatives. The CD276 polypeptides of the present disclosure also encompass “full-length,” unprocessed CD276 polypeptide as well as any form of CD276 polypeptide that results from processing in the cell. NCBI Reference Sequences NP_001019907.1, NP_001316557.1, NP_001316558.1, and NP_079516.1 provide exemplary human CD276 polypeptide sequences.

As used herein, the term “PVRIG” refers to “PVR related immunoglobulin domain containing (protein),” also known as “poliovirus receptor-related immunoglobulin domain-containing protein,” “transmembrane protein PVRIG,” “nectin-2 receptor,” or “CD112 receptor,” in Uniprot or GenBank database. The term “PVRIG” encompasses the PVRIG polypeptides, the PVRIG RNA transcripts, and the PVRIG genes. The term “PVRIG gene” refers to genes encoding PVRIG polypeptides. PVRIG is expressed in various cells and tissues including plasma and endothelial cells, among others. Examples of PVRIG genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “PVRIG gene” includes all natural variants of the PVRIG genes, including allelic variants (e.g., SNP variants) and mutations. NCBI Gene ID: 79037 and NCBI Reference Sequence NC_000007.14 (range 100218625 . . . 100221489) provide exemplary human PVRIG nucleic acid sequences. In certain embodiments, PVRIG gene expression is determined by the amounts of the mRNA transcripts. PVRIG gene encodes various transcript variants. In certain embodiments, the mRNA transcripts are splice variants, fragments or derivatives of all native and natural variants of the transcripts of the PVRIG genes. NCBI Reference Sequences NM_024070.3 and XM_011516575.2 provide exemplary human PVRIG mRNA transcript sequences. Examples of PVRIG polypeptides include any such native polypeptides from any vertebrate source as described above. In certain embodiments, PVRIG gene expression is determined by the amounts of the PVRIG polypeptides. In certain embodiments, the PVRIG polypeptides include all polypeptides encoded by the natural variants of the PVRIG genes and transcripts thereof, including allelic variants (e.g., SNP variants); splice variants; fragments; and derivatives. The PVRIG polypeptides of the present disclosure also encompass “full-length,” unprocessed PVRIG polypeptide as well as any form of PVRIG polypeptide that results from processing in the cell. NCBI Reference Sequences NP_076975.2 and XP_011514877.1 provide exemplary human PVRIG polypeptide sequences.

As used herein, the term “PVRL2” refers to “nectin cell adhesion molecule 2,” also known as “NECTIN2,” “nectin-2,” “poliovirus receptor-related (protein) 2,” “CD112,” or “Herpes virus entry mediator B,” in Uniprot or GenBank database. The term “PVRL2” encompasses the PVRL2 polypeptides, the PVRL2 RNA transcripts, and the PVRL2 genes. The term “PVRL2 gene” refers to genes encoding PVRL2 polypeptides. PVRL2 is expressed in various cells and tissues including plasma and endothelial cells, among others. Examples of PVRL2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “PVRL2 gene” includes all natural variants of the PVRL2 genes, including allelic variants (e.g., SNP variants) and mutations. NCBI Gene ID: 5819 and NCBI Reference Sequence NC_000019.10 (range 44846297 . . . 44889223) provide exemplary human PVRL2 nucleic acid sequences. In certain embodiments, PVRL2 gene expression is determined by the amounts of the mRNA transcripts. PVRL2 gene encodes various transcript variants. In certain embodiments, the mRNA transcripts are splice variants, fragments or derivatives of all native and natural variants of the transcripts of the PVRL2 genes. NCBI Reference Sequences NM_001042724.2 and NM_002856.3 provide exemplary human PVRL2 mRNA transcript sequences. Examples of PVRL2 polypeptides include any such native polypeptides from any vertebrate source as described above. In certain embodiments, PVRL2 gene expression is determined by the amounts of the PVRL2 polypeptides. In certain embodiments, the PVRL2 polypeptides include all polypeptides encoded by the natural variants of the PVRL2 genes and transcripts thereof, including allelic variants (e.g., SNP variants); splice variants; fragments; and derivatives. The PVRL2 polypeptides of the present disclosure also encompass “full-length,” unprocessed PVRL2 polypeptide as well as any form of PVRL2 polypeptide that results from processing in the cell. NCBI Reference Sequences NP_001036189.1 and NP_002847.1 provide exemplary human PVRL2 polypeptide sequences.

As used herein, the term “TIGIT” refers to “T cell immunoreceptor with Ig and ITIM domains,” also known as “V-set and immunoglobulin domain-containing protein 9,” “V-set and transmembrane domain-containing protein 3,” “VSIG9,” or “VSTM3,” in Uniprot or GenBank database. The term “TIGIT” encompasses the TIGIT polypeptides, the TIGIT RNA transcripts, and the TIGIT genes. The term “TIGIT gene” refers to genes encoding TIGIT polypeptides. TIGIT is expressed in various cells and tissues including plasma and endothelial cells, among others. Examples of TIGIT genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TIGIT gene” includes all natural variants of the TIGIT genes, including allelic variants (e.g., SNP variants) and mutations. NCBI Gene ID: 201633 and NCBI Reference Sequence NC_000003.12 (range 114291102 . . . 114329747) provide exemplary human TIGIT nucleic acid sequences. In certain embodiments, TIGIT gene expression is determined by the amounts of the mRNA transcripts. TIGIT gene encodes various transcript variants. In certain embodiments, the mRNA transcripts are splice variants, fragments or derivatives of all native and natural variants of the transcripts of the TIGIT genes. NCBI Reference Sequences NM_173799.4 and XM_024453388.1 provide exemplary human TIGIT mRNA transcript sequences. Examples of TIGIT polypeptides include any such native polypeptides from any vertebrate source as described above. In certain embodiments, TIGIT gene expression is determined by the amounts of the TIGIT polypeptides. In certain embodiments, the TIGIT polypeptides include all polypeptides encoded by the natural variants of the TIGIT genes and transcripts thereof, including allelic variants (e.g., SNP variants); splice variants; fragments; and derivatives. The TIGIT polypeptides of the present disclosure also encompass “full-length,” unprocessed TIGIT polypeptide as well as any form of TIGIT polypeptide that results from processing in the cell. NCBI Reference Sequences NP_776160.2 and XP_024309156.1 provide exemplary human TIGIT polypeptide sequences.

As used herein, the term “LAG3” refers to “lymphocyte activation gene 3 protein,” also known as “lymphocyte activating 3,” “CD223,” “lymphocyte-activation gene 3,” or “LAG-3,” in Uniprot or GenBank database. The term “LAG3” encompasses the LAG3 polypeptides, the LAG3 RNA transcripts, and the LAG3 genes. The term “LAG3 gene” refers to genes encoding LAG3 polypeptides. LAG3 is expressed in various cells and tissues including plasma and endothelial cells, among others. Examples of LAG3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “LAG3 gene” includes all natural variants of the LAG3 genes, including allelic variants (e.g., SNP variants) and mutations. NCBI Gene ID: 3902 and NCBI Reference Sequence NC_000012.12 (range 6772483 . . . 6778455) provide exemplary human LAG3 nucleic acid sequences. In certain embodiments, LAG3 gene expression is determined by the amounts of the mRNA transcripts. LAG3 gene encodes various transcript variants. In certain embodiments, the mRNA transcripts are splice variants, fragments or derivatives of all native and natural variants of the transcripts of the LAG3 genes. NCBI Reference Sequences NM_002286.6 and XM_011520956.1 provide exemplary human LAG3 mRNA transcript sequences. Examples of LAG3 polypeptides include any such native polypeptides from any vertebrate source as described above. In certain embodiments, LAG3 gene expression is determined by the amounts of the LAG3 polypeptides. In certain embodiments, the LAG3 polypeptides include all polypeptides encoded by the natural variants of the LAG3 genes and transcripts thereof, including allelic variants (e.g., SNP variants); splice variants; fragments; and derivatives. The LAG3 polypeptides of the present disclosure also encompass “full-length,” unprocessed LAG3 polypeptide as well as any form of LAG3 polypeptide that results from processing in the cell. NCBI Reference Sequences NP_002277.4 and XP_011519258.1 provide exemplary human LAG3 polypeptide sequences.

As used herein, the term “CSF1R” refers to “colony stimulating factor 1 receptor,” also known as “macrophage colony-stimulating factor 1 receptor,” “CD115,” “proto-oncogene c-Fms,” or “CSF-1 receptor,” in Uniprot or GenBank database. The term “CSF1R” encompasses the CSF1R polypeptides, the CSF1R RNA transcripts, and the CSF1R genes. The term “CSF1R gene” refers to genes encoding CSF1R polypeptides. CSF1R is expressed in various cells and tissues including plasma and endothelial cells, among others. Examples of CSF1R genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CSF1R gene” includes all natural variants of the CSF1R genes, including allelic variants (e.g., SNP variants) and mutations. NCBI Gene ID: 1436 and NCBI Reference Sequence NC_000005.10 (range 150053291 . . . 150113372, complement) provide exemplary human CSF1R nucleic acid sequences. In certain embodiments, CSF1R gene expression is determined by the amounts of the mRNA transcripts. CSF1R gene encodes various transcript variants. In certain embodiments, the mRNA transcripts are splice variants, fragments or derivatives of all native and natural variants of the transcripts of the CSF1R genes. NCBI Reference Sequences NM_001288705.3, NM_001349736.1, NM_001375320.1, NM_001375321.1, and NM_005211.3 provide exemplary human CSF1R mRNA transcript sequences. Examples of CSF1R polypeptides include any such native polypeptides from any vertebrate source as described above. In certain embodiments, CSF1R gene expression is determined by the amounts of the CSF1R polypeptides. In certain embodiments, the CSF1R polypeptides include all polypeptides encoded by the natural variants of the CSF1R genes and transcripts thereof, including allelic variants (e.g., SNP variants); splice variants; fragments; and derivatives. The CSF1R polypeptides of the present disclosure also encompass “full-length,” unprocessed CSF1R polypeptide as well as any form of CSF1R polypeptide that results from processing in the cell. NCBI Reference Sequences NP_001275634.1, NP_001336665.1, NP_001362249.1, NP_001362250.1, and NP_005202.2 provide exemplary human CSF1R polypeptide sequences.

As used herein, the term “PDGFRB” refers to “platelet derived growth factor receptor beta,” also known as “beta-type platelet-derived growth factor receptor,” “platelet-derived growth factor receptor 1,” “CD140 antigen-like family member B,” or “CD140b,” in Uniprot or GenBank database. The term “PDGFRB” encompasses the PDGFRB polypeptides, the PDGFRB RNA transcripts, and the PDGFRB genes. The term “PDGFRB gene” refers to genes encoding PDGFRB polypeptides. PDGFRB is expressed in various cells and tissues including plasma and endothelial cells, among others. Examples of PDGFRB genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “PDGFRB gene” includes all natural variants of the PDGFRB genes, including allelic variants (e.g., SNP variants) and mutations. NCBI Gene ID: 5159 and NCBI Reference Sequence NC_000005.10 (range 150113839 . . . 150155845, complement) provide exemplary human PDGFRB nucleic acid sequences. In certain embodiments, PDGFRB gene expression is determined by the amounts of the mRNA transcripts. PDGFRB gene encodes various transcript variants. In certain embodiments, the mRNA transcripts are splice variants, fragments or derivatives of all native and natural variants of the transcripts of the PDGFRB genes. NCBI Reference Sequences NM_001288705.3, NM_001355016.2, NM_001355017.2, and NM_002609.4 provide exemplary human PDGFRB mRNA transcript sequences. Examples of PDGFRB polypeptides include any such native polypeptides from any vertebrate source as described above. In certain embodiments, PDGFRB gene expression is determined by the amounts of the PDGFRB polypeptides. In certain embodiments, the PDGFRB polypeptides include all polypeptides encoded by the natural variants of the PDGFRB genes and transcripts thereof, including allelic variants (e.g., SNP variants); splice variants; fragments; and derivatives. The PDGFRB polypeptides of the present disclosure also encompass “full-length,” unprocessed PDGFRB polypeptide as well as any form of PDGFRB polypeptide that results from processing in the cell. NCBI Reference Sequences NM_001355016.2, NM_001355017.2, and NM_002609.4 provide exemplary human PDGFRB polypeptide sequences.

As used herein, the terms “TEK/TIE2”, “TEK,” and “TIE2” are used interchangeably to refer to “TEK receptor tyrosine kinase,” also known as “angiopoietin-1 receptor,” “Tyrosine-protein kinase receptor TEK,” “tyrosine-protein kinase receptor TIE-2,” “endothelial tyrosine kinase,” “tunica interna endothelial cell kinase,” or “CD202b,” in Uniprot or GenBank database. The term “TEK/TIE2” encompasses the TEK/TIE2 polypeptides, the TEK/TIE2 RNA transcripts, and the TEK/TIE2 genes. The term “TEK/TIE2 gene” refers to genes encoding TEK/TIE2 polypeptides. TEK/TIE2 is expressed in various cells and tissues including plasma and endothelial cells, among others. Examples of TEK/TIE2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TEK/TIE2 gene” includes all natural variants of the TEK/TIE2 genes, including allelic variants (e.g., SNP variants) and mutations. NCBI Gene ID: 7010 and NCBI Reference Sequence NC_000009.12 (range 27109141 . . . 27230178) provide exemplary human TEK/TIE2 nucleic acid sequences. In certain embodiments, TEK/TIE2 gene expression is determined by the amounts of the mRNA transcripts. TEK/TIE2 gene encodes various transcript variants. In certain embodiments, the mRNA transcripts are splice variants, fragments or derivatives of all native and natural variants of the transcripts of the TEK/TIE2 genes. NCBI Reference Sequences NM_000459.5, NM_001290077.1, NM_001290078.1, NM_001375475.1, and NM_001375476.1 provide exemplary human TEK/TIE2 mRNA transcript sequences. Examples of TEK/TIE2 polypeptides include any such native polypeptides from any vertebrate source as described above. In certain embodiments, TEK/TIE2 gene expression is determined by the amounts of the TEK/TIE2 polypeptides. In certain embodiments, the TEK/TIE2 polypeptides include all polypeptides encoded by the natural variants of the TEK/TIE2 genes and transcripts thereof, including allelic variants (e.g., SNP variants); splice variants; fragments; and derivatives. The TEK/TIE2 polypeptides of the present disclosure also encompass “full-length,” unprocessed TEK/TIE2 polypeptide as well as any form of TEK/TIE2 polypeptide that results from processing in the cell. NCBI Reference Sequences NP_000450.3, NP_001277006.1, NP_001277007.1, NP_001362404.1, and NP_001362405.1 provide exemplary human TEK/TIE2 polypeptide sequences.

As used herein, the term “FLT3” refers to “receptor-type tyrosine-protein kinase FLT3,” also known as “FL cytokine receptor,” “fetal liver kinase 2,” “fms-like tyrosine kinase 3,” “stem cell tyrosine kinase 1,” or “CD135,” in Uniprot or GenBank database. The term “FLT3” encompasses the FLT3 polypeptides, the FLT3 RNA transcripts, and the FLT3 genes. The term “FLT3 gene” refers to genes encoding FLT3 polypeptides. FLT3 is expressed in various cells and tissues including plasma and endothelial cells, among others. Examples of FLT3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “FLT3 gene” includes all natural variants of the FLT3 genes, including allelic variants (e.g., SNP variants) and mutations. NCBI Gene ID: 2322 and NCBI Reference Sequence NC_000013.11 (range 28003274 . . . 28100587, complement) provide exemplary human FLT3 nucleic acid sequences. In certain embodiments, FLT3 gene expression is determined by the amounts of the mRNA transcripts. FLT3 gene encodes various transcript variants. In certain embodiments, the mRNA transcripts are splice variants, fragments or derivatives of all native and natural variants of the transcripts of the FLT3 genes. NCBI Reference Sequences NM_004119.3, XM_017020486.1, XM_017020489.1, XM_017020487.1, XM_017020488.1, XM_011535015.2, XM_011535017.2, and XM_011535018.2 provide exemplary human FLT3 mRNA transcript sequences. Examples of FLT3 polypeptides include any such native polypeptides from any vertebrate source as described above. In certain embodiments, FLT3 gene expression is determined by the amounts of the FLT3 polypeptides. In certain embodiments, the FLT3 polypeptides include all polypeptides encoded by the natural variants of the FLT3 genes and transcripts thereof, including allelic variants (e.g., SNP variants); splice variants; fragments; and derivatives. The FLT3 polypeptides of the present disclosure also encompass “full-length,” unprocessed FLT3 polypeptide as well as any form of FLT3 polypeptide that results from processing in the cell. NCBI Reference Sequences NP_004110.2, XP_016875975.1, XP_016875978.1, XP_016875976.1, XP_016875977.1, XP_011533317.1, XP_011533319.1, and XP_011533320.1 provide exemplary human FLT3 polypeptide sequences.

As used herein, the term “CD40” refers to “tumor necrosis factor receptor superfamily member 5,” also known as “B cell surface antigen CD40,” “CD40L receptor,” “CD40 molecule, TNF receptor superfamily member 5,” or “TNFRSF5,” in Uniprot or GenBank database. The term “CD40” encompasses the CD40 polypeptides, the CD40 RNA transcripts, and the CD40 genes. The term “CD40 gene” refers to genes encoding CD40 polypeptides. CD40 is expressed in various cells and tissues including plasma and endothelial cells, among others. Examples of CD40 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “CD40 gene” includes all natural variants of the CD40 genes, including allelic variants (e.g., SNP variants) and mutations. NCBI Gene ID: 958 and NCBI Reference Sequence NC_000020.11 (range 46118242 . . . 46129858) provide exemplary human CD40 nucleic acid sequences. In certain embodiments, CD40 gene expression is determined by the amounts of the mRNA transcripts. CD40 gene encodes various transcript variants. In certain embodiments, the mRNA transcripts are splice variants, fragments or derivatives of all native and natural variants of the transcripts of the CD40 genes. NCBI Reference Sequences NM_001250.6, NM_001302753.2, NM_001322421.2, NM_001322422.2, NM_001362758.2, and NM_152854.4 provide exemplary human CD40 mRNA transcript sequences. Examples of CD40 polypeptides include any such native polypeptides from any vertebrate source as described above. In certain embodiments, CD40 gene expression is determined by the amounts of the CD40 polypeptides. In certain embodiments, the CD40 polypeptides include all polypeptides encoded by the natural variants of the CD40 genes and transcripts thereof, including allelic variants (e.g., SNP variants); splice variants; fragments; and derivatives. The CD40 polypeptides of the present disclosure also encompass “full-length,” unprocessed CD40 polypeptide as well as any form of CD40 polypeptide that results from processing in the cell. NCBI Reference Sequences NP_001241.1, NP_001289682.1, NP_001309350.1, NP_001309351.1, NP_001349687.1, and NP_690593.1 provide exemplary human CD40 polypeptide sequences.

As used herein, the term “TNFRSF1A” refers to “tumor necrosis factor receptor superfamily member 1A,” also known as “tumor necrosis factor binding protein 1,” “tumor necrosis factor receptor type 1,” “TNF-RI,” “CD120a,” “TNFR-I,” or “TNF-R1,” in Uniprot or GenBank database. The term “TNFRSF1A” encompasses the TNFRSF1A polypeptides, the TNFRSF1A RNA transcripts, and the TNFRSF1A genes. The term “TNFRSF1A gene” refers to genes encoding TNFRSF1A polypeptides. TNFRSF1A is expressed in various cells and tissues including plasma and endothelial cells, among others. Examples of TNFRSF1A genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TNFRSF1A gene” includes all natural variants of the TNFRSF1A genes, including allelic variants (e.g., SNP variants) and mutations. NCBI Gene ID: 7132 and NCBI Reference Sequence NC_000012.12 (range 6328771 . . . 6342076, complement) provide exemplary human TNFRSF1A nucleic acid sequences. In certain embodiments, TNFRSF1A gene expression is determined by the amounts of the mRNA transcripts. TNFRSF1A gene encodes various transcript variants. In certain embodiments, the mRNA transcripts are splice variants, fragments or derivatives of all native and natural variants of the transcripts of the TNFRSF1A genes. NCBI Reference Sequences NM_001065.4, NM_001346091.2, and NM_001346092.2 provide exemplary human TNFRSF1A mRNA transcript sequences. Examples of TNFRSF1A polypeptides include any such native polypeptides from any vertebrate source as described above. In certain embodiments, TNFRSF1A gene expression is determined by the amounts of the TNFRSF1A polypeptides. In certain embodiments, the TNFRSF1A polypeptides include all polypeptides encoded by the natural variants of the TNFRSF1A genes and transcripts thereof, including allelic variants (e.g., SNP variants); splice variants; fragments; and derivatives. The TNFRSF1A polypeptides of the present disclosure also encompass “full-length,” unprocessed TNFRSF1A polypeptide as well as any form of TNFRSF1A polypeptide that results from processing in the cell. NCBI Reference Sequences NP_001056.1, NP_001333020.1, and NP_001333021.1 provide exemplary human TNFRSF1A polypeptide sequences.

As used herein, the term “TNFRSF21” refers to “tumor necrosis factor receptor superfamily member 21,” also known as “TNFR-related death receptor 6,” “TNF receptor superfamily member 21,” “Death receptor 6,” or “CD358,” in Uniprot or GenBank database. The term “TNFRSF21” encompasses the TNFRSF21 polypeptides, the TNFRSF21 RNA transcripts, and the TNFRSF21 genes. The term “TNFRSF21 gene” refers to genes encoding TNFRSF21 polypeptides. TNFRSF21 is expressed in various cells and tissues including plasma and endothelial cells, among others. Examples of TNFRSF21 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TNFRSF21 gene” includes all natural variants of the TNFRSF21 genes, including allelic variants (e.g., SNP variants) and mutations. NCBI Gene ID: 27242 and NCBI Reference Sequence NC_000006.12 (range 47231532 . . . 47309910, complement) provide exemplary human TNFRSF21 nucleic acid sequences. In certain embodiments, TNFRSF21 gene expression is determined by the amounts of the mRNA transcripts. TNFRSF21 gene encodes various transcript variants. In certain embodiments, the mRNA transcripts are splice variants, fragments or derivatives of all native and natural variants of the transcripts of the TNFRSF21 genes. NCBI Reference Sequences NM_014452.5 and XM_017010744.2 provide exemplary human TNFRSF21 mRNA transcript sequences. Examples of TNFRSF21 polypeptides include any such native polypeptides from any vertebrate source as described above. In certain embodiments, TNFRSF21 gene expression is determined by the amounts of the TNFRSF21 polypeptides. In certain embodiments, the TNFRSF21 polypeptides include all polypeptides encoded by the natural variants of the TNFRSF21 genes and transcripts thereof, including allelic variants (e.g., SNP variants); splice variants; fragments; and derivatives. The TNFRSF21 polypeptides of the present disclosure also encompass “full-length,” unprocessed TNFRSF21 polypeptide as well as any form of TNFRSF21 polypeptide that results from processing in the cell. NCBI Reference Sequences NP_055267.1 and XP_016866233.1 provide exemplary human TNFRSF21 polypeptide sequences.

As used herein, the term “TNFRSF1B” refers to “tumor necrosis factor receptor superfamily member 1B,” also known as “tumor necrosis factor receptor 2,” “TNF receptor superfamily member 1B,” “tumor necrosis factor receptor type II,” “p80 TNF-alpha receptor,” or “CD120b,” in Uniprot or GenBank database. The term “TNFRSF1B” encompasses the TNFRSF1B polypeptides, the TNFRSF1B RNA transcripts, and the TNFRSF1B genes. The term “TNFRSF1B gene” refers to genes encoding TNFRSF1B polypeptides. TNFRSF1B is expressed in various cells and tissues including plasma and endothelial cells, among others. Examples of TNFRSF1B genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TNFRSF1B gene” includes all natural variants of the TNFRSF1B genes, including allelic variants (e.g., SNP variants) and mutations. NCBI Gene ID: 7133 and NCBI Reference Sequence NC_000001.11 (range 12166948 . . . 12209222) provide exemplary human TNFRSF1B nucleic acid sequences. In certain embodiments, TNFRSF1B gene expression is determined by the amounts of the mRNA transcripts. TNFRSF1B gene encodes various transcript variants. In certain embodiments, the mRNA transcripts are splice variants, fragments or derivatives of all native and natural variants of the transcripts of the TNFRSF1B genes. NCBI Reference Sequences NM_001066.3, XM_011542060.2, XM_011542063.2, XM_017002214.1, XM_017002215.1, and XM_017002211.1 provide exemplary human TNFRSF1B mRNA transcript sequences. Examples of TNFRSF1B polypeptides include any such native polypeptides from any vertebrate source as described above. In certain embodiments, TNFRSF1B gene expression is determined by the amounts of the TNFRSF1B polypeptides. In certain embodiments, the TNFRSF1B polypeptides include all polypeptides encoded by the natural variants of the TNFRSF1B genes and transcripts thereof, including allelic variants (e.g., SNP variants); splice variants; fragments; and derivatives. The TNFRSF1B polypeptides of the present disclosure also encompass “full-length,” unprocessed TNFRSF1B polypeptide as well as any form of TNFRSF1B polypeptide that results from processing in the cell. NCBI Reference Sequences NP_001057.1, XP_011540362.1, XP_011540365.1, XP_016857703.1, XP_016857704.1, and XP_016857700.1 provide exemplary human TNFRSF1B polypeptide sequences.

As used herein, the term “IFNAR1” refers to “interferon alpha/beta receptor 1,” also known as “interferon alpha and beta receptor subunit 1,” “cytokine receptor class-II member 1,” “cytokine receptor family 2 member 1,” “type I interferon receptor 1,” or “CRF2-1,” in Uniprot or GenBank database. The term “IFNAR1” encompasses the IFNAR1 polypeptides, the IFNAR1 RNA transcripts, and the IFNAR1 genes. The term “IFNAR1 gene” refers to genes encoding IFNAR1 polypeptides. IFNAR1 is expressed in various cells and tissues including plasma and endothelial cells, among others. Examples of IFNAR1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IFNAR1 gene” includes all natural variants of the IFNAR1 genes, including allelic variants (e.g., SNP variants) and mutations. NCBI Gene ID: 3454 and NCBI Reference Sequence NC_000021.9 (range 33324443 . . . 33360361) provide exemplary human IFNAR1 nucleic acid sequences. In certain embodiments, IFNAR1 gene expression is determined by the amounts of the mRNA transcripts. IFNAR1 gene encodes various transcript variants. In certain embodiments, the mRNA transcripts are splice variants, fragments or derivatives of all native and natural variants of the transcripts of the IFNAR1 genes. NCBI Reference Sequences NM_000629.3, XM_005260964.2, and XM_011529552.2 provide exemplary human IFNAR1 mRNA transcript sequences. Examples of IFNAR1 polypeptides include any such native polypeptides from any vertebrate source as described above. In certain embodiments, IFNAR1 gene expression is determined by the amounts of the IFNAR1 polypeptides. In certain embodiments, the IFNAR1 polypeptides include all polypeptides encoded by the natural variants of the IFNAR1 genes and transcripts thereof, including allelic variants (e.g., SNP variants); splice variants; fragments; and derivatives. The IFNAR1 polypeptides of the present disclosure also encompass “full-length,” unprocessed IFNAR1 polypeptide as well as any form of IFNAR1 polypeptide that results from processing in the cell. NCBI Reference Sequences NP_000620.2, XP_005261021.1, and XP_011527854.1 provide exemplary human IFNAR1 polypeptide sequences.

As used herein, the term “IFNAR2” refers to “interferon alpha and beta receptor subunit 2,” also known as “interferon alpha/beta receptor 2,” “interferon alpha binding protein,” “type I interferon receptor 2,” “IFN-alpha/beta receptor 2,” or “IFN-R-2,” in Uniprot or GenBank database. The term “IFNAR2” encompasses the IFNAR2 polypeptides, the IFNAR2 RNA transcripts, and the IFNAR2 genes. The term “IFNAR2 gene” refers to genes encoding IFNAR2 polypeptides. IFNAR2 is expressed in various cells and tissues including plasma and endothelial cells, among others. Examples of IFNAR2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IFNAR2 gene” includes all natural variants of the IFNAR2 genes, including allelic variants (e.g., SNP variants) and mutations. NCBI Gene ID: 3455 and NCBI Reference Sequence NC_000021.9 (range 33229895 . . . 33265664) provide exemplary human IFNAR2 nucleic acid sequences. In certain embodiments, IFNAR2 gene expression is determined by the amounts of the mRNA transcripts. IFNAR2 gene encodes various transcript variants. In certain embodiments, the mRNA transcripts are splice variants, fragments or derivatives of all native and natural variants of the transcripts of the IFNAR2 genes. NCBI Reference Sequences NM_000874.5, NM_001289125.3, NM_001289126.1, NM_001289128.1, NM_207584.3, and NM_207585.2 provide exemplary human IFNAR2 mRNA transcript sequences. Examples of IFNAR2 polypeptides include any such native polypeptides from any vertebrate source as described above. In certain embodiments, IFNAR2 gene expression is determined by the amounts of the IFNAR2 polypeptides. In certain embodiments, the IFNAR2 polypeptides include all polypeptides encoded by the natural variants of the IFNAR2 genes and transcripts thereof, including allelic variants (e.g., SNP variants); splice variants; fragments; and derivatives. The IFNAR2 polypeptides of the present disclosure also encompass “full-length,” unprocessed IFNAR2 polypeptide as well as any form of IFNAR2 polypeptide that results from processing in the cell. NCBI Reference Sequences NP_000865.2, NP_001276054.1, NP_001276055.1, NP_001276057.1, NP_997467.1, NP_997468.1 provide exemplary human IFNAR2 polypeptide sequences.

As used herein, the terms “TIM3” and “HAVCR2” are used interchangeably to refer to “hepatitis A virus cellular receptor 2,” also known as “T-cell immunoglobulin and mucin domain-containing protein 3,” “T-cell immunoglobulin mucin family member 3,” “T-cell membrane protein 3,” “TIMD-3,” or “CD366,” in Uniprot or GenBank database. The term “TIM3” encompasses the TIM3 polypeptides, the TIM3 RNA transcripts, and the TIM3 genes. The term “TIM3 gene” refers to genes encoding TIM3 polypeptides. TIM3 is expressed in various cells and tissues including plasma and endothelial cells, among others. Examples of TIM3 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TIM3 gene” includes all natural variants of the TIM3 genes, including allelic variants (e.g., SNP variants) and mutations. NCBI Gene ID: 84868 and NCBI Reference Sequence NC_000005.10 (range 157085832 . . . 157109044, complement) provide exemplary human TIM3 nucleic acid sequences. In certain embodiments, TIM3 gene expression is determined by the amounts of the mRNA transcripts. TIM3 gene encodes various transcript variants. In certain embodiments, the mRNA transcripts are splice variants, fragments or derivatives of all native and natural variants of the transcripts of the TIM3 genes. NCBI Reference Sequences NM_032782.5 and BC063431 provide exemplary human TIM3 mRNA transcript sequences. Examples of TIM3 polypeptides include any such native polypeptides from any vertebrate source as described above. In certain embodiments, TIM3 gene expression is determined by the amounts of the TIM3 polypeptides. In certain embodiments, the TIM3 polypeptides include all polypeptides encoded by the natural variants of the TIM3 genes and transcripts thereof, including allelic variants (e.g., SNP variants); splice variants; fragments; and derivatives. The TIM3 polypeptides of the present disclosure also encompass “full-length,” unprocessed TIM3 polypeptide as well as any form of TIM3 polypeptide that results from processing in the cell. NCBI Reference Sequences NP_116171.3 and AAH63431 provide exemplary human TIM3 polypeptide sequences.

As used herein, the term “VSIR” refers to “V-set immunoregulatory receptor,” also known as “V-type immunoglobulin domain-containing suppressor of T-cell activation,” “V-set domain-containing immunoregulatory receptor,” “stress-induced secreted protein-1,” “platelet receptor GI24,” or “sisp-1,” in Uniprot or GenBank database. The term “VSIR” encompasses the VSIR polypeptides, the VSIR RNA transcripts, and the VSIR genes. The term “VSIR gene” refers to genes encoding VSIR polypeptides. VSIR is expressed in various cells and tissues including plasma and endothelial cells, among others. Examples of VSIR genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “VSIR gene” includes all natural variants of the VSIR genes, including allelic variants (e.g., SNP variants) and mutations. NCBI Gene ID: 64115 and NCBI Reference Sequence NC_000010.11 (range 71747556 . . . 71773520, complement) provide exemplary human VSIR nucleic acid sequences. In certain embodiments, VSIR gene expression is determined by the amounts of the mRNA transcripts. VSIR gene encodes various transcript variants. In certain embodiments, the mRNA transcripts are splice variants, fragments or derivatives of all native and natural variants of the transcripts of the VSIR genes. NCBI Reference Sequence NM_022153.2 provides an exemplary human VSIR mRNA transcript sequence. Examples of VSIR polypeptides include any such native polypeptides from any vertebrate source as described above. In certain embodiments, VSIR gene expression is determined by the amounts of the VSIR polypeptides. In certain embodiments, the VSIR polypeptides include all polypeptides encoded by the natural variants of the VSIR genes and transcripts thereof, including allelic variants (e.g., SNP variants); splice variants; fragments; and derivatives. The VSIR polypeptides of the present disclosure also encompass “full-length,” unprocessed VSIR polypeptide as well as any form of VSIR polypeptide that results from processing in the cell. NCBI Reference Sequence NP_071436.1 provides an exemplary human VSIR polypeptide sequence.

As used herein, the term “IDO1” refers to “indoleamine 2,3-dioxygenase 1,” also known as “indoleamine-pyrrole 2,3-dioxygenase,” “IDO—1,” or “INDO,” in Uniprot or GenBank database. The term “IDO1” encompasses the IDO1 polypeptides, the IDO1 RNA transcripts, and the IDO1 genes. The term “IDO1 gene” refers to genes encoding IDO1 polypeptides. IDO1 is expressed in various cells and tissues including plasma and endothelial cells, among others. Examples of IDO1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IDO1 gene” includes all natural variants of the IDO1 genes, including allelic variants (e.g., SNP variants) and mutations. NCBI Gene ID: 3620 and NCBI Reference Sequence NC_000008.11 (range 39913891 . . . 39928790) provide exemplary human IDO1 nucleic acid sequences. In certain embodiments, IDO1 gene expression is determined by the amounts of the mRNA transcripts. IDO1 gene encodes various transcript variants. In certain embodiments, the mRNA transcripts are splice variants, fragments or derivatives of all native and natural variants of the transcripts of the IDO1 genes. NCBI Reference Sequence NM_002164.6 provides an exemplary human IDO1 mRNA transcript sequence. Examples of IDO1 polypeptides include any such native polypeptides from any vertebrate source as described above. In certain embodiments, IDO1 gene expression is determined by the amounts of the IDO1 polypeptides. In certain embodiments, the IDO1 polypeptides include all polypeptides encoded by the natural variants of the IDO1 genes and transcripts thereof, including allelic variants (e.g., SNP variants); splice variants; fragments; and derivatives. The IDO1 polypeptides of the present disclosure also encompass “full-length,” unprocessed IDO1 polypeptide as well as any form of IDO1 polypeptide that results from processing in the cell. NCBI Reference Sequence NP_002155.1 provides an exemplary human IDO1 polypeptide sequence.

As used herein, the term “TDO2” refers to “tryptophan 2,3-dioxygenase,” also known as “tryptamin 2,3-dioxygenase,” “tryptophan oxygenase,” “tryptophanase,” or “tryptophan pyrrolase,” in Uniprot or GenBank database. The term “TDO2” encompasses the TDO2 polypeptides, the TDO2 RNA transcripts, and the TDO2 genes. The term “TDO2 gene” refers to genes encoding TDO2 polypeptides. TDO2 is expressed in various cells and tissues including plasma and endothelial cells, among others. Examples of TDO2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TDO2 gene” includes all natural variants of the TDO2 genes, including allelic variants (e.g., SNP variants) and mutations. NCBI Gene ID: 6999 and NCBI Reference Sequence NC_000004.12 (range 155903696 . . . 155920406) provide exemplary human TDO2 nucleic acid sequences. In certain embodiments, TDO2 gene expression is determined by the amounts of the mRNA transcripts. TDO2 gene encodes various transcript variants. In certain embodiments, the mRNA transcripts are splice variants, fragments or derivatives of all native and natural variants of the transcripts of the TDO2 genes. NCBI Reference Sequence NM_005651.4 provides an exemplary human TDO2 mRNA transcript sequence. Examples of TDO2 polypeptides include any such native polypeptides from any vertebrate source as described above. In certain embodiments, TDO2 gene expression is determined by the amounts of the TDO2 polypeptides. In certain embodiments, the TDO2 polypeptides include all polypeptides encoded by the natural variants of the TDO2 genes and transcripts thereof, including allelic variants (e.g., SNP variants); splice variants; fragments; and derivatives. The TDO2 polypeptides of the present disclosure also encompass “full-length,” unprocessed TDO2 polypeptide as well as any form of TDO2 polypeptide that results from processing in the cell. NCBI Reference Sequence NP_005642.1 provides an exemplary human TDO2 polypeptide sequence.

As used herein, the term “EIF2AK2” refers to “eukaryotic translation initiation factor 2 alpha kinase 2,” also known as “eIF-2A protein kinase 2,” “P1/eIF-2A protein kinase,” “tyrosine-protein kinase EIF2AK2,” or “protein kinase R,” in Uniprot or GenBank database. The term “EIF2AK2” encompasses the EIF2AK2 polypeptides, the EIF2AK2 RNA transcripts, and the EIF2AK2 genes. The term “EIF2AK2 gene” refers to genes encoding EIF2AK2 polypeptides. EIF2AK2 is expressed in various cells and tissues including plasma and endothelial cells, among others. Examples of EIF2AK2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “EIF2AK2 gene” includes all natural variants of the EIF2AK2 genes, including allelic variants (e.g., SNP variants) and mutations. NCBI Gene ID: 5610 and NCBI Reference Sequence NC_000002.12 (range 37099210 . . . 37157065, complement) provide exemplary human EIF2AK2 nucleic acid sequences. In certain embodiments, EIF2AK2 gene expression is determined by the amounts of the mRNA transcripts. EIF2AK2 gene encodes various transcript variants. In certain embodiments, the mRNA transcripts are splice variants, fragments or derivatives of all native and natural variants of the transcripts of the EIF2AK2 genes. NCBI Reference Sequences NM_001135651.3, NM_001135652.2, and NM_002759.3 provide exemplary human EIF2AK2 mRNA transcript sequences. Examples of EIF2AK2 polypeptides include any such native polypeptides from any vertebrate source as described above. In certain embodiments, EIF2AK2 gene expression is determined by the amounts of the EIF2AK2 polypeptides. In certain embodiments, the EIF2AK2 polypeptides include all polypeptides encoded by the natural variants of the EIF2AK2 genes and transcripts thereof, including allelic variants (e.g., SNP variants); splice variants; fragments; and derivatives. The EIF2AK2 polypeptides of the present disclosure also encompass “full-length,” unprocessed EIF2AK2 polypeptide as well as any form of EIF2AK2 polypeptide that results from processing in the cell. NCBI Reference Sequences NP_001129123.1, NP_001129124.1, and NP_002750.1 provide exemplary human EIF2AK2 polypeptide sequences.

As used herein, the term “ACSS1” refers to “acyl-CoA synthetase short chain family member 1,” also known as “acetyl-coenzyme A synthetase 2-like, mitochondrial,” “acetate-CoA ligase 2,” or “propionate-CoA ligase,” in Uniprot or GenBank database. The term “ACSS1” encompasses the ACSS1 polypeptides, the ACSS1 RNA transcripts, and the ACSS1 genes. The term “ACSS1 gene” refers to genes encoding ACSS1 polypeptides. ACSS1 is expressed in various cells and tissues including plasma and endothelial cells, among others. Examples of ACSS1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ACSS1 gene” includes all natural variants of the ACSS1 genes, including allelic variants (e.g., SNP variants) and mutations. NCBI Gene ID: 84532 and NCBI Reference Sequence NC_0000020.11 (range 25006230 . . . 25058182, complement) provide exemplary human ACSS1 nucleic acid sequences. In certain embodiments, ACSS1 gene expression is determined by the amounts of the mRNA transcripts. ACSS1 gene encodes various transcript variants. In certain embodiments, the mRNA transcripts are splice variants, fragments or derivatives of all native and natural variants of the transcripts of the ACSS1 genes. NCBI Reference Sequences NM_001252675.1, NM_001252676.1, NM_001252677.1, and NM_032501.4 provide exemplary human ACSS1 mRNA transcript sequences. Examples of ACSS1 polypeptides include any such native polypeptides from any vertebrate source as described above. In certain embodiments, ACSS1 gene expression is determined by the amounts of the ACSS1 polypeptides. In certain embodiments, the ACSS1 polypeptides include all polypeptides encoded by the natural variants of the ACSS1 genes and transcripts thereof, including allelic variants (e.g., SNP variants); splice variants; fragments; and derivatives. The ACSS1 polypeptides of the present disclosure also encompass “full-length,” unprocessed ACSS1 polypeptide as well as any form of ACSS1 polypeptide that results from processing in the cell. NCBI Reference Sequences NP_001239604.1, NP_001239605.1, NP_001239606.1, and NP_115890.2 provide exemplary human ACSS1 polypeptide sequences.

As used herein, the term “ACSS2” refers to “acyl-CoA synthetase short chain family member 2,” also known as “acetyl-coenzyme A synthetase, cytoplasmic,” “acetyl-CoA synthetase 1,” or “acyl-activating enzyme,” in Uniprot or GenBank database. The term “ACSS2” encompasses the ACSS2 polypeptides, the ACSS2 RNA transcripts, and the ACSS2 genes. The term “ACSS2 gene” refers to genes encoding ACSS2 polypeptides. ACSS2 is expressed in various cells and tissues including plasma and endothelial cells, among others. Examples of ACSS2 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “ACSS2 gene” includes all natural variants of the ACSS2 genes, including allelic variants (e.g., SNP variants) and mutations. NCBI Gene ID: 55902 and NCBI Reference Sequence NC_0000020.11 (range 34874942 . . . 34927966) provide exemplary human ACSS2 nucleic acid sequences. In certain embodiments, ACSS2 gene expression is determined by the amounts of the mRNA transcripts. ACSS2 gene encodes various transcript variants. In certain embodiments, the mRNA transcripts are splice variants, fragments or derivatives of all native and natural variants of the transcripts of the ACSS2 genes. NCBI Reference Sequences NM_001076552.2, NM_001242393.1, and NM_018677.4 provide exemplary human ACSS2 mRNA transcript sequences. Examples of ACSS2 polypeptides include any such native polypeptides from any vertebrate source as described above. In certain embodiments, ACSS2 gene expression is determined by the amounts of the ACSS2 polypeptides. In certain embodiments, the ACSS2 polypeptides include all polypeptides encoded by the natural variants of the ACSS2 genes and transcripts thereof, including allelic variants (e.g., SNP variants); splice variants; fragments; and derivatives. The ACSS2 polypeptides of the present disclosure also encompass “full-length,” unprocessed ACSS2 polypeptide as well as any form of ACSS2 polypeptide that results from processing in the cell. NCBI Reference Sequences NP_001070020.2, NP_001229322.1, and NP_061147.1 provide exemplary human ACSS2 polypeptide sequences.

As used herein, the term “PAK4” refers to “p21 (RAC1) activated kinase 4,” also known as “serine/threonine-protein kinase PAK 4,” “p21 protein (Cdc42/Rac)-activated kinase 4,” “PAK-4,” or “p21(CDKN1A)-activated kinase 4,” in Uniprot or GenBank database. The term “PAK4” encompasses the PAK4 polypeptides, the PAK4 RNA transcripts, and the PAK4 genes. The term “PAK4 gene” refers to genes encoding PAK4 polypeptides. PAK4 is expressed in various cells and tissues including plasma and endothelial cells, among others. Examples of PAK4 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “PAK4 gene” includes all natural variants of the PAK4 genes, including allelic variants (e.g., SNP variants) and mutations. NCBI Gene ID: 10298 and NCBI Reference Sequence NC_0000019.10 (range 39125786 . . . 39182816) provide exemplary human PAK4 nucleic acid sequences. In certain embodiments, PAK4 gene expression is determined by the amounts of the mRNA transcripts. PAK4 gene encodes various transcript variants. In certain embodiments, the mRNA transcripts are splice variants, fragments or derivatives of all native and natural variants of the transcripts of the PAK4 genes. NCBI Reference Sequences NM_001014831.3, NM_001014832.2, NM_001014834.3, NM_001014835.2, and NM_005884.4 provide exemplary human PAK4 mRNA transcript sequences. Examples of PAK4 polypeptides include any such native polypeptides from any vertebrate source as described above. In certain embodiments, PAK4 gene expression is determined by the amounts of the PAK4 polypeptides. In certain embodiments, the PAK4 polypeptides include all polypeptides encoded by the natural variants of the PAK4 genes and transcripts thereof, including allelic variants (e.g., SNP variants); splice variants; fragments; and derivatives. The PAK4 polypeptides of the present disclosure also encompass “full-length,” unprocessed PAK4 polypeptide as well as any form of PAK4 polypeptide that results from processing in the cell. NCBI Reference Sequences NP_001014831.1, NP_001014832.1, NP_001014834.1, NP_001014835.1, and NP_005875.1 provide exemplary human PAK4 polypeptide sequences.

As used herein, the term “SPI1” refers to “Spi-1 proto-oncogene,” also known as “transcription factor PU.1,” “31 kDa transforming protein,” “hematopoietic transcription factor PU.1,” or “spleen focus forming virus (SFFV) proviral integration oncogene spi1,” in Uniprot or GenBank database. The term “SPI1” encompasses the SPI1 polypeptides, the SPI1 RNA transcripts, and the SPI1 genes. The term “SPI1 gene” refers to genes encoding SPI1 polypeptides. SPI1 is expressed in various cells and tissues including plasma and endothelial cells, among others. Examples of SPI1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “SPI1 gene” includes all natural variants of the SPI1 genes, including allelic variants (e.g., SNP variants) and mutations. NCBI Gene ID: 6688 and NCBI Reference Sequence NC_0000011.10 (range 47354859 . . . 47395640, complement) provide exemplary human SPI1 nucleic acid sequences. In certain embodiments, SPI1 gene expression is determined by the amounts of the mRNA transcripts. SPI1 gene encodes various transcript variants. In certain embodiments, the mRNA transcripts are splice variants, fragments or derivatives of all native and natural variants of the transcripts of the SPI1 genes. NCBI Reference Sequences NM_001080547.2, NM_003120.3, XM_011520307.1, and XM_017018173.1 provide exemplary human SPI1 mRNA transcript sequences. Examples of SPI1 polypeptides include any such native polypeptides from any vertebrate source as described above. In certain embodiments, SPI1 gene expression is determined by the amounts of the SPI1 polypeptides. In certain embodiments, the SPI1 polypeptides include all polypeptides encoded by the natural variants of the SPI1 genes and transcripts thereof, including allelic variants (e.g., SNP variants); splice variants; fragments; and derivatives. The SPI1 polypeptides of the present disclosure also encompass “full-length,” unprocessed SPI1 polypeptide as well as any form of SPI1 polypeptide that results from processing in the cell. NCBI Reference Sequences NP_001074016.1, NP_003111.2, XP_011518609.1, and XP_016873662.1 provide exemplary human SPI1 polypeptide sequences.

As used herein, the term “RFXAP” refers to “regulatory factor X associated protein,” also known as “RFX-associated protein,” or “RFX DNA-binding complex 36 kDa subunit,” in Uniprot or GenBank database. The term “RFXAP” encompasses the RFXAP polypeptides, the RFXAP RNA transcripts, and the RFXAP genes. The term “RFXAP gene” refers to genes encoding RFXAP polypeptides. RFXAP is expressed in various cells and tissues including plasma and endothelial cells, among others. Examples of RFXAP genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RFXAP gene” includes all natural variants of the RFXAP genes, including allelic variants (e.g., SNP variants) and mutations. NCBI Gene ID: 5994 and NCBI Reference Sequence NC_0000013.11 (range 36819222 . . . 36829104) provide exemplary human RFXAP nucleic acid sequences. In certain embodiments, RFXAP gene expression is determined by the amounts of the mRNA transcripts. RFXAP gene encodes various transcript variants. In certain embodiments, the mRNA transcripts are splice variants, fragments or derivatives of all native and natural variants of the transcripts of the RFXAP genes. NCBI Reference Sequences NM_000538.4 and BC026088 provide exemplary human RFXAP mRNA transcript sequences. Examples of RFXAP polypeptides include any such native polypeptides from any vertebrate source as described above. In certain embodiments, RFXAP gene expression is determined by the amounts of the RFXAP polypeptides. In certain embodiments, the RFXAP polypeptides include all polypeptides encoded by the natural variants of the RFXAP genes and transcripts thereof, including allelic variants (e.g., SNP variants); splice variants; fragments; and derivatives. The RFXAP polypeptides of the present disclosure also encompass “full-length,” unprocessed RFXAP polypeptide as well as any form of RFXAP polypeptide that results from processing in the cell. NCBI Reference Sequences NP_000529.1 and AAH26088.1 provide exemplary human RFXAP polypeptide sequences.

As used herein, the term “RFXANK” refers to “regulatory factor X associated ankyrin containing protein,” also known as “DNA-binding protein RFXANK,” “regulatory factor X subunit B,” or “ankyrin repeat family A protein 1,” in Uniprot or GenBank database. The term “RFXANK” encompasses the RFXANK polypeptides, the RFXANK RNA transcripts, and the RFXANK genes. The term “RFXANK gene” refers to genes encoding RFXANK polypeptides. RFXANK is expressed in various cells and tissues including plasma and endothelial cells, among others. Examples of RFXANK genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “RFXANK gene” includes all natural variants of the RFXANK genes, including allelic variants (e.g., SNP variants) and mutations. NCBI Gene ID: 8625 and NCBI Reference Sequence NC_0000019.10 (range 19192199 . . . 19201869) provide exemplary human RFXANK nucleic acid sequences. In certain embodiments, RFXANK gene expression is determined by the amounts of the mRNA transcripts. RFXANK gene encodes various transcript variants. In certain embodiments, the mRNA transcripts are splice variants, fragments or derivatives of all native and natural variants of the transcripts of the RFXANK genes. NCBI Reference Sequences NM_001278727.1, NM_001278728.1, NM_001370233.1, NM_001370234.1, NM_001370235.1, NM_001370236.1, NM_001370237.1, NM_001370238.1, NM_003721.4, and NM_134440.2 provide exemplary human RFXANK mRNA transcript sequences. Examples of RFXANK polypeptides include any such native polypeptides from any vertebrate source as described above. In certain embodiments, RFXANK gene expression is determined by the amounts of the RFXANK polypeptides. In certain embodiments, the RFXANK polypeptides include all polypeptides encoded by the natural variants of the RFXANK genes and transcripts thereof, including allelic variants (e.g., SNP variants); splice variants; fragments; and derivatives. The RFXANK polypeptides of the present disclosure also encompass “full-length,” unprocessed RFXANK polypeptide as well as any form of RFXANK polypeptide that results from processing in the cell. NCBI Reference Sequences NP_001265656.1, NP_001265657.1, NP_001357162.1, NP_001357163.1, NP_001357164.1, NP_001357165.1, NP_001357166.1, NP_001357167.1, NP_003712.1, and NP_604389.1 provide exemplary human RFXANK polypeptide sequences.

As used herein, the term “IRF8” refers to “interferon regulatory factor 8,” also known as “interferon consensus sequence binding protein 1,” “interferon consensus sequence binding protein,” or “ICSBP,” in Uniprot or GenBank database. The term “IRF8” encompasses the IRF8 polypeptides, the IRF8 RNA transcripts, and the IRF8 genes. The term “IRF8 gene” refers to genes encoding IRF8 polypeptides. IRF8 is expressed in various cells and tissues including plasma and endothelial cells, among others. Examples of IRF8 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “IRF8 gene” includes all natural variants of the IRF8 genes, including allelic variants (e.g., SNP variants) and mutations. NCBI Gene ID: 3394 and NCBI Reference Sequence NC_0000016.10 (range 85899162 . . . 85922609) provide exemplary human IRF8 nucleic acid sequences. In certain embodiments, IRF8 gene expression is determined by the amounts of the mRNA transcripts. IRF8 gene encodes various transcript variants. In certain embodiments, the mRNA transcripts are splice variants, fragments or derivatives of all native and natural variants of the transcripts of the IRF8 genes. NCBI Reference Sequences NM_001363907.1, NM_001363908.1, and NM_002163.4 provide exemplary human IRF8 mRNA transcript sequences. Examples of IRF8 polypeptides include any such native polypeptides from any vertebrate source as described above. In certain embodiments, IRF8 gene expression is determined by the amounts of the IRF8 polypeptides. In certain embodiments, the IRF8 polypeptides include all polypeptides encoded by the natural variants of the IRF8 genes and transcripts thereof, including allelic variants (e.g., SNP variants); splice variants; fragments; and derivatives. The IRF8 polypeptides of the present disclosure also encompass “full-length,” unprocessed IRF8 polypeptide as well as any form of IRF8 polypeptide that results from processing in the cell. NCBI Reference Sequences NP_001350836.1, NP_001350837.1, and NP_002154.1 provide exemplary human IRF8 polypeptide sequences.

As used herein, the term “NFYA” refers to “nuclear transcription factor Y subunit alpha,” also known as “CAAT-box DNA binding protein subunit A,” or “nuclear transcription factor Y subunit A,” in Uniprot or GenBank database. The term “NFYA” encompasses the NFYA polypeptides, the NFYA RNA transcripts, and the NFYA genes. The term “NFYA gene” refers to genes encoding NFYA polypeptides. NFYA is expressed in various cells and tissues including plasma and endothelial cells, among others. Examples of NFYA genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “NFYA gene” includes all natural variants of the NFYA genes, including allelic variants (e.g., SNP variants) and mutations. NCBI Gene ID: 4800 and NCBI Reference Sequence NC_0000006.12 (range 41072974 . . . 41102403) provide exemplary human NFYA nucleic acid sequences. In certain embodiments, NFYA gene expression is determined by the amounts of the mRNA transcripts. NFYA gene encodes various transcript variants. In certain embodiments, the mRNA transcripts are splice variants, fragments or derivatives of all native and natural variants of the transcripts of the NFYA genes. NCBI Reference Sequences NM_002505.5 and NM_021705.4 provide exemplary human NFYA mRNA transcript sequences. Examples of NFYA polypeptides include any such native polypeptides from any vertebrate source as described above. In certain embodiments, NFYA gene expression is determined by the amounts of the NFYA polypeptides. In certain embodiments, the NFYA polypeptides include all polypeptides encoded by the natural variants of the NFYA genes and transcripts thereof, including allelic variants (e.g., SNP variants); splice variants; fragments; and derivatives. The NFYA polypeptides of the present disclosure also encompass “full-length,” unprocessed NFYA polypeptide as well as any form of NFYA polypeptide that results from processing in the cell. NCBI Reference Sequences NP_002496.1 and NP_068351.1 provide exemplary human NFYA polypeptide sequences.

As used herein, the term “NFYC” refers to “nuclear transcription factor Y subunit gamma,” also known as “CAAT box DNA-binding protein subunit C,” or “nuclear transcription factor Y subunit C,” in Uniprot or GenBank database. The term “NFYC” encompasses the NFYC polypeptides, the NFYC RNA transcripts, and the NFYC genes. The term “NFYC gene” refers to genes encoding NFYC polypeptides. NFYC is expressed in various cells and tissues including plasma and endothelial cells, among others. Examples of NFYC genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “NFYC gene” includes all natural variants of the NFYC genes, including allelic variants (e.g., SNP variants) and mutations. NCBI Gene ID: 4802 and NCBI Reference Sequence NC_0000001.11 (range 40691699 . . . 40771603) provide exemplary human NFYC nucleic acid sequences. In certain embodiments, NFYC gene expression is determined by the amounts of the mRNA transcripts. NFYC gene encodes various transcript variants. In certain embodiments, the mRNA transcripts are splice variants, fragments or derivatives of all native and natural variants of the transcripts of the NFYC genes. NCBI Reference Sequences NM_001142587.2, NM_001142588.2, NM_001142589.2, NM_001142590.2, NM_001308114.1, NM_001308115.2, and NM_014223.5 provide exemplary human NFYC mRNA transcript sequences. Examples of NFYC polypeptides include any such native polypeptides from any vertebrate source as described above. In certain embodiments, NFYC gene expression is determined by the amounts of the NFYC polypeptides. In certain embodiments, the NFYC polypeptides include all polypeptides encoded by the natural variants of the NFYC genes and transcripts thereof, including allelic variants (e.g., SNP variants); splice variants; fragments; and derivatives. The NFYC polypeptides of the present disclosure also encompass “full-length,” unprocessed NFYC polypeptide as well as any form of NFYC polypeptide that results from processing in the cell. NCBI Reference Sequences NP_001136059.1, NP_001136060.1, NP_001136061.1, NP_001136062.1, NP_001295043.1, NP_001295044.1, and NP_055038.2 provide exemplary human NFYC polypeptide sequences.

As used herein, the term “LST1” refers to “leukocyte specific transcript 1,” also known as “leukocyte-specific transcript 1 protein,” “protein B144,” or “lymphocyte antigen 117,” in Uniprot or GenBank database. The term “LST1” encompasses the LST1 polypeptides, the LST1 RNA transcripts, and the LST1 genes. The term “LST1 gene” refers to genes encoding LST1 polypeptides. LST1 is expressed in various cells and tissues including plasma and endothelial cells, among others. Examples of LST1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “LST1 gene” includes all natural variants of the LST1 genes, including allelic variants (e.g., SNP variants) and mutations. NCBI Gene ID: 7940 and NCBI Reference Sequence NC_0000006.12 (range 31586185 . . . 31588909) provide exemplary human LST1 nucleic acid sequences. In certain embodiments, LST1 gene expression is determined by the amounts of the mRNA transcripts. LST1 gene encodes various transcript variants. In certain embodiments, the mRNA transcripts are splice variants, fragments or derivatives of all native and natural variants of the transcripts of the LST1 genes. NCBI Reference Sequences NM_001166538.1, NM_007161.3, NM_205837.3, NM_205838.3, NM_205839.3, and NM_205840.2 provide exemplary human LST1 mRNA transcript sequences. Examples of LST1 polypeptides include any such native polypeptides from any vertebrate source as described above. In certain embodiments, LST1 gene expression is determined by the amounts of the LST1 polypeptides. In certain embodiments, the LST1 polypeptides include all polypeptides encoded by the natural variants of the LST1 genes and transcripts thereof, including allelic variants (e.g., SNP variants); splice variants; fragments; and derivatives. The LST1 polypeptides of the present disclosure also encompass “full-length,” unprocessed LST1 polypeptide as well as any form of LST1 polypeptide that results from processing in the cell. NCBI Reference Sequences NP_001160010.1, NP_009092.3, NP_995309.2, NP_995310.2, NP_995311.2, NP_995312.2 provide exemplary human LST1 polypeptide sequences.

As used herein, the term “LTB” refers to “lymphotoxin beta,” also known as “tumor necrosis factor C,” “TNF-C,” or “tumor necrosis factor ligand superfamily member 3,” in Uniprot or GenBank database. The term “LTB” encompasses the LTB polypeptides, the LTB RNA transcripts, and the LTB genes. The term “LTB gene” refers to genes encoding LTB polypeptides. LTB is expressed in various cells and tissues including plasma and endothelial cells, among others. Examples of LTB genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “LTB gene” includes all natural variants of the LTB genes, including allelic variants (e.g., SNP variants) and mutations. NCBI Gene ID: 4050 and NCBI Reference Sequence NC_0000006.12 (range 31580558 . . . 31582425, complement) provide exemplary human LTB nucleic acid sequences. In certain embodiments, LTB gene expression is determined by the amounts of the mRNA transcripts. LTB gene encodes various transcript variants. In certain embodiments, the mRNA transcripts are splice variants, fragments or derivatives of all native and natural variants of the transcripts of the LTB genes. NCBI Reference Sequences NM_002341.2 and NM_009588.1 provide exemplary human LTB mRNA transcript sequences. Examples of LTB polypeptides include any such native polypeptides from any vertebrate source as described above. In certain embodiments, LTB gene expression is determined by the amounts of the LTB polypeptides. In certain embodiments, the LTB polypeptides include all polypeptides encoded by the natural variants of the LTB genes and transcripts thereof, including allelic variants (e.g., SNP variants); splice variants; fragments; and derivatives. The LTB polypeptides of the present disclosure also encompass “full-length,” unprocessed LTB polypeptide as well as any form of LTB polypeptide that results from processing in the cell. NCBI Reference Sequences NP_002332.1 and NP_033666.1 provide exemplary human LTB polypeptide sequences.

As used herein, the term “AIF1” refers to “allograft inflammatory factor 1,” also known as “protein G1,” “interferon gamma responsive transcript,” or “ionized calcium-binding adapter molecule 1,” in Uniprot or GenBank database. The term “AIF1” encompasses the AIF1 polypeptides, the AIF1 RNA transcripts, and the AIF1 genes. The term “AIF1 gene” refers to genes encoding AIF1 polypeptides. AIF1 is expressed in various cells and tissues including plasma and endothelial cells, among others. Examples of AIF1 genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “AIF1 gene” includes all natural variants of the AIF1 genes, including allelic variants (e.g., SNP variants) and mutations. NCBI Gene ID: 199 and NCBI Reference Sequence NC_0000006.12 (range 31615212 . . . 31617015) provide exemplary human AIF1 nucleic acid sequences. In certain embodiments, AIF1 gene expression is determined by the amounts of the mRNA transcripts. AIF1 gene encodes various transcript variants. In certain embodiments, the mRNA transcripts are splice variants, fragments or derivatives of all native and natural variants of the transcripts of the AIF1 genes. NCBI Reference Sequences NM_001318970.2, NM_001623.5, NM_032955.3, XM_017010332.1, and XM_005248870.4 provide exemplary human AIF1 mRNA transcript sequences. Examples of AIF1 polypeptides include any such native polypeptides from any vertebrate source as described above. In certain embodiments, AIF1 gene expression is determined by the amounts of the AIF1 polypeptides. In certain embodiments, the AIF1 polypeptides include all polypeptides encoded by the natural variants of the AIF1 genes and transcripts thereof, including allelic variants (e.g., SNP variants); splice variants; fragments; and derivatives. The AIF1 polypeptides of the present disclosure also encompass “full-length,” unprocessed AIF1 polypeptide as well as any form of AIF1 polypeptide that results from processing in the cell. NCBI Reference Sequences NP_001305899.1, NP_001614.3, NP_116573.1, XP_016865821.1, and XP_005248927.1 provide exemplary human AIF1 polypeptide sequences.

As used herein, the terms “TNF” and “TNF-α” are used interchangeably to refer to “tumor necrosis factor,” also known as “tumor necrosis factor ligand superfamily member 2,” or “TNF-alpha,” in Uniprot or GenBank database. The term “TNF” encompasses the TNF polypeptides, the TNF RNA transcripts, and the TNF genes. The term “TNF gene” refers to genes encoding TNF polypeptides. TNF is expressed in various cells and tissues including plasma and endothelial cells, among others. Examples of TNF genes encompass any such native gene from any vertebrate source, including mammals such as primates (e.g., humans and chimpanzees), dogs, cow, chicken, reptiles (e.g. clawed frog), and rodents (e.g., mice and rats), unless otherwise indicated. In certain embodiments, the term “TNF gene” includes all natural variants of the TNF genes, including allelic variants (e.g., SNP variants) and mutations. NCBI Gene ID: 7124 and NCBI Reference Sequence NC_0000006.12 (range 31575565 . . . 31578336) provide exemplary human TNF nucleic acid sequences. In certain embodiments, TNF gene expression is determined by the amounts of the mRNA transcripts. TNF gene encodes various transcript variants. In certain embodiments, the mRNA transcripts are splice variants, fragments or derivatives of all native and natural variants of the transcripts of the TNF genes. NCBI Reference Sequences NM_000594.4, M10988, and X01394 provide exemplary human TNF mRNA transcript sequences. Examples of TNF polypeptides include any such native polypeptides from any vertebrate source as described above. In certain embodiments, TNF gene expression is determined by the amounts of the TNF polypeptides. In certain embodiments, the TNF polypeptides include all polypeptides encoded by the natural variants of the TNF genes and transcripts thereof, including allelic variants (e.g., SNP variants); splice variants; fragments; and derivatives. The TNF polypeptides of the present disclosure also encompass “full-length,” unprocessed TNF polypeptide as well as any form of TNF polypeptide that results from processing in the cell. NCBI Reference Sequences NP_000585.2, AAA61198.1, and CAA25650.1 provide exemplary human TNF polypeptide sequences.

### 5.2 Methods of Using ADCs

Provided herein are methods for treating various cancers using an antibody drug conjugate (ADC). Also provided herein are methods for inducing or enhancing immunogenic cell death (ICD) in a cancer in a subject in need thereof using an ADC. Further provided herein are methods for inducing or enhancing bystander cell killing in a cancer in a subject in need thereof using an ADC. Additionally provided herein are methods for inducing immune cell migration to a cancer in a subject in need thereof using an ADC. Further provided herein are methods for increasing expression of one or more ADC Set I Marker genes in a cancer in a subject in need thereof using an ADC. In certain embodiments, the ADC comprises an antibody or antigen binding fragment thereof conjugated to one or more units of a cytotoxic agent via a linker. In certain embodiments, the ADC comprises an antibody or antigen binding fragment thereof conjugated to one or more units of a cytotoxic agent via a linker, wherein the cytotoxic agent is a tubulin disrupting agent. In some embodiments, the ADC comprises an anti-nectin-4 antibody or antigen binding fragment thereof conjugated to one or more units of a cytotoxic agent via a linker. In some embodiments, the ADC comprises an anti-nectin-4 antibody or antigen binding fragment thereof conjugated to one or more units of a cytotoxic agent via a linker, wherein the cytotoxic agent is a tubulin disrupting agent. In certain embodiments, the ADC comprises an antibody or antigen binding fragment thereof conjugated to one or more units of an auristatin via a linker. In some embodiments, the ADC comprises an anti-nectin-4 antibody or antigen binding fragment thereof conjugated to one or more units of an auristatin via a linker. In certain embodiments, the ADC comprises an antibody or antigen binding fragment thereof conjugated to one or more units of monomethyl auristatin E (MMAE) via a linker. In some embodiments, the ADC comprises an anti-nectin-4 antibody or antigen binding fragment thereof conjugated to one or more units of MMAE via a linker. In certain embodiments, the ADC comprises an antibody or antigen binding fragment thereof conjugated to one or more units of a cytotoxic agent via a linker, wherein the antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and wherein the antibody or antigen binding fragment thereof is conjugated to 1 to 20 units of the cytotoxic agent via a linker. In some embodiments, the ADC comprises an anti-nectin-4 antibody or antigen binding fragment thereof conjugated to one or more units of MMAE via a linker, wherein the antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and wherein the antibody or antigen binding fragment thereof is conjugated to 1 to 20 units of MMAE via a linker. In some specific embodiments, the ADC is enfortumab vedotin (also known as EV, anti-191P4D12-ADC, Ha22-2(2,4)6.1vcMMAE, ASG-22CE, ASG-22ME, ASG-22C₃E, AGS-22C₃E, or AGS-22M6E). In other embodiments, the ADC is administered three times every 28 day cycle. In some specific embodiments, the ADC is administered on Days 1, 8 and 15 of every 28 day cycle.

Without being bound or limited by the theory, the disclosure provides some embodiments based on the realization that MHCs on tumor cells make connections with T-cell receptors, activate the adaptive immune response, plays a crucial role for checkpoint response signaling, and thus serves as markers for the effectiveness of combining ADCs with agents causing ICD in general and checkpoint inhibitors in particular. The present disclosure provides that upregulated ADC Set I Markers, including WIC signature genes (such as MHC class I and class II), on cancer cells can activate the adaptive immune response, e.g. by displaying neoantigens on the cell surface after treatment by ADCs. Upregulation of ADC Set I Markers, including MHC signature genes, enhances/induces ICD after treatment with ADCs, enhances/induces the bystander cell killing effect induced by ADCs, enhances the efficacy of the ADC treatment, and enhances the efficacy for combining ADC treatment with immune checkpoint inhibitors.

In one aspect, provided herein is a method for treating cancer in a subject in need thereof comprising: (1) administering to the subject an antibody drug conjugate (ADC) comprising an antibody or antigen binding fragment thereof conjugated to one or more units of a cytotoxic agent via a linker, (2) determining an increase of expression of one or more ADC Set I Marker genes in the subject, and (3) (a) continue administering the ADC if the expression of the one or more ADC Set I Marker genes in the subject is increased compared to the expression of the one or more ADC Set I Marker genes in the subject before the administration of the ADC, or (b) discontinue administering the ADC if the expression of the one or more ADC Set I Marker genes in the subject is not increased compared to the expression of the one or more ADC Set I Marker genes in the subject before the administration of the ADC, wherein the one or more ADC Set I Marker genes comprise one or more major histocompatibility complex (MHC) signature genes, one or more toll-like receptor (TLR) family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In another aspect, provided herein is a method for treating cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3)(a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or (b) administering a second dose of the ADC at a higher amount than the first dose if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In yet another aspect, provided herein is a method for treating cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In a further aspect, provided herein is a method for treating cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor to the subject if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the checkpoint inhibitor in step (3)(a) is not administered in conjunction with the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In one aspect, provided herein is a method for inducing immunogenic cell death (ICD) in a cancer in a subject in need thereof comprising: (1) administering to the subject an ADC comprising an antibody or antigen binding fragment thereof conjugated to one or more units of a cytotoxic agent via a linker, (2) determining an increase of expression of one or more ADC Set I Marker genes in the subject, and (3) (a) continue administering the ADC if the expression of the one or more ADC Set I Marker genes in the subject is increased compared to the expression of the one or more ADC Set I Marker genes in the subject before the administration of the ADC, or (b) discontinue administering the ADC if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In another aspect, provided herein is a method for inducing ICD in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, (b) or administering a second dose of the ADC at a higher amount than the first dose if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In yet another aspect, provided herein is a method for inducing ICD in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, (b) or administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In a further aspect, provided herein is a method for inducing ICD in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor to the subject if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the checkpoint inhibitor in step (3)(a) is not administered in conjunction with the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In one aspect, provided herein is a method for inducing or enhancing bystanding cell killing in a cancer in a subject in need thereof comprising: (1) administering to the subject an ADC comprising an antibody or antigen binding fragment thereof conjugated to one or more units of a cytotoxic agent via a linker, (2) determining an increase of expression of one or more ADC Set I Marker genes in the subject, and (3) (a) continue administering the ADC if the expression of the one or more ADC Set I Marker genes in the subject is increased compared to the expression of the one or more ADC Set I Marker genes in the subject before the administration of the ADC, or (b) discontinue administering the ADC if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MIME via a linker.

In another aspect, provided herein is a method for inducing or enhancing bystanding cell killing in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, (b) or administering a second dose of the ADC at a higher amount than the first dose if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In yet another aspect, provided herein is a method for inducing or enhancing bystanding cell killing in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, (b) or administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In a further aspect, provided herein is a method for inducing or enhancing bystanding cell killing in a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor to the subject if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the checkpoint inhibitor in step (3)(a) is not administered in conjunction with the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In one aspect, provided herein is a method for inducing immune cell migration to a cancer in a subject in need thereof comprising: (1) administering to the subject an ADC comprising an antibody or antigen binding fragment thereof conjugated to one or more units of a cytotoxic agent via a linker, (2) determining an increase of expression of one or more ADC Set I Marker genes in the subject, and (3) (a) continue administering the ADC if the expression of the one or more ADC Set I Marker genes in the subject is increased compared to the expression of the one or more ADC Set I Marker genes in the subject before the administration of the ADC, or (b) discontinue administering the ADC if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In another aspect, provided herein is a method for inducing immune cell migration to a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or (b) administering a second dose of the ADC at a higher amount than the first dose if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In yet another aspect, provided herein is a method for inducing immune cell migration to a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In a further aspect, provided herein is a method for inducing immune cell migration to a cancer in a subject in need thereof comprising: (1) administering to the subject a first dose of an ADC comprising an antibody or antigen binding fragment thereof conjugated to one or more units of a cytotoxic agent via a linker, (2) determining an increase of expression of one or more ADC Set I Marker genes in the subject, and (3) (a) administering an immune checkpoint inhibitor to the subject if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the checkpoint inhibitor in step (3)(a) is not administered in conjunction with the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In one aspect, provided herein is a method for increasing expression of one or more ADC Set I Marker genes in a cancer in a subject in need thereof comprising: (1) administering to the subject an ADC comprising an antibody or antigen binding fragment thereof conjugated to one or more units of a cytotoxic agent via a linker, (2) determining an increase of the expression of the one or more ADC Set I Marker genes in the subject, and (3) (a) continue administering the ADC if the expression of the one or more ADC Set I Marker genes in the subject is increased compared to the expression of the one or more ADC Set I Marker genes in the subject before the administration of the ADC, or (b) discontinue administering the ADC if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MIME. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In another aspect, provided herein is a method for increasing expression of one or more ADC Set I Marker genes in a cancer in a subject in need thereof comprising: (1) administering to the subject a first dose of an ADC comprising an antibody or antigen binding fragment thereof conjugated to one or more units of a cytotoxic agent via a linker, (2) determining an increase of the expression of the one or more ADC Set I Marker genes in the subject, and (3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or (b) administering a second dose of the ADC at a higher amount than the first dose if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In yet another aspect, provided herein is a method for increasing expression of one or more ADC Set I Marker genes in a cancer in a subject in need thereof comprising: (1) administering to the subject a first dose of an ADC comprising an antibody or antigen binding fragment thereof conjugated to one or more units of a cytotoxic agent via a linker, (2) determining an increase of the expression of the one or more ADC Set I Marker genes in the subject, and (3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 7 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 8, and the antibody or antigen binding fragment thereof is conjugated to 1 to 20 units of MMAE via a linker.

In a further aspect, provided herein is a method for increasing expression of one or more ADC Set I Marker genes in a cancer in a subject in need thereof comprising: (1) administering to the subject a first dose of an ADC comprising an antibody or antigen binding fragment thereof conjugated to one or more units of a cytotoxic agent via a linker, (2) determining an increase of the expression of the one or more ADC Set I Marker genes in the subject, and (3) (a) administering an immune checkpoint inhibitor to the subject if the expression of the one or more ADC Set I Marker genes in the subject is increased compared to the expression of the one or more ADC Set I Marker genes in the subject before the administration of the ADC, or (b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC, wherein the checkpoint inhibitor in step (3)(a) is not administered in conjunction with the ADC, wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes. In some embodiments of the methods, the antibody or antigen binding fragment thereof of the ADC is an anti-nectin-4 antibody or antigen binding fragment thereof. In certain embodiments of the methods, the cytotoxic agent of the ADC is an auristatin. In one embodiment, the auristatin is MMAE. In other embodiments of the methods, the antibody or antigen binding fragment thereof of the ADC comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 22 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 23, and the antibody or antigen binding fragment thereof is conjugated to 1 to 20 units of MMAE via a linker. In yet other embodiments of the methods, the antibody or antigen binding fragment thereof of the ADC comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO: 22 and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO: 23, and the antibody or antigen binding fragment thereof is conjugated to 1 to 20 units of MMAE via a linker.

In various embodiments of the methods provided herein, including the methods provided in this Section (Section 5.2) such as the methods provided in this and the preceding 22 paragraphs, the methods further comprise a step of obtaining a sample from the subject. In various embodiments of the methods provided herein, including the methods provided in this Section (Section 5.2) such as the methods provided in this and the preceding 22 paragraphs, the increase in the subject as determined in the step (2) of the methods is determined in a sample from the subject. In various embodiments of the methods provided herein, including the methods provided in this Section (Section 5.2) such as the methods provided in this and the preceding 22 paragraphs, the increase, on which the administration of step (3)(a) is conditioned, is an increase of the expression of the one or more ADC Marker genes in a sample from the subject when compared to the expression of the one or more ADC Marker genes in a sample from the subject before the administration of the ADC. In some embodiments of the methods, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a blood sample. In certain embodiments, the sample is a serum sample. In further embodiments, the sample is plasma sample. In some embodiments, the sample is bodily fluid. In one embodiment, the sample is a tissue. In some embodiments, the sample is a cancer tissue. In certain embodiments, the sample is a tissue surrounding the cancer. In further embodiments, the sample is tissue fluid. In some embodiments, the sample is cancer tissue fluid. In some embodiments, the samples used for comparison for determining an increase of the one or more ADC Marker genes are corresponding samples before and after ADC administration, e.g. blood samples before and after ADC administration, serum samples before and after ADC administration, plasma samples before and after ADC administration, bodily fluid of the same type and/or location before and after ADC administration, tissues of the same type and/or location before and after ADC administration, cancer tissues of the same type, location, and/or origin before and after ADC administration, tissues surrounding the cancer of the same type and/or location before and after ADC administration, tissue fluid of the same type and/or location before and after ADC administration, cancer tissue fluid of the same type, location, and/or origin before and after ADC administration, etc.

In various embodiments of the methods provided herein, including the methods provided in this Section (Section 5.2) such as the methods provided in this and the preceding 23 paragraphs, the one or more ADC Set I Marker genes comprise or consist of any one of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes. The following embodiments of this paragraph specifically list out the embodiments provided with the preceding sentence. In one embodiment, the one or more ADC Set I Marker genes comprise or consist of one or more MHC signature genes. In another embodiment, the one or more ADC Set I Marker genes comprise or consist of one or more TLR family genes. In yet another embodiment, the one or more ADC Set I Marker genes comprise or consist of one or more interleukin receptor family genes. In a further embodiment, the one or more ADC Set I Marker genes comprise or consist of one or more immune checkpoint receptor genes. In one embodiment, the one or more ADC Set I Marker genes comprise or consist of one or more receptor tyrosin kinase genes. In another embodiment, the one or more ADC Set I Marker genes comprise or consist of one or more IFN receptor family genes. In yet another embodiment, the one or more ADC Set I Marker genes comprise or consist of one or more TNF family receptor genes. In a further embodiment, the one or more ADC Set I Marker genes comprise or consist of one or more inhibitory immunoreceptor genes. In one embodiment, the one or more ADC Set I Marker genes comprise or consist of one or more metabolic enzyme genes.

Similarly, in various embodiments of the methods provided herein, including the methods provided in this Section (Section 5.2) such as the methods provided in this and the preceding 24 paragraphs, the one or more ADC Set I Marker genes comprise or consist of any two of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In one embodiment, the one or more ADC Set I Marker genes comprise or consist of any three of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In another embodiment, the one or more ADC Set I Marker genes comprise or consist of any four of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In yet another embodiment, the one or more ADC Set I Marker genes comprise or consist of any five of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In a further embodiment, the one or more ADC Set I Marker genes comprise or consist of any six of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In one embodiment, the one or more ADC Set I Marker genes comprise or consist of any seven of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In another embodiment, the one or more ADC Set I Marker genes comprise or consist of any eight of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation. In yet another embodiment, the one or more ADC Set I Marker genes comprise or consist of any nine of the group consisting of: one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and one or more metabolic enzyme genes, in any combination or permutation.

In various embodiments of the methods provided herein, including the methods provided in this Section (Section 5.2) such as the methods provided in this and the preceding 25 paragraphs, the one or more ADC Set I Marker genes comprise or consist of one or more MHC signature genes. In certain embodiments of the methods provided herein, including the methods provided in this Section (Section 5.2) such as the methods provided in this and the preceding 24 paragraphs, the one or more MHC signature genes comprise or consist of one or more MHC class genes. In some embodiments of the methods provided herein, the one or more MHC class genes comprise or consist of one or more MHC class I genes. In one embodiment, the MHC signature genes comprise or consist of one or more genes selected from the group consisting of human leukocyte antigens-A (HLA-A), HLA-B, HLA-C, HLA-E, HLA-F, and Transporter 2, ATP binding cassette subfamily B member (TAP2). In another embodiment, the MHC class genes comprise or consist of one or more genes selected from the group consisting of HLA-A, HLA-B, HLA-C, HLA-E, HLA-F, and TAP2. In yet another embodiment, the MHC class I genes comprise or consist of one or more genes selected from the group consisting of HLA-A, HLA-B, HLA-C, HLA-E, HLA-F, and TAP2. In one embodiment, the MHC class I genes comprise or consist of HLA-A. In some embodiments, the MHC class I genes comprise or consist of HLA-B. In certain embodiments, the MHC class I genes comprise or consist of HLA-C. In other embodiments, the MHC class I genes comprise or consist of HLA-E. In yet other embodiments, the MHC class I genes comprise or consist of HLA-F. In further embodiments, the MHC class I genes comprise or consist of TAP2. In one embodiment, the MHC class I genes comprise or consist of HLA-A and HLA-B. In some embodiments, the MHC class I genes comprise or consist of HLA-A and HLA-C. In certain embodiments, the MHC class I genes comprise or consist of HLA-A and HLA-E. In other embodiments, the MHC class I genes comprise or consist of HLA-A and HLA-F. In yet other embodiments, the MHC class I genes comprise or consist of HLA-A and TAP2. In further embodiments, the MHC class I genes comprise or consist of HLA-B and HLA-C. In one embodiment, the MHC class I genes comprise or consist of HLA-B and HLA-E. In some embodiments, the MHC class I genes comprise or consist of HLA-B and HLA-F. In certain embodiments, the MHC class I genes comprise or consist of HLA-B and TAP2. In other embodiments, the MHC class I genes comprise or consist of HLA-C and HLA-E. In yet other embodiments, the MHC class I genes comprise or consist of HLA-C and HLA-F. In further embodiments, the MHC class I genes comprise or consist of HLA-C and TAP2. In one embodiment, the MHC class I genes comprise or consist of HLA-E and HLA-F. In some embodiments, the MHC class I genes comprise or consist of HLA-E and TAP2. In certain embodiments, the MHC class I genes comprise or consist of HLA-F and TAP2. In other embodiments, the MHC class I genes comprise or consist of HLA-A, HLA-B, and HLA-C. In yet other embodiments, the MHC class I genes comprise or consist of HLA-A, HLA-B and HLA-E. In further embodiments, the MHC class I genes comprise or consist of HLA-A, HLA-B, and HLA-F. In one embodiment, the MHC class I genes comprise or consist of HLA-A, HLA-B, and TAP2. In some embodiments, the MHC class I genes comprise or consist of HLA-A, HLA-C and HLA-E. In certain embodiments, the MHC class I genes comprise or consist of HLA-A, HLA-C, and HLA-F. In other embodiments, the MHC class I genes comprise or consist of HLA-A, HLA-C, and TAP2. In yet other embodiments, the MHC class I genes comprise or consist of HLA-A, HLA-E and HLA-F. In further embodiments, the MHC class I genes comprise or consist of HLA-A, HLA-E, and TAP2. In one embodiment, the MHC class I genes comprise or consist of HLA-A, HLA-F, and TAP2. In some embodiments, the MHC class I genes comprise or consist of HLA-B, HLA-C and HLA-E. In certain embodiments, the MHC class I genes comprise or consist of HLA-B, HLA-C, and HLA-F. In other embodiments, the MHC class I genes comprise or consist of HLA-B, HLA-C, and TAP2. In yet other embodiments, the MHC class I genes comprise or consist of HLA-B, HLA-E and HLA-F. In further embodiments, the MHC class I genes comprise or consist of HLA-B, HLA-E, and TAP2. In one embodiment, the MHC class I genes comprise or consist of HLA-B, HLA-F, and TAP2. In yet other embodiments, the MHC class I genes comprise or consist of HLA-C, HLA-E and HLA-F. In further embodiments, the MHC class I genes comprise or consist of HLA-C, HLA-E, and TAP2. In one embodiment, the MHC class I genes comprise or consist of HLA-C, HLA-F, and TAP2. In one embodiment, the MHC class I genes comprise or consist of HLA-E, HLA-F, and TAP2. In other embodiments, the MHC class I genes comprise or consist of HLA-A, HLA-B, HLA-C, and HLA-E. In further embodiments, the MHC class I genes comprise or consist of HLA-A, HLA-B, HLA-C, and HLA-F. In one embodiment, the MHC class I genes comprise or consist of HLA-A, HLA-B, HLA-C, and TAP2. In certain embodiments, the MHC class I genes comprise or consist of HLA-A, HLA-C, HLA-E, and HLA-F. In other embodiments, the MHC class I genes comprise or consist of HLA-A, HLA-C, HLA-E, and TAP2. In yet other embodiments, the MHC class I genes comprise or consist of HLA-A, HLA-E, HLA-F, and TAP2. In further embodiments, the MHC class I genes comprise or consist of HLA-B, HLA-C, HLA-E, and HLA-F. In one embodiment, the MHC class I genes comprise or consist of HLA-B, HLA-C, HLA-E, and TAP2. In some embodiments, the MHC class I genes comprise or consist of HLA-C, HLA-E, HLA-F, and TAP2. In certain embodiments, the MHC class I genes comprise or consist of HLA-A, HLA-B, HLA-C, HLA-E, and HLA-F. In other embodiments, the MHC class I genes comprise or consist of HLA-A, HLA-B, HLA-C, HLA-E, and TAP2. In yet other embodiments, the MHC class I genes comprise or consist of HLA-A, HLA-B, HLA-C, HLA-F, and TAP2. In further embodiments, the MHC class I genes comprise or consist of HLA-A, HLA-B, HLA-E, HLA-F, and TAP2. In one embodiment, the MHC class I genes comprise or consist of HLA-A, HLA-C, HLA-E, HLA-F, and TAP2. In yet other embodiments, the MHC class I genes comprise or consist of HLA-B, HLA-C, HLA-E, HLA-F, and TAP2. In further embodiments, the MHC class I genes comprise or consist of HLA-A, HLA-B, HLA-C, HLA-E, HLA-F, and TAP2. In some embodiments, the MHC class I genes comprise or consist of any one of HLA-A, HLA-B, HLA-C, HLA-E, HLA-F, and TAP2. In some embodiments, the MHC class I genes comprise or consist of any two of HLA-A, HLA-B, HLA-C, HLA-E, HLA-F, and TAP2, in any combination or permutation. In some embodiments, the MHC class I genes comprise or consist of any three of HLA-A, HLA-B, HLA-C, HLA-E, HLA-F, and TAP2, in any combination or permutation. In some embodiments, the MHC class I genes comprise or consist of any four of HLA-A, HLA-B, HLA-C, HLA-E, HLA-F, and TAP2, in any combination or permutation. In some embodiments, the MHC class I genes comprise or consist of any five of HLA-A, HLA-B, HLA-C, HLA-E, HLA-F, and TAP2, in any combination or permutation. In some embodiments, the MHC class I genes comprise or consist of all six of HLA-A, HLA-B, HLA-C, HLA-E, HLA-F, and TAP2, in any permutation.

In various embodiments of the methods provided herein, including the methods provided in this Section (Section 5.2) such as the methods provided in this and the preceding 26 paragraphs, the one or more ADC Set I Marker genes comprise or consist of one or more MHC signature genes. In certain embodiments of the methods provided herein, including the methods provided in this Section (Section 5.2) such as the methods provided in this and the preceding 25 paragraphs, the one or more MHC signature genes comprise or consist of one or more MHC class genes. In some embodiments of the methods provided herein, the one or more MHC class genes comprise or consist of one or more MHC class II genes. In one embodiment, the MHC signature genes comprise or consist of one or more genes selected from the group consisting of HLA-DMA, HLA-DMB, HLA-DRB1, HLA-DRA, and HLA-DPA1. In another embodiment, the MHC class genes comprise or consist of one or more genes selected from the group consisting of HLA-DMA, HLA-DMB, HLA-DRB1, HLA-DRA, and HLA-DPA1. In yet another embodiment, the MHC class II genes comprise or consist of one or more genes selected from the group consisting of HLA-DMA, HLA-DMB, HLA-DRB1, HLA-DRA, and HLA-DPA1. In one embodiment, the MHC class II genes comprise or consist of HLA-DMA. In some embodiments, the MHC class II genes comprise or consist of HLA-DMB. In certain embodiments, the MHC class II genes comprise or consist of HLA-DRB1. In other embodiments, the MHC class II genes comprise or consist of HLA-DRA. In yet other embodiments, the MHC class II genes comprise or consist of HLA-DPA1. In one embodiment, the MHC class II genes comprise or consist of HLA-DMA and HLA-DMB. In some embodiments, the MHC class II genes comprise or consist of HLA-DMA and HLA-DRB1. In certain embodiments, the MHC class II genes comprise or consist of HLA-DMA and HLA-DRA. In other embodiments, the MHC class II genes comprise or consist of HLA-DMA and HLA-DPA1. In further embodiments, the MHC class II genes comprise or consist of HLA-DMB and HLA-DRB1. In one embodiment, the MHC class II genes comprise or consist of HLA-DMB and HLA-DRA. In some embodiments, the MHC class II genes comprise or consist of HLA-DMB and HLA-DPA1. In other embodiments, the MHC class II genes comprise or consist of HLA-DRB1 and HLA-DRA. In yet other embodiments, the MHC class II genes comprise or consist of HLA-DRB1 and HLA-DPA1. In one embodiment, the MHC class II genes comprise or consist of HLA-DRA and HLA-DPA1. In other embodiments, the MHC class II genes comprise or consist of HLA-DMA, HLA-DMB, and HLA-DRB1. In yet other embodiments, the MHC class II genes comprise or consist of HLA-DMA, HLA-DMB and HLA-DRA. In further embodiments, the MHC class II genes comprise or consist of HLA-DMA, HLA-DMB, and HLA-DPA1. In some embodiments, the MHC class II genes comprise or consist of HLA-DMA, HLA-DRB1 and HLA-DRA. In certain embodiments, the MHC class II genes comprise or consist of HLA-DMA, HLA-DRB1, and HLA-DPA1. In yet other embodiments, the MHC class II genes comprise or consist of HLA-DMA, HLA-DRA and HLA-DPA1. In some embodiments, the MHC class II genes comprise or consist of HLA-DMB, HLA-DRB1 and HLA-DRA. In certain embodiments, the MHC class II genes comprise or consist of HLA-DMB, HLA-DRB1, and HLA-DPA1. In other embodiments, the MHC class II genes comprise or consist of HLA-DMB, HLA-DRA and HLA-DPA1. In yet other embodiments, the MHC class II genes comprise or consist of HLA-DRB1, HLA-DRA and HLA-DPA1. In other embodiments, the MHC class II genes comprise or consist of HLA-DMA, HLA-DMB, HLA-DRB1, and HLA-DRA. In further embodiments, the MHC class II genes comprise or consist of HLA-DMA, HLA-DMB, HLA-DRB1, and HLA-DPA1. In certain embodiments, the MHC class II genes comprise or consist of HLA-DMA, HLA-DRB1, HLA-DRA, and HLA-DPA1. In further embodiments, the MHC class II genes comprise or consist of HLA-DMB, HLA-DRB1, HLA-DRA, and HLA-DPA1. In certain embodiments, the MHC class II genes comprise or consist of HLA-DMA, HLA-DMB, HLA-DRB1, HLA-DRA, and HLA-DPA1. In some embodiments, the MHC class II genes comprise or consist of any one of HLA-DMA, HLA-DMB, HLA-DRB1, HLA-DRA, and HLA-DPA1. In some embodiments, the MHC class II genes comprise or consist of any two of HLA-DMA, HLA-DMB, HLA-DRB1, HLA-DRA, and HLA-DPA1, in any combination or permutation. In some embodiments, the MHC class II genes comprise or consist of any three of HLA-DMA, HLA-DMB, HLA-DRB1, HLA-DRA, and HLA-DPA1, in any combination or permutation. In some embodiments, the MHC class II genes comprise or consist of any four of HLA-DMA, HLA-DMB, HLA-DRB1, HLA-DRA, and HLA-DPA1, in any combination or permutation. In some embodiments, the MHC class II genes comprise or consist of all five of HLA-DMA, HLA-DMB, HLA-DRB1, HLA-DRA, and HLA-DPA1, in any combination or permutation. In some embodiments, the one or more MHC class genes comprise or consist of any number, in any combination or permutation of any MHC class I and MHC class II genes described in this Section (Section 5.2) including those described this and the preceding paragraph. In some embodiments, the MHC signature genes comprise or consist of any number, in any combination or permutation of any MHC class I and MHC class II genes described in this Section (Section 5.2) including those described this and the preceding paragraph. In some embodiments, the MHC signature genes do not comprise HLA-DPB1. In certain embodiments, the MHC class genes do not comprise HLA-DPB1. In other embodiments, the MHC class II genes do not comprise HLA-DPB1. In some embodiments, the MHC signature genes are not HLA-DPB1. In certain embodiments, the MHC class genes are not HLA-DPB1. In other embodiments, the MHC class II genes are not HLA-DPB1.

In various embodiments of the methods provided herein, including the methods provided in this Section (Section 5.2) such as the methods provided in this and the preceding 27 paragraphs, the one or more ADC Set I Marker genes comprise or consist of one or more MHC signature genes. In some embodiments of the methods provided herein, including the methods provided in this Section (Section 5.2) such as the methods provided in this and the preceding 26 paragraphs, the one or more MHC signature genes comprise or consist of one or more MHC class genes. In some embodiments of the methods provided herein, the one or more MHC class genes comprise or consist of one or more MHC class II genes. In one embodiment, the MHC signature genes comprise or consist of one or more genes selected from the group consisting of HLA-DMA, HLA-DMB, HLA-DRB, HLA-DRA, and HLA-DPA1. In another embodiment, the MHC class genes comprise or consist of one or more genes selected from the group consisting of HLA-DMA, HLA-DMB, HLA-DRB, HLA-DRA, and HLA-DPA1. In yet another embodiment, the MHC class II genes comprise or consist of one or more genes selected from the group consisting of HLA-DMA, HLA-DMB, HLA-DRB, HLA-DRA, and HLA-DPA1. In one embodiment, the MHC class II genes comprise or consist of HLA-DMA. In some embodiments, the MHC class II genes comprise or consist of HLA-DMB. In certain embodiments, the MHC class II genes comprise or consist of HLA-DRB. In other embodiments, the MHC class II genes comprise or consist of HLA-DRA. In yet other embodiments, the MHC class II genes comprise or consist of HLA-DPA1. In one embodiment, the MHC class II genes comprise or consist of HLA-DMA and HLA-DMB. In some embodiments, the MHC class II genes comprise or consist of HLA-DMA and HLA-DRB. In certain embodiments, the MHC class II genes comprise or consist of HLA-DMA and HLA-DRA. In other embodiments, the MHC class II genes comprise or consist of HLA-DMA and HLA-DPA1. In further embodiments, the MHC class II genes comprise or consist of HLA-DMB and HLA-DRB. In one embodiment, the MHC class II genes comprise or consist of HLA-DMB and HLA-DRA. In some embodiments, the MHC class II genes comprise or consist of HLA-DMB and HLA-DPA1. In other embodiments, the MHC class II genes comprise or consist of HLA-DRB and HLA-DRA. In yet other embodiments, the MHC class II genes comprise or consist of HLA-DRB and HLA-DPA1. In one embodiment, the MHC class II genes comprise or consist of HLA-DRA and HLA-DPA1. In other embodiments, the MHC class II genes comprise or consist of HLA-DMA, HLA-DMB, and HLA-DRB. In yet other embodiments, the MHC class II genes comprise or consist of HLA-DMA, HLA-DMB and HLA-DRA. In further embodiments, the MHC class II genes comprise or consist of HLA-DMA, HLA-DMB, and HLA-DPA1. In some embodiments, the MHC class II genes comprise or consist of HLA-DMA, HLA-DRB and HLA-DRA. In certain embodiments, the MHC class II genes comprise or consist of HLA-DMA, HLA-DRB, and HLA-DPA1. In yet other embodiments, the MHC class II genes comprise or consist of HLA-DMA, HLA-DRA and HLA-DPA1. In some embodiments, the MHC class II genes comprise or consist of HLA-DMB, HLA-DRB and HLA-DRA. In certain embodiments, the MHC class II genes comprise or consist of HLA-DMB, HLA-DRB, and HLA-DPA1. In other embodiments, the MHC class II genes comprise or consist of HLA-DMB, HLA-DRA and HLA-DPA1. In yet other embodiments, the MHC class II genes comprise or consist of HLA-DRB, HLA-DRA and HLA-DPA1. In other embodiments, the MHC class II genes comprise or consist of HLA-DMA, HLA-DMB, HLA-DRB, and HLA-DRA. In further embodiments, the MHC class II genes comprise or consist of HLA-DMA, HLA-DMB, HLA-DRB, and HLA-DPA1. In certain embodiments, the MHC class II genes comprise or consist of HLA-DMA, HLA-DRB, HLA-DRA, and HLA-DPA1. In further embodiments, the MHC class II genes comprise or consist of HLA-DMB, HLA-DRB, HLA-DRA, and HLA-DPA1. In certain embodiments, the MHC class II genes comprise or consist of HLA-DMA, HLA-DMB, HLA-DRB, HLA-DRA, and HLA-DPA1. In some embodiments, the MHC class II genes comprise or consist of any one of HLA-DMA, HLA-DMB, HLA-DRB, HLA-DRA, and HLA-DPA1. In some embodiments, the MHC class II genes comprise or consist of any two of HLA-DMA, HLA-DMB, HLA-DRB, HLA-DRA, and HLA-DPA1, in any combination or permutation. In some embodiments, the MHC class II genes comprise or consist of any three of HLA-DMA, HLA-DMB, HLA-DRB, HLA-DRA, and HLA-DPA1, in any combination or permutation. In some embodiments, the MHC class II genes comprise or consist of any four of HLA-DMA, HLA-DMB, HLA-DRB, HLA-DRA, and HLA-DPA1, in any combination or permutation. In some embodiments, the MHC class II genes comprise or consist of all five of HLA-DMA, HLA-DMB, HLA-DRB, HLA-DRA, and HLA-DPA1, in any combination or permutation. In some embodiments, the HLA-DRB comprises or is HLA-DRB1. In some embodiments, the HLA-DRB comprises or is HLA-DRB3. In some embodiments, the HLA-DRB comprises or is HLA-DRB4. In some embodiments, the HLA-DRB comprises or is HLA-DRB5. In some embodiments, the HLA-DRB comprises or consists of HLA-DRB1 and HLA-DRB3. In some embodiments, the HLA-DRB comprises or consists of HLA-DRB1 and HLA-DRB4. In some embodiments, the HLA-DRB comprises or consists of HLA-DRB1 and HLA-DRB5. In some embodiments, the HLA-DRB comprises or consists of HLA-DRB3 and HLA-DRB4. In some embodiments, the HLA-DRB comprises or consists of HLA-DRB3 and HLA-DRB5. In some embodiments, the HLA-DRB comprises or consists of HLA-DRB4 and HLA-DRB5. In some embodiments, the HLA-DRB comprises or consists of HLA-DRB1, HLA-DRB3, and HLA-DRB4. In some embodiments, the HLA-DRB comprises or consists of HLA-DRB1, HLA-DRB3, and HLA-DRB5. In some embodiments, the HLA-DRB comprises or consists of HLA-DRB1, HLA-DRB4, and HLA-DRB5. In some embodiments, the HLA-DRB comprises or consists of HLA-DRB3, HLA-DRB4, and HLA-DRB5. In some embodiments, the HLA-DRB comprises or consists of HLA-DRB1, HLA-DRB3, HLA-DRB4, and HLA-DRB5. In some embodiments, the one or more MHC class genes comprise or consist of any number, in any combination or permutation of any MHC class I and MHC class II genes described in this Section (Section 5.2) including those described this and the preceding two paragraphs. In some embodiments, the MHC signature genes comprise or consist of any number, in any combination or permutation of any MHC class I and MHC class II genes described in this Section (Section 5.2) including those described this and the preceding two paragraphs. In some embodiments, the MHC signature genes do not comprise HLA-DPB1. In certain embodiments, the MHC class genes do not comprise HLA-DPB1. In other embodiments, the MHC class II genes do not comprise HLA-DPB1. In some embodiments, the MHC signature genes are not HLA-DPB1. In certain embodiments, the MHC class genes are not HLA-DPB1. In other embodiments, the MHC class II genes are not HLA-DPB1.

In various embodiments of the methods provided herein, including the methods provided in this Section (Section 5.2) such as the methods provided in this and the preceding 28 paragraphs, the one or more ADC Set I Marker genes comprise or consist of one or more MHC signature genes. In some embodiments of the methods provided herein, including the methods provided in this Section (Section 5.2) such as the methods provided in this and the preceding 27 paragraphs, the one or more MHC signature genes comprise or consist of one or more MHC class genes. In some embodiments of the methods provided herein, the one or more MHC class genes comprise or consist of one or more MHC class III genes. In one embodiment, the MHC signature genes comprise or consist of one or more genes selected from the group consisting of LST1, LTB, AIF1, and TNF. In another embodiment, the MHC class genes comprise or consist of one or more genes selected from the group consisting of LST1, LTB, AIF1, and TNF. In yet another embodiment, the MHC class III genes comprise or consist of one or more genes selected from the group consisting of LST1, LTB, AIF1, and TNF. In one embodiment, the MHC class III genes comprise or consist of LST1. In some embodiments, the MHC class III genes comprise or consist of LTB. In certain embodiments, the MHC class III genes comprise or consist of AIF1. In other embodiments, the MHC class III genes comprise or consist of TNF. In one embodiment, the MHC class III genes comprise or consist of LST1 and LTB. In some embodiments, the MHC class III genes comprise or consist of LST1 and AIF1. In certain embodiments, the MHC class III genes comprise or consist of LST1 and TNF. In further embodiments, the MHC class III genes comprise or consist of LTB and AIF1. In one embodiment, the MHC class III genes comprise or consist of LTB and TNF. In other embodiments, the MHC class III genes comprise or consist of AIF1 and TNF. In yet other embodiments, the MHC class III genes comprise or consist of LST1, LTB, and AIF1. In some embodiments, the MHC class III genes comprise or consist of LST1, LTB and TNF. In certain embodiments, the MHC class III genes comprise or consist of LST1, AIF1 and TNF. In some embodiments, the MHC class III genes comprise or consist of LTB, AIF1 and TNF. In other embodiments, the MHC class III genes comprise or consist of LST1, LTB, AIF1, and TNF. In some embodiments, the MHC class III genes comprise or consist of any one of LST1, LTB, AIF1, and TNF. In some embodiments, the MHC class III genes comprise or consist of any two of LST1, LTB, AIF1, and TNF, in any combination or permutation. In some embodiments, the MHC class III genes comprise or consist of any three of LST1, LTB, AIF1, and TNF, in any combination or permutation. In some embodiments, the MHC class III genes comprise or consist of all four of LST1, LTB, AIF1, and TNF, in any permutation.

As the MHC class genes comprise MHC class I, MHC class II, and/or MHC class III genes, in some embodiments of the methods provided herein, including the methods provided in this Section (Section 5.2) such as the methods provided in this and the preceding 29 paragraphs, the disclosure provides that the one or more MHC class genes comprise or consist of one or more genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, and TNF. In certain embodiments, the one or more MHC class genes comprise or consist of 1, 2, 3, 4, 5, 6, 7, 8, 9, 10, 11, 12, 13, 14, or 15 genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, and TNF. In some embodiments, the one or more MHC signature genes comprise or consist of one or more genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, and TNF. In some embodiments, the one or more MHC signature genes comprise or consist of 1, 2, 3, 4, 5, 6, 7, 8, 9, 10, 11, 12, 13, 14, or 15 genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, and TNF.

In various embodiments of the methods provided herein, including the methods provided in this Section (Section 5.2) such as the methods provided in this and the preceding 30 paragraphs, the one or more ADC Set I Marker genes comprise or consist of one or more MHC signature genes. In some embodiments of the methods provided herein, including the methods provided in this Section (Section 5.2) such as the methods provided in this and the preceding 29 paragraphs, the one or more MHC signature genes comprise or consist of one or more MHC regulator genes. In some embodiments of the methods provided herein, the MHC signature genes comprise or consist of one or more genes selected from the group consisting of interferon regulatory factor IRF7 genes, nuclear factor kappa-light-chain-enhancer of activated B cells (NF-κB) family genes, signal transducer and activator of transcription (STAT) family genes, CTCF, CIITA, RFX transcription factor family genes, SPI1, and nuclear transcription factor Y (NFY) genes. In some embodiments of the methods provided herein, the MHC regulator genes comprise or consist of one or more genes selected from the group consisting of IRF genes, NF-κB family gene, STAT family gene, CTCF, CIITA, RFX transcription factor family genes, SPI1, and NFY genes. In one embodiment, the MHC regulator genes comprise or consist of IRF genes. In some embodiments, the MHC regulator genes comprise or consist of NF-κB family gene. In certain embodiments, the MHC regulator genes comprise or consist of STAT family gene. In other embodiments, the MHC regulator genes comprise or consist of NFY genes. In yet other embodiments, the MHC regulator genes comprise or consist of CTCF. In another embodiment, the MHC regulator genes comprise or consist of CIITA. In yet another embodiment, the MHC regulator genes comprise or consist of RFX transcription factor family genes. In one embodiment, the MHC regulator genes comprise or consist of IRF genes and NF-κB family gene. In some embodiments, the MHC regulator genes comprise or consist of IRF genes and STAT family gene. In certain embodiments, the MHC regulator genes comprise or consist of IRF genes and NFY genes. In other embodiments, the MHC regulator genes comprise or consist of IRF genes and CTCF. In some embodiments, the MHC regulator genes comprise or consist of IRF genes and CIITA. In other embodiments, the MHC regulator genes comprise or consist of IRF genes and RFX transcription factor family genes. In further embodiments, the MHC regulator genes comprise or consist of NF-κB family gene and STAT family gene. In one embodiment, the MHC regulator genes comprise or consist of NF-κB family gene and NFY genes. In some embodiments, the MHC regulator genes comprise or consist of NF-κB family gene and CTCF. In certain embodiments, the MHC regulator genes comprise or consist of NF-κB family gene and CIITA. In some embodiments, the MHC regulator genes comprise or consist of NF-κB family gene and RFX transcription factor family genes. In other embodiments, the MHC regulator genes comprise or consist of STAT family gene and NFY genes. In yet other embodiments, the MHC regulator genes comprise or consist of STAT family gene and CTCF. In some embodiments, the MHC regulator genes comprise or consist of STAT family gene and CIITA. In certain embodiments, the MHC regulator genes comprise or consist of STAT family gene and RFX transcription factor family genes. In one embodiment, the MHC regulator genes comprise or consist of NFY genes and CTCF. In another embodiment, the MHC regulator genes comprise or consist of NFY genes and CIITA. In one embodiment, the MHC regulator genes comprise or consist of NFY genes and RFX transcription factor family genes. In some embodiments, the MHC regulator genes comprise or consist of CTCF and CIITA. In certain embodiments, the MHC regulator genes comprise or consist of CTCF and RFX transcription factor family genes. In one embodiment, the MHC regulator genes comprise or consist of CIITA and RFX transcription factor family genes. In other embodiments, the MHC regulator genes comprise or consist of IRF genes, NF-κB family gene, and STAT family gene. In yet other embodiments, the MHC regulator genes comprise or consist of IRF genes, NF-κB family gene and NFY genes. In further embodiments, the MHC regulator genes comprise or consist of IRF genes, NF-κB family gene, and CTCF. In some embodiments, the MHC regulator genes comprise or consist of IRF genes, NF-κB family gene, and CIITA. In certain embodiments, the MHC regulator genes comprise or consist of IRF genes, NF-κB family gene, and RFX transcription factor family genes. In some embodiments, the MHC regulator genes comprise or consist of IRF genes, STAT family gene and NFY genes. In certain embodiments, the MHC regulator genes comprise or consist of IRF genes, STAT family gene, and CTCF. In some embodiments, the MHC regulator genes comprise or consist of IRF genes, STAT family gene, and CIITA. In other embodiments, the MHC regulator genes comprise or consist of IRF genes, STAT family gene, and RFX transcription factor family genes. In yet other embodiments, the MHC regulator genes comprise or consist of IRF genes, NFY genes and CTCF. In some embodiments, the MHC regulator genes comprise or consist of IRF genes, NFY genes and CIITA. In other embodiments, the MHC regulator genes comprise or consist of IRF genes, NFY genes and RFX transcription factor family genes. In some embodiments, the MHC regulator genes comprise or consist of IRF genes, CTCF and CIITA. In certain embodiments, the MHC regulator genes comprise or consist of IRF genes, CTCF and RFX transcription factor family genes. In other embodiments, the MHC regulator genes comprise or consist of IRF genes, CIITA and RFX transcription factor family genes. In some embodiments, the MHC regulator genes comprise or consist of NF-κB family gene, STAT family gene and NFY genes. In certain embodiments, the MHC regulator genes comprise or consist of NF-κB family gene, STAT family gene, and CTCF. In some embodiments, the MHC regulator genes comprise or consist of NF-κB family gene, STAT family gene, and CIITA. In certain embodiments, the MHC regulator genes comprise or consist of NF-κB family gene, STAT family gene, and RFX transcription factor family genes. In other embodiments, the MHC regulator genes comprise or consist of NF-κB family gene, NFY genes and CTCF. In yet other embodiments, the MHC regulator genes comprise or consist of NF-κB family gene, NFY genes and CIITA. In some embodiments, the MHC regulator genes comprise or consist of NF-κB family gene, NFY genes and RFX transcription factor family genes. In some embodiments, the MHC regulator genes comprise or consist of NF-κB family gene, CTCF and CIITA. In some embodiments, the MHC regulator genes comprise or consist of NF-κB family gene, CTCF and RFX transcription factor family genes. In some embodiments, the MHC regulator genes comprise or consist of NF-κB family gene, CIITA and RFX transcription factor family genes. In yet other embodiments, the MHC regulator genes comprise or consist of STAT family gene, NFY genes and CTCF. In other embodiments, the MHC regulator genes comprise or consist of STAT family gene, NFY genes and CIITA. In some embodiments, the MHC regulator genes comprise or consist of STAT family gene, NFY genes and RFX transcription factor family genes. In certain embodiments, the MHC regulator genes comprise or consist of STAT family gene, CTCF and CIITA. In other embodiments, the MHC regulator genes comprise or consist of STAT family gene, CTCF and RFX transcription factor family genes. In yet other embodiments, the MHC regulator genes comprise or consist of STAT family gene, CIITA and RFX transcription factor family genes. In some embodiments, the MHC regulator genes comprise or consist of NFY genes, CTCF and CIITA. In some embodiments, the MHC regulator genes comprise or consist of NFY genes, CTCF and RFX transcription factor family genes. In some embodiments, the MHC regulator genes comprise or consist of NFY genes, CIITA and RFX transcription factor family genes. In some embodiments, the MHC regulator genes comprise or consist of CTCF, CIITA and RFX transcription factor family genes. In other embodiments, the MHC regulator genes comprise or consist of IRF genes, NF-κB family gene, STAT family gene, and NFY genes. In further embodiments, the MHC regulator genes comprise or consist of IRF genes, NF-κB family gene, STAT family gene, and CTCF. In some embodiments, the MHC regulator genes comprise or consist of IRF genes, NF-κB family gene, STAT family gene, and CIITA. In other embodiments, the MHC regulator genes comprise or consist of IRF genes, NF-κB family gene, STAT family gene, and RFX transcription factor family genes. In some embodiments, the MHC regulator genes comprise or consist of IRF genes, NF-κB family gene, NFY genes, and CTCF. In some embodiments, the MHC regulator genes comprise or consist of IRF genes, NF-κB family gene, NFY genes, and CIITA. In some embodiments, the MHC regulator genes comprise or consist of IRF genes, NF-κB family gene, NFY genes, and RFX transcription factor family genes. In some embodiments, the MHC regulator genes comprise or consist of IRF genes, NF-κB family gene, CTCF, and CIITA. In some embodiments, the MHC regulator genes comprise or consist of IRF genes, NF-κB family gene, CTCF, and RFX transcription factor family genes. In some embodiments, the MHC regulator genes comprise or consist of IRF genes, NF-κB family gene, CIITA, and RFX transcription factor family genes. In certain embodiments, the MHC regulator genes comprise or consist of IRF genes, STAT family gene, NFY genes, and CTCF. In certain embodiments, the MHC regulator genes comprise or consist of IRF genes, STAT family gene, NFY genes, and CIITA. In some embodiments, the MHC regulator genes comprise or consist of IRF genes, STAT family gene, NFY genes, and RFX transcription factor family genes. In certain embodiments, the MHC regulator genes comprise or consist of IRF genes, STAT family gene, CTCF, and CIITA. In some embodiments, the MHC regulator genes comprise or consist of IRF genes, STAT family gene, CTCF, and RFX transcription factor family genes. In other embodiments, the MHC regulator genes comprise or consist of IRF genes, STAT family gene, CIITA, and RFX transcription factor family genes. In yet other embodiments, the MHC regulator genes comprise or consist of IRF genes, NFY genes, CTCF, and CIITA. In some embodiments, the MHC regulator genes comprise or consist of IRF genes, NFY genes, CTCF, and RFX transcription factor family genes. In certain embodiments, the MHC regulator genes comprise or consist of IRF genes, NFY genes, CIITA, and RFX transcription factor family genes. In other embodiments, the MHC regulator genes comprise or consist of IRF genes, CTCF, CIITA, and RFX transcription factor family genes. In further embodiments, the MHC regulator genes comprise or consist of NF-κB family gene, STAT family gene, NFY genes, and CTCF. In some embodiments, the MHC regulator genes comprise or consist of NF-κB family gene, STAT family gene, NFY genes, and CIITA. In certain embodiments, the MHC regulator genes comprise or consist of NF-κB family gene, STAT family gene, NFY genes, and RFX transcription factor family genes. In further embodiments, the MHC regulator genes comprise or consist of NF-κB family gene, STAT family gene, CTCF, and CIITA. In other embodiments, the MHC regulator genes comprise or consist of NF-κB family gene, STAT family gene, CTCF, and RFX transcription factor family genes. In some embodiments, the MHC regulator genes comprise or consist of NF-κB family gene, STAT family gene, CIITA, and RFX transcription factor family genes. In some embodiments, the MHC regulator genes comprise or consist of NF-κB family gene, NFY genes, CTCF, and CIITA. In some embodiments, the MHC regulator genes comprise or consist of NF-κB family gene, NFY genes, CTCF, and RFX transcription factor family genes. In some embodiments, the MHC regulator genes comprise or consist of NF-κB family gene, NFY genes, CIITA, and RFX transcription factor family genes. In some embodiments, the MHC regulator genes comprise or consist of NF-κB family gene, CTCF, CIITA, and RFX transcription factor family genes. In some embodiments, the MHC regulator genes comprise or consist of STAT family gene, NFY genes, CTCF, and CIITA. In some embodiments, the MHC regulator genes comprise or consist of STAT family gene, NFY genes, CTCF, and RFX transcription factor family genes. In some embodiments, the MHC regulator genes comprise or consist of STAT family gene, NFY genes, CIITA, and RFX transcription factor family genes. In some embodiments, the MHC regulator genes comprise or consist of STAT family gene, CTCF, CIITA, and RFX transcription factor family genes. In some embodiments, the MHC regulator genes comprise or consist of NFY genes, CTCF, CIITA, and RFX transcription factor family genes. In certain embodiments, the MHC regulator genes comprise or consist of IRF genes, NF-κB family gene, STAT family gene, NFY genes, and CTCF. In some embodiments, the MHC regulator genes comprise or consist of IRF genes, NF-κB family gene, STAT family gene, NFY genes, and CIITA. In certain embodiments, the MHC regulator genes comprise or consist of IRF genes, NF-κB family gene, STAT family gene, NFY genes, and RFX transcription factor family genes. In some embodiments, the MHC regulator genes comprise or consist of IRF genes, NF-κB family gene, STAT family gene, CTCF, and CIITA. In certain embodiments, the MHC regulator genes comprise or consist of IRF genes, NF-κB family gene, STAT family gene, CTCF, and RFX transcription factor family genes. In some embodiments, the MHC regulator genes comprise or consist of IRF genes, NF-κB family gene, STAT family gene, CIITA, and RFX transcription factor family genes. In certain embodiments, the MHC regulator genes comprise or consist of IRF genes, NF-κB family gene, NFY genes, CTCF, and CIITA. In some embodiments, the MHC regulator genes comprise or consist of IRF genes, NF-κB family gene, NFY genes, CTCF, and RFX transcription factor family genes. In certain embodiments, the MHC regulator genes comprise or consist of IRF genes, NF-κB family gene, NFY genes, CIITA, and RFX transcription factor family genes. In some embodiments, the MHC regulator genes comprise or consist of IRF genes, NF-κB family gene, CTCF, CIITA, and RFX transcription factor family genes. In certain embodiments, the MHC regulator genes comprise or consist of IRF genes, STAT family gene, NFY genes, CTCF, and CIITA. In some embodiments, the MHC regulator genes comprise or consist of IRF genes, STAT family gene, NFY genes, CTCF, and RFX transcription factor family genes. In certain embodiments, the MHC regulator genes comprise or consist of IRF genes, STAT family gene, NFY genes, CIITA, and RFX transcription factor family genes. In some embodiments, the MHC regulator genes comprise or consist of IRF genes, STAT family gene, CTCF, CIITA, and RFX transcription factor family genes. In certain embodiments, the MHC regulator genes comprise or consist of IRF genes, NFY genes, CTCF, CIITA, and RFX transcription factor family genes. In some embodiments, the MHC regulator genes comprise or consist of NF-κB family gene, STAT family gene, NFY genes, CTCF, and CIITA. In certain embodiments, the MHC regulator genes comprise or consist of NF-κB family gene, STAT family gene, NFY genes, CTCF, and RFX transcription factor family genes. In some embodiments, the MHC regulator genes comprise or consist of NF-κB family gene, STAT family gene, NFY genes, CIITA, and RFX transcription factor family genes. In some embodiments, the MHC regulator genes comprise or consist of NF-κB family gene, STAT family gene, CTCF, CIITA, and RFX transcription factor family genes. In certain embodiments, the MHC regulator genes comprise or consist of NF-κB family gene, NFY genes, CTCF, CIITA, and RFX transcription factor family genes. In some embodiments, the MHC regulator genes comprise or consist of STAT family gene, NFY genes, CTCF, CIITA, and RFX transcription factor family genes. In some embodiments, the MHC regulator genes comprise or consist of IRF genes, NF-κB family gene, STAT family gene, NFY genes, CTCF, and CIITA. In certain embodiments, the MHC regulator genes comprise or consist of IRF genes, NF-κB family gene, STAT family gene, NFY genes, CTCF, and RFX transcription factor family genes. In some embodiments, the MHC regulator genes comprise or consist of IRF genes, NF-κB family gene, STAT family gene, NFY genes, CIITA, and RFX transcription factor family genes. In certain embodiments, the MHC regulator genes comprise or consist of IRF genes, NF-κB family gene, STAT family gene, CTCF, CIITA, and RFX transcription factor family genes. In some embodiments, the MHC regulator genes comprise or consist of IRF genes, NF-κB family gene, NFY genes, CTCF, CIITA, and RFX transcription factor family genes. In certain embodiments, the MHC regulator genes comprise or consist of IRF genes, STAT family gene, NFY genes, CTCF, CIITA, and RFX transcription factor family genes. In some embodiments, the MHC regulator genes comprise or consist of NF-κB family gene, STAT family gene, NFY genes, CTCF, CIITA, and RFX transcription factor family genes. In some embodiments, the MHC regulator genes comprise or consist of any one of IRF genes, NF-κB family gene, STAT family gene, CTCF, CIITA, RFX transcription factor family genes, SPI1, and NFY genes. In some embodiments, the MHC regulator genes comprise or consist of any two of IRF genes, NF-κB family gene, STAT family gene, CTCF, CIITA, RFX transcription factor family genes, SPI1, and NFY genes, in any combination or permutation. In some embodiments, the MHC regulator genes comprise or consist of any three of IRF genes, NF-κB family gene, STAT family gene, CTCF, CIITA, RFX transcription factor family genes, SPI1, and NFY genes, in any combination or permutation. In some embodiments, the MHC regulator genes comprise or consist of any four of IRF genes, NF-κB family gene, STAT family gene, CTCF, CIITA, RFX transcription factor family genes, SPI1, and NFY genes, in any combination or permutation. In some embodiments, the MHC regulator genes comprise or consist of any five of IRF genes, NF-κB family gene, STAT family gene, CTCF, CIITA, RFX transcription factor family genes, SPI1, and NFY genes, in any combination or permutation. In some embodiments, the MHC regulator genes comprise or consist of any six of IRF genes, NF-κB family gene, STAT family gene, CTCF, CIITA, RFX transcription factor family genes, SPI1, and NFY genes, in any combination or permutation. In some embodiments, the MHC regulator genes comprise or consist of any seven of IRF genes, NF-κB family gene, STAT family gene, CTCF, CIITA, RFX transcription factor family genes, SPI1, and NFY genes, in any combination or permutation. In some embodiments, the MHC regulator genes comprise or consist of all eight of IRF genes, NF-κB family gene, STAT family gene, CTCF, CIITA, RFX transcription factor family genes, SPI1, and NFY genes, in any permutation. In one embodiment, the IRF genes comprise or consist of IRF7. In another embodiment, the IRF genes comprise or consist of IRF8. In yet another embodiment, the IRF genes comprise or consist of both IRF7 and IRF8. In one embodiment, the NF-κB family genes comprise or consist of NFKB1. In some embodiments, the NF-κB family genes comprise or consist of NFKB2. In certain embodiments, the NF-κB family genes comprise or consist of RELA. In other embodiments, the NF-κB family genes comprise or consist of REL. In yet other embodiments, the NF-κB family genes comprise or consist of RELB In one embodiment, the NF-κB family genes comprise or consist of NFKB1 and NFKB2. In some embodiments, the NF-κB family genes comprise or consist of NFKB1 and RELA In certain embodiments, the NF-κB family genes comprise or consist of NFKB1 and REL In other embodiments, the NF-κB family genes comprise or consist of NFKB1 and RELB In further embodiments, the NF-κB family genes comprise or consist of NFKB2 and RELA. In one embodiment, the NF-κB family genes comprise or consist of NFKB2 and REL. In some embodiments, the NF-κB family genes comprise or consist of NFKB2 and RELB. In other embodiments, the NF-κB family genes comprise or consist of RELA and REL In yet other embodiments, the NF-κB family genes comprise or consist of RELA and RELB. In one embodiment, the NF-κB family genes comprise or consist of REL and RELB In other embodiments, the NF-κB family genes comprise or consist of NFKB1, NFKB2, and RELA In yet other embodiments, the NF-κB family genes comprise or consist of NFKB1, NFKB2 and REL In further embodiments, the NF-κB family genes comprise or consist of NFKB1, NFKB2, and RELB. In some embodiments, the NF-κB family genes comprise or consist of NFKB1, RELA and REL. In certain embodiments, the NF-κB family genes comprise or consist of NFKB1, RELA, and RELB. In yet other embodiments, the NF-κB family genes comprise or consist of NFKB1, REL and RELB In some embodiments, the NF-κB family genes comprise or consist of NFKB2, RELA and REL In certain embodiments, the NF-κB family genes comprise or consist of NFKB2, RELA, and RELB. In other embodiments, the NF-κB family genes comprise or consist of NFKB2, REL and RELB. In yet other embodiments, the NF-κB family genes comprise or consist of RELA, REL and RELB In other embodiments, the NF-κB family genes comprise or consist of NFKB1, NFKB2, RELA, and REL. In further embodiments, the NF-κB family genes comprise or consist of NFKB1, NFKB2, RELA, and RELB In certain embodiments, the NF-κB family genes comprise or consist of NFKB1, RELA, REL, and RELB. In further embodiments, the NF-κB family genes comprise or consist of NFKB2, RELA, REL, and RELB In certain embodiments, the NF-κB family genes comprise or consist of NFKB1, NFKB2, RELA, REL, and RELB. In some embodiments, the STAT family genes comprise or consist of STAT2. In some embodiments, the STAT family genes comprise or consist of any one of STAT1, STAT2, STAT5, STAT4, STAT5, and STAT6. In some embodiments, the STAT family genes comprise or consist of any two of STAT1, STAT2, STAT3, STAT4, STAT5, and STAT6, in any combination or permutation. In some embodiments, the STAT family genes comprise or consist of any three of STAT1, STAT2, STAT3, STAT4, STAT5, and STAT6, in any combination or permutation. In some embodiments, the STAT family genes comprise or consist of any four of STAT1, STAT2, STAT3, STAT4, STAT5, and STAT6, in any combination or permutation. In some embodiments, the STAT family genes comprise or consist of any five of STAT1, STAT2, STAT3, STAT4, STAT5, and STAT6, in any combination or permutation. In some embodiments, the STAT family genes comprise or consist of all six of STAT1, STAT2, STAT3, STAT4, STAT5, and STAT6, in any combination or permutation. In one embodiment, the RFX transcription factor family genes comprise or consist of RFX1. In some embodiments, the RFX transcription factor family genes comprise or consist of RFX5. In certain embodiments, the RFX transcription factor family genes comprise or consist of RFX7. In other embodiments, the RFX transcription factor family genes comprise or consist of RFXAP. In yet other embodiments, the RFX transcription factor family genes comprise or consist of RFXANK. In one embodiment, the RFX transcription factor family genes comprise or consist of RFX1 and RFX5. In some embodiments, the RFX transcription factor family genes comprise or consist of RFX1 and RFX7. In certain embodiments, the RFX transcription factor family genes comprise or consist of RFX1 and RFXAP. In other embodiments, the RFX transcription factor family genes comprise or consist of RFX1 and RFXANK. In further embodiments, the RFX transcription factor family genes comprise or consist of RFX5 and RFX7. In one embodiment, the RFX transcription factor family genes comprise or consist of RFX5 and RFXAP. In some embodiments, the RFX transcription factor family genes comprise or consist of RFX5 and RFXANK. In other embodiments, the RFX transcription factor family genes comprise or consist of RFX7 and RFXAP. In yet other embodiments, the RFX transcription factor family genes comprise or consist of RFX7 and RFXANK. In one embodiment, the RFX transcription factor family genes comprise or consist of RFXAP and RFXANK. In other embodiments, the RFX transcription factor family genes comprise or consist of RFX1, RFX5, and RFX7. In yet other embodiments, the RFX transcription factor family genes comprise or consist of RFX1, RFX5 and RFXAP. In further embodiments, the RFX transcription factor family genes comprise or consist of RFX1, RFX5, and RFXANK. In some embodiments, the RFX transcription factor family genes comprise or consist of RFX1, RFX7 and RFXAP. In certain embodiments, the RFX transcription factor family genes comprise or consist of RFX1, RFX7, and RFXANK. In yet other embodiments, the RFX transcription factor family genes comprise or consist of RFX1, RFXAP and RFXANK. In some embodiments, the RFX transcription factor family genes comprise or consist of RFX5, RFX7 and RFXAP. In certain embodiments, the RFX transcription factor family genes comprise or consist of RFX5, RFX7, and RFXANK. In yet other embodiments, the RFX transcription factor family genes comprise or consist of RFX5, RFXAP and RFXANK. In yet other embodiments, the RFX transcription factor family genes comprise or consist of RFX7, RFXAP and RFXANK. In other embodiments, the RFX transcription factor family genes comprise or consist of RFX1, RFX5, RFX7, and RFXAP. In further embodiments, the RFX transcription factor family genes comprise or consist of RFX1, RFX5, RFX7, and RFXANK. In some embodiments, the RFX transcription factor family genes comprise or consist of RFX1, RFX5, RFXAP, and RFXANK. In certain embodiments, the RFX transcription factor family genes comprise or consist of RFX1, RFX7, RFXAP, and RFXANK. In further embodiments, the RFX transcription factor family genes comprise or consist of RFX5, RFX7, RFXAP, and RFXANK. In certain embodiments, the RFX transcription factor family genes comprise or consist of RFX1, RFX5, RFX7, RFXAP, and RFXANK. In one embodiment, the RFX transcription factor family genes do not comprise RFXAP. In another embodiment, the RFX transcription factor family genes do not comprise RFXANK. In yet another embodiment, the RFX transcription factor family genes do not comprise RFXAP or RFXANK. In one embodiment, the NFY genes comprise or consist of NFYA. In another embodiment, the NFY genes comprise or consist of NFYC. In yet another embodiment, the NFY genes comprise or consist of both NFYA and NFYC. In one embodiment, the one or more MHC regulator genes comprise or consist of SPI1. In some embodiments, the one or more MHC regulator genes comprise or consist of any number, in any combination or permutation of any MHC regulator genes described in this Section (Section 5.2) including those described this paragraph. In some embodiments, the MHC signature genes comprise or consist of any number, in any combination or permutation of any MHC regulator genes, MHC class I, MHC class II, and MHC class III genes described in this Section (Section 5.2) including those described this and the preceding three paragraphs.

In certain embodiments of the methods provided herein, including the methods provided in this Section (Section 5.2) such as the methods provided in this and the preceding 31 paragraphs, the one or more MHC signature genes comprise or consist of one or more MHC regulator genes. In some embodiments of the methods provided herein, including the methods provided in this Section (Section 5.2) such as the methods provided in this and the preceding 30 paragraphs, the one or more MHC signature genes comprise or consist of one or more genes selected from the group consisting of IRF7, NFKB2, RELA, STAT2, RFX1, RFX5, RFX7, CTCF, CIITA, RFXAP, RFXANK, SPI1, NFYA, NFYC, and IRF8. In other embodiments of the methods provided herein, the one or more MHC regulator genes comprise or consist of one or more genes selected from the group consisting of IRF7, NFKB2, RELA, STAT2, RFX1, RFX5, RFX7, CTCF, CIITA, RFXAP, RFXANK, SPI1, NFYA, NFYC, and IRF8. In some embodiments, the MHC regulator genes comprise or consist of any one of IRF7, NFKB2, RELA, STAT2, RFX1, RFX5, RFX7, CTCF, CIITA, RFXAP, RFXANK, SPI1, NFYA, NFYC, and IRF8. In some embodiments, the MHC regulator genes comprise or consist of any two of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any three of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any four of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any five of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any six of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any seven of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any eight of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any nine of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any ten of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any eleven of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any twelve of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any thirteen of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of any fourteen of IRF7, NFKB2, RELA, STAT2, RFX1, RFX5, RFX7, CTCF, CIITA, RFXAP, RFXANK, SPI1, NFYA, NFYC, and IRF8, in any combination or permutation. In some embodiments, the MHC regulator genes comprise or consist of all fifteen of IRF7, NFKB2, RELA, STAT2, RFX1, RFX5, RFX7, CTCF, CIITA, RFXAP, RFXANK, SPI1, NFYA, NFYC, and IRF8, in any permutation. In one embodiment, the RFX transcription factor family genes do not comprise RFXAP. In another embodiment, the RFX transcription factor family genes do not comprise RFXANK. In yet another embodiment, the RFX transcription factor family genes do not comprise RFXAP or RFXANK. In one embodiment, the one or more MHC regulator genes do not comprise RFXAP. In another embodiment, the one or more MHC regulator genes do not comprise RFXANK. In yet another embodiment, the one or more MHC regulator genes do not comprise RFXAP or RFXANK. In some embodiments, the one or more MHC regulator genes comprise or consist of any number, in any combination or permutation of any MHC regulator genes described in this Section (Section 5.2) including those described this paragraph. In some embodiments, the MHC signature genes comprise or consist of any number, in any combination or permutation of any MHC regulator genes, MHC class I, MHC class II, MHC and class III genes described in this Section (Section 5.2) including those described this and the preceding four paragraphs.

As the WIC signature genes comprise WIC class genes and MHC regulator genes, in some embodiments of the methods provided herein, including the methods provided in this Section (Section 5.2) such as the methods provided in this and the preceding 30 paragraphs, the disclosure provides that the one or more WIC signature genes comprise or consist of one or more genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, TNF, IRF7, NFKB2, RELA, STAT2, RFX1, RFX5, RFX7, CTCF, CIITA, RFXAP, RFXANK, SPI1, NFYA, NFYC, and IRF8. In certain embodiments, the one or more MHC signature genes comprise or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes selected from the group consisting of HLA-A, HLA-B, HLA-C, HLA-E, HLA-F, TAP2, HLA-DMA, HLA-DMB, HLA-DRB1, HLA-DRA, HLA-DPA1, LST1, LTB, AIF1, TNF, IRF7, NFKB2, RELA, STAT2, RFX1, RFX5, RFX7, CTCF, CIITA, RFXAP, RFXANK, SPI1, NFYA, NFYC, and IRF8.

In various embodiments of the methods provided herein, including the methods provided in this Section (Section 5.2) such as the methods provided in this and the preceding 33 paragraphs, the one or more ADC Set I Marker genes comprise or consist of one or more TLR family genes. In some embodiments of the methods provided herein, including the methods provided in this Section (Section 5.2) such as the methods provided in this and the preceding 31 paragraphs, the toll-like receptor family genes comprise or consist of one or more genes selected from the group consisting of TLR9, TLR8, and TLR7. In one embodiment, the toll-like receptor family genes comprise or consist of TLR9. In another embodiment, the toll-like receptor family genes comprise or consist of TLR8. In some embodiment, the toll-like receptor family genes comprise or consist of TLR7. In other embodiments, the toll-like receptor family genes comprise or consist of TLR9 and TLR8. In yet other embodiments, the toll-like receptor family genes comprise or consist of TLR9 and TLR7. In one embodiment, the toll-like receptor family genes comprise or consist of TLR8 and TLR7. In certain embodiment, the toll-like receptor family genes comprise or consist of TLR9, TLR8, and TLR7. In certain embodiments, the toll-like receptor family genes do not comprise or consist of TLR3. In some embodiment, the toll-like receptor family genes are not TLR3.

Additionally, in various embodiments of the methods provided herein, including the methods provided in this Section (Section 5.2) such as the methods provided in this and the preceding 34 paragraphs, the one or more ADC Set I Marker genes comprise or consist of one or more interleukin receptor family genes. In some embodiments of the methods provided herein, including the methods provided in this Section (Section 5.2) such as the methods provided in this and the preceding 32 paragraphs, the interleukin receptor family genes comprise or consist of one or more genes selected from the group consisting of IL2RA, IL2RB, IL2RG, IL21R, IL27R, IL1RN, IL17RA, IL3RA, IL1R1, IL17RC, IL20RA, and IL22RA1. In certain embodiments, the interleukin receptor family genes comprise or consist of 1 to 12 genes selected from the group consisting of IL2RA, IL2RB, IL2RG, IL21R, IL27R, IL1RN, IL17RA, IL3RA, IL1R1, IL17RC, IL20RA, and IL22RA1. In certain embodiments, the interleukin receptor family genes comprise or consist of 1, 2, 3, 4, 5, 6, 7, 8, 9, 10, 11, or 12 genes selected from the group consisting of IL2RA, IL2RB, IL2RG, IL21R, IL27R, IL1RN, IL17RA, IL3RA, IL1R1, IL17RC, IL20RA, and IL22RA1. In certain embodiments, the interleukin receptor family genes comprise or consist of any 1 gene selected from the group consisting of IL2RA, IL2RB, IL2RG, IL21R, IL27R, IL1RN, IL17RA, IL3RA, IL1R1, IL17RC, IL20RA, and IL22RA1. In certain embodiments, the interleukin receptor family genes comprise or consist of any 2 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 3 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 4 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 5 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 6 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 7 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 8 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 9 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 10 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 11 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In certain embodiments, the interleukin receptor family genes comprise or consist of any 12 genes selected from the group consisting of IL2RA, IL2RB, IL2RG, IL21R, IL27R, IL1RN, IL17RA, IL3RA, IL1R1, IL17RC, IL20RA, and IL22RA1, in any permutation or combination. In one specific embodiment, the interleukin receptor family genes consist of IL2RA. In another specific embodiment, the interleukin receptor family genes comprise IL2RA.

In some embodiments of the methods provided herein, including the methods provided in this Section (Section 5.2) such as the methods provided in this and the preceding 35 paragraphs, the one or more ADC Set I Marker genes comprise or consist of one or more immune checkpoint receptor genes. In certain embodiments of the methods provided herein, including the methods provided in this Section (Section 5.2) such as the methods provided in this and the preceding 33 paragraphs, the one or more immune checkpoint receptor genes comprise or consist of one or more B7 family genes. In some embodiments, the one or more immune checkpoint receptor genes comprise or consist of one or more Ig superfamily genes. In other embodiments, the one or more immune checkpoint receptor genes comprise or consist of both one or more B7 family genes and one or more Ig superfamily genes. In certain embodiments of the methods provided herein, the B7 family genes comprise or consist of one or more genes selected from the group consisting of VTCN1 and CD276. In one embodiment, the B7 family genes comprise or consist of VTCN1. In another embodiment, the B7 family genes comprise or consist of CD276. In other embodiments, the B7 family genes comprise or consist of VTCN1 and CD276. In one specific embodiment, the B7 family genes consist of VTCN1. In another specific embodiment, the B7 family genes comprise VTCN1. In some embodiments of the methods provided herein, the Ig superfamily genes comprise nectin family genes. In some embodiments of the methods provided herein, the Ig superfamily genes consist of nectin family genes. In one embodiment, the nectin family genes comprise or consist of PVRIG. In some embodiments, the nectin family genes comprise or consist of PVRL2. In certain embodiments, the nectin family genes comprise or consist of TIGIT. In certain embodiments, the nectin family genes comprise TIGIT. In certain embodiments, the nectin family genes consist of TIGIT. In one embodiment, the nectin family genes comprise or consist of PVRIG and PVRL2. In some embodiments, the nectin family genes comprise or consist of PVRIG and TIGIT. In further embodiments, the nectin family genes comprise or consist of PVRL2 and TIGIT. In yet other embodiments, the nectin family genes comprise or consist of PVRIG, PVRL2, and TIGIT. In some embodiments, the nectin family genes comprise or consist of any one of PVRIG, PVRL2, and TIGIT. In some embodiments, the nectin family genes comprise or consist of any two of PVRIG, PVRL2, and TIGIT, in any combination or permutation. In some embodiments, the nectin family genes comprise or consist of any three of PVRIG, PVRL2, and TIGIT, in any combination or permutation. In some embodiments of the methods provided herein, the Ig superfamily genes comprise LAG3. In certain embodiments, the Ig superfamily genes consist of LAG3. In some embodiments, the Ig superfamily genes comprise one or more nectin family genes and LAG3. In some embodiments, the Ig superfamily genes consist of one or more nectin family genes and LAG3. In one embodiment, the Ig superfamily genes comprise or consist of PVRIG. In some embodiments, the Ig superfamily genes comprise or consist of PVRL2. In certain embodiments, the Ig superfamily genes comprise or consist of TIGIT. In other embodiments, the Ig superfamily genes comprise or consist of LAG3. In one embodiment, the Ig superfamily genes comprise or consist of PVRIG and PVRL2. In some embodiments, the Ig superfamily genes comprise or consist of PVRIG and TIGIT. In certain embodiments, the Ig superfamily genes comprise or consist of PVRIG and LAG3. In further embodiments, the Ig superfamily genes comprise or consist of PVRL2 and TIGIT. In one embodiment, the Ig superfamily genes comprise or consist of PVRL2 and LAG3. In other embodiments, the Ig superfamily genes comprise or consist of TIGIT and LAG3. In yet other embodiments, the Ig superfamily genes comprise or consist of PVRIG, PVRL2, and TIGIT. In some embodiments, the Ig superfamily genes comprise or consist of PVRIG, PVRL2 and LAG3. In certain embodiments, the Ig superfamily genes comprise or consist of PVRIG, TIGIT and LAG3. In some embodiments, the Ig superfamily genes comprise or consist of PVRL2, TIGIT and LAG3. In other embodiments, the Ig superfamily genes comprise or consist of PVRIG, PVRL2, TIGIT, and LAG3. In some embodiments, the Ig superfamily genes comprise or consist of any one of PVRIG, PVRL2, TIGIT, and LAG3. In some embodiments, the Ig superfamily genes comprise or consist of any two of PVRIG, PVRL2, TIGIT, and LAG3, in any combination or permutation. In some embodiments, the Ig superfamily genes comprise or consist of any three of PVRIG, PVRL2, TIGIT, and LAG3, in any combination or permutation. In some embodiments, the Ig superfamily genes comprise or consist of any four of PVRIG, PVRL2, TIGIT, and LAG3, in any permutation.

In various embodiments of the methods provided herein, including the methods provided in this Section (Section 5.2) such as the methods provided in this and the preceding 36 paragraphs, the one or more ADC Set I Marker genes comprise or consist of one or more receptor tyrosin kinase genes. In some embodiments of the methods provided herein, including the methods provided in this Section (Section 5.2) such as the methods provided in this and the preceding 34 paragraphs, the receptor tyrosin kinase genes comprise or consist of one or more genes selected from the group consisting of CSF1R, PDGFRB, TEK/TIE2, and FLT3. In one embodiment, the receptor tyrosin kinase genes comprise or consist of CSF1R. In some embodiments, the receptor tyrosin kinase genes comprise or consist of PDGFRB. In certain embodiments, the receptor tyrosin kinase genes comprise or consist of TEK/TIE2. In other embodiments, the receptor tyrosin kinase genes comprise or consist of FLT3. In one embodiment, the receptor tyrosin kinase genes comprise or consist of CSF1R and PDGFRB. In some embodiments, the receptor tyrosin kinase genes comprise or consist of CSF1R and TEK/TIE2. In certain embodiments, the receptor tyrosin kinase genes comprise or consist of CSF1R and FLT3. In further embodiments, the receptor tyrosin kinase genes comprise or consist of PDGFRB and TEK/TIE2. In one embodiment, the receptor tyrosin kinase genes comprise or consist of PDGFRB and FLT3. In other embodiments, the receptor tyrosin kinase genes comprise or consist of TEK/TIE2 and FLT3. In yet other embodiments, the receptor tyrosin kinase genes comprise or consist of CSF1R, PDGFRB, and TEK/TIE2. In some embodiments, the receptor tyrosin kinase genes comprise or consist of CSF1R, PDGFRB and FLT3. In certain embodiments, the receptor tyrosin kinase genes comprise or consist of CSF1R, TEK/TIE2 and FLT3. In some embodiments, the receptor tyrosin kinase genes comprise or consist of PDGFRB, TEK/TIE2 and FLT3. In other embodiments, the receptor tyrosin kinase genes comprise or consist of CSF1R, PDGFRB, TEK/TIE2, and FLT3. In some embodiments, the receptor tyrosin kinase genes comprise or consist of any one of CSF1R, PDGFRB, TEK/TIE2, and FLT3. In some embodiments, the receptor tyrosin kinase genes comprise or consist of any two of CSF1R, PDGFRB, TEK/TIE2, and FLT3, in any combination or permutation. In some embodiments, the receptor tyrosin kinase genes comprise or consist of any three of CSF1R, PDGFRB, TEK/TIE2, and FLT3, in any combination or permutation. In some embodiments, the receptor tyrosin kinase genes comprise or consist of any four of CSF1R, PDGFRB, TEK/TIE2, and FLT3, in any permutation. In one specific embodiment, the receptor tyrosin kinase genes consist of CSF1R. In another specific embodiment, the receptor tyrosin kinase genes comprise CSF1R.

Additionally, in various embodiments of the methods provided herein, including the methods provided in this Section (Section 5.2) such as the methods provided in this and the preceding 37 paragraphs, the one or more ADC Set I Marker genes comprise or consist of one or more TNF family receptor genes. In some embodiments of the methods provided herein, including the methods provided in this Section (Section 5.2) such as the methods provided in this and the preceding 35 paragraphs, the TNF family receptor genes comprise or consist of one or more genes selected from the group consisting of CD40, TNFRSF1A, TNFRSF21, and TNFRSF1B. In one embodiment, the TNF family receptor genes comprise or consist of CD40. In some embodiments, the TNF family receptor genes comprise or consist of TNFRSF1A. In certain embodiments, the TNF family receptor genes comprise or consist of TNFRSF21. In other embodiments, the TNF family receptor genes comprise or consist of TNFRSF1B. In one embodiment, the TNF family receptor genes comprise or consist of CD40 and TNFRSF1A. In some embodiments, the TNF family receptor genes comprise or consist of CD40 and TNFRSF21. In certain embodiments, the TNF family receptor genes comprise or consist of CD40 and TNFRSF1B. In further embodiments, the TNF family receptor genes comprise or consist of TNFRSF1A and TNFRSF21. In one embodiment, the TNF family receptor genes comprise or consist of TNFRSF1A and TNFRSF1B. In other embodiments, the TNF family receptor genes comprise or consist of TNFRSF21 and TNFRSF1B. In yet other embodiments, the TNF family receptor genes comprise or consist of CD40, TNFRSF1A, and TNFRSF21. In some embodiments, the TNF family receptor genes comprise or consist of CD40, TNFRSF1A and TNFRSF1B. In certain embodiments, the TNF family receptor genes comprise or consist of CD40, TNFRSF21 and TNFRSF1B. In some embodiments, the TNF family receptor genes comprise or consist of TNFRSF1A, TNFRSF21 and TNFRSF1B. In other embodiments, the TNF family receptor genes comprise or consist of CD40, TNFRSF1A, TNFRSF21, and TNFRSF1B. In some embodiments, the TNF family receptor genes comprise or consist of any one of CD40, TNFRSF1A, TNFRSF21, and TNFRSF1B. In some embodiments, the TNF family receptor genes comprise or consist of any two of CD40, TNFRSF1A, TNFRSF21, and TNFRSF1B, in any combination or permutation. In some embodiments, the TNF family receptor genes comprise or consist of any three of CD40, TNFRSF1A, TNFRSF21, and TNFRSF1B, in any combination or permutation. In some embodiments, the TNF family receptor genes comprise or consist of any four of CD40, TNFRSF1A, TNFRSF21, and TNFRSF1B, in any permutation.

In certain embodiments of the methods provided herein, including the methods provided in this Section (Section 5.2) such as the methods provided in this and the preceding 38 paragraphs, the one or more ADC Set I Marker genes comprise or consist of one or more IFN receptor family genes. In some embodiments of the methods provided herein, including the methods provided in this Section (Section 5.2) such as the methods provided in this and the preceding 36 paragraphs, the IFN receptor family genes comprise or consist of one or more genes selected from the group consisting of IFNAR1 and IFNAR2. In one embodiment, the IFN receptor family genes comprise or consist of IFNAR1. In another embodiment, the IFN receptor family genes comprise or consist of IFNAR2. In other embodiments, the IFN receptor family genes comprise or consist of IFNAR1 and IFNAR2. In one specific embodiment, the IFN receptor family genes consist of IFNAR1. In another specific embodiment, the IFN receptor family genes comprise IFNAR1.

In various embodiments of the methods provided herein, including the methods provided in this Section (Section 5.2) such as the methods provided in this and the preceding 39 paragraphs, the one or more ADC Set I Marker genes comprise or consist of one or more inhibitory immunoreceptor genes. In some embodiments of the methods provided herein, including the methods provided in this Section (Section 5.2) such as the methods provided in this and the preceding 37 paragraphs, the inhibitory immunoreceptor genes comprise or consist of one or more genes selected from the group consisting of TIM3 and VSIR. In one embodiment, the inhibitory immunoreceptor genes comprise VSIR. In another embodiment, the inhibitory immunoreceptor genes consist of VSIR. In other embodiments, the inhibitory immunoreceptor genes comprise both TIM3 and VSIR. In yet another embodiment, the inhibitory immunoreceptor genes consist of both TIM3 and VSIR. In one specific embodiment, the inhibitory immunoreceptor genes consist of TIM3. In another specific embodiment, the inhibitory immunoreceptor genes comprise TIM3.

Additionally, in various embodiments of the methods provided herein, including the methods provided in this Section (Section 5.2) such as the methods provided in this and the preceding 40 paragraphs, the one or more ADC Set I Marker genes comprise or consist of one or more metabolic enzyme genes. In some embodiments of the methods provided herein, including the methods provided in this Section (Section 5.2) such as the methods provided in this and the preceding 38 paragraphs, the metabolic enzyme genes comprise or consist of one or more genes selected from the group consisting of indoleamine 2,3-dioxygenase 1 (IDO1), TDO2, EIF2AK2, ACSS1, and ACSS2. In one embodiment, the metabolic enzyme genes comprise or consist of IDO1. In some embodiments, the metabolic enzyme genes comprise or consist of TDO2. In certain embodiments, the metabolic enzyme genes comprise or consist of EIF2AK2. In other embodiments, the metabolic enzyme genes comprise or consist of ACSS1. In yet other embodiments, the metabolic enzyme genes comprise or consist of ACSS2. In one embodiment, the metabolic enzyme genes comprise or consist of IDO1 and TDO2. In some embodiments, the metabolic enzyme genes comprise or consist of IDO1 and EIF2AK2. In certain embodiments, the metabolic enzyme genes comprise or consist of IDO1 and ACSS1. In other embodiments, the metabolic enzyme genes comprise or consist of IDO1 and ACSS2. In further embodiments, the metabolic enzyme genes comprise or consist of TDO2 and EIF2AK2. In one embodiment, the metabolic enzyme genes comprise or consist of TDO2 and ACSS1. In some embodiments, the metabolic enzyme genes comprise or consist of TDO2 and ACSS2. In other embodiments, the metabolic enzyme genes comprise or consist of EIF2AK2 and ACSS1. In yet other embodiments, the metabolic enzyme genes comprise or consist of EIF2AK2 and ACSS2. In one embodiment, the metabolic enzyme genes comprise or consist of ACSS1 and ACSS2. In other embodiments, the metabolic enzyme genes comprise or consist of IDOL TDO2, and EIF2AK2. In yet other embodiments, the metabolic enzyme genes comprise or consist of IDO1, TDO2 and ACSS1. In further embodiments, the metabolic enzyme genes comprise or consist of IDO1, TDO2, and ACSS2. In some embodiments, the metabolic enzyme genes comprise or consist of IDO1, EIF2AK2 and ACSS1. In certain embodiments, the metabolic enzyme genes comprise or consist of IDOL EIF2AK2, and ACSS2. In yet other embodiments, the metabolic enzyme genes comprise or consist of IDO1, ACSS1 and ACSS2. In some embodiments, the metabolic enzyme genes comprise or consist of TDO2, EIF2AK2 and ACSS1. In certain embodiments, the metabolic enzyme genes comprise or consist of TDO2, EIF2AK2, and ACSS2. In yet other embodiments, the metabolic enzyme genes comprise or consist of TDO2, ACSS1 and ACSS2. In yet other embodiments, the metabolic enzyme genes comprise or consist of EIF2AK2, ACSS1 and ACSS2. In other embodiments, the metabolic enzyme genes comprise or consist of IDO1, TDO2, EIF2AK2, and ACSS1. In further embodiments, the metabolic enzyme genes comprise or consist of IDO1, TDO2, EIF2AK2, and ACSS2. In some embodiments, the metabolic enzyme genes comprise or consist of IDO1, TDO2, ACSS1, and ACSS2. In certain embodiments, the metabolic enzyme genes comprise or consist of IDOL EIF2AK2, ACSS1, and ACSS2. In further embodiments, the metabolic enzyme genes comprise or consist of TDO2, EIF2AK2, ACSS1, and ACSS2. In certain embodiments, the metabolic enzyme genes comprise or consist of IDOL TDO2, EIF2AK2, ACSS1, and ACSS2. In some embodiments, the metabolic enzyme genes comprise or consist of any one of IDO1, TDO2, EIF2AK2, ACSS1, and ACSS2. In some embodiments, the metabolic enzyme genes comprise or consist of any two of IDO1, TDO2, EIF2AK2, ACSS1, and ACSS2, in any combination or permutation. In some embodiments, the metabolic enzyme genes comprise or consist of any three of IDOL TDO2, EIF2AK2, ACSS1, and ACSS2, in any combination or permutation. In some embodiments, the metabolic enzyme genes comprise or consist of any four of IDO1, TDO2, EIF2AK2, ACSS1, and ACSS2, in any combination or permutation. In some embodiments, the metabolic enzyme genes comprise or consist of any five of IDOL TDO2, EIF2AK2, ACSS1, and ACSS2, in any combination or permutation. In one specific embodiment, the metabolic enzyme genes consist of IDO1. In another specific embodiment, the metabolic enzyme genes comprise IDO1.

In some embodiments of the methods provided herein, including the methods provided in this Section (Section 5.2) such as the methods provided in this and the preceding 39 paragraphs, the method further comprises determining an increase of the expression of one or more ADC Set II Marker genes in the subject compared to the expression of the one or more ADC Set II Marker genes in the subject before the administration of the ADC in step (1). In some embodiments of the methods provided herein, including the methods provided in this Section (Section 5.2) such as the methods provided in this and the preceding 39 paragraphs, the administration in step (3)(a) of the various methods is further conditioned on the increase of the expression of one or more ADC Set II Marker genes in the subject compared to the expression of the one or more ADC Set II Marker genes in the subject before the administration of the ADC in step (1).

As the various methods can be further conditioned on the increase of the expression of one or more ADC Set II Marker genes as described in the preceding paragraph, certain embodiments of the methods provided herein also include methods using various embodiments of the ADC Set II Marker genes. In some embodiments of the methods provided herein, including the methods provided in this Section (Section 5.2) such as the methods provided in this and the preceding paragraph, the one or more ADC Set II Marker genes comprise one or more genes selected from the group consisting of ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In one embodiment, the one or more ADC Set II Marker genes comprise one or more genes of any one type selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. For example, in some embodiments, the one or more ADC Set II Marker genes comprise one or more ER stress genes. In one embodiment, the one or more ADC Set II Marker genes comprise one or more ER/mitochondria ATPase genes. In one embodiment, the one or more ADC Set II Marker genes comprise one or more cell death genes. In one embodiment, the one or more ADC Set II Marker genes comprise one or more T cell stimulator genes. In one embodiment, the one or more ADC Set II Marker genes comprise one or more macrophage/innate immunity stimulator genes. In one embodiment, the one or more ADC Set II Marker genes comprise one or more chemoattractant genes. In one embodiment, the one or more ADC Set II Marker genes comprise one or more Rho GTPase genes. In one embodiment, the one or more ADC Set II Marker genes comprise one or more Rho GTPase regulator genes. In one embodiment, the one or more ADC Set II Marker genes comprise one or more mitotic arrest genes. In one embodiment, the one or more ADC Set II Marker genes comprise one or more siglec family genes. In one embodiment, or the one or more ADC Set II Marker genes comprise one or more GO positive autophagy regulator genes. In one embodiment, or the one or more ADC Set II Marker genes comprise one or more GTPase related kinase genes.

Similarly, in one embodiment, the one or more ADC Set II Marker genes comprise one or more genes of each of the two types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise one or more genes of each of the three types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In certain embodiments, the one or more ADC Set II Marker genes comprise one or more genes of each of the four types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise one or more genes of each of the five types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In yet other embodiments, the one or more ADC Set II Marker genes comprise one or more genes of each of the six types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some further embodiments, the one or more ADC Set II Marker genes comprise one or more genes of each of the seven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise one or more genes of each of the eight types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise one or more genes of each of the nine types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise one or more genes of each of the ten types selected from the group consisting of the following the eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise one or more genes of each of the eleven types selected from the group consisting of the following the eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise one or more genes of any one to eleven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise one or more genes of any one to eleven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes, and one or more genes of any other types of ADC Set II Marker genes.

Additionally, the one or more ADC Set II Marker genes comprise or consist of any one type selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. For example, in some embodiments, the one or more ADC Set II Marker genes comprise or consist of ER stress genes. In one embodiment, the one or more ADC Set II Marker genes comprise or consist of ER/mitochondria ATPase genes. In one embodiment, the one or more ADC Set II Marker genes comprise or consist of cell death genes. In one embodiment, the one or more ADC Set II Marker genes comprise or consist of T cell stimulator genes. In one embodiment, the one or more ADC Set II Marker genes comprise or consist of macrophage/innate immunity stimulator genes. In one embodiment, the one or more ADC Set II Marker genes comprise or consist of chemoattractant genes. In one embodiment, the one or more ADC Set II Marker genes comprise or consist of Rho GTPase genes. In one embodiment, the one or more ADC Set II Marker genes comprise or consist of Rho GTPase regulator genes. In one embodiment, the one or more ADC Set II Marker genes comprise or consist of mitotic arrest genes. In one embodiment, the one or more ADC Set II Marker genes comprise or consist of siglec family genes. In one embodiment, or the one or more ADC Set II Marker genes comprise or consist of GO positive autophagy regulator genes. In one embodiment, or the one or more ADC Set II Marker genes comprise or consist of GTPase related kinase genes.

Similarly, in one embodiment, the one or more ADC Set II Marker genes comprise or consist of two types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise or consist of three types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In certain embodiments, the one or more ADC Set II Marker genes comprise or consist of four types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise or consist of five types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In yet other embodiments, the one or more ADC Set II Marker genes comprise or consist of six types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some further embodiments, the one or more ADC Set II Marker genes comprise or consist of seven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise or consist of eight types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise or consist of nine types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise or consist of ten types selected from the group consisting of the following the eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In some embodiments, the one or more ADC Set II Marker genes comprise or consist of eleven types selected from the group consisting of the following the eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise or consist of any one to eleven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes. In other embodiments, the one or more ADC Set II Marker genes comprise or consist of any one to eleven types selected from the group consisting of the following eleven types of genes: ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes, and any other types of ADC Set II Marker genes.

Given the various embodiments of the methods involving the types of genes for ADC Set II Markers as described above and below, the disclosure provides further specific embodiments of for the ADC Set II Markers. In some embodiments of the methods provided herein, including the methods provided in this Section (Section 5.2) such as the methods provided in this and the preceding 4 paragraphs, the ER stress genes comprise or consist of one or more genes selected from the group consisting of XBP-1S, ERP29, TRAF2 and c-JUN. In one embodiment, the ER stress genes comprise or consist of XBP-1S. In another embodiment, the ER stress genes comprise or consist of ERP29. In some embodiment, the ER stress genes comprise or consist of TRAF2. In certain embodiment, the ER stress genes comprise or consist of c-JUN. In other embodiments, the ER stress genes comprise or consist of XBP-1S and ERP29. In yet other embodiments, the ER stress genes comprise or consist of XBP-1S and TRAF2. In further embodiments, the ER stress genes comprise or consist of XBP-1S and c-JUN. In one embodiment, the ER stress genes comprise or consist of ERP29 and TRAF2. In another embodiment, the ER stress genes comprise or consist of ERP29 and c-JUN. In some embodiment, the ER stress genes comprise or consist of TRAF2 and c-JUN. In certain embodiment, the ER stress genes comprise or consist of XBP-1S, ERP29, and TRAF2. In other embodiments, the ER stress genes comprise or consist of XBP-1S, ERP29, and c-JUN. In yet other embodiments, the ER stress genes comprise or consist of XBP-1S TRAF2, and c-JUN. In further embodiments, the ER stress genes comprise or consist of ERP29, TRAF2, and c-JUN. In some embodiments, the ER stress genes comprise or consist of XBP-1S, ERP29, TRAF2, and c-JUN. In one embodiment, the ER stress genes comprise or consist of any one of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK. In some embodiments, the ER stress genes comprise or consist of any two of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK. In some embodiments, the ER stress genes comprise or consist of any three of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any four of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any five of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any six of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any seven of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any eight of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any nine of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any ten of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any eleven of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any twelve of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any thirteen of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any fourteen of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any fifteen of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In some embodiments, the ER stress genes comprise or consist of any 1, 2, 3, 4, 5, 6, 7, 8, 9, 10, 11, 12, 13, 14, or 15 of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK, in any combination or permutation. In certain embodiments, the ER stress genes do not comprise or consist of XBP-1L. In some embodiment, the ER stress genes are not XBP-1L. In certain embodiments, the ER stress genes do not comprise or consist of EDEM2. In some embodiment, the ER stress genes are not EDEM2. In certain embodiments, the ER stress genes do not comprise or consist of EDEM2 or XBP-1L. In some embodiment, the ER stress genes are not EDEM2 or XBP-1L.

Similarly, in some embodiments of the methods provided herein, including the methods provided in this Section (Section 5.2) such as the methods provided in this and the preceding 5 paragraphs, the ER/mitochondria ATPase genes comprise or consist of one or more genes selected from the group consisting of ATP2A3, MT-ATP6, and MT-ATP8. In one embodiment, the ER/mitochondria ATPase genes comprise or consist of ATP2A3. In another embodiment, the ER/mitochondria ATPase genes comprise or consist of MT-ATP6. In some embodiment, the ER/mitochondria ATPase genes comprise or consist of MT-ATP8. In other embodiments, the ER/mitochondria ATPase genes comprise or consist of ATP2A3 and MT-ATP6. In yet other embodiments, the ER/mitochondria ATPase genes comprise or consist of ATP2A3 and MT-ATP8. In one embodiment, the ER/mitochondria ATPase genes comprise or consist of MT-ATP6 and MT-ATP8. In certain embodiment, the ER/mitochondria ATPase genes comprise or consist of ATP2A3, MT-ATP6, and MT-ATP8.

Continuing with the specific embodiments on specific ADC Set II Marker genes, in some embodiments of the methods provided herein, including the methods provided in this Section (Section 5.2) such as the methods provided in this and the preceding 6 paragraphs, the cell death genes comprise or consist of one or more genes selected from the group consisting of Bax, BCL2L1, BCL2L11, and BOK. In one embodiment, the cell death genes comprise or consist of BAX. In some embodiments, the cell death genes comprise or consist of BCL2L1. In certain embodiments, the cell death genes comprise or consist of BCL2L11. In other embodiments, the cell death genes comprise or consist of BOK. In one embodiment, the cell death genes comprise or consist of BAX and BCL2L1. In some embodiments, the cell death genes comprise or consist of BAX and BCL2L11. In certain embodiments, the cell death genes comprise or consist of BAX and BOK. In further embodiments, the cell death genes comprise or consist of BCL2L1 and BCL2L11. In one embodiment, the cell death genes comprise or consist of BCL2L1 and BOK. In other embodiments, the cell death genes comprise or consist of BCL2L11 and BOK. In yet other embodiments, the cell death genes comprise or consist of BAX, BCL2L1, and BCL2L11. In some embodiments, the cell death genes comprise or consist of BAX, BCL2L1 and BOK. In certain embodiments, the cell death genes comprise or consist of BAX, BCL2L11 and BOK. In some embodiments, the cell death genes comprise or consist of BCL2L1, BCL2L11 and BOK. In other embodiments, the cell death genes comprise or consist of BAX, BCL2L1, BCL2L11, and BOK. In some embodiments, the cell death genes comprise or consist of any one of BAX, BCL2L1, BCL2L11, and BOK. In some embodiments, the cell death genes comprise or consist of any two of BAX, BCL2L1, BCL2L11, and BOK, in any combination or permutation. In some embodiments, the cell death genes comprise or consist of any three of BAX, BCL2L1, BCL2L11, and BOK, in any combination or permutation. In some embodiments, the cell death genes comprise or consist of any four of BAX, BCL2L1, BCL2L11, and BOK, in any permutation. In certain embodiments, the cell death genes do not comprise or consist of FAS. In some embodiment, the cell death genes are not FAS.

Further continuing with the specific embodiments on specific ADC Set II Marker genes, in some embodiments of the methods provided herein, including the methods provided in this Section (Section 5.2) such as the methods provided in this and the preceding 7 paragraphs, the T cell stimulator genes comprise or consist of one or more genes selected from the group consisting of MIG (CXCL9) and IP10 (CXCL10). In one embodiment, the T cell stimulator genes comprise or consist of MIG (CXCL9). In another embodiment, the T cell stimulator genes comprise or consist of IP10 (CXCL10). In other embodiments, the T cell stimulator genes comprise or consist of MIG (CXCL9) and IP10 (CXCL10).

Similarly, in some embodiments of the methods provided herein, including the methods provided in this Section (Section 5.2) such as the methods provided in this and the preceding 8 paragraphs, the Macrophage/innate immunity stimulator genes comprise or consist of one or more genes selected from the group consisting of IL-1α and M-CSF (CSF). In one embodiment, the Macrophage/innate immunity stimulator genes comprise or consist of IL-1α. In another embodiment, the Macrophage/innate immunity stimulator genes comprise or consist of M-CSF (CSF). In other embodiments, the Macrophage/innate immunity stimulator genes comprise or consist of IL-1α and M-CSF (CSF).

Continuing with the specific embodiments on specific ADC Set II Marker genes, in some embodiments of the methods provided herein, including the methods provided in this Section (Section 5.2) such as the methods provided in this and the preceding 9 paragraphs, the chemoattractant genes comprise or consist of one or more genes selected from the group consisting of Eotaxin (CCL11), MIP1α, MIP1β and MCP1. In one embodiment, the chemoattractant genes comprise or consist of Eotaxin (CCL11). In another embodiment, the chemoattractant genes comprise or consist of MIP1α. In some embodiment, the chemoattractant genes comprise or consist of MIP1β. In certain embodiment, the chemoattractant genes comprise or consist of MCP1. In other embodiments, the chemoattractant genes comprise or consist of Eotaxin (CCL11) and MIP1a. In yet other embodiments, the chemoattractant genes comprise or consist of Eotaxin (CCL11) and MIP1β. In further embodiments, the chemoattractant genes comprise or consist of Eotaxin (CCL11) and MCP1. In one embodiment, the chemoattractant genes comprise or consist of MIP1α and MIP1β. In another embodiment, the chemoattractant genes comprise or consist of MIP1α and MCP1. In some embodiment, the chemoattractant genes comprise or consist of MIP10 and MCP1. In certain embodiment, the chemoattractant genes comprise or consist of Eotaxin (CCL11), MIP1a, and MIP113. In other embodiments, the chemoattractant genes comprise or consist of Eotaxin (CCL11), MIP1a, and MCP1. In yet other embodiments, the chemoattractant genes comprise or consist of Eotaxin (CCL11) MIP1β, and MCP1. In further embodiments, the chemoattractant genes comprise or consist of MIP1α, MIP1β, and MCP1. In some embodiments, the chemoattractant genes comprise or consist of Eotaxin (CCL11), MIP1α, MIP1β, and MCP1.

Similarly, in some embodiments of the methods provided herein, including the methods provided in this Section (Section 5.2) such as the methods provided in this and the preceding 10 paragraphs, the Rho GTPase genes comprise or consist of one or more genes selected from the group consisting of RhoB, RhoF, and RhoG. In one embodiment, the Rho GTPase genes comprise or consist of RhoB. In another embodiment, the Rho GTPase genes comprise or consist of RhoF. In some embodiment, the Rho GTPase genes comprise or consist of RhoG. In other embodiments, the Rho GTPase genes comprise or consist of RhoB and RhoF. In yet other embodiments, the Rho GTPase genes comprise or consist of RhoB and RhoG. In one embodiment, the Rho GTPase genes comprise or consist of RhoF and RhoG. In certain embodiment, the Rho GTPase genes comprise or consist of RhoB, RhoF, and RhoG. In certain embodiments, the Rho GTPase genes do not comprise or consist of any one, two, or three genes selected from the group consisting of CDC42, RhoA, and RhoC. In some embodiment, the ER stress genes are not any one, two, three genes selected from the group consisting of CDC42, RhoA, and RhoC. In certain embodiments, the Rho GTPase genes do not comprise any one of CDC42, RhoA, and RhoC.

Continuing with the specific embodiments on specific ADC Set II Marker genes, in some embodiments of the methods provided herein, including the methods provided in this Section (Section 5.2) such as the methods provided in this and the preceding 11 paragraphs, the Rho GTPase regulator genes comprise or consist of one or more genes selected from the group consisting of DAP2IP, ARHGEF18, ARHGEF5, and RASAL1. In one embodiment, the Rho GTPase regulator genes comprise or consist of DAP2IP. In another embodiment, the Rho GTPase regulator genes comprise or consist of ARHGEF18. In some embodiment, the Rho GTPase regulator genes comprise or consist of ARHGEF5. In certain embodiment, the Rho GTPase regulator genes comprise or consist of RASAL1. In other embodiments, the Rho GTPase regulator genes comprise or consist of DAP2IP and ARHGEF18. In yet other embodiments, the Rho GTPase regulator genes comprise or consist of DAP2IP and ARHGEF5. In further embodiments, the Rho GTPase regulator genes comprise or consist of DAP2IP and RASAL1. In one embodiment, the Rho GTPase regulator genes comprise or consist of ARHGEF18 and ARHGEF5. In another embodiment, the Rho GTPase regulator genes comprise or consist of ARHGEF18 and RASAL1. In some embodiment, the Rho GTPase regulator genes comprise or consist of ARHGEF5 and RASAL1. In certain embodiment, the Rho GTPase regulator genes comprise or consist of DAP2IP, ARHGEF18, and ARHGEF5. In other embodiments, the Rho GTPase regulator genes comprise or consist of DAP2IP, ARHGEF18, and RASAL1. In yet other embodiments, the Rho GTPase regulator genes comprise or consist of DAP2IP ARHGEF5, and RASAL1. In further embodiments, the Rho GTPase regulator genes comprise or consist of ARHGEF18, ARHGEF5, and RASAL1. In some embodiments, the Rho GTPase regulator genes comprise or consist of DAP2IP, ARHGEF18, ARHGEF5, and RASAL1.

Still continuing with the specific embodiments on specific ADC Set II Marker genes, in some embodiments of the methods provided herein, including the methods provided in this Section (Section 5.2) such as the methods provided in this and the preceding 12 paragraphs, the GTPase related genes comprise or consist of ROCK1. In one embodiment, the GTPase related genes comprise or consist of PAK4. In another embodiment, the GTPase related genes comprise or consist of both ROCK1 and PAK4.

Further continuing with the specific embodiments on specific ADC Set II Marker genes, in some embodiments of the methods provided herein, including the methods provided in this Section (Section 5.2) such as the methods provided in this and the preceding 13 paragraphs, the mitotic arrest genes comprise or consist of one or more genes selected from the group consisting of CCND1, CDKN1A, GADD45B, E4F1, CDC14B, and DAPK1. In one embodiment, the mitotic arrest genes comprise or consist of CCND1. In some embodiments, the mitotic arrest genes comprise or consist of CDKN1A. In certain embodiments, the mitotic arrest genes comprise or consist of GADD45B. In other embodiments, the mitotic arrest genes comprise or consist of E4F1. In yet other embodiments, the mitotic arrest genes comprise or consist of CDC14B. In another embodiment, the mitotic arrest genes comprise or consist of DAPK1. In one embodiment, the mitotic arrest genes comprise or consist of CCND1 and CDKN1A. In some embodiments, the mitotic arrest genes comprise or consist of CCND1 and GADD45B. In certain embodiments, the mitotic arrest genes comprise or consist of CCND1 and E4F1. In other embodiments, the mitotic arrest genes comprise or consist of CCND1 and CDC14B. In some embodiments, the mitotic arrest genes comprise or consist of CCND1 and DAPK1. In further embodiments, the mitotic arrest genes comprise or consist of CDKN1A and GADD45B. In one embodiment, the mitotic arrest genes comprise or consist of CDKN1A and E4F1. In some embodiments, the mitotic arrest genes comprise or consist of CDKN1A and CDC14B. In certain embodiments, the mitotic arrest genes comprise or consist of CDKN1A and DAPK1. In other embodiments, the mitotic arrest genes comprise or consist of GADD45B and E4F1. In yet other embodiments, the mitotic arrest genes comprise or consist of GADD45B and CDC14B. In some embodiments, the mitotic arrest genes comprise or consist of GADD45B and DAPK1. In one embodiment, the mitotic arrest genes comprise or consist of E4F1 and CDC14B. In another embodiment, the mitotic arrest genes comprise or consist of E4F1 and DAPK1. In another embodiment, the mitotic arrest genes comprise or consist of CDC14B and DAPK1. In other embodiments, the mitotic arrest genes comprise or consist of CCND1, CDKN1A, and GADD45B. In yet other embodiments, the mitotic arrest genes comprise or consist of CCND1, CDKN1A and E4F1. In further embodiments, the mitotic arrest genes comprise or consist of CCND1, CDKN1A, and CDC14B. In some embodiments, the mitotic arrest genes comprise or consist of CCND1, CDKN1A, and DAPK1. In some embodiments, the mitotic arrest genes comprise or consist of CCND1, GADD45B and E4F1. In certain embodiments, the mitotic arrest genes comprise or consist of CCND1, GADD45B, and CDC14B. In some embodiments, the mitotic arrest genes comprise or consist of CCND1, GADD45B, and DAPK1. In yet other embodiments, the mitotic arrest genes comprise or consist of CCND1, E4F1 and CDC14B. In some embodiments, the mitotic arrest genes comprise or consist of CCND1, E4F1 and DAPK1. In some embodiments, the mitotic arrest genes comprise or consist of CCND1, CDC14B and DAPK1. In some embodiments, the mitotic arrest genes comprise or consist of CDKN1A, GADD45B and E4F1. In certain embodiments, the mitotic arrest genes comprise or consist of CDKN1A, GADD45B, and CDC14B. In some embodiments, the mitotic arrest genes comprise or consist of CDKN1A, GADD45B, and DAPK1. In other embodiments, the mitotic arrest genes comprise or consist of CDKN1A, E4F1 and CDC14B. In yet other embodiments, the mitotic arrest genes comprise or consist of CDKN1A, E4F1 and DAPK1. In some embodiments, the mitotic arrest genes comprise or consist of CDKN1A, CDC14B and DAPK1. In yet other embodiments, the mitotic arrest genes comprise or consist of GADD45B, E4F1 and CDC14B. In other embodiments, the mitotic arrest genes comprise or consist of GADD45B, E4F1 and DAPK1. In certain embodiments, the mitotic arrest genes comprise or consist of GADD45B, CDC14B and DAPK1. In some embodiments, the mitotic arrest genes comprise or consist of E4F1, CDC14B and DAPK1. In other embodiments, the mitotic arrest genes comprise or consist of CCND1, CDKN1A, GADD45B, and E4F1. In further embodiments, the mitotic arrest genes comprise or consist of CCND1, CDKN1A, GADD45B, and CDC14B. In some embodiments, the mitotic arrest genes comprise or consist of CCND1, CDKN1A, GADD45B, and DAPK1. In some embodiments, the mitotic arrest genes comprise or consist of CCND1, CDKN1A, E4F1, and CDC14B. In some embodiments, the mitotic arrest genes comprise or consist of CCND1, CDKN1A, E4F1, and DAPK1. In some embodiments, the mitotic arrest genes comprise or consist of CCND1, CDKN1A, CDC14B, and DAPK1. In certain embodiments, the mitotic arrest genes comprise or consist of CCND1, GADD45B, E4F1, and CDC14B. In certain embodiments, the mitotic arrest genes comprise or consist of CCND1, GADD45B, E4F1, and DAPK1. In certain embodiments, the mitotic arrest genes comprise or consist of CCND1, GADD45B, CDC14B, and DAPK1. In yet other embodiments, the mitotic arrest genes comprise or consist of CCND1, E4F1, CDC14B, and DAPK1. In further embodiments, the mitotic arrest genes comprise or consist of CDKN1A, GADD45B, E4F1, and CDC14B. In some embodiments, the mitotic arrest genes comprise or consist of CDKN1A, GADD45B, E4F1, and DAPK1. In further embodiments, the mitotic arrest genes comprise or consist of CDKN1A, GADD45B, CDC14B, and DAPK1. In some embodiments, the mitotic arrest genes comprise or consist of CDKN1A, E4F1, CDC14B, and DAPK1. In some embodiments, the mitotic arrest genes comprise or consist of GADD45B, E4F1, CDC14B, and DAPK1. In certain embodiments, the mitotic arrest genes comprise or consist of CCND1, CDKN1A, GADD45B, E4F1, and CDC14B. In some embodiments, the mitotic arrest genes comprise or consist of CCND1, CDKN1A, GADD45B, E4F1, and DAPK1. In some embodiments, the mitotic arrest genes comprise or consist of CCND1, CDKN1A, GADD45B, CDC14B, and DAPK1. In certain embodiments, the mitotic arrest genes comprise or consist of CCND1, CDKN1A, E4F1, CDC14B, and DAPK1. In certain embodiments, the mitotic arrest genes comprise or consist of CCND1, GADD45B, E4F1, CDC14B, and DAPK1. In some embodiments, the mitotic arrest genes comprise or consist of CDKN1A, GADD45B, E4F1, CDC14B, and DAPK1. In some embodiments, the mitotic arrest genes comprise or consist of CCND1, CDKN1A, GADD45B, E4F1, CDC14B, and DAPK1. In some embodiments, the mitotic arrest genes comprise or consist of any one of CCND1, CDKN1A, GADD45B, E4F1, CDC14B, and DAPK1. In some embodiments, the mitotic arrest genes comprise or consist of any two of CCND1, CDKN1A, GADD45B, E4F1, CDC14B, and DAPK1, in any combination or permutation. In some embodiments, the mitotic arrest genes comprise or consist of any three of CCND1, CDKN1A, GADD45B, E4F1, CDC14B, and DAPK1, in any combination or permutation. In some embodiments, the mitotic arrest genes comprise or consist of any four of CCND1, CDKN1A, GADD45B, E4F1, CDC14B, and DAPK1, in any combination or permutation. In some embodiments, the mitotic arrest genes comprise or consist of any five of CCND1, CDKN1A, GADD45B, E4F1, CDC14B, and DAPK1, in any combination or permutation. In some embodiments, the mitotic arrest genes comprise or consist of any six of CCND1, CDKN1A, GADD45B, E4F1, CDC14B, and DAPK1, in any combination or permutation. In certain embodiments, the mitotic arrest genes do not comprise or consist of DDIAS. In some embodiment, the mitotic arrest genes are not DDIAS. In certain embodiments, the mitotic arrest genes do not comprise or consist of CDK1. In some embodiment, the mitotic arrest genes are not CDK1. In certain embodiments, the mitotic arrest genes do not comprise or consist of CDK1 or DDIAS. In some embodiment, the mitotic arrest genes are not CDK1 or DDIAS.

Continuing with the specific embodiments on specific ADC Set II Marker genes, in some embodiments of the methods provided herein, including the methods provided in this Section (Section 5.2) such as the methods provided in this and the preceding 14 paragraphs, the siglec family genes comprise or consist of siglec1.

Further continuing with the specific embodiments on specific ADC Set II Marker genes, in some embodiments of the methods provided herein, including the methods provided in this Section (Section 5.2) such as the methods provided in this and the preceding 15 paragraphs, the GO positive autophagy regulator genes comprise or consist of one or more genes selected from the group consisting of BCL2L11, ROCK1, TSC1, TSC2, BAG3, MFN2, RIPK1, RIPK4, HDAC6, STK11, ULK1, FOXO1, FOXO3, and MUL1. In certain embodiments, the GO positive autophagy regulator genes comprise or consist of 1, 2, 3, 4, 5, 6, 7, 8, 9, 10, 11, 12, 13, or 14 genes selected from the group consisting of BCL2L11, ROCK1, TSC1, TSC2, BAG3, MFN2, RIPK1, RIPK4, HDAC6, STK11, ULK1, FOXO1, FOXO3, and MULL In certain embodiments, the GO positive autophagy regulator genes comprise or consist of any 1 gene selected from the group consisting of BCL2L11, ROCK1, TSC1, TSC2, BAG3, MFN2, RIPK1, RIPK4, HDAC6, STK11, ULK1, FOXO1, FOXO3, and MULL In certain embodiments, the GO positive autophagy regulator genes comprise or consist of any 2 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 3 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 4 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 5 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 6 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 7 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 8 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 9 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 10 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 11 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 12 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 13 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes comprise or consist of any 14 genes selected from the group consisting of BCL2L11, ROCK1, TSC1, TSC2, BAG3, MFN2, RIPK1, RIPK4, HDAC6, STK11, ULK1, FOXO1, FOXO3, and MUL1, in any permutation or combination. In certain embodiments, the GO positive autophagy regulator genes do not comprise or consist of BNIP3. In some embodiment, the GO positive autophagy regulator genes are not BNIP3. In certain embodiments, the GO positive autophagy regulator genes do not comprise or consist of BNIP3L. In some embodiment, the GO positive autophagy regulator genes are not BNIP3L. In certain embodiments, the GO positive autophagy regulator genes do not comprise or consist of BNIP3L or BNIP3. In some embodiment, the GO positive autophagy regulator genes are not BNIP3L or BNIP3.

Still further continuing with the specific embodiments on specific ADC Set II Marker genes, in some embodiments of the methods provided herein, including the methods provided in this Section (Section 5.2) such as the methods provided in this and the preceding 17 paragraphs, the one or more ADC Set II Marker genes comprise or consist of one or more genes selected from the group consisting of XBP-1S, ERP29, TRAF2, c-JUN, BCL2L11, BCAP31, SERINC3, DAP2IP, ERN1, ATF6, NCK2, PPP1R15A, UBQLN2, BAG6, BOK, ATP2A3, MT-ATP6, MT-ATP8, Bax, BCL2L1, MIG (CXCL9), IP10 (CXCL10), IL-1α, M-CSF (CSF), Eotaxin (CCL11), MIP1α, MIP1β, MCP1, RhoB, RhoF, RhoG, DAP2IP, ARHGEF18, ARHGEF5, RASAL1, CCND1, CDKN1A, GADD45B, E4F1, CDC14B, DAPK1, siglec1, BCL2L11, ROCK1, TSC1, TSC2, BAG3, MFN2, RIPK1, RIPK4, HDAC6, STK11, ULK1, FOXO1, FOXO3, and MULL In some embodiments, the one or more ADC Set II Marker genes comprise or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 genes selected from the group consisting of XBP-1S, ERP29, TRAF2, c-JUN, BCL2L11, BCAP31, SERINC3, DAP2IP, ERN1, ATF6, NCK2, PPP1R15A, UBQLN2, BAG6, BOK, ATP2A3, MT-ATP6, MT-ATP8, Bax, BCL2L1, MIG (CXCL9), IP10 (CXCL10), IL-1α, M-CSF (CSF), Eotaxin (CCL11), MIP1α, MIP1β, MCP1, RhoB, RhoF, RhoG, DAP2IP, ARHGEF18, ARHGEF5, RASAL1, CCND1, CDKN1A, GADD45B, E4F1, CDC14B, DAPK1, siglec1, BCL2L11, ROCK1, TSC1, TSC2, BAG3, MFN2, RIPK1, RIPK4, HDAC6, STK11, ULK1, FOXO1, FOXO3, and MUL1. In certain embodiments, the one or more ADC Set II Marker genes do not comprise any one or more genes selected from the group consisting of XBP-1L, EDEM2, FAS, CDC42, RhoA, RhoC, DDIAS, CDK1, BNIP3, and BNIP3L. In some embodiments, the one or more ADC Set II Marker genes do not comprise any 1, 2, 3, 4, 5, 6, 7, 8, 9, or 10 genes selected from the group consisting of XBP-1L, EDEM2, FAS, CDC42, RhoA, RhoC, DDIAS, CDK1, BNIP3, and BNIP3L.

Additionally, in some embodiments of the methods provided herein, including the methods provided in this Section (Section 5.2) such as the methods provided in this and the preceding 18 paragraphs, the one or more ADC Set II Marker genes comprise or consist of one or more genes selected from genes whose expression changes in response to MMAE or general class of auristatins as disclosed in WO2019183438 or US20190290775A1, which is herein incorporated in its entirety by reference. In other embodiments, the one or more ADC Set II Marker genes comprise or consist of one or more genes selected from genes whose expression changes in response to MMAE or general class of auristatins as disclosed in WO2019183438 or US20190290775A1, in any permutation or combination of the ADC Set II Marker genes provided herein.

In various embodiments of the methods provided herein, including the methods provided in this Section (Section 5.2) such as the methods provided in this and the preceding 60 paragraphs, the increase in any of the gene expression is an increase of 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, or more. In some embodiments, the increase in any of the gene expression is a 10% increase. In some embodiments, the increase in any of the gene expression is a 20% increase. In some embodiments, the increase in any of the gene expression is a 30% increase. In some embodiments, the increase in any of the gene expression is a 40% increase. In some embodiments, the increase in any of the gene expression is a 50% increase. In some embodiments, the increase in any of the gene expression is a 60% increase. In some embodiments, the increase in any of the gene expression is a 70% increase. In some embodiments, the increase in any of the gene expression is an 80% increase. In some embodiments, the increase in any of the gene expression is a 90% increase. In some embodiments, the increase in any of the gene expression is a 100% increase. In some embodiments, the increase in any of the gene expression is a 110% increase. In some embodiments, the increase in any of the gene expression is a 120% increase. In some embodiments, the increase in any of the gene expression is a 130% increase. In some embodiments, the increase in any of the gene expression is a 140% increase. In some embodiments, the increase in any of the gene expression is a 150% increase. In some embodiments, the increase in any of the gene expression is a 160% increase. In some embodiments, the increase in any of the gene expression is a 170% increase. In some embodiments, the increase in any of the gene expression is a 180% increase. In some embodiments, the increase in any of the gene expression is a 190% increase. In some embodiments, the increase in any of the gene expression is a 200% increase. In some embodiments, the increase in any of the gene expression is a 250% increase. In some embodiments, the increase in any of the gene expression is a 300% increase. In some embodiments, the increase in any of the gene expression is a 350% increase. In some embodiments, the increase in any of the gene expression is a 400% increase. In some embodiments, the increase in any of the gene expression is a 450% increase. In some embodiments, the increase in any of the gene expression is a 500% increase. In some embodiments, the increase in any of the gene expression is a 600% increase. In some embodiments, the increase in any of the gene expression is a 650% increase. In some embodiments, the increase in any of the gene expression is a 700% increase. In some embodiments, the increase in any of the gene expression is a 750% increase. In some embodiments, the increase in any of the gene expression is an 800% increase. In some embodiments, the increase in any of the gene expression is an 850% increase. In some embodiments, the increase in any of the gene expression is a 900% increase. In some embodiments, the increase in any of the gene expression is a 950% increase. In some embodiments, the increase in any of the gene expression is a 1000% increase. In some embodiments, the increase in any of the gene expression is an increase of at least 10%. In some embodiments, the increase in any of the gene expression is an increase of at least 20%. In some embodiments, the increase in any of the gene expression is an increase of at least 30%. In some embodiments, the increase in any of the gene expression is an increase of at least 40%. In some embodiments, the increase in any of the gene expression is an increase of at least 50%. In some embodiments, the increase in any of the gene expression is an increase of at least 60%. In some embodiments, the increase in any of the gene expression is an increase of at least 70%. In some embodiments, the increase in any of the gene expression is an increase of at least 80%. In some embodiments, the increase in any of the gene expression is an increase of at least 90%. In some embodiments, the increase in any of the gene expression is an increase of at least 100%. In some embodiments, the increase in any of the gene expression is an increase of at least 110%. In some embodiments, the increase in any of the gene expression is an increase of at least 120%. In some embodiments, the increase in any of the gene expression is an increase of at least 130%. In some embodiments, the increase in any of the gene expression is an increase of at least 140%. In some embodiments, the increase in any of the gene expression is an increase of at least 150%. In some embodiments, the increase in any of the gene expression is an increase of at least 160%. In some embodiments, the increase in any of the gene expression is an increase of at least 170%. In some embodiments, the increase in any of the gene expression is an increase of at least 180%. In some embodiments, the increase in any of the gene expression is an increase of at least 190%. In some embodiments, the increase in any of the gene expression is an increase of at least 210%. In some embodiments, the increase in any of the gene expression is an increase of at least 250%. In some embodiments, the increase in any of the gene expression is an increase of at least 300%. In some embodiments, the increase in any of the gene expression is an increase of at least 350%. In some embodiments, the increase in any of the gene expression is an increase of at least 400%. In some embodiments, the increase in any of the gene expression is an increase of at least 450%. In some embodiments, the increase in any of the gene expression is an increase of at least 500%. In some embodiments, the increase in any of the gene expression is an increase of at least 550%. In some embodiments, the increase in any of the gene expression is an increase of at least 600%. In some embodiments, the increase in any of the gene expression is an increase of at least 650%. In some embodiments, the increase in any of the gene expression is an increase of at least 700%. In some embodiments, the increase in any of the gene expression is an increase of at least 750%. In some embodiments, the increase in any of the gene expression is an increase of at least 800%. In some embodiments, the increase in any of the gene expression is an increase of at least 850%. In some embodiments, the increase in any of the gene expression is an increase of at least 900%. In some embodiments, the increase in any of the gene expression is an increase of at least 950%. In some embodiments, the increase in any of the gene expression is an increase of at least 1000%. In some embodiments, the increase in any of the gene expression is an increase of 10% to 1000%. In some embodiments, the increase in any of the gene expression is an increase of 20% to 1000%. In some embodiments, the increase in any of the gene expression is an increase of 30% to 1000%. In some embodiments, the increase in any of the gene expression is an increase of 40% to 1000%. In some embodiments, the increase in any of the gene expression is an increase of 50% to 1000%. In some embodiments, the increase in any of the gene expression is an increase of 60% to 1000%. In some embodiments, the increase in any of the gene expression is an increase of 70% to 1000%. In some embodiments, the increase in any of the gene expression is an increase of 80% to 1000%. In some embodiments, the increase in any of the gene expression is an increase of 90% to 1000%. In some embodiments, the increase in any of the gene expression is an increase of 100% to 1000%. In some embodiments, the increase in any of the gene expression is an increase of 110% to 1000%. In some embodiments, the increase in any of the gene expression is an increase of 120% to 1000%. In some embodiments, the increase in any of the gene expression is an increase of 130% to 1000%. In some embodiments, the increase in any of the gene expression is an increase of 140% to 1000%. In some embodiments, the increase in any of the gene expression is an increase of 150% to 1000%. In some embodiments, the increase in any of the gene expression is an increase of 160% to 1000%. In some embodiments, the increase in any of the gene expression is an increase of 170% to 1000%. In some embodiments, the increase in any of the gene expression is an increase of 180% to 1000%. In some embodiments, the increase in any of the gene expression is an increase of 190% to 1000%. In some embodiments, the increase in any of the gene expression is an increase of 210% to 1000%. In some embodiments, the increase in any of the gene expression is an increase of 250% to 1000%. In some embodiments, the increase in any of the gene expression is an increase of 300% to 1000%. In some embodiments, the increase in any of the gene expression is an increase of 350% to 1000%. In some embodiments, the increase in any of the gene expression is an increase of 400% to 1000%. In some embodiments, the increase in any of the gene expression is an increase of 450% to 1000%. In some embodiments, the increase in any of the gene expression is an increase of 500% to 1000%. In some embodiments, the increase in any of the gene expression is an increase of 10% to 800%. In some embodiments, the increase in any of the gene expression is an increase of 20% to 800%. In some embodiments, the increase in any of the gene expression is an increase of 30% to 800%. In some embodiments, the increase in any of the gene expression is an increase of 40% to 800%. In some embodiments, the increase in any of the gene expression is an increase of 50% to 800%. In some embodiments, the increase in any of the gene expression is an increase of 60% to 800%. In some embodiments, the increase in any of the gene expression is an increase of 70% to 800%. In some embodiments, the increase in any of the gene expression is an increase of 80% to 800%. In some embodiments, the increase in any of the gene expression is an increase of 90% to 800%. In some embodiments, the increase in any of the gene expression is an increase of 100% to 800%. In some embodiments, the increase in any of the gene expression is an increase of 110% to 800%. In some embodiments, the increase in any of the gene expression is an increase of 120% to 800%. In some embodiments, the increase in any of the gene expression is an increase of 130% to 800%. In some embodiments, the increase in any of the gene expression is an increase of 140% to 800%. In some embodiments, the increase in any of the gene expression is an increase of 150% to 800%. In some embodiments, the increase in any of the gene expression is an increase of 160% to 800%. In some embodiments, the increase in any of the gene expression is an increase of 170% to 800%. In some embodiments, the increase in any of the gene expression is an increase of 180% to 800%. In some embodiments, the increase in any of the gene expression is an increase of 190% to 800%. In some embodiments, the increase in any of the gene expression is an increase of 210% to 800%. In some embodiments, the increase in any of the gene expression is an increase of 250% to 800%. In some embodiments, the increase in any of the gene expression is an increase of 300% to 800%. In some embodiments, the increase in any of the gene expression is an increase of 350% to 800%. In some embodiments, the increase in any of the gene expression is an increase of 400% to 800%. In some embodiments, the increase in any of the gene expression is an increase of 450% to 800%. In some embodiments, the increase in any of the gene expression is an increase of 500% to 800%. In some embodiments, the increase in any of the gene expression is an increase of 10% to 500%. In some embodiments, the increase in any of the gene expression is an increase of 20% to 500%. In some embodiments, the increase in any of the gene expression is an increase of 30% to 500%. In some embodiments, the increase in any of the gene expression is an increase of 40% to 500%. In some embodiments, the increase in any of the gene expression is an increase of 50% to 500%. In some embodiments, the increase in any of the gene expression is an increase of 60% to 500%. In some embodiments, the increase in any of the gene expression is an increase of 70% to 500%. In some embodiments, the increase in any of the gene expression is an increase of 80% to 500%. In some embodiments, the increase in any of the gene expression is an increase of 90% to 500%. In some embodiments, the increase in any of the gene expression is an increase of 100% to 500%. In some embodiments, the increase in any of the gene expression is an increase of 110% to 500%. In some embodiments, the increase in any of the gene expression is an increase of 120% to 500%. In some embodiments, the increase in any of the gene expression is an increase of 130% to 500%. In some embodiments, the increase in any of the gene expression is an increase of 140% to 500%. In some embodiments, the increase in any of the gene expression is an increase of 150% to 500%. In some embodiments, the increase in any of the gene expression is an increase of 160% to 500%. In some embodiments, the increase in any of the gene expression is an increase of 170% to 500%. In some embodiments, the increase in any of the gene expression is an increase of 180% to 500%. In some embodiments, the increase in any of the gene expression is an increase of 190% to 500%. In some embodiments, the increase in any of the gene expression is an increase of 210% to 500%. In some embodiments, the increase in any of the gene expression is an increase of 250% to 500%. In some embodiments, the increase in any of the gene expression is an increase of 300% to 500%. In some embodiments, the increase in any of the gene expression is an increase of 350% to 500%. In some embodiments, the increase in any of the gene expression is an increase of 400% to 500%. In some embodiments, the increase in any of the gene expression is an increase of 450% to 500%. In some embodiments, the increase in any of the gene expression is an increase of 10% to 300%. In some embodiments, the increase in any of the gene expression is an increase of 20% to 300%. In some embodiments, the increase in any of the gene expression is an increase of 30% to 300%. In some embodiments, the increase in any of the gene expression is an increase of 40% to 300%. In some embodiments, the increase in any of the gene expression is an increase of 50% to 300%. In some embodiments, the increase in any of the gene expression is an increase of 60% to 300%. In some embodiments, the increase in any of the gene expression is an increase of 70% to 300%. In some embodiments, the increase in any of the gene expression is an increase of 80% to 300%. In some embodiments, the increase in any of the gene expression is an increase of 90% to 300%. In some embodiments, the increase in any of the gene expression is an increase of 100% to 300%. In some embodiments, the increase in any of the gene expression is an increase of 110% to 300%. In some embodiments, the increase in any of the gene expression is an increase of 120% to 300%. In some embodiments, the increase in any of the gene expression is an increase of 130% to 300%. In some embodiments, the increase in any of the gene expression is an increase of 140% to 300%. In some embodiments, the increase in any of the gene expression is an increase of 150% to 300%. In some embodiments, the increase in any of the gene expression is an increase of 160% to 300%. In some embodiments, the increase in any of the gene expression is an increase of 170% to 300%. In some embodiments, the increase in any of the gene expression is an increase of 180% to 300%. In some embodiments, the increase in any of the gene expression is an increase of 190% to 300%. In some embodiments, the increase in any of the gene expression is an increase of 210% to 300%. In some embodiments, the increase in any of the gene expression is an increase of 250% to 300%. In some embodiments, the increase in any of the gene expression is an increase of 10% to 200%. In some embodiments, the increase in any of the gene expression is an increase of 20% to 200%. In some embodiments, the increase in any of the gene expression is an increase of 30% to 200%. In some embodiments, the increase in any of the gene expression is an increase of 40% to 200%. In some embodiments, the increase in any of the gene expression is an increase of 50% to 200%. In some embodiments, the increase in any of the gene expression is an increase of 60% to 200%. In some embodiments, the increase in any of the gene expression is an increase of 70% to 200%. In some embodiments, the increase in any of the gene expression is an increase of 80% to 200%. In some embodiments, the increase in any of the gene expression is an increase of 90% to 200%. In some embodiments, the increase in any of the gene expression is an increase of 100% to 200%. In some embodiments, the increase in any of the gene expression is an increase of 110% to 200%. In some embodiments, the increase in any of the gene expression is an increase of 120% to 200%. In some embodiments, the increase in any of the gene expression is an increase of 130% to 200%. In some embodiments, the increase in any of the gene expression is an increase of 140% to 200%. In some embodiments, the increase in any of the gene expression is an increase of 150% to 200%. In some embodiments, the increase in any of the gene expression is an increase of 160% to 200%. In some embodiments, the increase in any of the gene expression is an increase of 170% to 200%. In some embodiments, the increase in any of the gene expression is an increase of 180% to 200%. In some embodiments, the increase in any of the gene expression is an increase of 190% to 200%. In some embodiments, the increase in any of the gene expression is an increase of 10% to 100%. In some embodiments, the increase in any of the gene expression is an increase of 20% to 100%. In some embodiments, the increase in any of the gene expression is an increase of 30% to 100%. In some embodiments, the increase in any of the gene expression is an increase of 40% to 100%. In some embodiments, the increase in any of the gene expression is an increase of 50% to 100%. In some embodiments, the increase in any of the gene expression is an increase of 60% to 100%. In some embodiments, the increase in any of the gene expression is an increase of 70% to 100%. In some embodiments, the increase in any of the gene expression is an increase of 80% to 100%. In some embodiments, the increase in any of the gene expression is an increase of 90% to 100%.

In various embodiments of the methods provided herein, including the methods provided in this Section (Section 5.2) such as the methods provided in this and the preceding 61 paragraphs, the increase in any of the gene expression is an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 0, 95, 100 fold or more. In some embodiments, the increase in any of the gene expression is a 1 fold increase. In some embodiments, the increase in any of the gene expression is a 2 fold increase. In some embodiments, the increase in any of the gene expression is a 3 fold increase. In some embodiments, the increase in any of the gene expression is a 4 fold increase. In some embodiments, the increase in any of the gene expression is a 5 fold increase. In some embodiments, the increase in any of the gene expression is a 6 fold increase. In some embodiments, the increase in any of the gene expression is a 7 fold increase. In some embodiments, the increase in any of the gene expression is an 8 fold increase. In some embodiments, the increase in any of the gene expression is a 9 fold increase. In some embodiments, the increase in any of the gene expression is a 10 fold increase. In some embodiments, the increase in any of the gene expression is an 11 fold increase. In some embodiments, the increase in any of the gene expression is a 12 fold increase. In some embodiments, the increase in any of the gene expression is a 13 fold increase. In some embodiments, the increase in any of the gene expression is a 14 fold increase. In some embodiments, the increase in any of the gene expression is a 15 fold increase. In some embodiments, the increase in any of the gene expression is a 16 fold increase. In some embodiments, the increase in any of the gene expression is a 17 fold increase. In some embodiments, the increase in any of the gene expression is an 18 fold increase. In some embodiments, the increase in any of the gene expression is a 19 fold increase. In some embodiments, the increase in any of the gene expression is a 20 fold increase. In some embodiments, the increase in any of the gene expression is a 21 fold increase. In some embodiments, the increase in any of the gene expression is a 22 fold increase. In some embodiments, the increase in any of the gene expression is a 23 fold increase. In some embodiments, the increase in any of the gene expression is a 24 fold increase. In some embodiments, the increase in any of the gene expression is a 25 fold increase. In some embodiments, the increase in any of the gene expression is a 26 fold increase. In some embodiments, the increase in any of the gene expression is a 27 fold increase. In some embodiments, the increase in any of the gene expression is a 28 fold increase. In some embodiments, the increase in any of the gene expression is a 29 fold increase. In some embodiments, the increase in any of the gene expression is a 30 fold increase. In some embodiments, the increase in any of the gene expression is a 35 fold increase. In some embodiments, the increase in any of the gene expression is a 40 fold increase. In some embodiments, the increase in any of the gene expression is a 45 fold increase. In some embodiments, the increase in any of the gene expression is a 50 fold increase. In some embodiments, the increase in any of the gene expression is a 60 fold increase. In some embodiments, the increase in any of the gene expression is a 65 fold increase. In some embodiments, the increase in any of the gene expression is a 70 fold increase. In some embodiments, the increase in any of the gene expression is a 75 fold increase. In some embodiments, the increase in any of the gene expression is an 80 fold increase. In some embodiments, the increase in any of the gene expression is an 85 fold increase. In some embodiments, the increase in any of the gene expression is a 90 fold increase. In some embodiments, the increase in any of the gene expression is a 95 fold increase. In some embodiments, the increase in any of the gene expression is a 100 fold increase. In some embodiments, the increase in any of the gene expression is at least a 1 fold increase. In some embodiments, the increase in any of the gene expression is at least a 2 fold increase. In some embodiments, the increase in any of the gene expression is at least a 3 fold increase. In some embodiments, the increase in any of the gene expression is at least a 4 fold increase. In some embodiments, the increase in any of the gene expression is at least a 5 fold increase. In some embodiments, the increase in any of the gene expression is at least a 6 fold increase. In some embodiments, the increase in any of the gene expression is at least a 7 fold increase. In some embodiments, the increase in any of the gene expression is at least an 8 fold increase. In some embodiments, the increase in any of the gene expression is at least a 9 fold increase. In some embodiments, the increase in any of the gene expression is at least a 10 fold increase. In some embodiments, the increase in any of the gene expression is at least an 11 fold increase. In some embodiments, the increase in any of the gene expression is at least a 12 fold increase. In some embodiments, the increase in any of the gene expression is at least a 13 fold increase. In some embodiments, the increase in any of the gene expression is at least a 14 fold increase. In some embodiments, the increase in any of the gene expression is at least a 15 fold increase. In some embodiments, the increase in any of the gene expression is at least a 16 fold increase. In some embodiments, the increase in any of the gene expression is at least a 17 fold increase. In some embodiments, the increase in any of the gene expression is at least an 18 fold increase. In some embodiments, the increase in any of the gene expression is at least a 19 fold increase. In some embodiments, the increase in any of the gene expression is at least a 20 fold increase. In some embodiments, the increase in any of the gene expression is at least a 21 fold increase. In some embodiments, the increase in any of the gene expression is at least a 22 fold increase. In some embodiments, the increase in any of the gene expression is at least a 23 fold increase. In some embodiments, the increase in any of the gene expression is at least a 24 fold increase. In some embodiments, the increase in any of the gene expression is at least a 25 fold increase. In some embodiments, the increase in any of the gene expression is at least a 26 fold increase. In some embodiments, the increase in any of the gene expression is at least a 27 fold increase. In some embodiments, the increase in any of the gene expression is at least a 28 fold increase. In some embodiments, the increase in any of the gene expression is at least a 29 fold increase. In some embodiments, the increase in any of the gene expression is at least a 30 fold increase. In some embodiments, the increase in any of the gene expression is at least a 35 fold increase. In some embodiments, the increase in any of the gene expression is at least a 40 fold increase. In some embodiments, the increase in any of the gene expression is at least a 45 fold increase. In some embodiments, the increase in any of the gene expression is at least a 50 fold increase. In some embodiments, the increase in any of the gene expression is at least a 60 fold increase. In some embodiments, the increase in any of the gene expression is at least a 65 fold increase. In some embodiments, the increase in any of the gene expression is at least a 70 fold increase. In some embodiments, the increase in any of the gene expression is at least a 75 fold increase. In some embodiments, the increase in any of the gene expression is at least an 80 fold increase. In some embodiments, the increase in any of the gene expression is at least an 85 fold increase. In some embodiments, the increase in any of the gene expression is at least a 90 fold increase. In some embodiments, the increase in any of the gene expression is at least a 95 fold increase. In some embodiments, the increase in any of the gene expression is at least a 100 fold increase. In some embodiments, the increase in any of the gene expression is a 1 to 100 fold increase. In some embodiments, the increase in any of the gene expression is a 2 to 100 fold increase. In some embodiments, the increase in any of the gene expression is a 3 to 100 fold increase. In some embodiments, the increase in any of the gene expression is a 4 to 100 fold increase. In some embodiments, the increase in any of the gene expression is a 5 to 100 fold increase. In some embodiments, the increase in any of the gene expression is a 6 to 100 fold increase. In some embodiments, the increase in any of the gene expression is a 7 to 100 fold increase. In some embodiments, the increase in any of the gene expression is an 8 to 100 fold increase. In some embodiments, the increase in any of the gene expression is a 9 to 100 fold increase. In some embodiments, the increase in any of the gene expression is a 10 to 100 fold increase. In some embodiments, the increase in any of the gene expression is an 11 to 100 fold increase. In some embodiments, the increase in any of the gene expression is a 12 to 100 fold increase. In some embodiments, the increase in any of the gene expression is a 13 to 100 fold increase. In some embodiments, the increase in any of the gene expression is a 14 to 100 fold increase. In some embodiments, the increase in any of the gene expression is a 15 to 100 fold increase. In some embodiments, the increase in any of the gene expression is a 16 to 100 fold increase. In some embodiments, the increase in any of the gene expression is a 17 to 100 fold increase. In some embodiments, the increase in any of the gene expression is an 18 to 100 fold increase. In some embodiments, the increase in any of the gene expression is a 19 to 100 fold increase. In some embodiments, the increase in any of the gene expression is a 20 to 100 fold increase. In some embodiments, the increase in any of the gene expression is a 21 to 100 fold increase. In some embodiments, the increase in any of the gene expression is a 22 to 100 fold increase. In some embodiments, the increase in any of the gene expression is a 23 to 100 fold increase. In some embodiments, the increase in any of the gene expression is a 24 to 100 fold increase. In some embodiments, the increase in any of the gene expression is a 25 to 100 fold increase. In some embodiments, the increase in any of the gene expression is a 26 to 100 fold increase. In some embodiments, the increase in any of the gene expression is a 27 to 100 fold increase. In some embodiments, the increase in any of the gene expression is a 28 to 100 fold increase. In some embodiments, the increase in any of the gene expression is a 29 to 100 fold increase. In some embodiments, the increase in any of the gene expression is a 30 to 100 fold increase. In some embodiments, the increase in any of the gene expression is a 35 to 100 fold increase. In some embodiments, the increase in any of the gene expression is a 40 to 100 fold increase. In some embodiments, the increase in any of the gene expression is a 45 to 100 fold increase. In some embodiments, the increase in any of the gene expression is a 50 to 100 fold increase. In some embodiments, the increase in any of the gene expression is a 60 to 100 fold increase. In some embodiments, the increase in any of the gene expression is a 65 to 100 fold increase. In some embodiments, the increase in any of the gene expression is a 70 to 100 fold increase. In some embodiments, the increase in any of the gene expression is a 75 to 100 fold increase. In some embodiments, the increase in any of the gene expression is an 80 to 100 fold increase. In some embodiments, the increase in any of the gene expression is an 85 to 100 fold increase. In some embodiments, the increase in any of the gene expression is a 90 to 100 fold increase. In some embodiments, the increase in any of the gene expression is a 95 to 100 fold increase. In some embodiments, the increase in any of the gene expression is a 1 to 80 fold increase. In some embodiments, the increase in any of the gene expression is a 2 to 80 fold increase. In some embodiments, the increase in any of the gene expression is a 3 to 80 fold increase. In some embodiments, the increase in any of the gene expression is a 4 to 80 fold increase. In some embodiments, the increase in any of the gene expression is a 5 to 80 fold increase. In some embodiments, the increase in any of the gene expression is a 6 to 80 fold increase. In some embodiments, the increase in any of the gene expression is a 7 to 80 fold increase. In some embodiments, the increase in any of the gene expression is an 8 to 80 fold increase. In some embodiments, the increase in any of the gene expression is a 9 to 80 fold increase. In some embodiments, the increase in any of the gene expression is a 10 to 80 fold increase. In some embodiments, the increase in any of the gene expression is an 11 to 80 fold increase. In some embodiments, the increase in any of the gene expression is a 12 to 80 fold increase. In some embodiments, the increase in any of the gene expression is a 13 to 80 fold increase. In some embodiments, the increase in any of the gene expression is a 14 to 80 fold increase. In some embodiments, the increase in any of the gene expression is a 15 to 80 fold increase. In some embodiments, the increase in any of the gene expression is a 16 to 80 fold increase. In some embodiments, the increase in any of the gene expression is a 17 to 80 fold increase. In some embodiments, the increase in any of the gene expression is an 18 to 80 fold increase. In some embodiments, the increase in any of the gene expression is a 19 to 80 fold increase. In some embodiments, the increase in any of the gene expression is a 20 to 80 fold increase. In some embodiments, the increase in any of the gene expression is a 21 to 80 fold increase. In some embodiments, the increase in any of the gene expression is a 22 to 80 fold increase. In some embodiments, the increase in any of the gene expression is a 23 to 80 fold increase. In some embodiments, the increase in any of the gene expression is a 24 to 80 fold increase. In some embodiments, the increase in any of the gene expression is a 25 to 80 fold increase. In some embodiments, the increase in any of the gene expression is a 26 to 80 fold increase. In some embodiments, the increase in any of the gene expression is a 27 to 80 fold increase. In some embodiments, the increase in any of the gene expression is a 28 to 80 fold increase. In some embodiments, the increase in any of the gene expression is a 29 to 80 fold increase. In some embodiments, the increase in any of the gene expression is a 30 to 80 fold increase. In some embodiments, the increase in any of the gene expression is a 35 to 80 fold increase. In some embodiments, the increase in any of the gene expression is a 40 to 80 fold increase. In some embodiments, the increase in any of the gene expression is a 45 to 80 fold increase. In some embodiments, the increase in any of the gene expression is a 50 to 80 fold increase. In some embodiments, the increase in any of the gene expression is a 60 to 80 fold increase. In some embodiments, the increase in any of the gene expression is a 65 to 80 fold increase. In some embodiments, the increase in any of the gene expression is a 70 to 80 fold increase. In some embodiments, the increase in any of the gene expression is a 75 to 80 fold increase. In some embodiments, the increase in any of the gene expression is a 1 to 50 fold increase. In some embodiments, the increase in any of the gene expression is a 2 to 50 fold increase. In some embodiments, the increase in any of the gene expression is a 3 to 50 fold increase. In some embodiments, the increase in any of the gene expression is a 4 to 50 fold increase. In some embodiments, the increase in any of the gene expression is a 5 to 50 fold increase. In some embodiments, the increase in any of the gene expression is a 6 to 50 fold increase. In some embodiments, the increase in any of the gene expression is a 7 to 50 fold increase. In some embodiments, the increase in any of the gene expression is an 8 to 50 fold increase. In some embodiments, the increase in any of the gene expression is a 9 to 50 fold increase. In some embodiments, the increase in any of the gene expression is a 10 to 50 fold increase. In some embodiments, the increase in any of the gene expression is an 11 to 50 fold increase. In some embodiments, the increase in any of the gene expression is a 12 to 50 fold increase. In some embodiments, the increase in any of the gene expression is a 13 to 50 fold increase. In some embodiments, the increase in any of the gene expression is a 14 to 50 fold increase. In some embodiments, the increase in any of the gene expression is a 15 to 50 fold increase. In some embodiments, the increase in any of the gene expression is a 16 to 50 fold increase. In some embodiments, the increase in any of the gene expression is a 17 to 50 fold increase. In some embodiments, the increase in any of the gene expression is an 18 to 50 fold increase. In some embodiments, the increase in any of the gene expression is a 19 to 50 fold increase. In some embodiments, the increase in any of the gene expression is a 20 to 50 fold increase. In some embodiments, the increase in any of the gene expression is a 21 to 50 fold increase. In some embodiments, the increase in any of the gene expression is a 22 to 50 fold increase. In some embodiments, the increase in any of the gene expression is a 23 to 50 fold increase. In some embodiments, the increase in any of the gene expression is a 24 to 50 fold increase. In some embodiments, the increase in any of the gene expression is a 25 to 50 fold increase. In some embodiments, the increase in any of the gene expression is a 26 to 50 fold increase. In some embodiments, the increase in any of the gene expression is a 27 to 50 fold increase. In some embodiments, the increase in any of the gene expression is a 28 to 50 fold increase. In some embodiments, the increase in any of the gene expression is a 29 to 50 fold increase. In some embodiments, the increase in any of the gene expression is a 30 to 50 fold increase. In some embodiments, the increase in any of the gene expression is a 35 to 50 fold increase. In some embodiments, the increase in any of the gene expression is a 40 to 50 fold increase. In some embodiments, the increase in any of the gene expression is a 45 to 50 fold increase. In some embodiments, the increase in any of the gene expression is a 1 to 40 fold increase. In some embodiments, the increase in any of the gene expression is a 2 to 40 fold increase. In some embodiments, the increase in any of the gene expression is a 3 to 40 fold increase. In some embodiments, the increase in any of the gene expression is a 4 to 40 fold increase. In some embodiments, the increase in any of the gene expression is a 5 to 40 fold increase. In some embodiments, the increase in any of the gene expression is a 6 to 40 fold increase. In some embodiments, the increase in any of the gene expression is a 7 to 40 fold increase. In some embodiments, the increase in any of the gene expression is an 8 to 40 fold increase. In some embodiments, the increase in any of the gene expression is a 9 to 40 fold increase. In some embodiments, the increase in any of the gene expression is a 10 to 40 fold increase. In some embodiments, the increase in any of the gene expression is an 11 to 40 fold increase. In some embodiments, the increase in any of the gene expression is a 12 to 40 fold increase. In some embodiments, the increase in any of the gene expression is a 13 to 40 fold increase. In some embodiments, the increase in any of the gene expression is a 14 to 40 fold increase. In some embodiments, the increase in any of the gene expression is a 15 to 40 fold increase. In some embodiments, the increase in any of the gene expression is a 16 to 40 fold increase. In some embodiments, the increase in any of the gene expression is a 17 to 40 fold increase. In some embodiments, the increase in any of the gene expression is an 18 to 40 fold increase. In some embodiments, the increase in any of the gene expression is a 19 to 40 fold increase. In some embodiments, the increase in any of the gene expression is a 20 to 40 fold increase. In some embodiments, the increase in any of the gene expression is a 21 to 40 fold increase. In some embodiments, the increase in any of the gene expression is a 22 to 40 fold increase. In some embodiments, the increase in any of the gene expression is a 23 to 40 fold increase. In some embodiments, the increase in any of the gene expression is a 24 to 40 fold increase. In some embodiments, the increase in any of the gene expression is a 25 to 40 fold increase. In some embodiments, the increase in any of the gene expression is a 26 to 40 fold increase. In some embodiments, the increase in any of the gene expression is a 27 to 40 fold increase. In some embodiments, the increase in any of the gene expression is a 28 to 40 fold increase. In some embodiments, the increase in any of the gene expression is a 29 to 40 fold increase. In some embodiments, the increase in any of the gene expression is a 30 to 40 fold increase. In some embodiments, the increase in any of the gene expression is a 35 to 40 fold increase. In some embodiments, the increase in any of the gene expression is a 1 to 30 fold increase. In some embodiments, the increase in any of the gene expression is a 2 to 30 fold increase. In some embodiments, the increase in any of the gene expression is a 3 to 30 fold increase. In some embodiments, the increase in any of the gene expression is a 4 to 30 fold increase. In some embodiments, the increase in any of the gene expression is a 5 to 30 fold increase. In some embodiments, the increase in any of the gene expression is a 6 to 30 fold increase. In some embodiments, the increase in any of the gene expression is a 7 to 30 fold increase. In some embodiments, the increase in any of the gene expression is an 8 to 30 fold increase. In some embodiments, the increase in any of the gene expression is a 9 to 30 fold increase. In some embodiments, the increase in any of the gene expression is a 10 to 30 fold increase. In some embodiments, the increase in any of the gene expression is an 11 to 30 fold increase. In some embodiments, the increase in any of the gene expression is a 12 to 30 fold increase. In some embodiments, the increase in any of the gene expression is a 13 to 30 fold increase. In some embodiments, the increase in any of the gene expression is a 14 to 30 fold increase. In some embodiments, the increase in any of the gene expression is a 15 to 30 fold increase. In some embodiments, the increase in any of the gene expression is a 16 to 30 fold increase. In some embodiments, the increase in any of the gene expression is a 17 to 30 fold increase. In some embodiments, the increase in any of the gene expression is an 18 to 30 fold increase. In some embodiments, the increase in any of the gene expression is a 19 to 30 fold increase. In some embodiments, the increase in any of the gene expression is a 20 to 30 fold increase. In some embodiments, the increase in any of the gene expression is a 21 to 30 fold increase. In some embodiments, the increase in any of the gene expression is a 22 to 30 fold increase. In some embodiments, the increase in any of the gene expression is a 23 to 30 fold increase. In some embodiments, the increase in any of the gene expression is a 24 to 30 fold increase. In some embodiments, the increase in any of the gene expression is a 25 to 30 fold increase. In some embodiments, the increase in any of the gene expression is a 26 to 30 fold increase. In some embodiments, the increase in any of the gene expression is a 27 to 30 fold increase. In some embodiments, the increase in any of the gene expression is a 28 to 30 fold increase. In some embodiments, the increase in any of the gene expression is a 29 to 30 fold increase. In some embodiments, the increase in any of the gene expression is a 1 to 20 fold increase. In some embodiments, the increase in any of the gene expression is a 2 to 20 fold increase. In some embodiments, the increase in any of the gene expression is a 3 to 20 fold increase. In some embodiments, the increase in any of the gene expression is a 4 to 20 fold increase. In some embodiments, the increase in any of the gene expression is a 5 to 20 fold increase. In some embodiments, the increase in any of the gene expression is a 6 to 20 fold increase. In some embodiments, the increase in any of the gene expression is a 7 to 20 fold increase. In some embodiments, the increase in any of the gene expression is an 8 to 20 fold increase. In some embodiments, the increase in any of the gene expression is a 9 to 20 fold increase. In some embodiments, the increase in any of the gene expression is a 10 to 20 fold increase. In some embodiments, the increase in any of the gene expression is an 11 to 20 fold increase. In some embodiments, the increase in any of the gene expression is a 12 to 20 fold increase. In some embodiments, the increase in any of the gene expression is a 13 to 20 fold increase. In some embodiments, the increase in any of the gene expression is a 14 to 20 fold increase. In some embodiments, the increase in any of the gene expression is a 15 to 20 fold increase. In some embodiments, the increase in any of the gene expression is a 16 to 20 fold increase. In some embodiments, the increase in any of the gene expression is a 17 to 20 fold increase. In some embodiments, the increase in any of the gene expression is an 18 to 20 fold increase. In some embodiments, the increase in any of the gene expression is a 19 to 20 fold increase. In some embodiments, the increase in any of the gene expression is a 1 to 10 fold increase. In some embodiments, the increase in any of the gene expression is a 2 to 10 fold increase. In some embodiments, the increase in any of the gene expression is a 3 to 10 fold increase. In some embodiments, the increase in any of the gene expression is a 4 to 10 fold increase. In some embodiments, the increase in any of the gene expression is a 5 to 10 fold increase. In some embodiments, the increase in any of the gene expression is a 6 to 10 fold increase. In some embodiments, the increase in any of the gene expression is a 7 to 10 fold increase. In some embodiments, the increase in any of the gene expression is an 8 to 10 fold increase. In some embodiments, the increase in any of the gene expression is a 9 to 10 fold increase. In some embodiments, the increase in any of the gene expression is a 1 to 5 fold increase. In some embodiments, the increase in any of the gene expression is a 2 to 5 fold increase. In some embodiments, the increase in any of the gene expression is a 3 to 5 fold increase. In some embodiments, the increase in any of the gene expression is a 4 to 5 fold increase.

In various aspects or embodiments of the methods provided herein, including the methods provided in this Section (Section 5.2) such as the methods provided in this and the preceding 62 paragraphs, the methods involve administration of an immune checkpoint inhibitor as provided in the method. As used herein, the term “immune checkpoint inhibitor” or “checkpoint inhibitor” refers to molecules that totally or partially reduce, inhibit, interfere with or modulate one or more checkpoint proteins. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Other exemplary checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In certain embodiments, the checkpoint inhibitor for the methods provided herein can be an inhibitor against a checkpoint protein that correlated with ICD. In some embodiments, the checkpoint inhibitor for the methods provided herein can be an inhibitor against a checkpoint protein that correlated with ICD. In other embodiments, the checkpoint inhibitor for the methods provided herein can be an inhibitor against a checkpoint protein that upregulated with the treatment of anti-nectin-4 ADCs. In further embodiments, the checkpoint inhibitor for the methods provided herein can be an inhibitors or activators against a checkpoint protein that upregulated with the treatment of anti-nectin-4 ADCs, which checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. In yet further embodiments, the checkpoint inhibitor for the methods provided herein can be an inhibitors or activators against a checkpoint protein that upregulated with the treatment of anti-nectin-4 ADCs, which include a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below.

In some embodiments, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In certain embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®). In a further embodiment, the PD-L1 inhibitor is avelumab.

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is a VTCN inhibitor. In one embodiment, the VTCN inhibitor is FPA150.

In one embodiment, the checkpoint inhibitor is an IDO inhibitor. In one embodiment, the IDO inhibitor is INCB024360. In another embodiment, the IDO inhibitor is indoximod. In one embodiment, the IDO inhibitor is epacadostat. In another embodiment, the IDO inhibitor is BMS986205. In yet another embodiment, the IDO inhibitor is Navoximod. In one embodiment, the IDO inhibitor is PF-06840003. In another embodiment, the IDO inhibitor is KHK2455. In yet another embodiment, the IDO inhibitor is RG70099. In one embodiment, the IDO inhibitor is IOM-E. In another embodiment, the IDO inhibitor is or IOM-D.

In some embodiments, the checkpoint inhibitor is a TIGIT inhibitor. In certain embodiments, the TIGIT inhibitor is an anti-TIGIT antibody. In one embodiment, the TIGIT inhibitor is MTIG7192A. In another embodiment, the TIGIT inhibitor is BMS-986207. In yet another embodiment, the TIGIT inhibitor is OMP-313M32. In one embodiment, the TIGIT inhibitor is MK-7684. In another embodiment, the TIGIT inhibitor is AB154. In yet another embodiment, the TIGIT inhibitor is CGEN-15137. In one embodiment, the TIGIT inhibitor is SEA-TIGIT. In another embodiment, the TIGIT inhibitor is ASP8374. In yet another embodiment, the TIGIT inhibitor is AJUD008.

In some embodiments, the checkpoint inhibitor is a VSIR inhibitor. In certain embodiments, the VSIR inhibitor is an anti-VSIR antibody. In one embodiment, the VSIR inhibitor is MTIG7192A. In another embodiment, the VSIR inhibitor is CA-170. In yet another embodiment, the VSIR inhibitor is JNJ 61610588. In one embodiment, the VSIR inhibitor is HMBD-002.

In some embodiments, the checkpoint inhibitor is a TIM3 inhibitor. In certain embodiments, the TIM3 inhibitor is an anti-TIM3 antibody. In one embodiment, the TIM3 inhibitor is AJUD009.

In some embodiments, the checkpoint inhibitor is a CD25 (IL2RA) inhibitor. In certain embodiments, the CD25 (IL2RA) inhibitor is an anti-CD25 (IL2RA) antibody. In one embodiment, the CD25 (IL2RA) inhibitor is daclizumab. In another embodiment, the CD25 (IL2RA) inhibitor is basiliximab.

In some embodiments, the checkpoint inhibitor is an IFNAR1 inhibitor. In certain embodiments, the IFNAR1 inhibitor is an anti-IFNAR1 antibody. In one embodiment, the IFNAR1 inhibitor is anifrolumab. In another embodiment, the IFNAR1 inhibitor is sifalimumab.

In some embodiments, the checkpoint inhibitor is a CSF1R inhibitor. In certain embodiments, the CSF1R inhibitor is an anti-CSF1R antibody. In one embodiment, the CSF1R inhibitor is pexidartinib. In another embodiment, the CSF1R inhibitor is emactuzumab. In yet another embodiment, the CSF1R inhibitor is cabiralizumab. In one embodiment, the CSF1R inhibitor is ARRY-382. In another embodiment, the CSF1R inhibitor is BLZ945. In yet another embodiment, the CSF1R inhibitor is AJUD010. In one embodiment, the CSF1R inhibitor is AMG820. In another embodiment, the CSF1R inhibitor is IMC-CS4. In yet another embodiment, the CSF1R inhibitor is JNJ-40346527. In one embodiment, the CSF1R inhibitor is PLX5622. In another embodiment, the CSF1R inhibitor is FPA008.

In some embodiments, the checkpoint inhibitor is a therapeutic agent targeting HLA. In certain embodiments, the therapeutic agent targeting HLA is an anti-HLA antibody. In one embodiment, the therapeutic agent targeting HLA is GSK01. In another embodiment, the therapeutic agent targeting HLA is IMC-C₁₀₃C. In yet another embodiment, the therapeutic agent targeting HLA is IMC-F106C. In one embodiment, the therapeutic agent targeting HLA is IMC-G107C. In another embodiment, the therapeutic agent targeting HLA is ABBV-184.

In certain embodiments, the immune checkpoint inhibitors provided herein include two or more of the checkpoint inhibitors described herein (including checkpoint inhibitors of the same or different class). Moreover, the methods described herein can be used in combination with one or more second active agents as described herein where appropriate for treating diseases described herein and understood in the art.

In some embodiments, the checkpoint inhibitor is administered after the administration of the ADCs provided herein. In other embodiments, the checkpoint inhibitor is administered simultaneously (e.g., in the same dosing period) with the ADCs provided herein. In yet other embodiments, the checkpoint inhibitor is administered after the administration of the ADCs provided herein.

In some embodiments, the amount of the checkpoint inhibitor for the various methods provided herein can be determined by standard clinical techniques. In certain embodiments, the amount of the checkpoint inhibitor for the various methods are provided in Section 5.6.

In the various methods provided herein including those described in the preceding 89 paragraphs: the ADCs and immune checkpoint inhibitors that can be used are described in this Section (Section 5.2), Section 5.3, and Section 6; selection of specific patient populations and/or specific cancers for treatment by the methods provided herein is described in this Section (Section 5.2) and Section 5.9; dosing regimens and pharmaceutical composition for administering the ADCs and immune checkpoint inhibitors are described in this Section (Section 5.2), Section 5.6, Section 5.4 and Section 5.7 below; the biomarkers that can be used for identifying the therapeutic agents, selecting the patients, determining the outcome of these methods, and/or serving as criteria in any way for these methods are described herein and exemplified in this Section (Section 5.2), Section 5.9, and Section 6; therapeutic outcomes for the methods provided herein can be improvement of the biomarkers described herein, for example, those described and exemplified in this Section (Section 5.2), Section 5.9, and Section 6; assays for corroborating the applicability or suitability of the various biomarkers provided herein for the methods are described in Section 5.8. Therefore, a person skilled in the art would understand that the methods provided herein include all permutations and combinations of the patients, therapeutic agents, dosing regiments, biomarkers, therapeutic outcomes, corroborating assays as described above and below.

### 5.3 Antibody Drug Conjugates for the Methods

In certain embodiments of the methods provided herein, including the methods provided in Section 5.2, the ADC used in the methods comprises or consists of any anti-cancer antibody or antigen binding fragment conjugated to a cytotoxic agent. In some embodiments of the methods provided herein, including the methods provided in Section 5.2, the ADC used in the methods comprises or consists of any antibody or antigen binding fragment that binds a cancer specific marker, wherein the antibody or antigen binding fragment is conjugated to a cytotoxic agent. In other embodiments of the methods provided herein, including the methods provided in Section 5.2, the ADC used in the methods comprises or consists of any antibody or antigen binding fragment conjugated to a cytotoxic agent. In one embodiment, the ADC comprises an antibody or antigen binding fragment conjugated to a cytotoxic agent, wherein the antibody or antigen binding fragment specifically binds a intracellular molecule, a disease marker, a neoantigen, or a cell surface molecule (e.g. a cell surface receptor or receptor complex). In some embodiments, the ADC comprises an antibody or antigen binding fragment conjugated to a cytotoxic agent, wherein the antibody or antigen binding fragment specifically binds disease marker. In other embodiments, the ADC comprises an antibody or antigen binding fragment conjugated to a cytotoxic agent, wherein the antibody or antigen binding fragment specifically binds an intracellular molecule. In certain embodiments, the ADC comprises an antibody or antigen binding fragment conjugated to a cytotoxic agent, wherein the antibody or antigen binding fragment specifically binds a cell surface molecule. In another embodiment, the ADC comprises an antibody or antigen binding fragment conjugated to a cytotoxic agent, wherein the antibody or antigen binding fragment specifically binds a cell surface receptor. In yet another embodiment, the ADC comprises an antibody or antigen binding fragment conjugated to a cytotoxic agent, wherein the antibody or antigen binding fragment specifically binds a cell surface receptor complex. In some embodiments, the ADC comprises an antibody or antigen binding fragment conjugated to a cytotoxic agent, wherein the antibody or antigen binding fragment specifically binds a marker for a cancer. In certain embodiments, the ADC comprises an antibody or antigen binding fragment conjugated to a cytotoxic agent, wherein the antibody or antigen binding fragment specifically binds a neoantigen. In further embodiments, the ADC comprises an antibody or antigen binding fragment conjugated to a cytotoxic agent, wherein the antibody or antigen binding fragment specifically binds a cancer antigen.

Specifically, in some embodiments, the ADC used in the methods provided herein comprises an antibody or antigen binding fragment conjugated to a cytotoxic agent, wherein the antibody or antigen binding fragment specifically binds a target selected from the group consisting of: CD1, CD1a, CD2, CD3, CD4, CD5, CD8, CD10, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD47, CD52, CD54, CD55, CD59, CD64, CD66a-e, CD67, CD70, CD70L, CD74, CD79a, CD80, CD83, CD95, CD100, CD101, CD102, CD103, CD104, CD105, CD106, CD107a, CD107b, CD108, CD109, CD111, CD112, CD113, CD116, CD117, CD118, CD119, CD11a, CD11b, CD11c, CD120a, CD121a, CD121b, CD122, CD123, CD124, CD125, CD126, CD127, CD13, CD130, CD131, CD132, CD133, CD135, CD136, CD137, CD138, CD140a, CD140b, CD141, CD142, CD143, CD144, CD146, CD147, CD148, CD150, CD151, CD154, CD155, CD156a, CD156b, CD156c, CD157, CD158b2, CD158e, CD158f1, CD158h, CD158i, CD159a, CD160, CD161, CD162, CD163, CD164, CD166, CD167b, CD169, CD16a, CD16b, CD170, CD171, CD172a, CD172b, CD172g, CD180, CD181, CD183, CD185, CD194, CD197, CD1b, CD1c, CD1d, CD2, CD200, CD201, CD202b, CD203c, CD204, CD205, CD206, CD208, CD213a1, CD213a2, CD217, CD218a, CD220, CD221, CD222, CD224, CD226, CD228, CD229, CD230, CD232, CD239, CD243, CD244, CD249, CD26, CD265, CD267, CD269, CD27, CD272, CD273, CD274, CD275, CD276, CD279, CD28, CD280, CD281, CD282, CD283, CD284, CD289, CD294, CD295, CD298, CD302, CD304, CD305, CD307, CD31, CD312, CD315, CD316, CD317, CD318, CD319, CD32, CD321, CD322, CD324, CD325, CD326, CD327, CD328, CD331, CD332, CD333, CD334, CD337, CD339, CD34, CD340, CD344, CD35, CD36, CD39, CD3d, CD3g, CD41, CD42d, CD44, CD48, CD49a, CD49b, CD49c, CD49d, CD49e, CD49f, CD5, CD50, CD51, CD53, CD56, CD58, CD6, CD61, CD62L, CD62P, CD63, CD68, CD69, CD7, CD71, CD72, CD73, CD79b, CD82, CD84, CD85f, CD85i, CD85j, CD86, CD87, CD89, CD90, CD91, CD92, CD96, CD97, CD98, CDw210a, CDw210b, PSMA, CEACAM5, CEACAM-6, MUC1, MUC2, MUC3, MUC4, MUC5, MUC5ac, MUC16, PAM4 antigen, NCA-95, NCA-90, Ia, HM1.24, EGP-1 (TROP-2), EGP-2, HLA-DR, tenascin, Le(y), T101, TAC, Tn antigen, Thomson-Friedenreich antigens, TRAIL receptor (R1 and R2), VEGFR, EGFR, P1GF, complement factors C3, C3a, C3b, C5a, C5, HER2, EGFR, Mesothelin antigen, Trop-2 (M1S1, TACSTD2 or GA733-1) antigen, HER3, DLL3 antigen, GPNMB antigen, CD79b, GCC antigen, NaPi2b antigen, CA6 antigen, BCMA antigen, SLMAMF7 (CS1) antigen, TIM1 antigen, FOLR1 antigen, CanAg antigen, EphA2 antigen, SLTRK6 antigen, HGFR antigen, FGFR2 antigen, C4.4a (LYPD3), uPAR, p-cadherin (cadherin 3), 5T4 (TPBG) antigen, STEAP1 antigen, PTK4 antigen, Ephrin-A4 (EFNA4) antigen, LIV1 (SLC39A6 or ZIP6) antigen, TENB2 antigen, ETBR antigen, integrin alphavbeta3, crypto antigen, SLC44A4 antigen, LY6E antigen, AXL (UFO) antigen, LAMP-1 antigen, and MN/CA IX antigen.

In further specific embodiments, the ADC used in the methods provided herein comprises an anti-nectin-4 antibody or antigen binding fragment as provided in Subsection 5.3.1 conjugated to a cytotoxic agent. In yet another embodiment, the ADC used in the methods provided herein comprises an antibody or antigen binding fragment as provided in Subsection 5.3.1 conjugated to MMAE.

In certain embodiments of the methods provided herein, including the methods provided in Section 5.2, the ADC used in the methods comprises any antibody or antigen binding fragment as provided in this Section (Section 5.3) including Subsection 5.3.1 conjugated to a cytotoxic agent as provided in this Section (Section 5.3) including Subsection 5.3.2, in any combination or permutation.

In various embodiments of the methods provided herein, including the methods provided in Section 5.2, the ADC used in the methods comprises an antibody or antigen binding fragment as provided herein, including in this Section (Section 5.3) with further disclosures in Subsection 5.3.1, conjugated to one or more units of cytotoxic agents (drug units, or D) as provided herein, including in this Section (Section 5.3) with further disclosures in Subsection 5.3.2. In some embodiments, the cytotoxic agents (drug units, or D) can be covalently linked directly or via a linker unit (LU).

In some embodiments, the antibody drug conjugate compound has the following formula:

L-(LU-D)ₚ (I)
or a pharmaceutically acceptable salt or solvate thereof; wherein:
L is the antibody unit, and
(LU-D) is a linker unit-drug unit moiety, wherein:
LU- is a linker unit, and
D is a drug unit having cytostatic or cytotoxic activity against a target cell; and
p is an integer from 1 to 20.

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}-Y_{y}-D)ₚ (II)
or a pharmaceutically acceptable salt or solvate thereof, wherein:
L is the Antibody unit and
-Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2;
D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Subsection 5.3.1, conjugated to one or more units of cytotoxic agents (drug units, or D) as provided herein, including in this Section (Section 5.3) with further disclosures in Subsection 5.3.2. In certain embodiments, the cytotoxic agents (drug units, or D) can be covalently linked directly or via a linker unit (LU).

In some embodiments, the antibody drug conjugate compound has the following formula:

L-(LU-D)ₚ (I)
or a pharmaceutically acceptable salt or solvate thereof; wherein:
L is the antibody unit, e.g., the anti-nectin-4 antibody or an antigen binding fragment
thereof as provided in Subsection 5.3.1 below, and
(LU-D) is a linker unit-drug unit moiety, wherein:
LU- is a linker unit, and
D is a drug unit having cytostatic or cytotoxic activity against a target cell; and
p is an integer from 1 to 20.

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}-Y_{y}-D)ₚ (II)
or a pharmaceutically acceptable salt or solvate thereof, wherein:
L is the Antibody unit, e.g., the anti-nectin-4 antibody or an antigen binding fragment
thereof as provided in Subsection 5.3.1 below; and
-Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2;
D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE. In some specific embodiments of the methods provided herein, including the methods provided in Section 5.2, the cytotoxic agent as part of any of the ADCs provided herein for the methods comprises, consists of, or is MMAF.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In exemplary embodiments, p is from 2 to 8.

### 5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO: 22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMN
WVRQAPGKGLEWVSYISSSSSTIYYADSVKGRFTI
SRDNAKNSLSLQMNSLRDEDTAVYYCARAYYYGMD
VWGQGTTVTVSS
SEQ ID NO: 23
DIQMTQSPSSVSASVGDRVTITCRASQGISGWLAW
YQQKPGKAPKFLIYAASTLQSGVPSRFSGSGSGTD
FTLTISSLQPEDFATYYCQQANSFPPTFGGGTKVE
IKR
SEQ ID NO: 7
MELGLCWVFLVAILEGVQCEVQLVESGGGLVQPGG
SLRLSCAASGFTFSSYNMNWVRQAPGKGLEWVSYI
SSSSSTIYYADSVKGRFTISRDNAKNSLSLQMNSL
RDEDTAVYYCARAYYYGMDVWGQGTTVTVSSASTK
GPSVFPLAPSSKSTSGGTAALGCLVKDYFPEPVTV
SWNSGALTSGVHTFPAVLQSSGLYSLSSVVTVPSS
SLGTQTYICNVNHKPSNTKVDKRVEPKSCDKTHTC
PPCPAPELLGGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKALPAP
IEKTISKAKGQPREPQVYTLPPSREEMTKNQVSLT
CLVKGFYPSDIAVEWESNGQPENNYKTTPPVLDSD
GSFFLYSKLTVDKSRWQQGNVFSCSVMHEALHNHY
TQKSLSLSPGK
SEQ ID NO: 8
MDMRVPAQLLGLLLLWFPGSRCDIQMTQSPSSVSA
SVGDRVTITCRASQGISGWLAWYQQKPGKAPKFLI
YAASTLQSGVPSRFSGSGSGTDFTLTISSLQPEDF
ATYYCQQANSFPPTFGGGTKVEIKRTVAAPSVFIF
PPSDEQLKSGTASVVCLLNNFYPREAKVQWKVDNA
LQSGNSQESVTEQDSKDSTYSLSSTLTLSKADYEK
HKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor Classification (ANARCI). For example, the heavy chain CDR sequences within SEQ ID NO:22, and the light chain CDR sequences within SEQ ID NO:23 according to Kabat numbering as determined by ANARCI are listed in Table 5 below.

TABLE 5

| | VH of SEQ ID NO: 22 | VL of SEQ ID NO: 23 |
|---|---|---|
| CDR1 | SYNMN | RASQGISGWLA |
| | (SEQ ID NO: 9) | (SEQ ID NO: 12) |
| CDR2 | YISSSSSTIYYADSVKG | AASTLQS |
| | (SEQ ID NO: 10) | (SEQ ID NO: 13) |
| CDR3 | AYYYGMDV | QQANSFPPT |
| | (SEQ ID NO: 11) | (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 6 below.

TABLE 6

| | VH of SEQ ID NO: 22 | VL of SEQ ID NO: 23 |
|---|---|---|
| CDR1 | GFTFSSYN | QGISGW |
| | (SEQ ID NO: 16) | (SEQ ID NO: 19) |
| CDR2 | ISSSSSTI | AAS |
| | (SEQ ID NO: 17) | (SEQ ID NO: 20) |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:NO:12, CDR-L2 comprising an amino acid sequence of SEQ ID NO:NO:13, and CDR-L3 comprising an amino acid sequence of SEQ ID NO:NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO:18, CDR-L1 comprising an amino acid sequence of SEQ ID NO:NO:19, CDR-L2 comprising an amino acid sequence of SEQ ID NO:NO:20, and CDR-L3 comprising an amino acid sequence of SEQ ID NO:NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10, CDR-H3 consisting of an amino acid sequence of SEQ ID NO:11, CDR-L1 consisting of an amino acid sequence of SEQ ID NO:NO:12, CDR-L2 consisting of an amino acid sequence of SEQ ID NO:NO:13, and CDR-L3 consisting of an amino acid sequence of SEQ ID NO:NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:NO:19, CDR-L2 consisting of an amino acid sequence of SEQ ID NO:NO:20, and CDR-L3 consisting of an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1, CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-L1, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the invention, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphysm observed in human. In some embodiments, the antibody or antigen binding fragment thereof provided herein binds to epitopes in 191P4D12 that are common between the 191P4D12 polymorphysm observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the invention can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, Va. 20108 on 18 Aug. 2010 and assigned Accession number PTA-11267.

### 5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulindisrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the group consisting of: an anthracycline (e.g., doxorubicin and daunorubicin (formerly daunomycin)); a taxan (e.g., paclitaxel (Taxol) and docetaxel (Taxotere); an antimetabolite (e.g., methotrexate, 6-mercaptopurine, 6-thioguanine, cytarabine, 5-fluorouracil and decarbazine); or an alkylating agent (e.g., mechlorethamine, thioepa chlorambucil, melphalan, carmustine (BCNU), lomustine (CCNU), cyclothosphamide, busulfan, dibromomannitol, streptozotocin, mitomycin C, cisdichlorodiamine platinum (II) (DDP) and cisplatin); an antibiotic (e.g., actinomycin D, bleomycin, mithramycin, and anthramycin (AMC)); an auristatin molecule (e.g., auristatin PHE, bryostatin 1, solastatin 10, auristatin E, auristatin F, monomethyl auristatin E (MMAE) and monomethylauristatin F (MMAF)); a hormone (e.g., glucocorticoids, progestins, androgens, and estrogens); a nucleoside analoge (e.g. Gemcitabine); a DNA-repair enzyme inhibitor (e.g., etoposide and topotecan); a kinase inhibitor (e.g., compound ST1571, also known as Gleevec or imatinib mesylate); maytansine; paclitaxel; cytochalasin B; gramicidin D; ethidium bromide; emetine; mitomycin; etoposide; tenoposide; vincristine; vinblastine; colchicine; doxorubicin; daunorubicin; dihydroxy anthracin dione; mitoxantrone; 1-dehydrotestosterone; glucorticoids; procaine; tetracaine; lidocaine; propranolol; puromycin; a farnesyl transferase inhibitor (e.g., R115777, BMS-214662, and those disclosed by, for example, U.S. Pat. No. 6,458,935); a topoisomerase inhibitor (e.g., camptothecin, irinotecan, SN-38, topotecan, 9-aminocamptothecin, GG-211 (GI 147211), DX-8951f, IST-622, rubitecan, pyrazoloacridine, XR-5000, saintopin, UCE6, UCE1022, TAN-1518A, TAN 1518B, KT6006, KT6528, ED-110, NB-506, ED-110, NB-506, fagaronine, coralyne, beta-lapachone and rebeccamycin); a DNA minor groove binder (e.g., Hoescht dye 33342 and Hoechst dye 33258); adenosine deaminase inhibitors (e.g., Fludarabine phosphate and 2-Chlorodeoxyadenosine); and pharmaceutically acceptable salts, solvates, clathrates, prodrugs, analogs, and homologs thereof. In some embodiments of the various methods provided herein, including the methods provided in Section 5.2, the cytotoxic agent as part of any of the ADCs provided herein for the methods comprises, consists of, or is any agent selected from the group consisting of: auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, glucocorticoid, other chemotherapeutic agents, and pharmaceutically acceptable salts, solvates, clathrates, prodrugs, analogs, and homologs thereof, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷.

In some embodiments, the ADC comprises an antibody or antigen binding fragment thereof conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug unit can be attached to the antibody through the N (amino) terminus or the C (carboxyl) terminus of the peptidic drug unit (WO 02/088172).

Exemplary auristatin embodiments include the N-terminus linked monomethylauristatin drug units DE and DF, disclosed in “Senter et al, Proceedings of the American Association for Cancer Research, Volume 45, Abstract Number 623, presented Mar. 28, 2004 and described in United States Patent Publication No. 2005/0238649, the disclosure of which is expressly incorporated by reference in its entirety.

In some embodiments, the auristatin is MMAE (wherein the wavy line indicates the covalent attachment to a linker of an antibody drug conjugate).

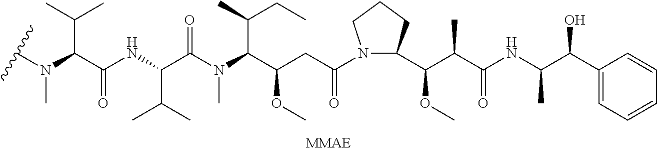 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12):

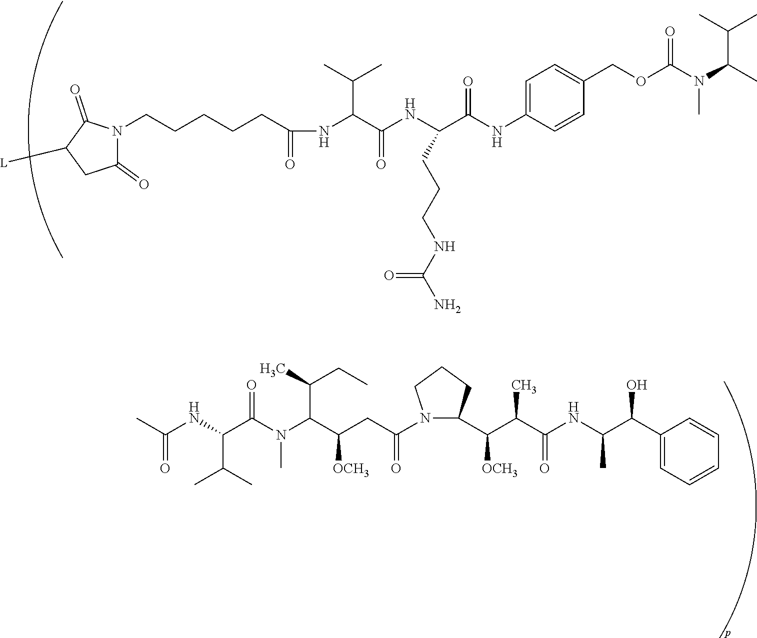

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lübke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 191P4D12-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody drug conjugate, are cleaved when the antibody drug conjugate presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In other non-mutually exclusive embodiments, the linker promotes cellular internalization. In certain embodiments, the linker promotes cellular internalization when conjugated to the therapeutic agent (i.e., in the milieu of the linker-therapeutic agent moiety of the antibody-drug conjugate compound as described herein). In yet other embodiments, the linker promotes cellular internalization when conjugated to both the auristatin compound and the anti-191P4D12 antibody or antigen binding fragment thereof.

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-
wherein: -A- is a stretcher unit,
a is 0 or 1,
each -W- is independently an amino acid unit,
w is an integer ranging from 0 to 12,
-Y- is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0.

### 5.3.3.1 Stretcher Unit

The stretcher unit (A), when present, is capable of linking an antibody unit to an amino acid unit (-W-), if present, to a spacer unit (-Y-), if present; or to a drug unit (-D). Useful functional groups that can be present on an anti-191P4D12 antibody or an antigen binding fragment thereof (e.g. Ha22-2(2,4)6.1), either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-191P4D12 antibody or an antigen binding fragment thereof. In another embodiment, sulfhydryl groups can be generated by reaction of an amino group of a lysine moiety of an anti-191P4D12 antibody or an antigen binding fragment with 2-iminothiolane (Traut's reagent) or other sulfhydryl generating reagents. In certain embodiments, the anti-191P4D12 antibody or antigen binding fragment thereof is a recombinant antibody and is engineered to carry one or more lysines. In certain other embodiments, the recombinant anti-191P4D12 antibody is engineered to carry additional sulfhydryl groups, e.g., additional cysteines.

In one embodiment, the stretcher unit forms a bond with a sulfur atom of the antibody unit. The sulfur atom can be derived from a sulfhydryl group of an antibody. Representative stretcher units of this embodiment are depicted within the square brackets of Formulas Ma and Mb below, wherein L-, -W-, -Y-, -D, w and y are as defined above, and R¹⁷ is selected from alkylene-, alkenylene-, alkynylene-, carbocyclo-, alkylene)-, (C₁-C₈ alkenylene)-, alkynylene)-, -arylene-, alkylene-arylene-, alkenylene-arylene, alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, -heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted.

In some embodiments, R¹⁷ is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

It is to be understood from all the exemplary embodiments that even where not denoted expressly, 1 to 20 drug units can be linked to an antibody unit (p=1-20).

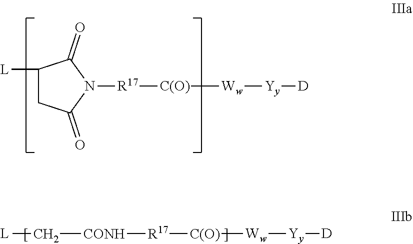

An illustrative stretcher unit is that of Formula Ma wherein R¹⁷ is —(CH₂)₅—:

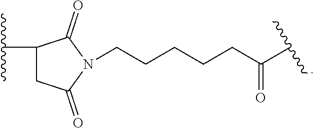 [SMILES: *C(=O)CCCCCN1C(=O)CC(*)C1=O]

Another illustrative stretcher unit is that of Formula IIIa wherein R¹⁷ is —(CH₂CH₂O)ᵣ—CH₂—; and r is 2:

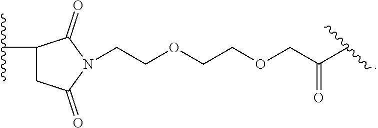 [SMILES: *C(=O)COCCOCCN1C(=O)CC(*)C1=O]

An illustrative Stretcher unit is that of Formula Ma wherein R¹⁷ is arylene- or arylene-C₁-C₁₀ alkylene-. In some embodiments, the aryl group is an unsubstituted phenyl group.

Still another illustrative stretcher unit is that of Formula IIIb wherein R¹⁷ is —(CH₂)₅—:

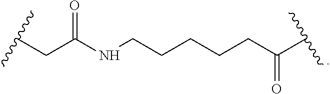 [SMILES: *CC(=O)NCCCCCC(*)=O]

In certain embodiments, the stretcher unit is linked to the antibody unit via a disulfide bond between a sulfur atom of the antibody unit and a sulfur atom of the stretcher unit. A representative stretcher unit of this embodiment is depicted within the square brackets of Formula IV, wherein R¹⁷, L-, -W-, -Y-, -D, w and y are as defined above.

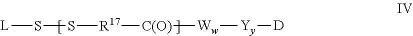

It should be noted that throughout this application, the S moiety in the formula below refers to a sulfur atom of the antibody unit, unless otherwise indicated by context.

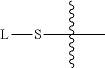

In certain of the structural descriptions of sulfur linked ADC herein the antibody is represented as “L”. It could also be indicated as “Ab-S”. The inclusion of “S” merely indicated the sulfur-linkage feature, and does not indicate that a particular sulfur atom bears multiple linker-drug moieties. The left parentheses of the structures using the “Ab-S” description can also be placed to the left of the sulfur atom, between Ab and S, which would be an equivalent description of the ADC of the invention described throughout herein.

In yet other embodiments, the stretcher contains a reactive site that can form a bond with a primary or secondary amino group of an antibody unit. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas Va and Vb, wherein —R¹⁷—, L-, -W-, -Y-, -D, w and y are as defined above;

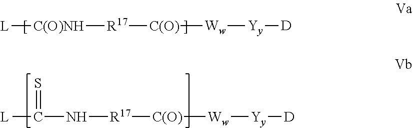

In some embodiments, the stretcher contains a reactive site that is reactive to a modified carbohydrate's (—CHO) group that can be present on an antibody unit. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative stretcher units of this embodiment are depicted within the square brackets of Formulas VIa, VIb, and VIc, wherein —R¹⁷—, L-, -W-, -Y-, -D, w and y are as defined as above.

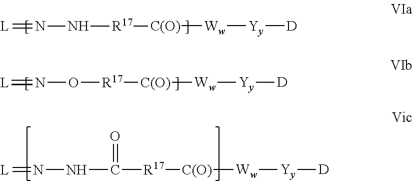

### 5.3.3.2 Amino Acid Unit

The amino acid unit (-W-), when present, links the stretcher unit to the spacer unit if the spacer unit is present, links the stretcher unit to the drug unit if the spacer unit is absent, and links the antibody unit to the drug unit if the stretcher unit and spacer unit are absent.

W_{w}-can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each -W- unit independently has the formula denoted below in the square brackets, and w is an integer ranging from 0 to 12:

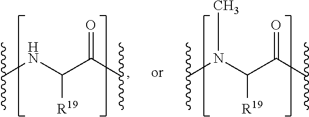

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

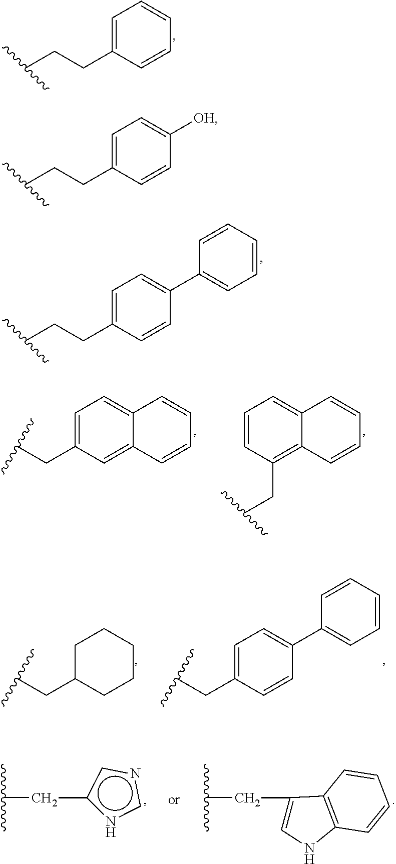

In some embodiments, the amino acid unit can be enzymatically cleaved by one or more enzymes, including a cancer or tumor-associated protease, to liberate the drug unit (-D), which in one embodiment is protonated in vivo upon release to provide a drug (D).

In certain embodiments, the amino acid unit comprises natural amino acids. In other embodiments, the amino acid unit comprises non-natural amino acids. Illustrative Ww units are represented by Formulas VII-IX below:

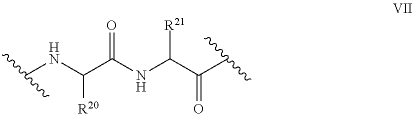

wherein R²⁰ and R²¹ are as follows:

| R²⁰ | R²¹ |
|---|---|
| Benzyl | (CH₂)₄NH₂; |
| Methyl | (CH₂)₄NH₂; |
| Isopropyl | (CH₂)₄NH₂; |
| Isopropyl | (CH₂)₃NHCONH₂; |
| Benzyl | (CH₂)₃NHCONH₂; |
| Isobutyl | (CH₂)₃NHCONH₂; |
| sec-butyl | (CH₂)₃NHCONH₂; |
| 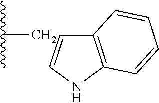 | (CH₂)₃NHCONH₂; |
| Benzyl | methyl; |
| Benzyl | (CH₂)₃NHC(═NH)NH; |

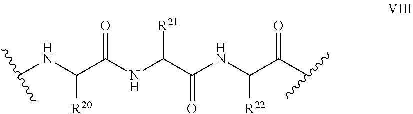

wherein R²⁰, R²¹ and R²² are as follows:

| R²⁰ | R²¹ | R²² |
|---|---|---|
| Benzyl | benzyl | (CH₂)₄NH₂; |
| Isopropyl | benzyl | (CH₂)₄NH₂; and |
| H | benzyl | (CH₂)₄NH₂; |

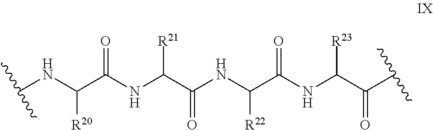

wherein R²⁰, R²¹, R²² and R²³ are as follows:

| R²⁰ | R²¹ | R²² | R²³ |
|---|---|---|---|
| H | benzyl | isobutyl | H; and |
| Methyl | isobutyl | methyl | isobutyl. |

Exemplary amino acid units include, but are not limited to, units of Formula VII above where: R²⁰ is benzyl and R²¹ is —(CH₂)₄NH₂; R²⁰ is isopropyl and R²¹ is —(CH₂)₄NH₂, or R²⁰ is isopropyl and R²¹ is —(CH₂)₃NHCONH₂.

Another exemplary amino acid unit is a unit of Formula VIII wherein R²⁰ is benzyl, R²¹ is benzyl, and R²² is —(CH₂)₄NH₂.

Useful -W_{w}- units can be designed and optimized in their selectivity for enzymatic cleavage by a particular enzyme, for example, a tumor-associated protease. In one embodiment, a -W_{w}- unit is that whose cleavage is catalyzed by cathepsin B, C and D, or a plasmin protease.

In one embodiment, -W_{w}- is a dipeptide, tripeptide, tetrapeptide or pentapeptide. When R¹⁹, R²⁰, R²¹, R²² or R²³ is other than hydrogen, the carbon atom to which R¹⁹, R²⁰, R²¹, R²² or R²³ is attached is chiral.

Each carbon atom to which R¹⁹, R²⁰, R²¹, R²² or R²³ is attached is independently in the (S) or (R) configuration.

In one specific embodiment, the amino acid unit is valine-citrulline (vc or Val-Cit). In another specific embodiment, the amino acid unit is phenylalanine-lysine (i.e., fk). In yet another specific embodiment, the amino acid unit is N-methylvaline-citrulline. In yet another specific embodiment, the amino acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

### 5.3.3.3 Spacer Unit

The spacer unit (-Y-), when present, links an amino acid unit to the drug unit when an amino acid unit is present. Alternately, the spacer unit links the stretcher unit to the drug unit when the amino acid unit is absent. The spacer unit also links the drug unit to the antibody unit when both the amino acid unit and stretcher unit are absent.

Spacer units are of two general types: non self-immolative or self-immolative. A non self-immolative spacer unit is one in which part or all of the spacer unit remains bound to the drug unit after cleavage, particularly enzymatic, of an amino acid unit from the antibody drug conjugate. Examples of a non self-immolative spacer unit include, but are not limited to a (glycine-glycine) spacer unit and a glycine spacer unit (both depicted in Scheme 1) (infra). When a conjugate containing a glycine-glycine spacer unit or a glycine Spacer unit undergoes enzymatic cleavage via an enzyme (e.g., a tumor-cell associated-protease, a cancer-cell-associated protease or a lymphocyte-associated protease), a glycine-glycine-drug unit or a glycine-drug unit is cleaved from L-Aa-Ww-. In one embodiment, an independent hydrolysis reaction takes place within the target cell, cleaving the glycine-drug unit bond and liberating the drug.

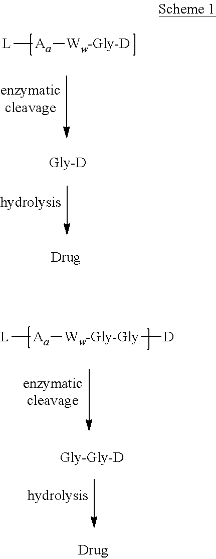

In some embodiments, a non self-immolative spacer unit (-Y-) is -Gly-. In some embodiments, a non self-immolative spacer unit (-Y-) is -Gly-Gly-.

In one embodiment, the spacer unit is absent (-Y_{y}- where y=0).

Alternatively, an antibody drug conjugate containing a self-immolative spacer unit can release -D. As used herein, the term “self-immolative spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, -Y_{y}- is a p-aminobenzyl alcohol (PAB) unit (see Schemes 2 and 3) whose phenylene portion is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

In some embodiments, -Y- is a PAB group that is linked to -Ww- via the amino nitrogen atom of the PAB group, and connected directly to -D via a carbonate, carbamate or ether group. Without being bound by any particular theory or mechanism, Scheme 2 depicts a possible mechanism of Drug release of a PAB group which is attached directly to -D via a carbamate or carbonate group as described by Toki et al., 2002, J. Org. Chem. 67:1866-1872.

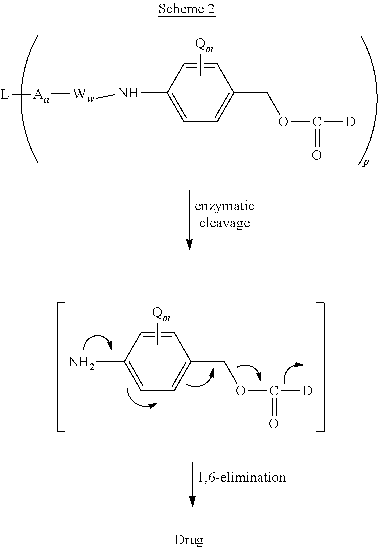

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Without being bound by any particular theory or mechanism, Scheme 3 depicts a possible mechanism of drug release of a PAB group which is attached directly to -D via an ether or amine linkage, wherein D includes the oxygen or nitrogen group that is part of the drug unit.

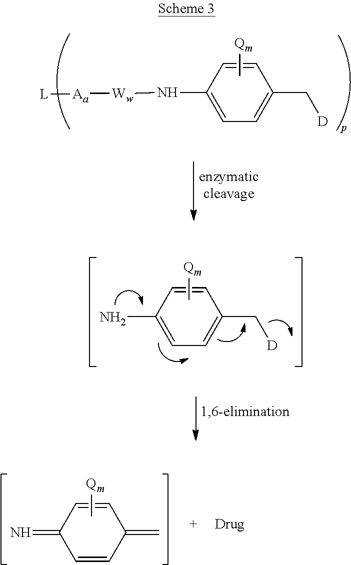

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867). Elimination of amine-containing drugs that are substituted at the α-position of glycine (Kingsbury et al., 1984, 1 Med. Chem. 27:1447) are also examples of self-immolative spacers.

In one embodiment, the spacer unit is a branched bis(hydroxymethyl)-styrene (BHMS) unit as depicted in Scheme 4, which can be used to incorporate and release multiple drugs.

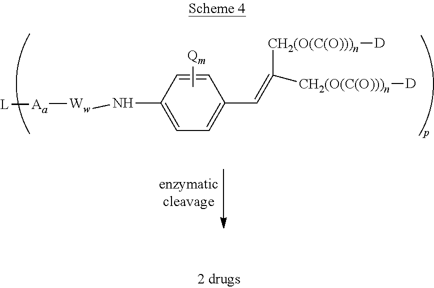

In Scheme 4, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; n is 0 or 1; and p ranges ranging from 1 to about 20. The alkyl, alkenyl and alkynyl groups, whether alone or as part of another group, can be optionally substituted.

In some embodiments, the -D units are the same. In yet another embodiment, the -D moieties are different.

In one aspect, spacer units (-Y_{y}-) are represented by Formulas X-XII:

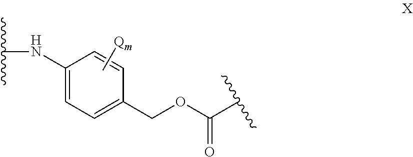

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

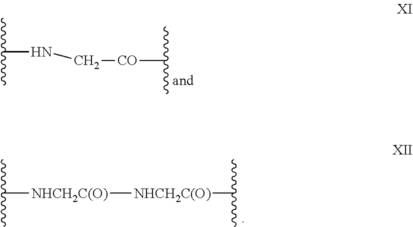

Embodiments of the Formula I and II comprising antibody-drug conjugate compounds can include:

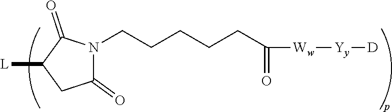

wherein w and y are each 0, 1 or 2, and,

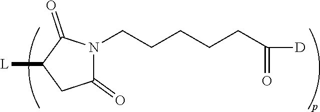

wherein w and y are each 0,

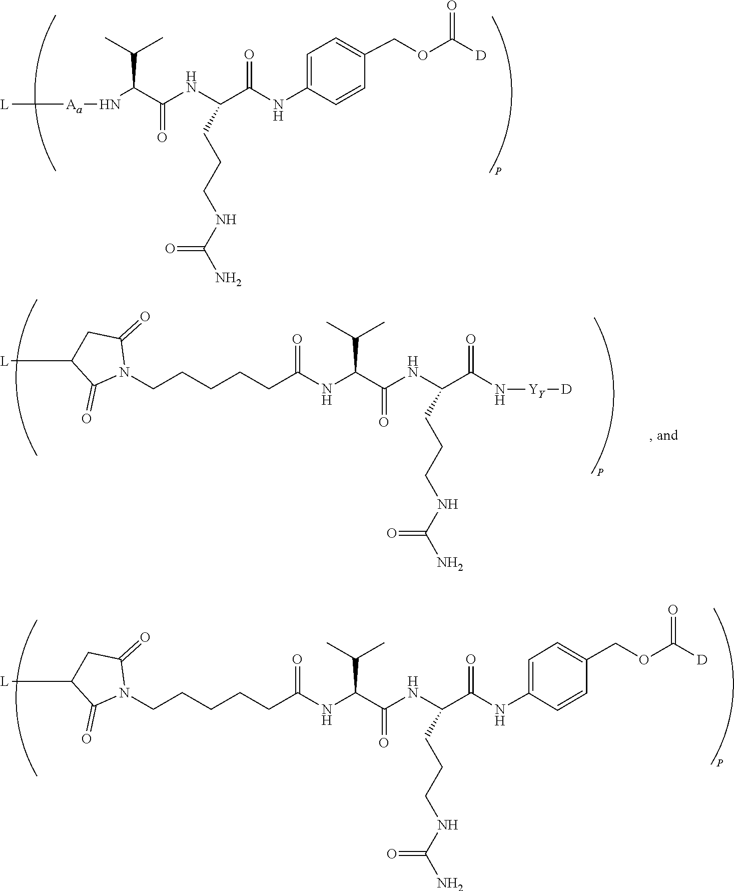

### 5.3.3.4 Drug Loading

Drug loading is represented by p and is the average number of drug units per antibody in a molecule. Drug loading can range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

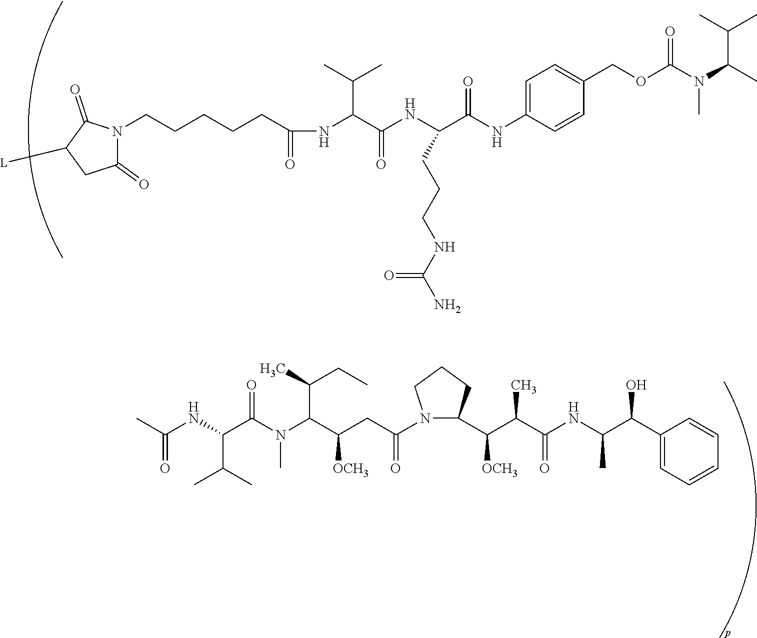

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1κ) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is the a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, ASG-22C3E. The enfortumab vedotin includes an anti-191P4D12 antibody, wherein the antibody or antigen binding fragment thereof comprises a heavy chain comprising amino acid residue 20 to amino acid residue 466 of SEQ ID NO: 7 and a light chain comprising amino acid residue 23 to amino acid residue 236 of SEQ ID NO:8.

Enfortumab vedotin is a Nectin-4 directed antibody -drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

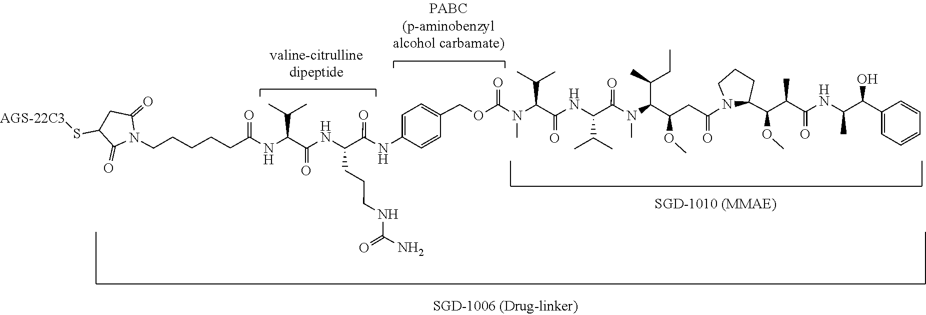

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises

L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein

has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments,

the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

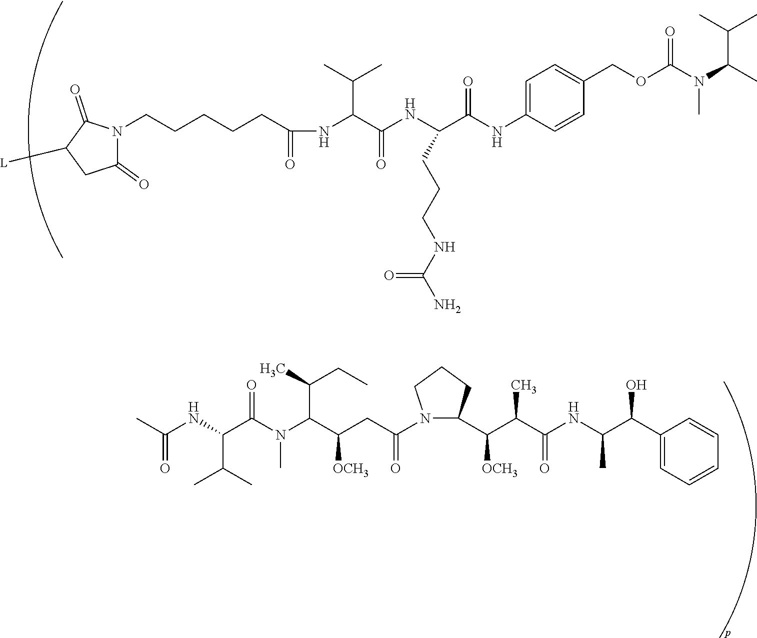

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

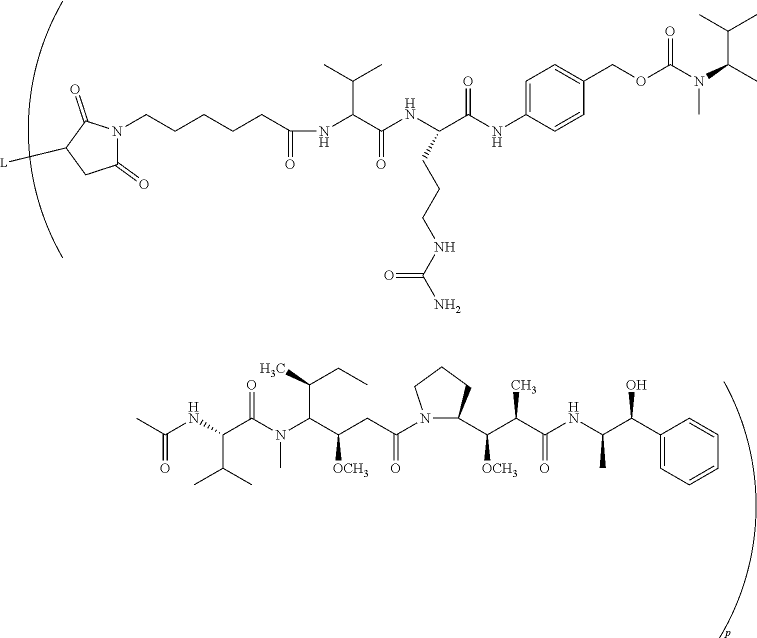

wherein L- represents the antibody or antigen binding fragment thereof (e.g. anti-nectin-4 antibody or antigen binding fragment thereof) and p is from 1 to10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

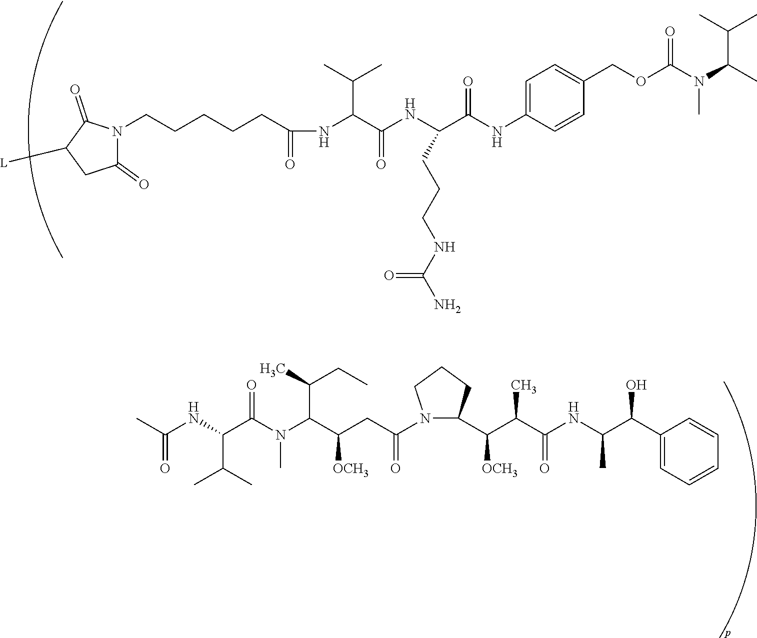

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

### 5.5 Methods for a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the chemotherapeutic agent is administered separately. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation, used in combination with the pharmaceutical composition provided herein, can be either external or internal to the patient being treated. When the source is external to the patient, the therapy is known as external beam radiation therapy (EBRT). When the source of radiation is internal to the patient, the treatment is called brachytherapy (BT).

The above described therapeutic regimens can be further combined with additional cancer treating agents and/or regimes, for example additional chemotherapy, cancer vaccines, signal transduction inhibitors, agents useful in treating abnormal cell growth or cancer, antibodies (e.g. Anti-CTLA-4 antibodies as described in WO/2005/092380 (Pfizer)) or other ligands that inhibit tumor growth by binding to IGF-1R, and cytokines.

When the mammal is subjected to additional chemotherapy, chemotherapeutic agents described above can be used. Additionally, growth factor inhibitors, biological response modifiers, anti-hormonal therapy, selective estrogen receptor modulators (SERMs), angiogenesis inhibitors, and anti-androgens can be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3-′-(trifluoromethyl)propionanilide) can be used.

In some embodiments, the pharmaceutical provided herein in used in combination with a second therapeutic agent, e.g., for treating a cancer.

### 5.6 Doses for the Immune Checkpoint Inhibitors

In some embodiments, the amount of the checkpoint inhibitor for the various methods provided herein be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

### 5.7 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses can be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses can be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment.

In other more specific embodiments, the antibody drug conjugate formulated in the pharmaceutical composition provided herein is administered at a dose of about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment.

In some embodiments of the various methods provided herein, the ADC is administered at a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments, the ADC is administered at a dose of about 1 to about 10 mg/kg of the subject's body weight. In certain embodiments, the ADC is administered at a dose of about 1 to about 5 mg/kg of the subject's body weight. In other embodiments, the ADC is administered at a dose of about 1 to about 2.5 mg/kg of the subject's body weight. In further embodiments, the ADC is administered at a dose of about 1 to about 1.25 mg/kg of the subject's body weight. In some embodiments, the ADC is administered at a dose of about 0.25 mg/kg of the subject's body weight. In some embodiments, the ADC is administered at a dose of about 0.5 mg/kg of the subject's body weight. In some embodiments, the ADC is administered at a dose of about 0.75 mg/kg of the subject's body weight. In some embodiments, the ADC is administered at a dose of about 1.0 mg/kg of the subject's body weight. In some embodiments, the ADC is administered at a dose of about 1.25 mg/kg of the subject's body weight. In some embodiments, the ADC is administered at a dose of about 1.5 mg/kg of the subject's body weight. In some embodiments, the ADC is administered at a dose of about 1.75 mg/kg of the subject's body weight. In some embodiments, the ADC is administered at a dose of about 2.0 mg/kg of the subject's body weight. In some embodiments, the ADC is administered at a dose of about 2.25 mg/kg of the subject's body weight. In some embodiments, the ADC is administered at a dose of about 2.5 mg/kg of the subject's body weight.

In certain embodiments of the various methods provided herein, the ADC is administered at a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In some embodiments, the ADC is administered at a dose of 1 to 10 mg/kg of the subject's body weight. In certain embodiments, the ADC is administered at a dose of 1 to 5 mg/kg of the subject's body weight. In other embodiments, the ADC is administered at a dose of 1 to 2.5 mg/kg of the subject's body weight. In further embodiments, the ADC is administered at a dose of 1 to 1.25 mg/kg of the subject's body weight. In some embodiments, the ADC is administered at a dose of 0.25 mg/kg of the subject's body weight. In some embodiments, the ADC is administered at a dose of 0.5 mg/kg of the subject's body weight. In some embodiments, the ADC is administered at a dose of 0.75 mg/kg of the subject's body weight. In some embodiments, the ADC is administered at a dose of 1.0 mg/kg of the subject's body weight. In some embodiments, the ADC is administered at a dose of 1.25 mg/kg of the subject's body weight. In some embodiments, the ADC is administered at a dose of 1.5 mg/kg of the subject's body weight. In some embodiments, the ADC is administered at a dose of 1.75 mg/kg of the subject's body weight. In some embodiments, the ADC is administered at a dose of 2.0 mg/kg of the subject's body weight. In some embodiments, the ADC is administered at a dose of 2.25 mg/kg of the subject's body weight. In some embodiments, the ADC is administered at a dose of 2.5 mg/kg of the subject's body weight.

In some embodiments of the various methods provided herein, including those methods requiring a first and a second dose, the first dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the first dose of the ADC is a dose of about 1 to about 10 mg/kg of the subject's body weight. In some embodiments of the various methods provided herein, including those methods requiring a first and a second dose, the first dose of the ADC is a dose of about 1 to about 5 mg/kg of the subject's body weight. In some embodiments of the various methods provided herein, including those methods requiring a first and a second dose, the first dose of the ADC is a dose of about 1 to about 2.5 mg/kg of the subject's body weight. In some embodiments of the various methods provided herein, including those methods requiring a first and a second dose, the first dose of the ADC is a dose of about 1 to about 1.25 mg/kg of the subject's body weight. In some embodiments of the various methods provided herein, including those methods requiring a first and a second dose, the first dose of the ADC is a dose of about 0.25 mg/kg of the subject's body weight. In some embodiments of the various methods provided herein, including those methods requiring a first and a second dose, the first dose of the ADC is a dose of about 0.5 mg/kg of the subject's body weight. In some embodiments, the first dose of the ADC is a dose of about 0.75 mg/kg of the subject's body weight. In some embodiments, the first dose of the ADC is a dose of about 1.0 mg/kg of the subject's body weight. In some embodiments, the first dose of the ADC is a dose of about 1.25 mg/kg of the subject's body weight. In some embodiments, the first dose of the ADC is a dose of about 1.5 mg/kg of the subject's body weight. In some embodiments, the first dose of the ADC is a dose of about 1.75 mg/kg of the subject's body weight. In some embodiments, the first dose of the ADC is a dose of about 2.0 mg/kg of the subject's body weight. In some embodiments, the first dose of the ADC is a dose of about 2.25 mg/kg of the subject's body weight. In some embodiments, the first dose of the ADC is a dose of or about 2.5 mg/kg of the subject's body weight.

In certain embodiments of the various methods provided herein, including those methods requiring a first and a second dose, the first dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the first dose of the ADC is a dose of 1 to 10 mg/kg of the subject's body weight. In some embodiments of the various methods provided herein, including those methods requiring a first and a second dose, the first dose of the ADC is a dose of 1 to 5 mg/kg of the subject's body weight. In some embodiments of the various methods provided herein, including those methods requiring a first and a second dose, the first dose of the ADC is a dose of 1 to 2.5 mg/kg of the subject's body weight. In some embodiments of the various methods provided herein, including those methods requiring a first and a second dose, the first dose of the ADC is a dose of 1 to 1.25 mg/kg of the subject's body weight. In some embodiments of the various methods provided herein, including those methods requiring a first and a second dose, the first dose of the ADC is a dose of 0.25 mg/kg of the subject's body weight. In some embodiments of the various methods provided herein, including those methods requiring a first and a second dose, the first dose of the ADC is a dose of 0.5 mg/kg of the subject's body weight. In some embodiments, the first dose of the ADC is a dose of 0.75 mg/kg of the subject's body weight. In some embodiments, the first dose of the ADC is a dose of 1.0 mg/kg of the subject's body weight. In some embodiments, the first dose of the ADC is a dose of 1.25 mg/kg of the subject's body weight. In some embodiments, the first dose of the ADC is a dose of 1.5 mg/kg of the subject's body weight. In some embodiments, the first dose of the ADC is a dose of 1.75 mg/kg of the subject's body weight. In some embodiments, the first dose of the ADC is a dose of 2.0 mg/kg of the subject's body weight. In some embodiments, the first dose of the ADC is a dose of 2.25 mg/kg of the subject's body weight. In some embodiments, the first dose of the ADC is a dose of 2.5 mg/kg of the subject's body weight.

In some embodiments of the various methods provided herein, including those methods requiring a first and a second dose, the second dose of the ADC is lower than the first dose by about 0.1 mg/kg to about 2 mg/kg of the subject's body weight. In some embodiments of the various methods provided herein, including those methods requiring a first and a second dose, the second dose of the ADC is lower than the first dose by about 0.1 mg/kg of the subject's body weight. In some embodiments of the various methods provided herein, including those methods requiring a first and a second dose, the second dose of the ADC is lower than the first dose by about 0.2 mg/kg of the subject's body weight. In some embodiments of the various methods provided herein, including those methods requiring a first and a second dose, the second dose of the ADC is lower than the first dose by about 0.25 mg/kg of the subject's body weight. In some embodiments of the various methods provided herein, including those methods requiring a first and a second dose, the second dose of the ADC is lower than the first dose by about 0.3 mg/kg of the subject's body weight. In some embodiments of the various methods provided herein, including those methods requiring a first and a second dose, the second dose of the ADC is lower than the first dose by about 0.4 mg/kg of the subject's body weight. In some embodiments of the various methods provided herein, including those methods requiring a first and a second dose, the second dose of the ADC is lower than the first dose by about 0.5 mg/kg of the subject's body weight. In some embodiments of the various methods provided herein, including those methods requiring a first and a second dose, the second dose of the ADC is lower than the first dose by about 0.6 mg/kg of the subject's body weight. In some embodiments of the various methods provided herein, including those methods requiring a first and a second dose, the second dose of the ADC is lower than the first dose by about 0.7 mg/kg of the subject's body weight. In some embodiments of the various methods provided herein, including those methods requiring a first and a second dose, the second dose of the ADC is lower than the first dose by about 0.75 mg/kg of the subject's body weight. In some embodiments of the various methods provided herein, including those methods requiring a first and a second dose, the second dose of the ADC is lower than the first dose by about 0.8 mg/kg of the subject's body weight. In some embodiments of the various methods provided herein, including those methods requiring a first and a second dose, the second dose of the ADC is lower than the first dose by about 0.9 mg/kg of the subject's body weight. In some embodiments of the various methods provided herein, including those methods requiring a first and a second dose, the second dose of the ADC is lower than the first dose by about 1 mg/kg of the subject's body weight. In some embodiments of the various methods provided herein, including those methods requiring a first and a second dose, the second dose of the ADC is lower than the first dose by about 1.1 mg/kg of the subject's body weight. In some embodiments of the various methods provided herein, including those methods requiring a first and a second dose, the second dose of the ADC is lower than the first dose by about 1.2 mg/kg of the subject's body weight. In some embodiments of the various methods provided herein, including those methods requiring a first and a second dose, the second dose of the ADC is lower than the first dose by about 1.25 mg/kg of the subject's body weight. In some embodiments of the various methods provided herein, including those methods requiring a first and a second dose, the second dose of the ADC is lower than the first dose by about 1.3 mg/kg of the subject's body weight. In some embodiments of the various methods provided herein, including those methods requiring a first and a second dose, the second dose of the ADC is lower than the first dose by about 1.4 mg/kg of the subject's body weight. In some embodiments of the various methods provided herein, including those methods requiring a first and a second dose, the second dose of the ADC is lower than the first dose by about 1.5 mg/kg of the subject's body weight. In some embodiments of the various methods provided herein, including those methods requiring a first and a second dose, the second dose of the ADC is lower than the first dose by about 1.6 mg/kg of the subject's body weight. In some embodiments of the various methods provided herein, including those methods requiring a first and a second dose, the second dose of the ADC is lower than the first dose by about 1.7 mg/kg of the subject's body weight. In some embodiments of the various methods provided herein, including those methods requiring a first and a second dose, the second dose of the ADC is lower than the first dose by about 1.75 mg/kg of the subject's body weight. In some embodiments of the various methods provided herein, including those methods requiring a first and a second dose, the second dose of the ADC is lower than the first dose by about 1.8 mg/kg of the subject's body weight. In some embodiments of the various methods provided herein, including those methods requiring a first and a second dose, the second dose of the ADC is lower than the first dose by about 1.9 mg/kg of the subject's body weight. In some embodiments of the various methods provided herein, including those methods requiring a first and a second dose, the second dose of the ADC is lower than the first dose by about 2 mg/kg of the subject's body weight.

In certain embodiments of the various methods provided herein, including those methods requiring a first and a second dose, the second dose of the ADC is lower than the first dose by 0.1 mg/kg to 2 mg/kg of the subject's body weight. In some embodiments of the various methods provided herein, including those methods requiring a first and a second dose, the second dose of the ADC is lower than the first dose by 0.1 mg/kg of the subject's body weight. In some embodiments of the various methods provided herein, including those methods requiring a first and a second dose, the second dose of the ADC is lower than the first dose by 0.2 mg/kg of the subject's body weight. In some embodiments of the various methods provided herein, including those methods requiring a first and a second dose, the second dose of the ADC is lower than the first dose by 0.25 mg/kg of the subject's body weight. In some embodiments of the various methods provided herein, including those methods requiring a first and a second dose, the second dose of the ADC is lower than the first dose by 0.3 mg/kg of the subject's body weight. In some embodiments of the various methods provided herein, including those methods requiring a first and a second dose, the second dose of the ADC is lower than the first dose by 0.4 mg/kg of the subject's body weight. In some embodiments of the various methods provided herein, including those methods requiring a first and a second dose, the second dose of the ADC is lower than the first dose by 0.5 mg/kg of the subject's body weight. In some embodiments of the various methods provided herein, including those methods requiring a first and a second dose, the second dose of the ADC is lower than the first dose by 0.6 mg/kg of the subject's body weight. In some embodiments of the various methods provided herein, including those methods requiring a first and a second dose, the second dose of the ADC is lower than the first dose by 0.7 mg/kg of the subject's body weight. In some embodiments of the various methods provided herein, including those methods requiring a first and a second dose, the second dose of the ADC is lower than the first dose by 0.75 mg/kg of the subject's body weight. In some embodiments of the various methods provided herein, including those methods requiring a first and a second dose, the second dose of the ADC is lower than the first dose by 0.8 mg/kg of the subject's body weight. In some embodiments of the various methods provided herein, including those methods requiring a first and a second dose, the second dose of the ADC is lower than the first dose by 0.9 mg/kg of the subject's body weight. In some embodiments of the various methods provided herein, including those methods requiring a first and a second dose, the second dose of the ADC is lower than the first dose by 1 mg/kg of the subject's body weight. In some embodiments of the various methods provided herein, including those methods requiring a first and a second dose, the second dose of the ADC is lower than the first dose by 1.1 mg/kg of the subject's body weight. In some embodiments of the various methods provided herein, including those methods requiring a first and a second dose, the second dose of the ADC is lower than the first dose by 1.2 mg/kg of the subject's body weight. In some embodiments of the various methods provided herein, including those methods requiring a first and a second dose, the second dose of the ADC is lower than the first dose by 1.25 mg/kg of the subject's body weight. In some embodiments of the various methods provided herein, including those methods requiring a first and a second dose, the second dose of the ADC is lower than the first dose by 1.3 mg/kg of the subject's body weight. In some embodiments of the various methods provided herein, including those methods requiring a first and a second dose, the second dose of the ADC is lower than the first dose by 1.4 mg/kg of the subject's body weight. In some embodiments of the various methods provided herein, including those methods requiring a first and a second dose, the second dose of the ADC is lower than the first dose by 1.5 mg/kg of the subject's body weight. In some embodiments of the various methods provided herein, including those methods requiring a first and a second dose, the second dose of the ADC is lower than the first dose by 1.6 mg/kg of the subject's body weight. In some embodiments of the various methods provided herein, including those methods requiring a first and a second dose, the second dose of the ADC is lower than the first dose by 1.7 mg/kg of the subject's body weight. In some embodiments of the various methods provided herein, including those methods requiring a first and a second dose, the second dose of the ADC is lower than the first dose by 1.75 mg/kg of the subject's body weight. In some embodiments of the various methods provided herein, including those methods requiring a first and a second dose, the second dose of the ADC is lower than the first dose by 1.8 mg/kg of the subject's body weight. In some embodiments of the various methods provided herein, including those methods requiring a first and a second dose, the second dose of the ADC is lower than the first dose by 1.9 mg/kg of the subject's body weight. In some embodiments of the various methods provided herein, including those methods requiring a first and a second dose, the second dose of the ADC is lower than the first dose by 2 mg/kg of the subject's body weight.

In some embodiments of the various methods provided herein, including those methods requiring a first and a second dose, the second dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the second dose of the ADC is a dose of about 1 to about 10 mg/kg of the subject's body weight. In some embodiments of the various methods provided herein, including those methods requiring a first and a second dose, the second dose of the ADC is a dose of about 1 to about 5 mg/kg of the subject's body weight. In some embodiments of the various methods provided herein, including those methods requiring a first and a second dose, the second dose of the ADC is a dose of about 1 to about 2.5 mg/kg of the subject's body weight. In some embodiments of the various methods provided herein, including those methods requiring a first and a second dose, the second dose of the ADC is a dose of about 1 to about 1.25 mg/kg of the subject's body weight. In some embodiments of the various methods provided herein, including those methods requiring a first and a second dose, the second dose of the ADC is a dose of about 0.25 mg/kg of the subject's body weight. In some embodiments of the various methods provided herein, including those methods requiring a first and a second dose, the second dose of the ADC is a dose of about 0.5 mg/kg of the subject's body weight. In some embodiments of the various methods provided herein, including those methods requiring a first and a second dose, the second dose of the ADC is a dose of about 0.75 mg/kg of the subject's body weight. In some embodiments of the various methods provided herein, including those methods requiring a first and a second dose, the second dose of the ADC is a dose of about 1.0 mg/kg of the subject's body weight. In some embodiments of the various methods provided herein, including those methods requiring a first and a second dose, the second dose of the ADC is a dose of about 1.25 mg/kg of the subject's body weight. In some embodiments of the various methods provided herein, including those methods requiring a first and a second dose, the second dose of the ADC is a dose of about 1.5 mg/kg of the subject's body weight. In some embodiments of the various methods provided herein, including those methods requiring a first and a second dose, the second dose of the ADC is a dose of about 1.75 mg/kg of the subject's body weight. In some embodiments of the various methods provided herein, including those methods requiring a first and a second dose, the second dose of the ADC is a dose of about 2.0 mg/kg of the subject's body weight. In some embodiments of the various methods provided herein, including those methods requiring a first and a second dose, the second dose of the ADC is a dose of about 2.25 mg/kg of the subject's body weight. In some embodiments of the various methods provided herein, including those methods requiring a first and a second dose, the second dose of the ADC is a dose of about 2.5 mg/kg of the subject's body weight.

In certain embodiments of the various methods provided herein, including those methods requiring a first and a second dose, the second dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the second dose of the ADC is a dose of 1 to 10 mg/kg of the subject's body weight. In some embodiments of the various methods provided herein, including those methods requiring a first and a second dose, the second dose of the ADC is a dose of 1 to 5 mg/kg of the subject's body weight. In some embodiments of the various methods provided herein, including those methods requiring a first and a second dose, the second dose of the ADC is a dose of 1 to 2.5 mg/kg of the subject's body weight. In some embodiments of the various methods provided herein, including those methods requiring a first and a second dose, the second dose of the ADC is a dose of 1 to 1.25 mg/kg of the subject's body weight. In some embodiments of the various methods provided herein, including those methods requiring a first and a second dose, the second dose of the ADC is a dose of 0.25 mg/kg of the subject's body weight. In some embodiments of the various methods provided herein, including those methods requiring a first and a second dose, the second dose of the ADC is a dose of 0.5 mg/kg of the subject's body weight. In some embodiments of the various methods provided herein, including those methods requiring a first and a second dose, the second dose of the ADC is a dose of 0.75 mg/kg of the subject's body weight. In some embodiments of the various methods provided herein, including those methods requiring a first and a second dose, the second dose of the ADC is a dose of 1.0 mg/kg of the subject's body weight. In some embodiments of the various methods provided herein, including those methods requiring a first and a second dose, the second dose of the ADC is a dose of 1.25 mg/kg of the subject's body weight. In some embodiments of the various methods provided herein, including those methods requiring a first and a second dose, the second dose of the ADC is a dose of 1.5 mg/kg of the subject's body weight. In some embodiments of the various methods provided herein, including those methods requiring a first and a second dose, the second dose of the ADC is a dose of 1.75 mg/kg of the subject's body weight. In some embodiments of the various methods provided herein, including those methods requiring a first and a second dose, the second dose of the ADC is a dose of 2.0 mg/kg of the subject's body weight. In some embodiments of the various methods provided herein, including those methods requiring a first and a second dose, the second dose of the ADC is a dose of 2.25 mg/kg of the subject's body weight. In some embodiments of the various methods provided herein, including those methods requiring a first and a second dose, the second dose of the ADC is a dose of 2.5 mg/kg of the subject's body weight.

In certain embodiments of the various methods provided herein, including those methods requiring a first and a second dose, the second dose of the ADC is identical to the first dose of the ADC.

In some embodiments of the methods provided herein, the ADC is administered by an intravenous (IV) injection or infusion. In one embodiment, the first dose of the ADC is administered by an IV injection. In another embodiment, the first dose of the ADC is administered by an IV infusion. In yet another embodiment, the second dose of the ADC is administered by an IV injection. In yet another embodiment, the second dose of the ADC is administered by an IV injection infusion. In one embodiment, the first dose of the ADC is administered by an IV injection and the second dose of the ADC is administered by an IV injection. In another embodiment, the first dose of the ADC is administered by an IV infusion and the second dose of the ADC is administered by an IV injection. In yet another embodiment, the second dose of the ADC is administered by an IV injection and the second dose of the ADC is administered by an IV injection infusion. In yet another embodiment, the second dose of the ADC is administered by an IV injection infusion and the second dose of the ADC is administered by an IV injection infusion.

In certain embodiments of the methods provided herein, the ADC is administered by an IV injection or infusion three times every four-week cycle. In some embodiments of the methods provided herein, the first dose of the ADC is administered by an IV injection or infusion three times every four-week cycle. In some embodiments of the methods provided herein, the second dose of the ADC is administered by an IV injection or infusion three times every four-week cycle. In some embodiments of the methods provided herein, the first dose of the ADC is administered by an IV injection or infusion three times every four-week cycle and the second dose of the ADC is administered by an IV injection or infusion three times every four-week cycle.

In some embodiments of the methods provided herein, the ADC is administered by an IV injection or infusion on Days 1, 8 and 15 of every four-week cycle. In some embodiments, the first dose of ADC is administered by an IV injection or infusion on Days 1, 8 and 15 of every four-week cycle. In some embodiments, the second dose of ADC is administered by an IV injection or infusion on Days 1, 8 and 15 of every four-week cycle. In some embodiments, the first dose of ADC is administered by an IV injection or infusion on Days 1, 8 and 15 of every four-week cycle and the second dose of ADC is administered by an IV injection or infusion on Days 1, 8 and 15 of every four-week cycle.

In certain embodiments of the methods provided herein, the ADC is administered by an IV injection or infusion over about 30 minutes three times every four-week cycle. In some embodiments, the first dose of the ADC is administered by an IV injection or infusion over about 30 minutes three times every four-week cycle. In some embodiments, the second dose of the ADC is administered by an IV injection or infusion over about 30 minutes three times every four-week cycle. In some embodiments, the first dose of the ADC is administered by an IV injection or infusion over about 30 minutes three times every four-week cycle and the second dose of the ADC is administered by an IV injection or infusion over about 30 minutes three times every four-week cycle.

In some embodiments of the methods provided herein, the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle. In some embodiments of the methods provided herein, the second dose of the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle and the second dose of the ADC is administered by an IV injection or infusion over about 30 minutes on Days 1, 8 and 15 of every four-week cycle.

### 5.8 Methods for Determining the Expression of Various Marker Genes

The disclosure provides that the expression of any of the marker genes provided herein can be determined by various methods known in the field. In some embodiments, the expression of the marker genes can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D′haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene -mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000— which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistrcy (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3,3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9, 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9, 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, Mass. USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, Mass. USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford Ill.; Santa Cruz Biotechnology, Dallas Tex.; Invitrogen, Carlsbad Calif.; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway N.J.), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.8) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 μm and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm² is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number >8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

### 5.9 Cancers and Cancer Patients to Which the Methods Are Applicable

The disclosure provides that the methods provided herein can be used to treat patient with various cancers. In certain embodiments of the various methods provided herein, the subject is a human. In one embodiment, the subject is a human (a patient) having cancer. In another embodiment, the subject is a human (a patient) having a disease or disorder. In yet another embodiment, the subject is a subject having cancer.

In some embodiments of the methods provided herein, the cancer is bladder cancer, urothelial cancer, gastric cancer, esophageal cancer, head cancer, neck cancer, squamous non-small cell lung cancer (NSCLC), non-squamous NSCLC, breast cancer, ovarian cancer, cervical cancer, biliary cancer and cholangiocarcinomas, pancreatic cancer, squamous cell carcinoma of the vulva and penis, prostate adenocarcinoma, or endometrial carcinoma. In one embodiment, the cancer is bladder cancer. In another embodiment, the cancer is urothelial cancer. In another embodiment, the cancer is gastric cancer. In yet another embodiment, the cancer is esophageal cancer. In a further embodiment, the cancer is head cancer. In one embodiment, the cancer is neck cancer. In another embodiment, the cancer is NSCLC. In yet another embodiment, the cancer is non-squamous NSCLC. In a further embodiment, the cancer is breast cancer. In one embodiment, the cancer is ovarian cancer. In another embodiment, the cancer is cervical cancer. In a further embodiment, the cancer is biliary cancer. In one embodiment, the cancer is cholangiocarcinomas. In another embodiment, the cancer is biliary cancer and cholangiocarcinomas. In yet another embodiment, the cancer is pancreatic cancer. In a further embodiment, the cancer is squamous cell carcinoma of the vulva. In one embodiment, the cancer is squamous cell carcinoma of the penis. In another embodiment, the cancer is squamous cell carcinoma of the vulva and penis. In a further embodiment, the cancer is prostate adenocarcinoma. In one embodiment, the cancer is endometrial carcinoma.

In some embodiments of the methods provided herein, the cancer is locally advanced cancer. In one embodiment, the cancer is locally advanced bladder cancer. In another embodiment, the cancer is locally advanced urothelial cancer. In another embodiment, the cancer is locally advanced gastric cancer. In yet another embodiment, the cancer is locally advanced esophageal cancer. In a further embodiment, the cancer is locally advanced head cancer. In one embodiment, the cancer is locally advanced neck cancer. In another embodiment, the cancer is locally advanced NSCLC. In yet another embodiment, the cancer is locally advanced non-squamous NSCLC. In a further embodiment, the cancer is locally advanced breast cancer. In one embodiment, the cancer is locally advanced ovarian cancer. In another embodiment, the cancer is locally advanced cervical cancer. In a further embodiment, the cancer is locally advanced biliary cancer. In one embodiment, the cancer is locally advanced cholangiocarcinomas. In another embodiment, the cancer is locally advanced biliary cancer and cholangiocarcinomas. In yet another embodiment, the cancer is locally advanced pancreatic cancer. In a further embodiment, the cancer is locally advanced squamous cell carcinoma of the vulva. In one embodiment, the cancer is locally advanced squamous cell carcinoma of the penis. In another embodiment, the cancer is locally advanced squamous cell carcinoma of the vulva and penis. In a further embodiment, the cancer is locally advanced prostate adenocarcinoma. In one embodiment, the cancer is locally advanced endometrial carcinoma.

In some embodiments of the methods provided herein, the cancer is metastatic cancer. In one embodiment, the cancer is metastatic bladder cancer. In another embodiment, the cancer is metastatic urothelial cancer. In another embodiment, the cancer is metastatic gastric cancer. In yet another embodiment, the cancer is metastatic esophageal cancer. In a further embodiment, the cancer is metastatic head cancer. In one embodiment, the cancer is metastatic neck cancer. In another embodiment, the cancer is metastatic NSCLC. In yet another embodiment, the cancer is metastatic non-squamous NSCLC. In a further embodiment, the cancer is metastatic breast cancer. In one embodiment, the cancer is metastatic ovarian cancer. In another embodiment, the cancer is metastatic cervical cancer. In a further embodiment, the cancer is metastatic biliary cancer. In one embodiment, the cancer is metastatic cholangiocarcinomas. In another embodiment, the cancer is metastatic biliary cancer and cholangiocarcinomas. In yet another embodiment, the cancer is metastatic pancreatic cancer. In a further embodiment, the cancer is metastatic squamous cell carcinoma of the vulva. In one embodiment, the cancer is metastatic squamous cell carcinoma of the penis. In another embodiment, the cancer is metastatic squamous cell carcinoma of the vulva and penis. In a further embodiment, the cancer is metastatic prostate adenocarcinoma. In one embodiment, the cancer is metastatic endometrial carcinoma.

In some specific embodiments of the methods provided herein, the breast cancer is ER negative, PR negative, and HER2 negative (ER−/PR−/HER2−) breast cancer. In one specific embodiment, the breast cancer is ER−/PR−/HER2− and locally advanced breast cancer. In another specific embodiment, the breast cancer is ER−/PR−/HER2− and metastatic breast cancer. In yet another specific embodiment, the breast cancer is hormone receptor positive and human epidermal growth factor receptor 2 negative (HR+/HER2−) breast cancer. In a further specific embodiment, the breast cancer is HR+/HER2− and locally advanced breast cancer. In one embodiment, the breast cancer is HR+/HER2− and metastatic breast cancer.

In some specific embodiments, the urothelial cancer is papillary urothelial carcinoma or flat urothelial carcinoma. In certain embodiments, the urothelial cancer is papillary urothelial carcinoma. In some embodiments, the urothelial cancer is flat urothelial carcinoma. In certain embodiments, the urothelial cancer is locally advanced papillary urothelial carcinoma. In some embodiments, the urothelial cancer is locally advanced flat urothelial carcinoma. In certain embodiments, the urothelial cancer is metastatic papillary urothelial carcinoma. In some embodiments, the urothelial cancer is metastatic flat urothelial carcinoma.

In other specific embodiments, the bladder cancer is non-muscle-invasive bladder cancer (NMIBC) or muscle-invasive bladder cancer. In one embodiment, the bladder cancer is NMIBC. In another embodiment, the bladder cancer is muscle-invasive bladder cancer. In yet another embodiment, the bladder cancer is locally advanced NMIBC. In a further embodiment, the bladder cancer is locally advanced muscle-invasive bladder cancer. In another embodiment, the bladder cancer is metastatic NMIBC. In one embodiment, the bladder cancer is metastatic muscle-invasive bladder cancer. In another embodiment, the muscle-invasive bladder cancer is squamous cell carcinoma, adenocarcinoma, small cell carcinoma, or sarcoma. In yet another embodiment, the muscle-invasive bladder cancer is squamous cell carcinoma. In a further embodiment, the muscle-invasive bladder cancer is adenocarcinoma. In one embodiment, the muscle-invasive bladder cancer is small cell carcinoma. In another embodiment, the muscle-invasive bladder cancer is sarcoma.

In certain embodiments, the methods provided herein are used for the treatment of breast cancer in a subject. In some embodiments, the breast cancer is hormone receptor positive and human epidermal growth factor receptor 2 negative (HR+/HER2−) breast cancer. In some embodiments, the breast cancer is estrogen receptor (ER) positive and/or progesterone receptor (PR) positive, and HER2 negative. In some embodiments, the breast cancer is ER positive, PR positive, and HER2 negative. In some embodiments, the breast cancer is ER positive and HER2 negative. In some embodiments, the breast cancer is PR positive and HER2 negative. In some embodiments, the breast cancers, including for example, the HR+/HER2− breast cancer, the ER positive, PR positive, and HER2 negative breast cancer, the ER positive and HER2 negative breast cancer, the PR positive and HER2 negative breast cancer are confirmed histologically, cytologically, or both histologically and cytologically. In some embodiments, the histological, cytological, or both histological and cytological confirmation are conducted per American Society of Clinical Oncology/College of American Pathologists (ASCO/CAP) guidelines based on the most recently analyzed tissue.

In some embodiments, the hormone receptor positive and human epidermal growth factor receptor 2 negative (HR+/HER2−) breast cancer is locally advanced or metastatic (including malignant or metastatic malignant) breast cancer. In some embodiments, the ER positive and/or progesterone receptor (PR) positive, and HER2 negative breast cancer is locally advanced or metastatic (including malignant or metastatic malignant) breast cancer. In some embodiments, the ER positive, PR positive, and HER2 negative breast cancer is locally advanced or metastatic (including malignant or metastatic malignant) breast cancer. In some embodiments, the ER positive and HER2 negative breast cancer is locally advanced or metastatic (including malignant or metastatic malignant) breast cancer. In some embodiments, the PR positive and HER2 negative breast cancer is locally advanced or metastatic (including malignant or metastatic malignant) breast cancer. In some embodiments, the locally advanced or metastatic (including malignant or metastatic malignant) breast cancers, including for example, the HR+/HER2− breast cancer, the ER positive, PR positive, and HER2 negative breast cancer, the ER positive and HER2 negative breast cancer, the PR positive and HER2 negative breast cancer are confirmed histologically, cytologically, or both histologically and cytologically. In some embodiments, such histological, cytological, or both histological and cytological confirmation are conducted per American Society of Clinical Oncology/College of American Pathologists (ASCO/CAP) guidelines based on the most recently analyzed tissue.

In some embodiments, subjects having breast cancer and treated with the methods provided herein have received >1 line of endocrine therapy and a cyclin-dependent kinase (CDK) 4/6 inhibitor in the metastatic (including malignant or metastatic malignant) or locally advanced setting. In some embodiments, subjects having breast cancer and treated with the methods provided herein have received prior treatment with a taxane or anthracycline in any setting. In some embodiments, subjects having breast cancer and treated with the methods provided herein have a deleterious germline mutation in breast cancer susceptibility gene (BRCA)1 or 2 must have been treated with a poly ADP ribose polymerase (PARP) inhibitor.

In some specific embodiments, subjects treated with the methods provided herein have histologically or cytologically-confirmed HR+/HER2− breast cancers that are defined as ER positive and/or progesterone receptor (PR) positive, and HER2 negative as per American Society of Clinical Oncology/College of American Pathologists (ASCO/CAP) guidelines based on the most recently analyzed tissue; have a locally advanced or metastatic (including malignant or metastatic malignant) disease; have received ≥1 line of endocrine therapy and a cyclin-dependent kinase (CDK) 4/6 inhibitor in the metastatic (including malignant or metastatic malignant) or locally advanced setting; have received prior treatment with a taxane or anthracycline in any setting; and/or have a deleterious germline mutation in breast cancer susceptibility gene (BRCA)1 or 2 must have been treated with a poly ADP ribose polymerase (PARP) inhibitor.

In certain embodiments, the methods provided herein are used for the treatment of triple negative breast cancer (TNBC) in a subject. In some embodiments, the TNBC is histologically and/or cytologically-confirmed TNBC. In some embodiments, the TNBC is determined according to TNBC histology (ER-negative/PR-negative/HER2-negative) as per ASCO/CAP guidelines based on the most recently analyzed tissue. In some embodiments, the TNBC is locally advanced or metastatic. In some embodiments, subjects having TNBC and treated with the methods provided herein have had ≥2 lines of systemic therapy. In some embodiments, subjects having TNBC and treated with the methods provided herein have had ≥2 lines of systemic therapy, including a taxane in any setting. In some embodiments, subjects having TNBC and treated with the methods provided herein have a deleterious germline mutation in BRCA1, BRCA2, or both BRCA1 and BRCA2. In some embodiments, subjects having TNBC and treated with the methods provided herein have been treated with a PARP inhibitor. In some embodiments, the subjects treated by the methods provided herein for TNBC have any permutation or combination of the characteristics described in this paragraph.

In some specific embodiments, subjects treated with the methods provided herein have histologically or cytologically-confirmed TNBC that is defined as unequivocal TNBC histology (ER-negative/PR-negative/HER2-negative) as per ASCO/CAP guidelines based on the most recently analyzed tissue; have locally advanced or metastatic (including malignant or metastatic malignant) disease; have had ≥2 lines of systemic therapy, including a taxane in any setting; have had a deleterious germline mutation in BRCA1 or BRCA2 or both; and/or have been treated with a PARP inhibitor.

In certain embodiments, the methods provided herein are used for the treatment of squamous non-small cell lung cancer (NSCLC) in a subject. In some embodiments, the squamous NSCLC is histologically- and/or cytologically-confirmed squamous NSCLC. In some embodiments, the squamous NSCLC is locally advanced or metastatic. In some embodiments, subjects having squamous NSCLC and treated with the methods provided herein have progressed or relapsed following platinum-based therapy, including e.g. platinum therapy administered in the adjuvant setting if relapse occurred within 12 months after completion. In some embodiments, subjects having squamous NSCLC and treated with the methods provided herein have received prior therapy with an anti-programmed cell death protein-1 (PD-1) or anti-programmed cell death-ligand 1 (PD-L1) if eligible based on subject's tumor PD-1 or PD-L1 expression and local treatment guidelines.

In some specific embodiments, subjects treated with the methods provided herein have histologically- or cytologically-confirmed squamous NSCLC; have a locally advanced or metastatic (including malignant or metastatic malignant) disease; have progressed or relapsed following platinum-based therapy including e.g. platinum therapy administered in the adjuvant setting counts as a regimen if relapse occurred within 12 months after completion; and/or have received prior therapy with an anti-programmed cell death protein-1 (PD-1) or anti-programmed cell death-ligand 1 (PD-L1) if eligible based on subject's tumor PD-1 or PD-L1 expression and local treatment guidelines.

In certain embodiments, the methods provided herein are used for the treatment of non-squamous NSCLC in a subject. In some embodiments, the squamous NSCLC is histologically- and/or cytologically-confirmed squamous NSCLC. In some embodiments, the squamous NSCLC is epidermal growth factor receptor (EGFR) wild type and anaplastic lymphoma kinase (ALK) wild type. In some embodiments, the squamous NSCLC is EGFR wild type and ALK wild type by local laboratory standards. In some embodiments, the non-squamous NSCLC is locally advanced or metastatic. In some embodiments, subjects having squamous NSCLC and treated with the methods provided herein have progressed or relapsed following platinum-based therapy in the metastatic (including malignant or metastatic malignant) or locally advanced setting including e.g. platinum therapy administered in the adjuvant setting if relapse occurred within 12 months after completion. In some embodiments, subjects having squamous NSCLC and treated with the methods provided herein have received an anti-PD-1 or anti-PD-L1 therapy if eligible based on subject's tumor PD-1 or PD-L1 expression and local treatment guidelines.

In some specific embodiments, subjects treated with the methods provided herein have histologically or cytologically-confirmed non-squamous NSCLC that is EGFR wild type and ALK wild type by local laboratory standards; have locally advanced or metastatic (including malignant or metastatic malignant) disease; have progressed or relapsed following platinum-based therapy in the metastatic (including malignant or metastatic malignant) or locally advanced setting including e.g. platinum therapy administered in the adjuvant setting if relapse occurred within 12 months after completion; have received an anti-PD-1 or anti-PD-L1 therapy if eligible based on subject's tumor PD-1 or PD-L1 expression and local treatment guidelines.

In certain embodiments, the methods provided herein are used for the treatment of head and neck cancer in a subject. In some embodiments, the head and neck cancer is histologically- and/or cytologically-confirmed head and neck cancer. In some embodiments, the head and neck cancer is locally advanced or metastatic. In some embodiments, subjects having head and neck cancer and treated with the methods provided herein have progressed or relapsed following platinum containing regimen in the metastatic (including malignant or metastatic malignant) or locally advanced setting, which platinum containing regimen does not include platinum regimens administered as part of multimodal therapy in the curative setting unless the subject relapsed or progressed within 6 months after completion. In some embodiments, subjects having head and neck cancer and treated with the methods provided herein have received an anti-PD-1 or anti-PD-L1 therapy if eligible based on subject's tumor PD-1 or PD-L1 expression and local treatment guidelines.

In some specific embodiments, subjects treated with the methods provided herein have histologically- or cytologically-confirmed head and neck cancer; have a locally advanced or metastatic (including malignant or metastatic malignant) disease; have progressed or relapsed following platinum containing regimen in the metastatic (including malignant or metastatic malignant) or locally advanced setting, which does not include platinum regimens administered as part of multimodal therapy in the curative setting unless the subject relapsed or progressed within 6 months after completion; have received an anti-PD-1 or anti-PD-L1 therapy if eligible based on subject's tumor PD-1 or PD-L1 expression and local treatment guidelines.

In certain embodiments, the methods provided herein are used for the treatment of gastric or esophageal cancer in a subject. In some embodiments, the gastric or esophageal cancer is histologically- and/or cytologically-confirmed gastric or esophageal cancer. In some embodiments, the gastric or esophageal cancer is locally advanced or metastatic. In some embodiments, subjects having head and neck cancer and treated with the methods provided herein have progressed or relapsed following chemotherapy regimens that included a fluoropyrimidine and a platinum for locally advanced disease or metastatic (including malignant or metastatic malignant) disease, which chemotherapy regimens do not include neoadjuvant or adjuvant regimens unless the subject relapsed or progressed within 6 months after completion. In some embodiments, subjects having head and neck cancer and treated with the methods provided herein have received HER2 directed therapy if the subjects have HER2 positive cancer. In some embodiments, subjects having head and neck cancer and treated with the methods provided herein have HER2 positive cancer and have received HER2 directed therapy.

In some specific embodiments, subjects treated with the methods provided herein have histologically- or cytologically-confirmed gastric or esophageal cancer; have locally advanced or metastatic (including malignant or metastatic malignant) disease; have progressed or relapsed following chemotherapy regimens that included a fluoropyrimidine and a platinum for locally advanced disease or metastatic (including malignant or metastatic malignant) disease, which chemotherapy regimens do not include neoadjuvant or adjuvant regimens unless the subject relapsed or progressed within 6 months after completion; have HER2 positive cancer and have received HER2 directed therapy. In another specific embodiments, subjects treated with the methods provided herein have histologically- or cytologically-confirmed gastric or esophageal cancer; have locally advanced or metastatic (including malignant or metastatic malignant) disease; have progressed or relapsed following chemotherapy regimens that included a fluoropyrimidine and a platinum for locally advanced disease or metastatic (including malignant or metastatic malignant) disease, which chemotherapy regimens do not include neoadjuvant or adjuvant regimens unless the subject relapsed or progressed within 6 months after completion.

In certain embodiments, the methods provided herein are used for treating subjects having cancers that express nectin-4 RNA, express nectin-4 protein, or express both nectin-4 RNA and nectin-4 protein. In certain embodiments, the methods provided herein are used for treating subjects having cancers that express both nectin-4 RNA and nectin-4 protein, including for example, squamous NSCLC, non-squamous NSCLC, Gastric (GEJ) cancer, esophageal cancer, HNSCC, NSCLC-adenocarcinoma, head and neck cancer (e.g. head & neck cancer-squamous), and breast cancer (including HR+/HER2− breast cancer and TNBC). In some embodiments, the nectin-4 RNA expression in the cancers is determined by polynucleotide hybridization, sequencing (assessing the relative abundance of the sequences), and/or PCR (including RT-PCR). In some embodiments, the nectin-4 protein expression in the cancers is determined by IHC, analysis in fluorescence-activated cell sorting (FACS), and/or western blotting. In some embodiments, the nectin-4 protein expression in the cancers is determined by 2 methods of IHC.

In certain embodiments, the methods provided herein are used for treating subjects having cancers, wherein the cancers express nectin-4 RNA, express nectin-4 protein, or express both nectin-4 RNA and nectin-4 protein, and wherein the cancers are sensitive to cytotoxic agents (such as Vinca and MMAE) blocking microtubule polymerization. In certain embodiments, the methods provided herein are used for treating subjects having cancers that express both nectin-4 RNA and nectin-4 protein and that are sensitive to cytotoxic agents (such as Vinca and MMAE) blocking microtubule polymerization, which cancers include for example, squamous NSCLC, non-squamous NSCLC, Gastric (GEJ) cancer, esophageal cancer, HNSCC, NSCLC-adenocarcinoma, head and neck cancer (e.g. head & neck cancer-squamous), and breast cancer (including HR+/HER2− breast cancer and TNBC).

In some embodiments, the subjects that can be treated in the methods provided herein are subjects having solid tumors, including, for example, subjects having hormone receptor positive and human epidermal growth factor receptor 2 negative (HR+/HER2−) breast cancer, subjects having ER negative, PR negative, and HER2 negative (ER−/PR−/HER2−) breast cancer, subjects having NSCLC, subjects having non-squamous NSCLC, subjects having head cancer, subjects having neck cancer, subjects having head and neck cancer, subjects having gastric cancer, subjects having esophageal cancer, and/or subjects having gastric or esophageal cancer.

In certain embodiments, the subjects that can be treated in the methods provided herein further include subjects having solid tumors that are locally advanced, metastatic (including metastatic malignant), and both locally advanced and metastatic solid tumors. In some embodiments, the solid tumors that can be treated in the methods provided herein are advanced HR+/HER2− breast cancer, advanced ER−/PR−/HER2− breast cancer, advanced NSCLC, advanced non-squamous NSCLC, advanced head cancer, advanced neck cancer, advanced head and neck cancer, advanced gastric cancer, advanced esophageal cancer, and/or advanced gastric and esophageal cancer. In other embodiments, the solid tumors that can be treated in the methods provided herein are metastatic (including malignant or metastatic malignant) HR+/HER2− breast cancer, metastatic (including malignant or metastatic malignant) ER−/PR−/HER2− breast cancer, metastatic (including malignant or metastatic malignant) NSCLC, metastatic (including malignant or metastatic malignant) non-squamous NSCLC, metastatic (including malignant or metastatic malignant) head cancer, metastatic (including malignant or metastatic malignant) neck cancer, metastatic (including malignant or metastatic malignant) head and neck cancer, metastatic (including malignant or metastatic malignant) gastric cancer, metastatic (including malignant or metastatic malignant) esophageal cancer, and/or metastatic (including malignant or metastatic malignant) gastric and esophageal cancer.

In some embodiments, the locally advanced, metastatic (including metastatic malignant), and both locally advanced and metastatic (including malignant or metastatic malignant) solid tumors are confirmed histologically, cytologically, or both histologically and cytologically.

In some embodiments, the subjects that can be treated in the methods provided herein progressed or relapsed following one or more other treatments for cancer. The one or more treatments, following which the subjects have progressed or relapsed, include, for example, one or more lines of an endocrine therapy, a cyclin-dependent kinase (CDK) 4/6 inhibitor (including in metastatic or locally advanced setting), a treatment with taxane, a treatment with anthracycline, a poly ADP ribose polymerase (PARP) inhibitor, a platinum-based therapy, a therapy with an inhibitor of programmed cell death protein-1 (PD-1), an inhibitor of programmed cell death-ligand 1 (PD-L1), a chemotherapy that included a fluoropyrimidine, a HER2 directed therapy, and/or any permutation or combination of two or more of the therapies provided in this paragraph and those described herein.

In certain embodiments, the subjects that can be treated in the methods provided herein have previously received at least two, three, four, five, or six lines of systemic therapies. Such systemic therapies can be any treatment using substances that travel through the bloodstream, reaches and affects cells all over the body. Such systemic therapies can be those described in the preceding paragraph (paragraph [00894]). In one embodiment, such systemic therapy is a taxane.

In certain embodiments, the subjects that can be treated in the methods provided herein have progressed or relapsed other treatments for cancer within 1, 2, 3, 4, 5, 6, 7, 8, 9, 10, 11, 12, 13, 14, 15, 16, 17, 18, 19, 20, 21, 22, 23, or 24 month after the other treatments, including for example and not by way of limitation, any or any combination of the treatments described in the second paragraph before this present paragraph (paragraph [00894]). In some particular embodiment, the subjects have progressed or relapsed within 6 months after the platinum-based therapy or the chemotherapy that included a fluoropyrimidine. In other particular embodiments, the subjects have progressed or relapsed within 6 months after a platinum-based therapy. In further embodiments, the subjects have progressed or relapsed within 12 months after a platinum-based therapy.

In some embodiments, the subjects that can be treated in the methods provided herein have already received one or more other treatments for cancer. The one or more treatments that the subjects have received, include, for example, one or more lines of an endocrine therapy, a cyclin-dependent kinase (CDK) 4/6 inhibitor (including in metastatic or locally advanced setting), a treatment with a taxane, a treatment with anthracycline, a poly ADP ribose polymerase (PARP) inhibitor, a platinum-based therapy, a therapy with an inhibitor of programmed cell death protein-1 (PD-1), an inhibitor of programmed cell death-ligand 1 (PD-L1), a chemotherapy that included a fluoropyrimidine, a HER2 directed therapy, and/or any permutation or combination of two or more of the therapies provided in this paragraph and those described herein. In one embodiment, the subject has received an immune checkpoint inhibitor therapy and received a chemotherapy. In another embodiment, the subject has received an immune checkpoint inhibitor therapy. In yet another embodiment, the subject has received a chemotherapy.

In some embodiments, the subjects that can be treated in the methods provided herein have any combination or permutation of having received one or more other treatments for cancer as described in the preceding paragraph (paragraph [00897]) and having progressed or relapsed following one or more other treatments for cancer as described in the fourth paragraph before this present paragraph (paragraph [00894]).

In some embodiments, the subjects having cancer that can be treated in the methods provided herein have certain phenotypic or genotypic characteristics. In one embodiment, the subjects have HR+/HER2− breast cancer that is also estrogen receptor (ER) positive and HER2 negative. In one embodiment, the subjects have HR+/HER2− breast cancer that is also progesterone receptor (PR) positive and HER2 negative. In one embodiment, the subjects have HR+/HER2− breast cancer that is also estrogen receptor (ER) positive, progesterone receptor (PR) positive and HER2 negative. In one embodiment, the subjects have a deleterious germline mutation in breast cancer susceptibility gene (BRCA)1, BRCA2, or both BRCA1 and BRCA2. In one embodiment, the subjects have ER negative, PR negative, and HER2 negative (ER−/PR−/HER2−) breast cancer. In one embodiment, the subjects have wild-type epidermal growth factor receptor (EGFR). In one embodiment, the subjects have wild-type anaplastic lymphoma kinase (ALK). In one embodiment, the subjects have both wild-type epidermal growth factor receptor (EGFR) and wild-type anaplastic lymphoma kinase (ALK). In some embodiments, the subjects have any permutation and combination of the phenotypic or genotypic characteristics described herein.

In some embodiments, the phenotypic or genotypic characteristics are determined histologically, cytologically, or both histologically and cytologically. In one embodiment, the HR+/HER2− breast cancer that is also estrogen receptor (ER) positive and HER2 negative are determined histologically, cytologically, or both histologically and cytologically. In one embodiment, the HR+/HER2− breast cancer that is also progesterone receptor (PR) positive and HER2 negative are determined histologically, cytologically, or both histologically and cytologically. In one embodiment, the HR+/HER2− breast cancer that is also estrogen receptor (ER) positive, progesterone receptor (PR) positive and HER2 negative are determined histologically, cytologically, or both histologically and cytologically. In one embodiment, the deleterious germline mutation in breast cancer susceptibility gene (BRCA)1, BRCA2, or both BRCA1 and BRCA2 are determined histologically, cytologically, or both histologically and cytologically. In one embodiment, the ER negative, PR negative, and HER2 negative (ER−/PR−/HER2−) breast cancer are determined histologically, cytologically, or both histologically and cytologically. In one embodiment, the wild-type epidermal growth factor receptor (EGFR) are determined histologically, cytologically, or both histologically and cytologically. In one embodiment, the wild-type anaplastic lymphoma kinase (ALK) are determined histologically, cytologically, or both histologically and cytologically. In one embodiment, both wild-type epidermal growth factor receptor (EGFR) and wild-type anaplastic lymphoma kinase (ALK) are determined histologically, cytologically, or both histologically and cytologically.

In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference.

In some embodiments, the phenotypic or genotypic characteristics are determined by sequencing including the next generation sequencing (e.g. NGS from Illumina, Inc), DNA hybridization, and/or RNA hybridization. In one embodiment, the HR+/HER2− breast cancer that is also estrogen receptor (ER) positive and HER2 negative are determined by sequencing including the next generation sequencing (e.g. NGS from Illumina, Inc), DNA hybridization, and/or RNA hybridization. In one embodiment, the HR+/HER2− breast cancer that is also progesterone receptor (PR) positive and HER2 negative are determined by sequencing including the next generation sequencing (e.g. NGS from Illumina, Inc), DNA hybridization, and/or RNA hybridization. In one embodiment, the HR+/HER2− breast cancer that is also estrogen receptor (ER) positive, progesterone receptor (PR) positive and HER2 negative are determined by sequencing including the next generation sequencing (e.g. NGS from Illumina, Inc), DNA hybridization, and/or RNA hybridization. In one embodiment, the deleterious germline mutation in breast cancer susceptibility gene (BRCA)1, BRCA2, or both BRCA1 and BRCA2 are determined by sequencing including the next generation sequencing (e.g. NGS from Illumina, Inc), DNA hybridization, and/or RNA hybridization. In one embodiment, the ER negative, PR negative, and HER2 negative (ER−/PR−/HER2−) breast cancer are determined by sequencing including the next generation sequencing (e.g. NGS from Illumina, Inc), DNA hybridization, and/or RNA hybridization. In one embodiment, the wild-type epidermal growth factor receptor (EGFR) are determined by sequencing including the next generation sequencing (e.g. NGS from Illumina, Inc), DNA hybridization, and/or RNA hybridization. In one embodiment, the wild-type anaplastic lymphoma kinase (ALK) are determined by sequencing including the next generation sequencing (e.g. NGS from Illumina, Inc), DNA hybridization, and/or RNA hybridization. In one embodiment, both wild-type epidermal growth factor receptor (EGFR) and wild-type anaplastic lymphoma kinase (ALK) are determined by sequencing including the next generation sequencing (e.g. NGS from Illumina, Inc), DNA hybridization, and/or RNA hybridization.

In some embodiments, the nectin-4 RNA expression in the cancers is determined by polynucleotide hybridization, sequencing (assessing the relative abundance of the sequences), and/or PCR (including RT-PCR). In some embodiments, the nectin-4 protein expression in the cancers is determined by IHC, analysis in fluorescence-activated cell sorting (FACS), and/or western blotting. In some embodiments, the nectin-4 protein expression in the cancers is determined by more than one method. In some embodiments, the nectin-4 protein expression in the cancers is determined by two methods of IHC.

In some embodiments, the locally advanced or metastatic urothelial cancers are confirmed histologically, cytologically, or both histologically and cytologically. In some embodiments, the locally advanced or metastatic bladder cancers are confirmed histologically, cytologically, or both histologically and cytologically

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments can become apparent to individuals working in the art, and it is expected that those skilled artisans can employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the invention have been described. Nevertheless, it will be understood that various modifications can be made without departing from the spirit and scope of the invention. Accordingly, the descriptions are intended to illustrate but not limit the scope of invention described in the claims.

### 6. EXAMPLES

The examples in this Section (i.e., Section 6) are offered by way of illustration, and not by way of limitation.

### 6.1 Example 1— Assays for Identifying the Genes Associated with ICD Induced by ADCs

This example provides some assays used for identifying marker genes associated with ICD induced by ADCs, marker genes associated with efficacy of ADC treatment, and/or marker genes associated with efficacy for combining ADC treatment with one or more immune checkpoint inhibitors.

T-24 and UM-UC-3 cells were purchased from ATCC and cultured using the recommended media conditions. The T-24 hNectin-4 (human nectin-4) and the UM-UC-3 Nectin-4 cells were generated by transducing parental cells with lentivirus containing the human Nectin-4 using the pRCDCMEP-CMV-hNectin-4 EF1-Puro construct and selected using puromycin. The T-24 Nectin-4 (clone 1A9) cells were implanted into nude mice and passaged via trocar, allowed to reach approximately 200 mm³ tumor volume, and subsequently treated with a single IP dose of enfortumab vedotin (3 mg/kg) or non-binding ADC (3 mg/kg) with 7 animals per treatment group. Follow-up ICD studies with this model involved collecting tumors 5 days post treatment for downstream analysis by RNA-seq, flow, immunohistochemistry (IHC), and Luminex. Tumors were fixed in formalin and prepared as FFPE tissue blocks. Blocks were cut at 4 μm and immunohistochemistry was performed using F4/80, CD11c. The immunohistochemically stained slides sections were scanned with a Leica AT2 digital whole slide scanner, and the images were analyzed with Visiopharm software by use of custom-made algorithms for Nectin 4, CD11c and F4/80 staining. The algorithms were optimized on the basis of staining intensity and background staining. Percent positive staining was calculated for Nectin 4 and positive cells per mm² was calculated for F480 and CD11c.

Sections of tumor were lysed in Cell Lysis Buffer 2 (R&D Systems®, Catalog #895347). The cytokines and chemokines from the tumor samples were measured using the MILLIPLEX MAP mouse cytokine/chemokine magnetic bead panel (Millipore) and read on the LUMINEX MAGPIX system.

For the RNA-seq analysis RNA from flash frozen tumors was isolated using the TRIZOL Plus RNA Purification Kit (Life Technologies) according to the manufacturer's protocol yielding high quality RNA (average RNA integrity number >8). RNA selection method was using Poly(A) selection and the mRNA Library Prep Kit from Illumina and read on the Hi-Seq 2×150 bp, single index (Illumina). The sequence reads were mapped to the human and mouse transcriptome and total reads per million were determined.

RNA-seq differential gene expression analysis indicated that EV treated cells produce gene signatures consistent with microtubule disruption, ER stress, and immunogenic cell death (see Examples 2 and 3 below). RNA gene signatures from 1267 differentially regulated genes were used to identify signatures that went up or down between the EV treatment vs untreated samples (see Examples 2 and 3 below). The p-value is calculated using the Wilcoxon test. Gene signatures and genes associated with each signature were collected using the GSEA MSIG Database (gsea-msigdb.org).

### 6.2 Example 2— ADCs Induce Both Cell Death to the Targeted Cells and ICD

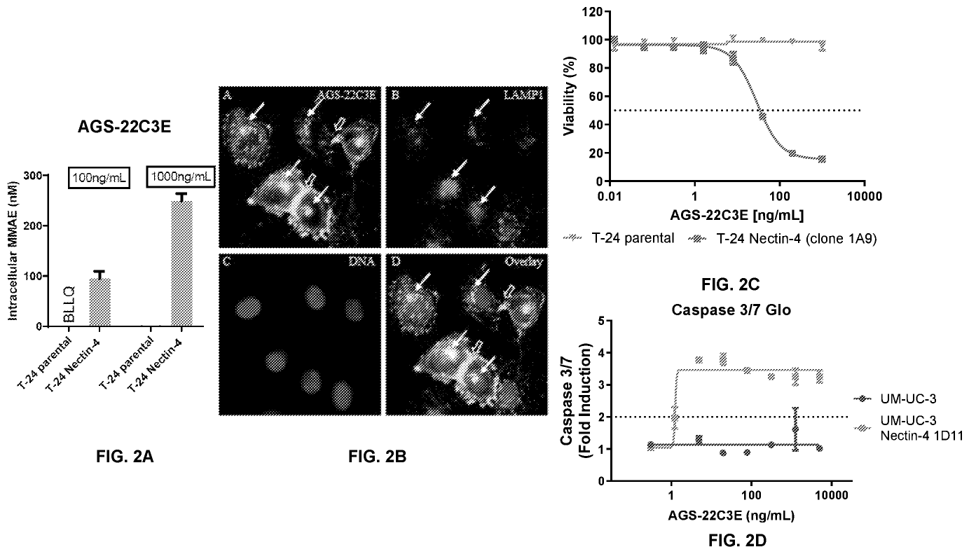

To determine the intracellular delivery of the drug moiety of the ADCs, the T-24 and UM-UC-3 bladder cancer cells were transduced with human Nectin-4. To measure the intracellular concentration of MMAE delivered by enfortumab vedotin, T-24 parental and T-24 Nectin-4 (clone1A9) cell lines were treated for 24 hours with 100 and 1,000 ng/mL ADC. Mass spectrometry analysis (LC-MS/MS) was then used to determine that enfortumab vedotin released 95 nM MMAE and 249 nM MMAE in T-24 Nectin-4 (clone:1A9) cells at 100 ng/mL (IC50 concentration) and 1,000 ng/mL (IC90 concentration) dose levels, respectively (FIG. 2A).

To determine the internalization and localization of the anti-nectin-4 ADC (enfortumab vedotin), T-24 Nectin-4 (clone:1A9) cells were stained after 2 hours of treatment with enfortumab vedotin and stained for enfortumab vedotin, lysosomal marker LAMP1 and Hoescht DNA stain (FIG. 2B). White arrows or merged yellow staining show the areas where enfortumab vedotin is colocalized with LAMP1 vesicles (FIG. 2B).

To determine the cytotoxicity induced by the anti-nectin-4 ADC (enfortumab vedotin also known as AGS-22C3E), T-24 Nectin-4 (clone:1A9) cells, UM-UC-3 Nectin-4 cells, and corresponding parent control cells were treated with enfortumab vedotin and cell viability was measured 120 hours post-treatment using CELL TITER-GLO (FIG. 2C). Enfortumab vedotin directly kills the T-24 Nectin-4 model while the parental T-24 cell line lacking Nectin-4 is insensitive to enfortumab vedotin (FIG. 2C). Additionally caspase 3/7 induction in response to the anti-nectin-4 ADC (AGS-22C3E) treatment was measured in UM-UC-3 Nectin-4 cells (FIG. 2D). The anti-nectin-4 ADC induced caspase 3/7 in UM-UC-3 Nectin-4 cells but not in the parental UM-UC-3 cell line lacking Nectin-4 (FIG. 2D). Table 7 below summarizes the cell surface expression of Nectin-4 and the cytotoxicity to enfortumab vedotin.

TABLE 7
cell surface expression of Nectin-4 and
the cytotoxicity to enfortumab vedotin

| | Cell Surface | | |
|---|---|---|---|
| | Nectin-4 | Enfortumab vedotin | |
| Cell line | expression | Cytotoxicity IC50 | |
| T-24 | <2000 | >1000 | ng/mL |
| T-24 Nectin-4 Clone 1A9 | 700,000 | 85 | ng/mL |
| UM-UC-3 | <2000 | >1000 | ng/mL |
| UM-UC-3 Nectin-4 Clone 1D11 | 680,000 | 6 | ng/mL |

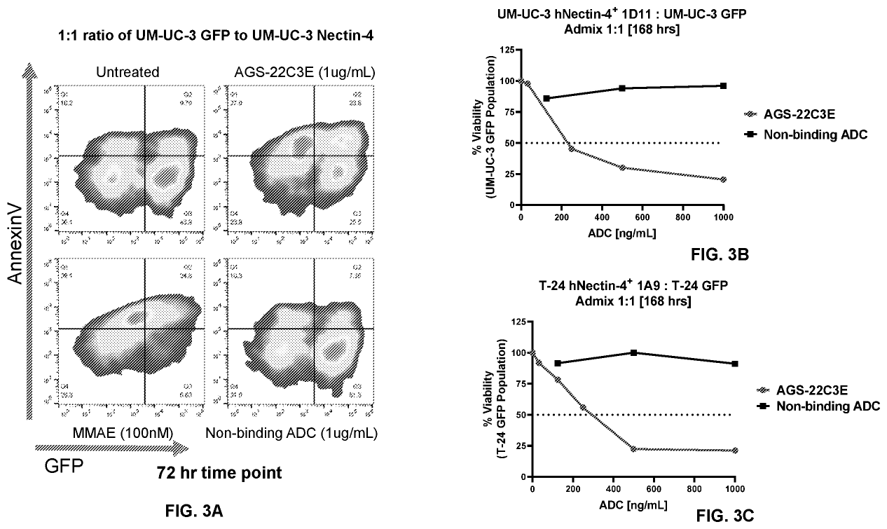

ADCs can also induced bystander cell killing, for example ICD, of antigen negative cancer cells. The bystander cell killing effect can be cell killing of antigen negative cancer cells through a targeted delivery of drug to antigen positive cancer cells. To determine the bystander cell killing, for example ICD, UM-UC-3 Nectin-4 (clone1D11) bladder cancer cells were co-cultured with GFP⁺-Nectin-4⁻ bladder cells at a 1:1 ratio and treated drug for 72 hours. Cell death was measured by Annexin V staining between the two populations. A targeted delivery of drug to antigen positive cells (GFP negative) induced bystander cell killing of antigen negative cancer cells (GFP positive) (FIG. 3A). Additionally, the percentage of viable cells in Q3 from FIG. 3A representing the Nectin-4 negative population was determined after 168 hours of treatment in a 1:1 co-culture with varying concentrations of enfortumab vedotin or non-binding ADC control for the UM-UC-3 and T-24 bladder cells. In both a 1:1 mixture (168 hrs) of UM-UC-3 expressing human nectin-4 (clone 1D11): UM-UC-3 expressing GFP (FIG. 3B) and a 1:1 mixture (168 hrs) of T-24 expressing human nectin-4 (clone 1A9): T-24 expressing GFP (FIG. 3C), a targeted delivery of drug to antigen positive cells (GFP negative) induced bystander cell killing of antigen negative cancer cells (GFP positive) (FIGS. 3B and 3C).

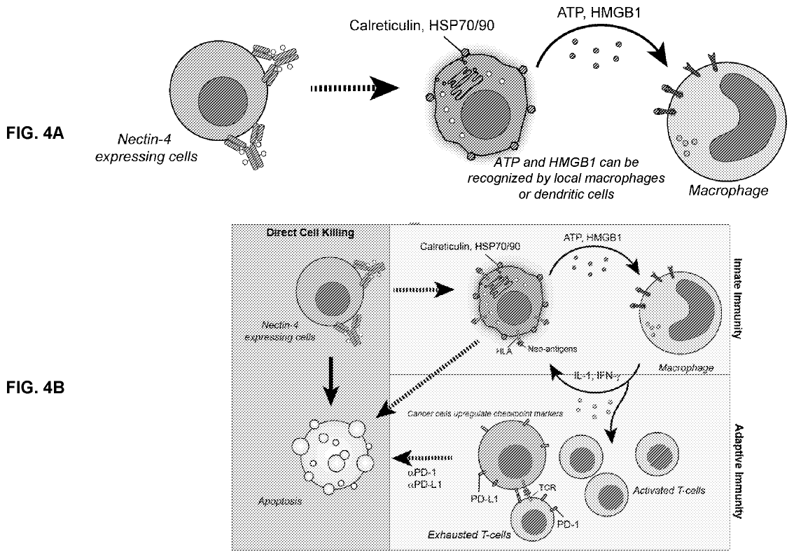

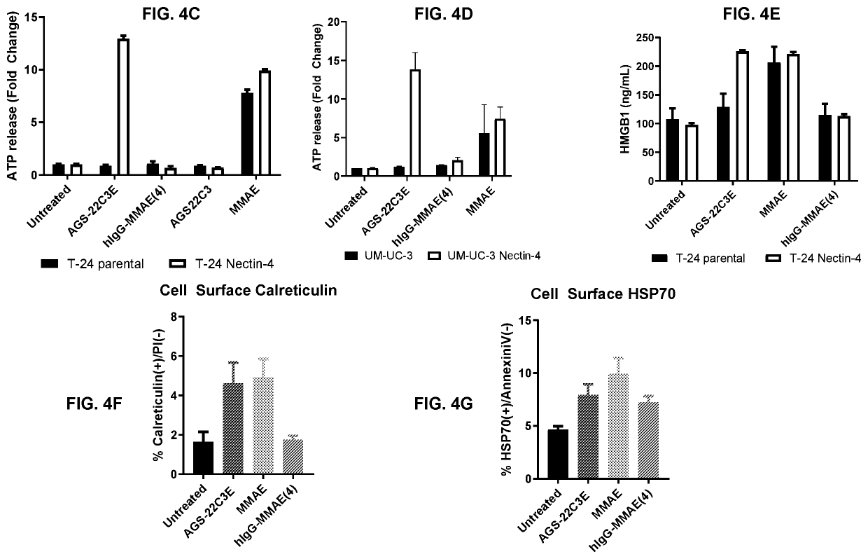

Some exemplary mechanisms for bystanding cell killing, e.g. ICD, were shown in FIGS. 4A and 4B. To determine the changes in the level of some of the ICD mediators shown in FIGS. 4A and 4B, extracellular release of ATP was determined 48 hours post-treatment with EV (1 mg/mL), Nectin-4 Ab (enfortumab, 1 mg/mL), MMAE (100 nM), and ADC control (hIgG-MMAE(4), 1 mg/mL) in control T-24 cells, T-24 cells expressing nectin-4, control UM-UC-3 cells, and UM-UC-3 cells (FIGS. 4C and 4D). Similarly, extracellular release of HMGB1 was determined after 48 hours of treatment with EV (1 mg/mL), Nectin-4 Ab (enfortumab, 1 mg/mL), MMAE (100 nM), and ADC control (hIgG-MMAE(4), 1 mg/mL) in control T-24 cells and T-24 cells expressing nectin-4 (FIG. 4E). Additionally, the percentage of T-24 Nectin-4 (clone1A9) cells containing calreticulin on the cell surface (calreticulin+) and propridium iodide negative (PI−) was determined 48 hours post-treatment with enfortumab vedotin (EV, 1 mg/mL), MMAE (100 nM) and ADC control (hIgG-MMAE(4), 1 mg/mL) (FIG. 4F). Also determined was the percentage of T-24 Nectin-4 (clone1A9) cells that stained HSP70 on the cell surface and were Annexin V negative after treatment with drugs for 48 hours (FIG. 4G). The anti-nectin-4 ADC (EV) induced hallmarks of early immunogenic cell death, including ATP release, HMGB1 release, surface calreticulin, and surface HSP70. These ICD markers promote immune cell activation and recruitment.

### 6.3 Example 3— Marker Genes Associated with ICD Induced by ADCs, Efficacy of ADC Treatment, and/or Efficacy for Combining ADC Treatment with Immune Checkpoint Inhibitors

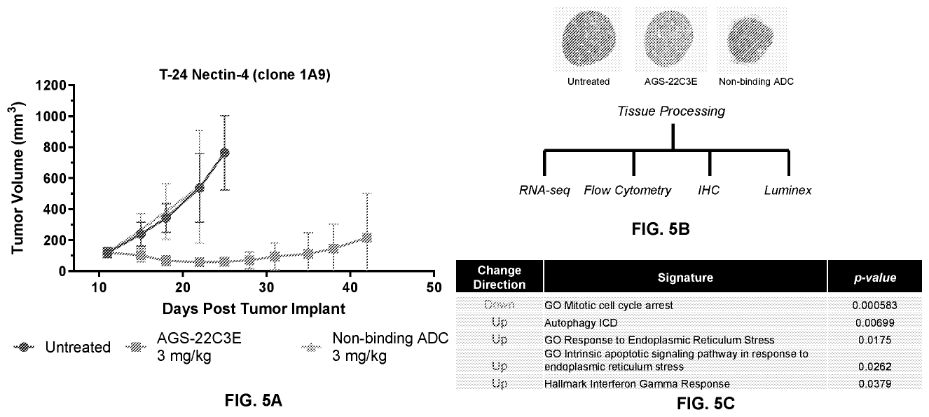

To determine whether the anti-nectin-4 ADC induced ICD and identify the markers associated with the ICD induced by the anti-nectin-4 ICD, the assays as described in Example 1 (Section 6.1) were performed. Briefly, the T-24 Nectin-4 (clone 1A9) cells were implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. The treatment with an anti-nectin-4 ADC (EV) blocked the tumor growth (FIG. 5A). Tumors from each treatment were stained for Nectin-4 as shown in FIG. 5B and follow-up ICD studies with this model involved collecting tumors 5 days post treatment for downstream analysis by RNA-seq, flow, immunohistochemistry (IHC), and Luminex as shown in FIG. 5B. RNA-seq differential gene expression analysis indicated that EV treated cells produce gene signatures consistent with microtubule disruption, ER stress, and immunogenic cell death, for example those marker genes as shown in FIG. 5C. RNA gene signatures from 1267 differentially regulated genes were used to identify signatures that went up or down between the EV treatment vs untreated samples (n=7) (FIG. 5C).

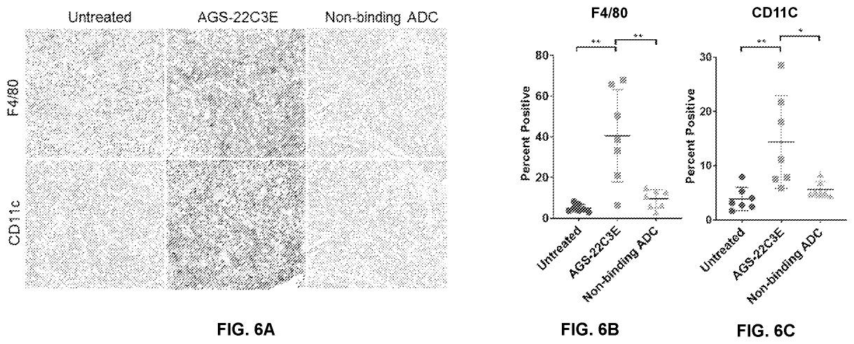

Further applying the assays described in Example 1 (Section 6.1), tumors from the T-24 Nectin-4 (clone1A9) xenograft were collected at Day5 post-treatment and divided for downstream analyses such as IHC, flow cytometry, cytokine analysis and RNA-seq. Enriched immune cell infiltration was seen by F4/80 and CD11c IHC staining in the Enfortumab vedotin treatment group compared to untreated or non-binding ADC control (FIG. 6A). Dissociated tumors were stained for immune infiltration by determining the percentage of F4/80 (FIG. 6B) or CD11C (FIG. 6C) positive cells in CD45 expressing cells by flow cytometry. Results in FIG. 6B and FIG. 6C confirm the enrichment of immune cell infiltration in the enfortumab vedotin treatment group compared to untreated or non-binding ADC control.

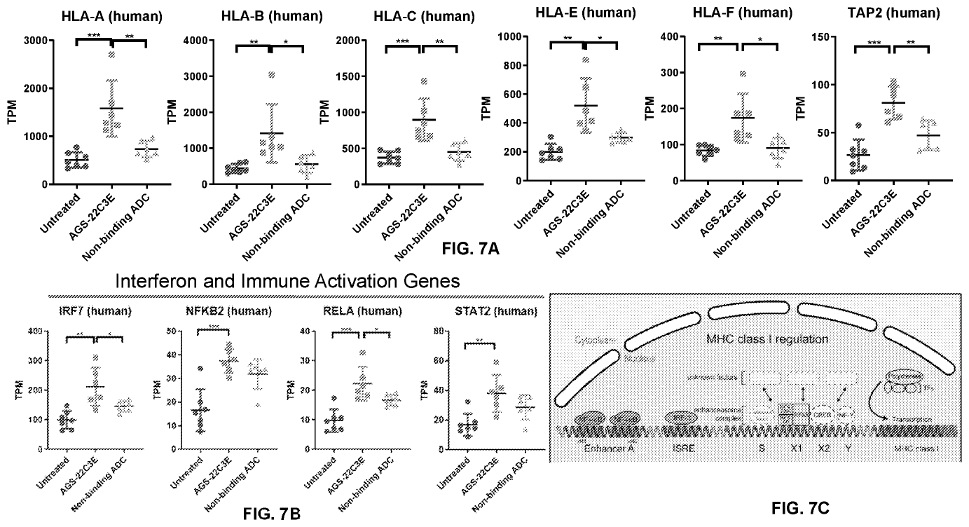

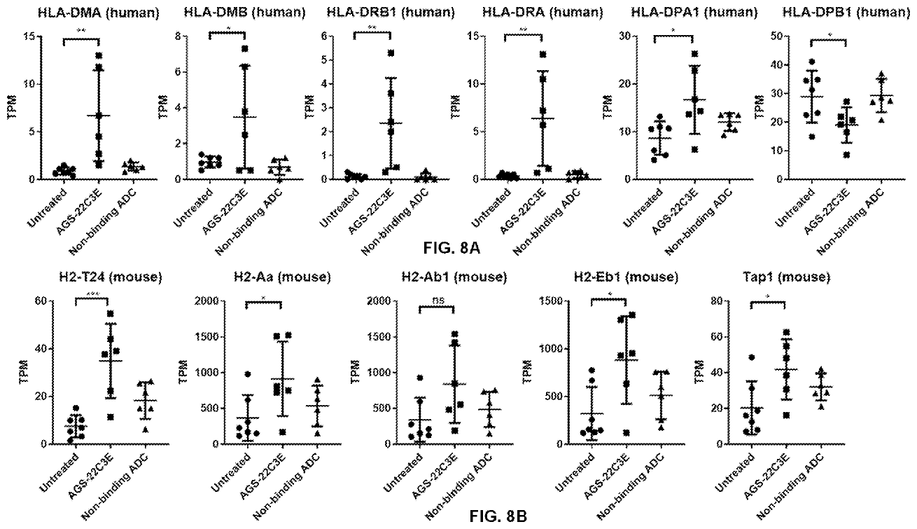

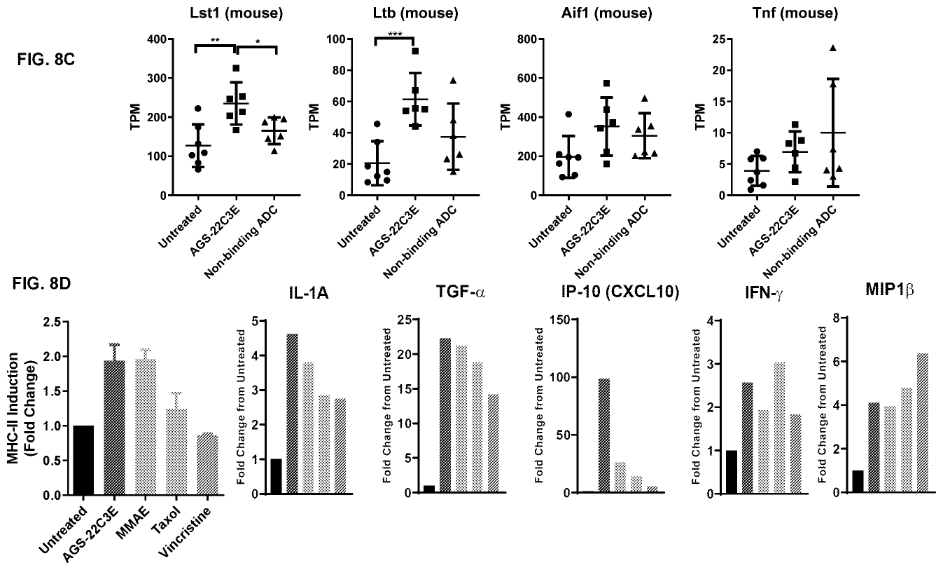

Without being bound or limited by the theory, the present disclosure provides that upregulated HLA (class I and class II) on cancer cells can activate the adaptive immune response by displaying neoantigens on the cell surface after treatment by ADCs. Upregulation of HLAs enhance/induce ICD after treatment with ADCs, enhance/induce the bystander cell killing effect induced by ADCs, enhance the efficacy of the ADC treatment, and enhance the efficacy for combining ADC treatment with immune checkpoint inhibitors. To further determine the MHC class gene markers for ICD induced by ADCs, efficacy of ADC treatment, and/or efficacy for combining ADC treatment with immune checkpoint inhibitors, the assays described in Example 1 (Section 6.1) were performed and tumors were collected post-treatment and divided for downstream analyses such as IHC, flow cytometry, cytokine analysis and RNA-seq, as described in Example 1 (Section 6.1). RNA-seq gene transcripts identified MHC class I genes including the transporter TAP2 genes upregulated upon treatment with enfortumab vedotin compared to untreated or non-binding ADC (FIG. 7A). Upregulation of MHC genes can allow neo-antigens to be presented where MHC class I genes activate CD8 to prime the adaptive immune response. RNA-seq gene transcripts also identified MHC regulator genes such as interferon and immune activation transcriptional regulators from the human transcriptome to be upregulated upon treatment with enfortumab vedotin compared to untreated or non-binding ADC (FIG. 7B). The factors known to drive MHC class I gene regulation are known to promote gene expression (see e.g. FIG. 7C). Additionally, RNA-seq gene transcripts identified MHC class II genes to be upregulated upon treatment with enfortumab vedotin compared to untreated or non-binding ADC (FIG. 8A). Similarly RNA-seq gene transcripts identified MHC class II genes from the mouse transcriptome to be upregulated upon treatment with enfortumab vedotin compared to untreated or non-binding ADC (FIG. 8B). RNA-seq gene transcripts also identified MHC class III genes from the mouse transcriptome to be upregulated upon treatment with enfortumab vedotin compared to untreated or non-binding ADC (FIG. 8C). Upregulation of MHC genes can allow neo-antigens to be presented where MHC class II genes activate CD4 T-cells to prime the adaptive immune response. These results show that the anti-nectin-4 ADC (enfortumab vedotin) induces upregulation of MHC class I, class II, and class III genes.

To confirm that ADC-treated tumors stimulate and activate human macrophages, the following assay was performed; T-24 Nectin-4 (clone1A9) cells were treated with drugs as indicated for 24 hours. Cell debris material was collected and incubated with macrophages from PBMCs. Macrophages were collected and stained for activation markers by flow cytometry such as cell surface expression of MHC-II. Cytokine profiling was performed using Luminex Human Cytokine array. The results as shown in FIG. 8D demonstrate that the anti-nectin-4 ADC (enfortumab vedotin) treated cells activate and stimulate macrophages, and stimulate the release of cytokines.

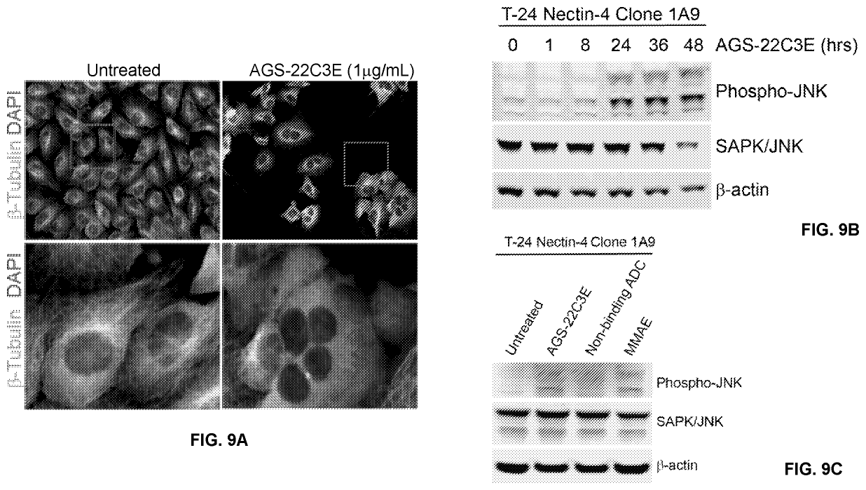

To confirm that ADC treatment disrupt microtubules and induces ER stress, T-24Nectin-4 (clone1A9) cells were treated with enfortumab vedotin (EV) for 48 hours and stained with β-tubulin for microtubules and DAPI, a nuclear DNA stain. FIG. 9A demonstrates that the anti-nectin-4 ADC (EV also known as AGS-22C3E) disrupts microtubules. FIGS. 9B and 9C demonstrate that the treatment with the anti-nectin-4 ADC (EV also known as AGS-22C3E) or MMAE but not with non-binding ADC control activates phospho-JNK.

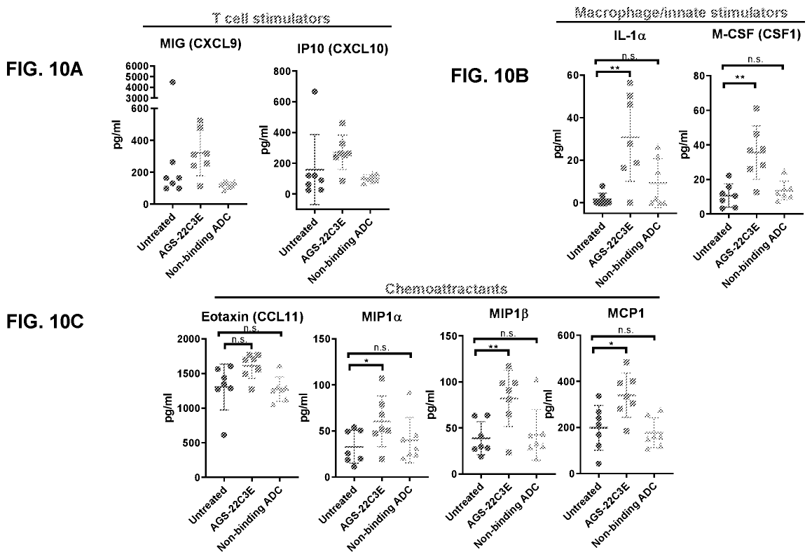

To identify T cell stimulator gene markers, macrophage/innate immunity stimulator gene markers, chemoattractant gene markers for ICD induced by ADCs, efficacy of ADC treatment, and/or efficacy for combining ADC treatment with immune checkpoint inhibitors, the assays described in Example 1 (Section 6.1) were performed and tumors were collected post-treatment and divided for downstream analyses such as IHC, flow cytometry, cytokine analysis and RNA-seq, as described in Example 1 (Section 6.1). Briefly, tumors collected 5-day post-treatment were processed and mouse cytokines were measured using the Luminex mouse cytokine kit. Cytokines released by macrophages and dendritic cells such as IL-1α and M-CSF (CSF1) were significantly elevated in the EV treatment group (FIG. 10B). Other cytokines such as T-cell stimulators (MIG & IP10) and chemoattractants (Eotaxin, MIP1α, and MIP1β & MCP1) were also elevated in this analysis (FIGS. 10A and 10C). RNA-seq analysis confirms elevated gene transcripts associated with these cytokines (data not shown).

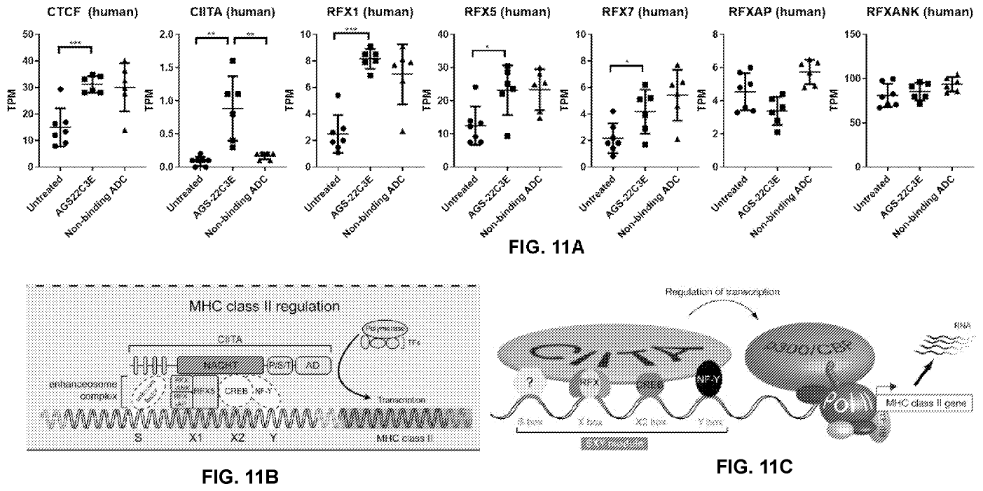

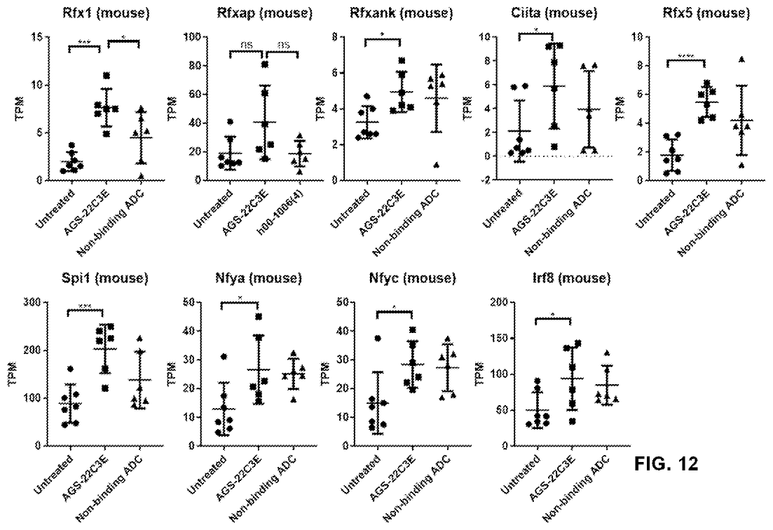

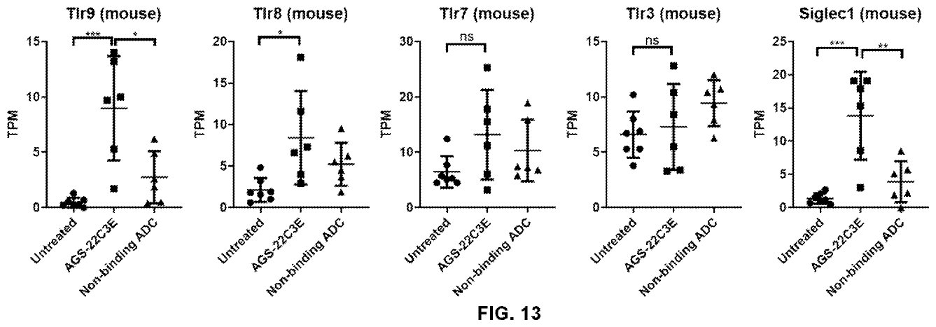

To determine other MHC regulator gene markers and toll-like receptor or siglec family gene markers for ICD induced by ADCs, efficacy of ADC treatment, and/or efficacy for combining ADC treatment with immune checkpoint inhibitors, the assays described in Example 1 (Section 6.1) were performed and tumors were collected post-treatment and divided for downstream analyses such as IHC, flow cytometry, cytokine analysis and RNA-seq, as described in Example 1 (Section 6.1). RNA-seq gene transcripts identified interferon and immune activation transcriptional regulators from the human transcriptome to be upregulated upon treatment with enfortumab vedotin compared to untreated or non-binding ADC (FIG. 11A). RNA-seq gene transcripts also identified interferon and immune activation transcriptional regulators from the mouse transcriptome to be upregulated upon treatment with enfortumab vedotin compared to untreated or non-binding ADC (FIG. 12). The transcriptional regulatory factors above are known to promote MHC class II gene expression. FIG. 11B and FIG. 11C provide exemplary regulation of MHC class II gene. RNA-seq gene transcripts additionally identified certain innate Toll-like receptors or siglec1 from the mouse transcriptome to be upregulated upon treatment with enfortumab vedotin compared to untreated or non-binding ADC (FIG. 13).

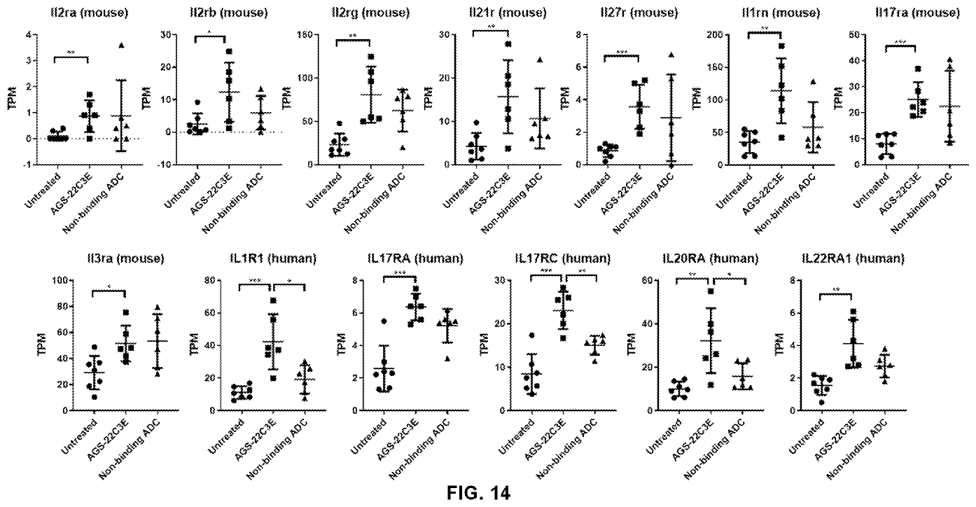

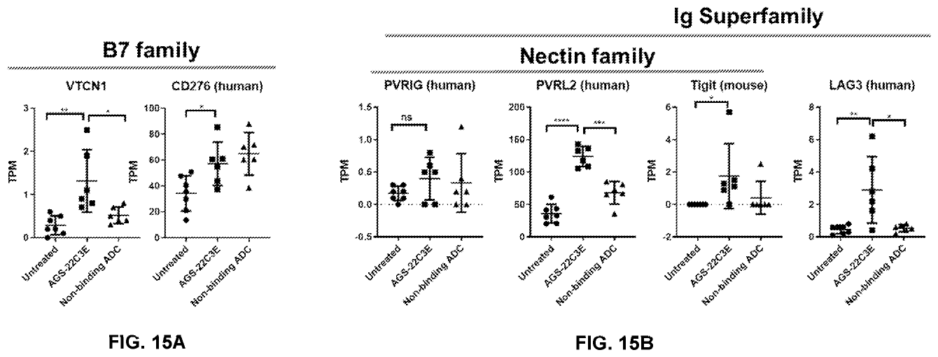

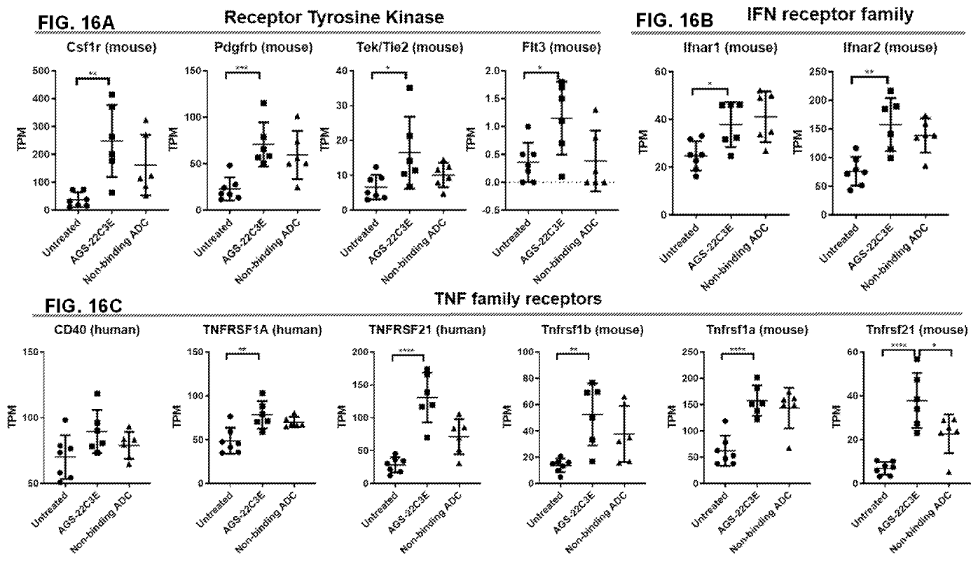

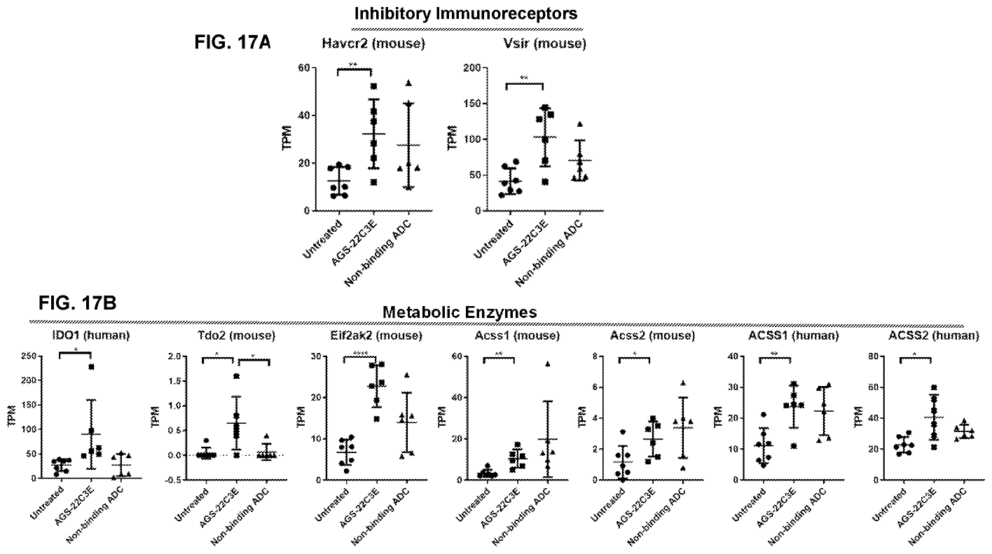

Additional studies were performed to determine the interleukin receptor family gene markers, B7 family gene markers, Ig superfamily gene markers (including nectin family gene markers), receptor tyrosin kinase gene markers, TNF family receptor gene markers, IFN receptor family gene markers, inhibitory immunoreceptor gene markers, and metabolic enzyme gene markers for ICD induced by ADCs, efficacy of ADC treatment, and/or efficacy for combining ADC treatment with immune checkpoint inhibitors. Such studies can also identify the therapeutic targets and anti-cancer treatments that can be combined with an anti-nectin-4 ADC. Briefly, the assays described in Example 1 (Section 6.1) were performed. RNA-seq gene transcripts identified interleukin receptor family genes to be upregulated upon treatment with enfortumab vedotin (AGS-22C3E) compared to untreated or non-binding ADC (FIG. 14). RNA-seq gene transcripts also identified B7 family genes (FIG. 15A), Ig superfamily genes (FIG. 15B) (including nectin family genes and other Ig superfamily genes such as LAGS), receptor tyrosin kinase genes (FIG. 16A), IFN receptor family genes (FIG. 16B), TNF family receptor genes (FIG. 16C), inhibitory immunoreceptor genes (FIG. 17A), and metabolic enzyme genes (FIG. 17B) to be upregulated upon treatment with enfortumab vedotin (AGS-22C3E) compared to untreated or non-binding ADC. Among, the upregulated genes, RNA-seq gene transcripts also identified a list of therapeutic targets upregulated upon EV treatment that can be combined with EV as potential combination partners (FIGS. 14, 15A-15B, 16A-16C, and 17A-17B). Table 8 lists some exemplary therapeutic targets identified that can be combined with and anti-nectin-4 ADC as well as potential drugs against these therapeutic targets.

TABLE 8
Therapeutic targets and potential drugs
for combination with anti-Nectin-4 ADCs

| Target | Drug Name | Target | Drug Name |
|---|---|---|---|
| IDO1 | Epacadostat | HLA (MHC) | GSK01 |
| | BMS986205 | | IMC-C103C |
| | Navoximod | | IMC-F106C |
| | PF-06840003 | | IMC-G107C |
| | KHK2455 | | ABBV-184 |
| | RG70099 | | |
| | IOM-E | | |
| | IOM-D | | |
| TIGIT | MTIG7192A | IL2RA | Daclizumab |
| | BMS-986207 | | Basiliximab |
| | OMP-313M32 | | |
| | MK-7684 | | |
| | AB154 | | |
| | CGEN-15137 | | |
| | SEA-TIGIT | | |
| | ASP8374 | | |
| | AJUD008 | | |
| VISTA (VSIR) | CA-170 | IFNAR1 | Anifrolumab |
| | JNJ 61610588 | | Sifalimumab |
| | HMBD-002 | | |
| TIM3 | AJUD009 | CSF1R | Pexidartinib |
| | | | Emactuzumab |
| | | | Cabiralizumab |
| | | | ARRY-382 |
| | | | BLZ945 |
| | | | AJUD010 |
| | | | AMG820 |
| | | | IMC-CS4 |
| | | | JNJ-40346527 |
| | | | PLX5622 |
| | | | FPA008 |
| VTCN1 | FPA150 | | |

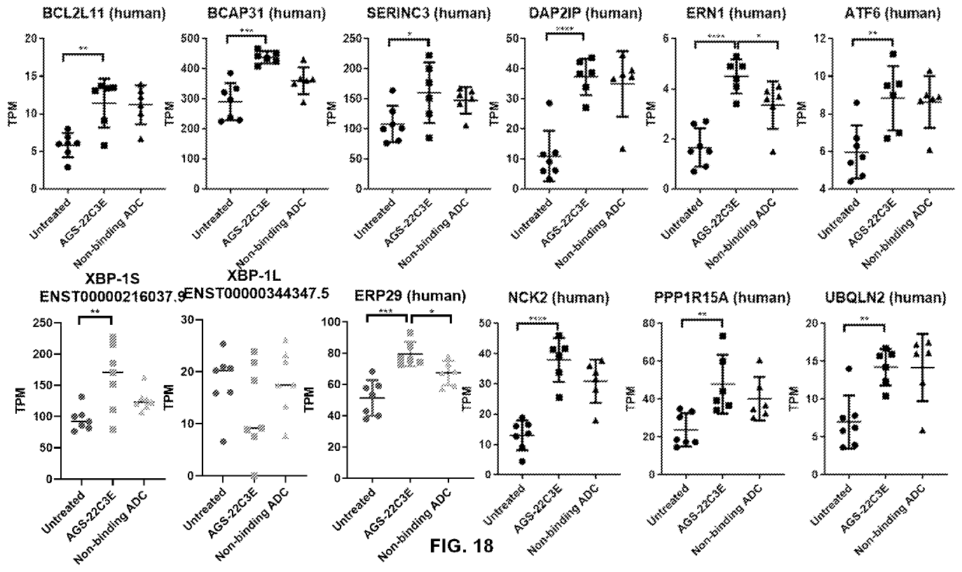

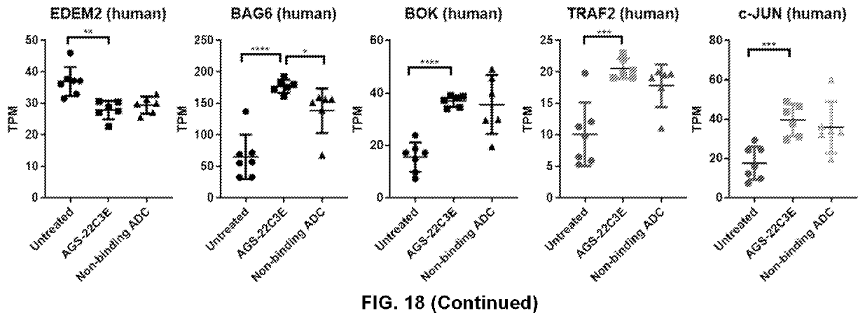

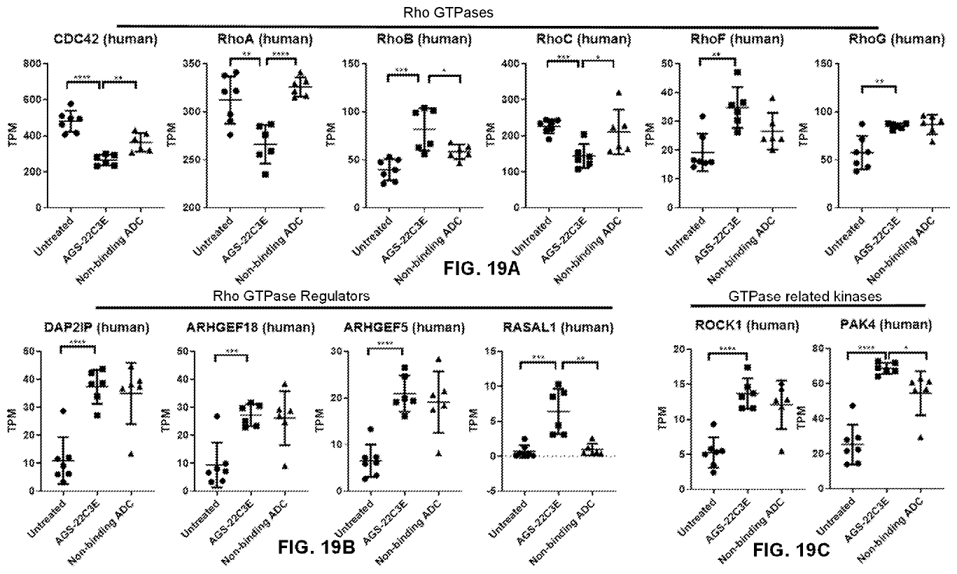

To identify ER stress gene markers, Rho GTPase gene markers, Rho GTPase regulator gene markers, and GTPase related kinase gene markers for ICD induced by ADCs, efficacy of ADC treatment, and/or efficacy for combining ADC treatment with immune checkpoint inhibitors, the assays described in Example 1 (Section 6.1) were performed and tumors were collected post-treatment and divided for downstream analyses such as IHC, flow cytometry, cytokine analysis and RNA-seq, as described in Example 1 (Section 6.1). RNA-seq gene transcripts identified genes associated with the GO positive regulation of response to endoplasmic reticulum stress (GO: 1902237) to be upregulated upon treatment with enfortumab vedotin (AGS-22C3E) compared to untreated or non-binding ADC (FIG. 18). RNA-seq gene transcripts also identified Rho GTPases known for regulating the actin cytoskeleton (FIG. 19A), Rho GTPase regulators (FIG. 19B), and GTPase related kinase (FIG. 19C) to be upregulated upon treatment with enfortumab vedotin (AGS22C3E) compared to untreated or non-binding ADC.

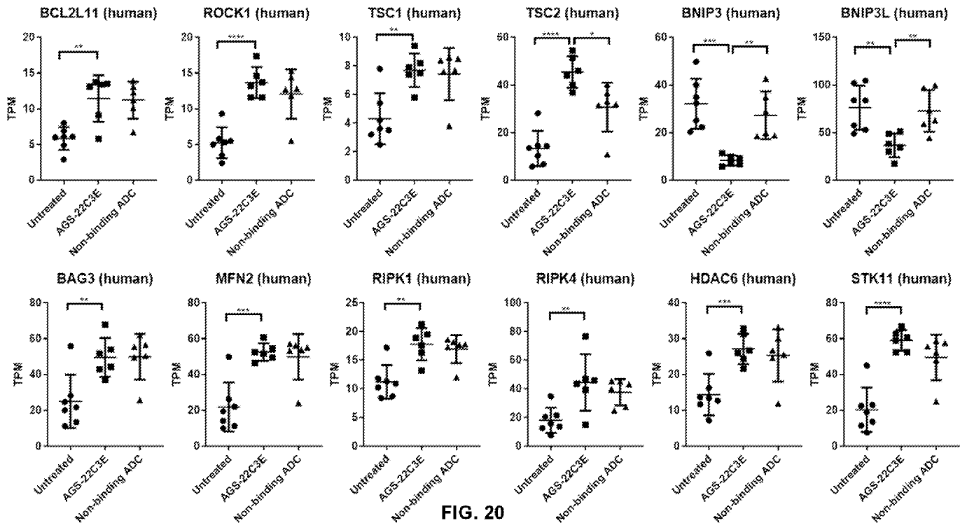

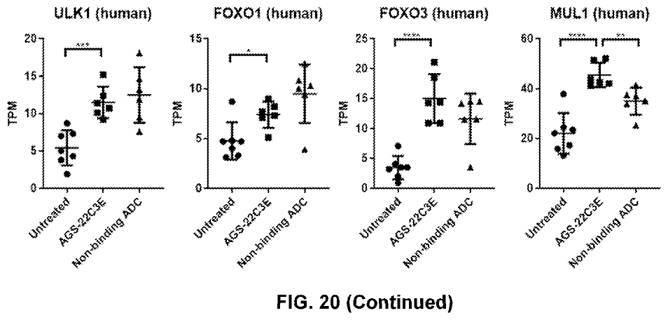

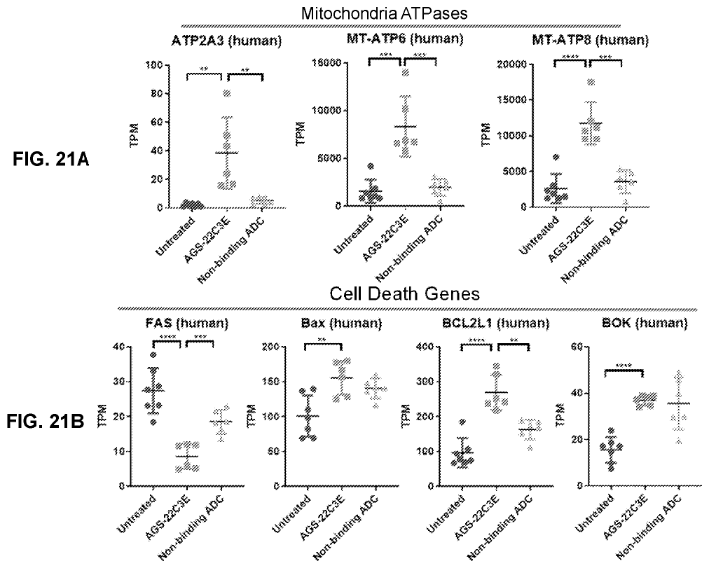

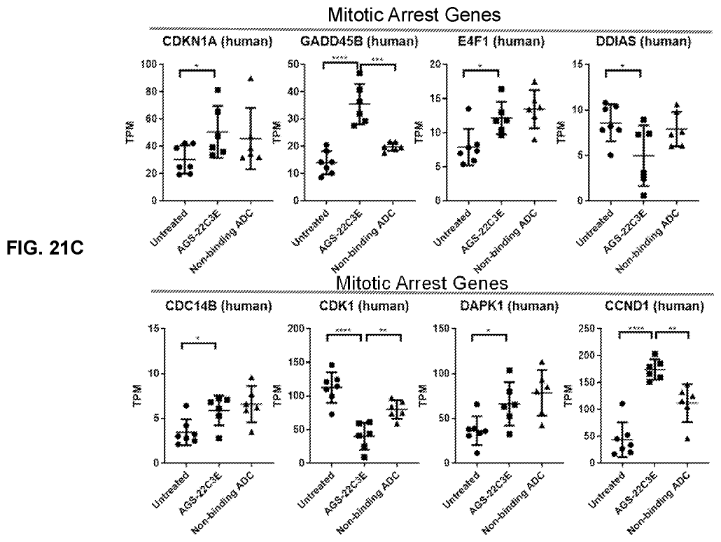

To identify GO positive autophagy regulator gene markers, ER/Mitochondria ATPase gene markers, cell death gene markers, and mitotic arrest gene markers for ICD induced by ADCs, efficacy of ADC treatment, and/or efficacy for combining ADC treatment with immune checkpoint inhibitors, the assays described in Example 1 (Section 6.1) were performed and tumors were collected post-treatment and divided for downstream analyses such as IHC, flow cytometry, cytokine analysis and RNA-seq, as described in Example 1 (Section 6.1). RNA-seq gene transcripts identified genes associated with the GO positive regulation of autophagy (GO: 0010508) to be upregulated upon treatment with enfortumab vedotin (as AGS-22C3E in FIG. 20) compared to untreated or non-binding ADC (FIG. 20). RNA-seq gene transcripts also identified ER/Mitochondria ATPase genes (FIG. 21A), cell death genes (FIG. 21B), and mitotic arrest genes (FIG. 21C) to be upregulated upon treatment with enfortumab vedotin (as AGS-22C3E in FIGS. 21A to 21C) compared to untreated or non-binding ADC.

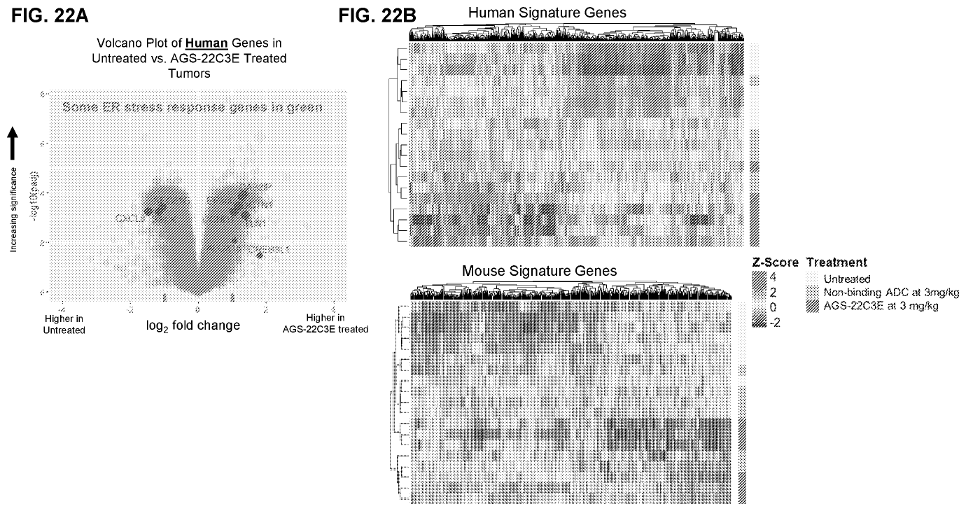

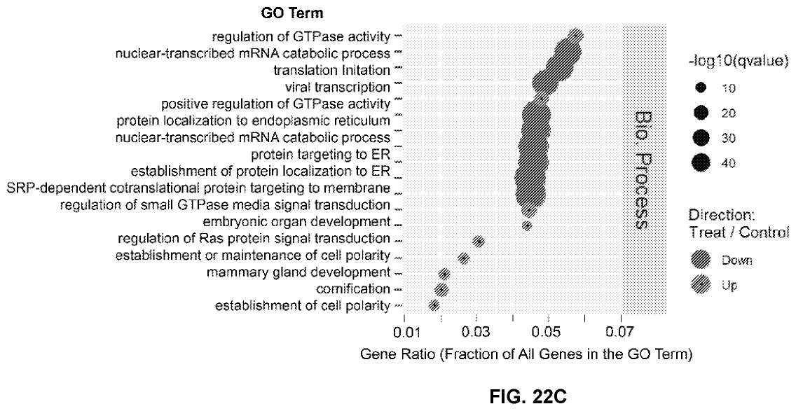

For an overview of the changes gene expression in untreated and anti-nectin-4 ADC (enfortumab vedotin or EV) treated tumors, additional analyses of the gene expression changes were performed. For example, Volcano Plot of human gene expression in untreated and anti-nectin-4 ADC (AGS-22C3E) treated tumors is presented in FIG. 22A, with some ER stress response genes in green. The analyses also identified a panel of 736 human genes relevant to ER stress and microtubule formation and another panel of 539 mouse genes relevant to immune cell populations and inflammatory response (FIG. 22B). The analyses also identified the biological processes changed upon anti-nectin-4 ADC (AGS-22C3E) treatment comparing with the untreated as determined from the human transcriptome (FIG. 22C).

What is claimed is:

1. A method for treating cancer in a subject in need thereof comprising:
(1) administering to the subject an antibody drug conjugate (ADC) comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
(b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more major histocompatibility complex (MHC) signature genes, one or more toll-like receptor (TLR) family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

2. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
(b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

3. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

4. A method for treating cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

5. A method for inducing immunogenic cell death (ICD) in a cancer in a subject in need thereof comprising:
(1) administering to the subject an ADC comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
(b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

6. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC,
(b) or administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

7. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC,
(b) or administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

8. A method for inducing ICD in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

9. A method for inducing immune cell migration to a cancer in a subject in need thereof comprising:
(1) administering to the subject an ADC comprising an antibody or antigen binding fragment thereof conjugated to one or more units of a cytotoxic agent via a linker,
(2) determining an increase of expression of one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
(b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

10. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
(b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

11. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

12. A method for inducing immune cell migration to a cancer in a subject in need thereof comprising:
(1) administering to the subject a first dose of an ADC comprising an antibody or antigen binding fragment thereof conjugated to one or more units of a cytotoxic agent via a linker,
(2) determining an increase of expression of one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

13. A method for increasing expression of one or more ADC Set I Marker genes in a cancer in a subject in need thereof comprising:
(1) administering to the subject an ADC comprising an antibody or antigen binding fragment thereof conjugated to one or more units of a cytotoxic agent via a linker,
(2) determining an increase of the expression of the one or more ADC Set I Marker genes in the subject, and
(3) (a) continue administering the ADC if the expression of the one or more ADC Set I Marker genes in the subject is increased compared to the expression of the one or more ADC Set I Marker genes in the subject before the administration of the ADC, or
(b) discontinue administering the ADC if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

14. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering a second dose of the ADC at the same or lower amount than the first dose if the expression of the one or more ADC Set I Marker genes in the subject is increased compared to the expression of the one or more ADC Set I Marker genes in the subject before the administration of the ADC, or
(b) administering a second dose of the ADC at a higher amount than the first dose if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

15. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering an immune checkpoint inhibitor in conjunction with the administration of a second dose of the ADC if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

16. A method for increasing expression of one or more ADC Set I Marker genes in a cancer in a subject in need thereof comprising:
(1) administering to the subject a first dose of an ADC comprising an antibody or antigen binding fragment thereof conjugated to one or more units of a cytotoxic agent via a linker,
(2) determining an increase of the expression of the one or more ADC Set I Marker genes in the subject, and
(3) (a) administering an immune checkpoint inhibitor to the subject if the expression of the one or more ADC Set I Marker genes in the subject is increased compared to the expression of the one or more ADC Set I Marker genes in the subject before the administration of the ADC, or
(b) administering a second dose of the ADC without the immune checkpoint inhibitor if the expression of the one or more ADC Set I Marker genes in the subject is not increased compared to the expression of the one or more ADC Set I Marker genes in the subject before the administration of the ADC,
wherein the checkpoint inhibitor in step (3)(a) is not administered in conjunction with the ADC
wherein the one or more ADC Set I Marker genes comprise one or more MHC signature genes, one or more TLR family genes, one or more interleukin receptor family genes, one or more immune checkpoint receptor genes, one or more receptor tyrosin kinase genes, one or more IFN receptor family genes, one or more TNF family receptor genes, one or more inhibitory immunoreceptor genes, and/or one or more metabolic enzyme genes.

17. The method of any one of claims 1 to 16, wherein the antibody or antigen binding fragment thereof is an anti-nectin-4 antibody or antigen binding fragment thereof.

18. The method of any one of claims 1 to 17, wherein the cytotoxic agent is a tubulin disrupting agent.

19. The method of claim 18, wherein the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane.

20. The method of claim 18 or 19, wherein the tubulin disrupting agent is an auristatin.

21. The method of claim 19 or 20, wherein the auristatin is monomethyl auristatin E (MMAE), monomethyl auristatin F (MMAF), AFP, or auristain T.

22. The method of any one of claims 19 to 21, wherein the auristatin is MMAE.

23. The method of any one of claims 1 to 22, wherein the antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO: 22 and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO: 23, and wherein the antibody or antigen binding fragment thereof is conjugated to 1 to 20 units of MMAE via a linker.

24. The method of any one of claims 1 to 23, wherein the one or more ADC Set I Marker genes comprise one or more MHC signature genes.

25. The method of any one of claims 1 to 23, wherein the one or more ADC Set I Marker genes consist of one or more MHC signature genes.

26. The method of any one of claims 1 to 25, wherein the one or more MHC signature genes comprise one or more MHC class genes.

27. The method of claim 26, wherein the one or more MHC class genes comprise one or more MHC class I genes.

28. The method of claim 27, wherein the one or more MHC class I genes comprise one or more genes selected from the group consisting of human leukocyte antigens-A (HLA-A), HLA-B, HLA-C, HLA-E, HLA-F, and Transporter 2, ATP binding cassette subfamily B member (TAP2).

29. The method of any one of claims 26 to 28, wherein the one or more MHC class genes comprise one or more MHC class II genes.

30. The method of claim 29, wherein the one or more MHC class II genes comprise one or more genes selected from the group consisting of HLA-DMA, HLA-DMB, HLA-DRB1, HLA-DRA, and HLA-DPA1.

31. The method of any one of claims 26 to 30, wherein the one or more MHC class genes or the one or more MHC class II genes do not comprise HLA-DPB1.

32. The method of any one of claims 26 to 30, wherein the MHC signature gene, the MHC class gene or the MHC class II gene is not HLA-DPB1.

33. The method of any one of claims 26 to 32, wherein the one or more MHC class genes comprise one or more MHC class III genes.

34. The method of claim 33, wherein the one or more MHC class III genes comprise one or more genes selected from the group consisting of LST1, LTB, AIF1, and TNF.

35. The method of any one of claims 1 to 34, wherein the one or more MHC signature genes comprise one or more MHC regulator genes.

36. The method of claim 35, wherein the one or more MHC regulator genes comprise one or more genes selected from the group consisting of interferon regulatory factor (IRF) genes, nuclear factor kappa-light-chain-enhancer of activated B cells (NF-κB) family genes, signal transducer and activator of transcription (STAT) family genes, CTCF, CIITA, RFX transcription factor family genes, SPI1, and nuclear transcription factor Y (NFY) genes.

37. The method of claim 36, wherein the NF-κB family genes comprise one or more genes selected from the group consisting of nuclear factor kappa B subunit 1 (NFKB1), NFKB2, RELA, RELB, and REL.

38. The method of claim 36 or 37, wherein the NF-κB family genes comprise NFKB2, RELA, or both NFKB2 and RELA.

39. The method of any one of claims 36 to 38, wherein the STAT family genes comprise one or more genes selected from the group consisting of STAT1, STAT2, STAT3, STAT4, STAT5, and STAT6.

40. The method of any one of claims 36 to 39, wherein the STAT family gene is STAT2.

41. The method of any one of claims 36 to 40, wherein the RFX transcription factor family genes comprise one or more genes selected from the group consisting of RFX1, RFX5, RFX7, RFXAP and RFXANK.

42. The method of any one of claims 36 to 41, wherein the IRF genes comprise IRF7, IRF8, or both IRF7 and IRF8.

43. The method of any one of claims 35 to 42, wherein the one or more MHC regulator genes comprise CTCF.

44. The method of any one of claims 35 to 43, wherein the one or more MHC regulator genes comprise CIITA.

45. The method of any one of claims 35 to 44, wherein the one or more MHC regulator genes comprise SPI1.

46. The method of any one of claims 36 to 45, wherein the NFY genes comprise NFYA, NFYC, or both NFYA and NFYC.

47. The method of any one of claims 1 to 46, wherein the one or more ADC Set I Marker genes comprise one or more TLR family genes.

48. The method of any one of claims 1 to 47, wherein the one or more TLR family genes comprise one or more genes selected from the group consisting of TLR9, TLR8, and TLR7.

49. The method of any one of claims 1 to 48, wherein the one or more TLR family genes do not comprise TLR3.

50. The method of any one of claims 1 to 49, wherein the one or more ADC Set I Marker genes comprise one or more interleukin receptor family genes.

51. The method of any one of claims 1 to 50, wherein the one or more interleukin receptor family genes comprise one or more genes selected from the group consisting of IL2RA, IL2RB, IL2RG, IL21R, IL27R, IL1RN, IL17RA, IL3RA, IL1R1, IL17RC, IL20RA, and IL22RA1.

52. The method of any one of claims 1 to 51, wherein the one or more interleukin receptor family genes comprise IL2RA.

53. The method of any one of claims 1 to 52, wherein the one or more interleukin receptor family genes consist of IL2RA.

54. The method of any one of claims 1 to 53, wherein the one or more ADC Set I Marker genes comprise one or more immune checkpoint receptor genes.

55. The method of any one of claims 1 to 54, wherein one or more immune checkpoint receptor genes comprise one or more B7 family genes, one or more Ig superfamily genes, or both one or more B7 family genes and one or more Ig superfamily genes.

56. The method of claim 55, wherein the B7 family genes comprise VTCN1, CD276, or both VTCN1 and CD276.

57. The method of claim 55 or 56, wherein the B7 family genes comprise VTCN1.

58. The method of any one of claims 55 to 57, wherein the B7 family genes consist of VTCN1.

59. The method of claim 55, wherein the Ig superfamily genes comprise nectin family genes.

60. The method of claim 55 or 59, wherein the Ig superfamily genes consist of nectin family genes.

61. The method of claim 55 or 59, wherein the Ig superfamily genes consist of LAG3 and nectin family genes.

62. The method of any one of claims 59 to 61, wherein the nectin family genes comprise one or more genes selected from the group consisting of PVRIG, PVRL2, and TIGIT.

63. The method of any one of claims 59 to 62, wherein the nectin family genes comprise TIGIT.

64. The method of any one of claims 59 to 63, wherein the nectin family genes consist of TIGIT.

65. The method of any one of claims 55 to 64, wherein the Ig superfamily genes comprise LAG3.

66. The method of any one of claims 55 to 58, wherein the Ig superfamily genes consist of LAG3.

67. The method of any one of claims 1 to 66, wherein the one or more ADC Set I Marker genes comprise one or more receptor tyrosin kinase genes.

68. The method of any one of claims 1 to 67, wherein the receptor tyrosin kinase genes comprise one or more genes selected from the group consisting of CSF1R, PDGFRB, TEK/TIE2, and FLT3.

69. The method of any one of claims 1 to 68, wherein the receptor tyrosin kinase genes consist of CSF1R.

70. The method of any one of claims 1 to 68, wherein the receptor tyrosin kinase genes comprise CSF1R.

71. The method of any one of claims 1 to 70, wherein the one or more ADC Set I Marker genes comprise one or more TNF family receptor genes.

72. The method of any one of claims 1 to 71, wherein the TNF family receptor genes comprise one or more genes selected from the group consisting of CD40, TNFRSF1A, TNFRSF21, and TNFRSF1B.

73. The method of any one of claims 1 to 72, wherein the one or more ADC Set I Marker genes comprise one or more IFN receptor family genes.

74. The method of any one of claims 1 to 73, wherein the IFN receptor family genes comprise IFNAR1, IFNAR2, or both IFNAR1 and IFNAR2.

75. The method of any one of claims 1 to 74, wherein the IFN receptor family genes consist of IFNAR1.

76. The method of any one of claims 1 to 74, wherein the IFN receptor family genes comprise IFNAR1.

77. The method of any one of claims 1 to 76, wherein the one or more ADC Set I Marker genes comprise one or more inhibitory immunoreceptor genes.

78. The method of any one of claims 1 to 77, wherein the inhibitory immunoreceptor genes comprise TIM3, VSIR, or both TIM3 and VSIR.

79. The method of any one of claims 1 to 78, wherein the inhibitory immunoreceptor genes comprise VSIR.

80. The method of any one of claims 1 to 78, wherein the inhibitory immunoreceptor genes consist of VSIR.

81. The method of any one of claims 1 to 79, wherein the inhibitory immunoreceptor genes comprise TIM3.

82. The method of any one of claims 1 to 78, wherein the inhibitory immunoreceptor genes consist of TIM3.

83. The method of any one of claims 1 to 82, wherein the one or more ADC Set I Marker genes comprise one or more metabolic enzyme genes.

84. The method of any one of claims 1 to 83, wherein the metabolic enzyme genes comprise one or more genes selected from the group consisting of indoleamine 2,3-dioxygenase 1 (IDO1), TDO2, EIF2AK2, ACSS1, and ACSS2.

85. The method of any one of claims 1 to 84, wherein the metabolic enzyme genes consist of IDO1.

86. The method of any one of claims 1 to 84, wherein the metabolic enzyme genes comprise IDO1.

87. The method of any one of claims 1 to 86, wherein the method further comprises determining an increase of expression of one or more ADC Set II Marker genes in the subject compared to the expression of the one or more ADC Set II Marker genes in the subject before the administration of the ADC in step (1).

88. The method of 87, wherein the administration in step (3)(a) is further conditioned on the increase of the expression of the one or more ADC Set II Marker genes as determined in claim 87.

89. The method of claim 87 or 88, wherein the one or more ADC Set II Marker genes comprise one or more genes selected from the group consisting of ER stress genes, ER/mitochondria ATPase genes, cell death genes, T cell stimulator genes, macrophage/innate immunity stimulator genes, chemoattractant genes, Rho GTPase genes, Rho GTPase regulator genes, mitotic arrest genes, siglec family genes, GO positive autophagy regulator genes, and GTPase related kinase genes.

90. The method of claim 89, wherein the ER stress genes comprise one or more genes selected from the group consisting of XBP-1S, ERP29, TRAF2, c-JUN, BCL2L11, BCAP31, SERINC3, DAP2IP, ERN1, ATF6, NCK2, PPP1R15A, UBQLN2, BAG6, and BOK.

91. The method of claim 89 or 90, wherein the ER stress genes do not comprise EDEM2 or XBP-1L.

92. The method of any one of claims 89 to 91, wherein the ER/mitochondria ATPase genes comprise one or more genes selected from the group consisting of ATP2A3, MT-ATP6, and MT-ATP8.

93. The method of any one of claims 89 to 92, wherein the cell death genes comprise one or more genes selected from the group consisting of Bax, BCL2L1, BCL2L11, and BOK.

94. The method of any one of claims 89 to 93, wherein the cell death genes do not comprise FAS.

95. The method of any one of claims 89 to 94, wherein the T cell stimulator genes comprise MIG (CXCL9), IP10 (CXCL10), or both MIG and IP10.

96. The method of any one of claims 89 to 95, wherein the macrophage/innate immunity stimulator genes comprise IL-1α, M-CSF (CSF), or both IL-1α and M-CSF.

97. The method of any one of claims 89 to 96, wherein the chemoattractant genes comprise one or more genes selected from the group consisting of Eotaxin (CCL11), MIP1α, MIP1β, and MCP1.

98. The method of any one of claims 89 to 97, wherein the Rho GTPase genes comprise one or more genes selected from the group consisting of RhoB, RhoF, and RhoG.

99. The method of any one of claims 89 to 98, wherein the Rho GTPase genes do not comprise any one of CDC42, RhoA, and RhoC.

100. The method of any one of claims 89 to 99, wherein the Rho GTPase regulator genes comprise one or more genes selected from the group consisting of DAP2IP, ARHGEF18, ARHGEF5, and RASAL1.

101. The method of any one of claims 89 to 100, wherein the mitotic arrest genes comprise one or more genes selected from the group consisting of CCND1, CDKN1A, GADD45B, E4F1, CDC14B, and DAPK1.

102. The method of any one of claims 89 to 101, wherein the mitotic arrest genes do not comprise DDIAS or CDK1.

103. The method of any one of claims 89 to 102, wherein the siglec family genes comprise siglec1.

104. The method of any one of claims 89 to 103, wherein the GO positive autophagy regulator genes comprise one or more genes selected from the group consisting of BCL2L11, ROCK1, TSC1, TSC2, BAG3, MFN2, RIPK1, RIPK4, HDAC6, STK11, ULK1, FOXO1, FOXO3, and MUL1.

105. The method of any one of claims 89 to 104, wherein the GO positive autophagy regulator genes do not comprise BNIP3 or BNIP3L.

106. The method of any one of claims 89 to 105, wherein the GTPase related kinase genes comprise ROCK1, PAK4, or both ROCK1 and PAK4.

107. The method of any one of claims 1 to 106, wherein the increase in any of the gene expression is an increase of 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, or more.

108. The method of any one of claims 1 to 106, wherein the increase in any of the gene expression is an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 fold or more.

109. The method of any one of claims 3, 4, 7, 8, 11, 12, and 15 to 108, wherein the immune checkpoint inhibitor is a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or an therapeutic agent targeting HLA.

110. The method of any one of claims 3, 4, 7, 8, 11, 12, and 15 to 109, wherein the immune checkpoint inhibitor is an anti-PD-1 antibody.

111. The method of claim 110, wherein the anti-PD-1 antibody is BGB-A317, nivolumab, pembrolizumab, cemiplimab, CT-011, camrelizumab, sintilimab, tislelizumab, TSR-042, PDR001, or toripalimab.

112. The method of any one of claims 3, 4, 7, 8, 11, 12, and 15 to 109, wherein the immune checkpoint inhibitor is an anti-PD-L1 antibody.

113. The method of claim 112, wherein the anti-PD-L1 antibody is durvalumab, BMS-936559, atezolizumab, MEDI4736, or avelumab.

114. The method of any one of claims 3, 4, 7, 8, 11, 12, and 15 to 109, wherein the immune checkpoint inhibitor is an anti-PD-L2 antibody.

115. The method of claim 114, wherein the anti-PD-L2 antibody is rHIgM12B7A.

116. The method of any one of claims 3, 4, 7, 8, 11, 12, and 15 to 109, wherein the immune checkpoint inhibitor is a VTCN1 inhibitor.

117. The method of claim 116, wherein the VTCN1 inhibitor is FPA150.

118. The method of any one of claims 3, 4, 7, 8, 11, 12, and 15 to 109, wherein the immune checkpoint inhibitor is an IDO1 inhibitor.

119. The method of claim 118, wherein the IDO1 inhibitor is Epacadostat, BMS986205, Navoximod, PF-06840003, KHK2455, RG70099, IOM-E, or IOM-D.

120. The method of any one of claims 3, 4, 7, 8, 11, 12, and 15 to 109, wherein the immune checkpoint inhibitor is a TIGIT inhibitor.

121. The method of claim 120, wherein the a TIGIT inhibitor is MTIG7192A, BMS-986207, OMP-313M32, MK-7684, AB154, CGEN-15137, SEA-TIGIT, ASP8374, or AJUD008.

122. The method of any one of claims 3, 4, 7, 8, 11, 12, and 15 to 109, wherein the immune checkpoint inhibitor is a VSIR inhibitor.

123. The method of claim 122, wherein the VSIR inhibitor is CA-170, JNJ 61610588, or HMBD-002.

124. The method of any one of claims 3, 4, 7, 8, 11, 12, and 15 to 109, wherein the immune checkpoint inhibitor is a TIM3 inhibitor.

125. The method of claim 124, wherein the TIM3 inhibitor is AJUD009.

126. The method of any one of claims 3, 4, 7, 8, 11, 12, and 15 to 109, wherein the immune checkpoint inhibitor is a CD25 (IL2RA) inhibitor.

127. The method of claim 126, wherein the CD25 (IL2RA) inhibitor is daclizumab or basiliximab.

128. The method of any one of claims 3, 4, 7, 8, 11, 12, and 15 to 109, wherein the immune checkpoint inhibitor is an IFNAR1 inhibitor.

129. The method of claim 128, wherein the IFNAR1 inhibitor is anifrolumab or sifalimumab.

130. The method of any one of claims 3, 4, 7, 8, 11, 12, and 15 to 109, wherein the immune checkpoint inhibitor is a CSF1R inhibitor.

131. The method of claim 130, wherein the CSF1R inhibitor is pexidartinib, emactuzumab, cabiralizumab, ARRY-382, BLZ945, AJUD010, AMG820, IMC-CS4, JNJ-40346527, PLX5622, or FPA008.

132. The method of any one of claims 3, 4, 7, 8, 11, 12, and 15 to 109, wherein the immune checkpoint inhibitor is a therapeutic agent targeting HLA.

133. The method of claim 132, wherein the therapeutic agent targeting HLA is GSK01, IMC-C103C, IMC-F106C, IMC-G107C, or ABBV-184.

134. The method of any one of claims 1 to 133, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

135. The method of any one of claims 1 to 134, wherein the antibody or antigen binding fragment thereof comprises a heavy chain variable region comprising the amino acid sequence of SEQ ID NO:22 and a light chain variable region comprising the amino acid sequence of SEQ ID NO:23.

136. The method of any one of claims 1 to 135, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

137. The method of any one of claims 1 to 136, wherein the antigen binding fragment is an Fab, F(ab′)2, Fv or scFv.

138. The method of any one of claims 1 to 137, wherein the antibody is a fully human antibody.

139. The method of any one of claims 1 to 138, wherein the antibody or antigen binding fragment thereof is recombinantly produced.

140. The method of any one of claims 1 to 139, wherein the ADC has the following structure: 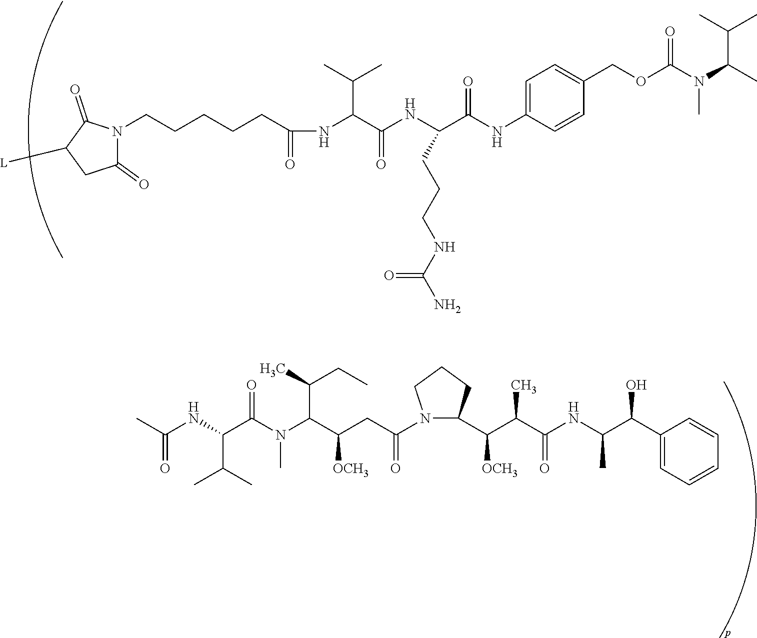
wherein L- represents the antibody or antigen binding fragment thereof and p is from 1 to 10.

141. The method of claim 140, wherein p is from 2 to 8.

142. The method of claim 140 or 141, wherein p is from 3 to 5.

143. The method of any one of claims 1 to 139, wherein the antibody or antigen binding fragment is conjugated to each unit of MMAE via a linker.

144. The method of claim 143, wherein the linker is an enzyme-cleavable linker, and wherein the linker forms a bond with a sulfur atom of the antibody or antigen binding fragment thereof.

145. The method of claim 143 or 144, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

146. The method of claim 145, wherein the stretcher unit has the structure of Formula (1) below; the amino acid unit is valine-citrulline; and the spacer unit is a PAB group comprising the structure of Formula (2) below:

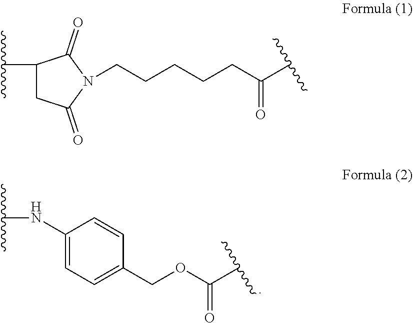 [SMILES: *C(=O)CCCCCN1C(=O)CC(*)C1=O ; *Nc1ccc(COC(*)=O)cc1]

147. The method of claim 145 or 146, wherein the stretcher unit forms a bond with a sulfur atom of the antibody or antigen binding fragment thereof; and wherein the spacer unit is linked to MMAE via a carbamate group.

148. The method of any one of claims 1 to 139 and 143 to 147, wherein the ADC comprises from 1 to 20 units of MMAE per antibody or antigen binding fragment thereof.

149. The method of any one of claims 1 to 139 and 143 to 148, wherein the ADC comprises from 1 to 10 units of MMAE per antibody or antigen binding fragment thereof.

150. The method of any one of claims 1 to 139 and 143 to 149, wherein the ADC comprises from 2 to 8 units of MMAE per antibody or antigen binding fragment thereof.

151. The method of any one of claims 1 to 139 and 143 to 150, wherein the ADC comprises from 3 to 5 units of MMAE per antibody or antigen binding fragment thereof.

152. The method of any one of claims 1, 5, 9, 13, and 17 to 151, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

153. The method of any one of claims 1, 5, 9, 13, and 17 to 152, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

154. The method of any one of claims 1, 5, 9, 13, and 17 to 153, wherein the ADC is administered at a dose of about 1 mg/kg of the subject's body weight.

155. The method of any one of claims 1, 5, 9, 13, and 17 to 153, wherein the ADC is administered at a dose of about 1.25 mg/kg of the subject's body weight.

156. The method of any one of claims 2 to 4, 6 to 8, 10 to 12, 14 to 151, wherein the first dose of the ADC is a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

157. The method of claim 156, wherein the first dose of the ADC is a dose of about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

158. The method of claim 156 or 157, wherein the first dose of ADC is a dose of about 1 mg/kg of the subject's body weight.

159. The method of claim 156 or 157, wherein the first dose of ADC is a dose of about 1.25 mg/kg of the subject's body weight.

160. The method of any one of claims 156 to 159, wherein the second dose of the ADC is lower than the first dose by about 0.1 mg/kg to about 1 mg/kg of the subject's body weight.

161. The method of any one of claims 156 to 160, wherein the second dose of the ADC is lower than the first dose by about 0.1 mg/kg, about 0.2 mg/kg, about 0.25 mg/kg, about 0.3 mg/kg, about 0.4 mg/kg, about 0.5 mg/kg, about 0.6 mg/kg, about 0.7 mg/kg, about 0.75 mg/kg, about 0.8 mg/kg, about 0.9 mg/kg, or about 1 mg/kg of the subject's body weight.

162. The method of any one of claims 156 to 161, wherein the second dose of the ADC is lower than the first dose by about 0.25 mg/kg of the subject's body weight.

163. The method of any one of claims 156 to 161, wherein the second dose of the ADC is lower than the first dose by about 0.5 mg/kg of the subject's body weight.

164. The method of any one of claims 156 to 161, wherein the second dose of the ADC is lower than the first dose by about 0.75 mg/kg of the subject's body weight.

165. The method of any one of claims 156 to 161, wherein the second dose of the ADC is lower than the first dose by about 1.0 mg/kg of the subject's body weight.

166. The method of any one of claims 156 to 165, wherein the second dose of the ADC is a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, or about 2.25 mg/kg of the subject's body weight.

167. The method of any one of claims 156 to 166, wherein the second dose of the ADC is identical to the first dose of the ADC.

168. The method of any one of claims 1 to 166, wherein the ADC is administered by an intravenous (IV) injection or infusion.

169. The method of any one of claims 1 to 168, wherein the ADC is administered by an IV injection or infusion three times every four-week cycle.

170. The method of any one of claims 1 to 169, wherein the ADC is administered by an IV injection or infusion on Days 1, 8 and 15 of every four-week cycle.

171. The method of any one of claims 1 to 170, wherein the ADC is administered by an IV injection or infusion over about 30 minutes three times every four-week cycle.

172. The method of any one of claims 1 to 171, wherein the ADC is administered by an IV injection or infusion over about 30 minutes on Days 1, 8 and 15 of every four-week cycle.

173. The method of any one of claims 1 to 172, wherein the ADC is formulated in a pharmaceutical composition comprising L-histidine, polysorbate-20 (TWEEN-20), and trehalose dehydrate.

174. The method of any one of claims 1 to 173, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

175. The method of any one of claims 1 to 173, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

176. The method of any one of claims 1 to 175, wherein the cancer is bladder cancer, urothelial cancer, gastric cancer, esophageal cancer, head cancer, neck cancer, squamous non-small cell lung cancer (NSCLC), non-squamous NSCLC, breast cancer, ovarian cancer, cervical cancer, biliary cancer and cholangiocarcinomas, pancreatic cancer, squamous cell carcinoma of the vulva and penis, prostate adenocarcinoma, or endometrial carcinoma.

177. The method of any one of claims 1 to 176, wherein the cancer is locally advanced cancer.

178. The method of any one of claims 1 to 176, wherein the cancer is metastatic cancer.

179. The method of any one of claims 176 to 178, wherein the breast cancer is ER negative, PR negative, and HER2 negative (ER−/PR−/HER2−) breast cancer.

180. The method of any one of claims 176 to 179, wherein the breast cancer is hormone receptor positive and human epidermal growth factor receptor 2 negative (HR+/HER2−) breast cancer.

181. The method of any one of claims 176 to 178, wherein the urothelial cancer is papillary urothelial carcinoma or flat urothelial carcinoma.

182. The method of any one of claims 176 to 178, wherein the bladder cancer is non-muscle-invasive bladder cancer (NMIBC) or muscle-invasive bladder cancer.

183. The method of claim 182, wherein the muscle-invasive bladder cancer is squamous cell carcinoma, adenocarcinoma, small cell carcinoma, or sarcoma.


SEQUENCE LISTING (23 sequences)

SEQ ID NO: 1 - DNA, 3464 bp, Homo sapiens
  CDS (264)...(1796)
  misc_feature (1)...(3464): 191P4D12
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  misc_feature (1)...(510): 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  CDS (32)...(1432)
  misc_feature (1)...(1432): Ha22-2(2,4)6.1 heavy chain
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  misc_feature (1)...(466): Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  CDS (25)...(735)
  misc_feature (1)...(735): Ha22-2(2,4)6.1 light chain
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  misc_feature (1)...(236): Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  misc_feature (1)...(466): Ha22-2(2,4)6.1 heavy chain

SEQ ID NO: 8 - protein, 236 aa, Homo sapiens; identical to SEQ ID NO: 6
  misc_feature (1)...(236): Ha22-2(2,4)6.1 light chain

SEQ ID NO: 9 - protein, 5 aa, Homo sapiens
  CDR1 of heavy chain
SYNMN

SEQ ID NO: 10 - protein, 17 aa, Homo sapiens
  CDR2 of heavy chain
YISSSSSTIYYADSVKG

SEQ ID NO: 11 - protein, 8 aa, Homo sapiens
  CDR3 of heavy chain
AYYYGMDV

SEQ ID NO: 12 - protein, 11 aa, Homo sapiens
  CDR1 of light chain
RASQGISGWLA

SEQ ID NO: 13 - protein, 7 aa, Homo sapiens
  CDR2 of light chain
AASTLQS

SEQ ID NO: 14 - protein, 9 aa, Homo sapiens
  CDR3 of light chain
QQANSFPPT

SEQ ID NO: 15 - protein, 4 aa, Artificial Sequence
  linker
GFLG

SEQ ID NO: 16 - protein, 8 aa, Artificial Sequence
  VH CDR1 according to IMGT
GFTFSSYN

SEQ ID NO: 17 - protein, 8 aa, Artificial Sequence
  VH CDR2 according to IMGT
ISSSSSTI

SEQ ID NO: 18 - protein, 10 aa, Artificial Sequence
  VH CDR3 according to IMGT
ARAYYYGMDV

SEQ ID NO: 19 - protein, 6 aa, Artificial Sequence
  VL CDR1 according to IMGT
QGISGW

SEQ ID NO: 20 - protein, 3 aa, Artificial Sequence
  VL CDR2 according to IMGT
AAS

SEQ ID NO: 21 - protein, 9 aa, Artificial Sequence
  VL CDR3 according to IMGT
QQANSFPPT

SEQ ID NO: 22 - protein, 117 aa, Artificial Sequence
  heavy chain variable region (VH), the 20th amino acid (glutamic acid) to the 136th amino acid (serine) of SEQ ID NO7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, Artificial Sequence
  light chain variable region (VL), the 23rd amino acid (aspartic acid) to the 130th amino acid (arginine) of SEQ ID NO8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR